(19) United States
(12) Patent Application Publication
(10) Pub. No.: US 2012/0231006 A1
(43) Pub. Date: Sep. 13, 2012

(54) ANTI-ORAI1 ANTIGEN BINDING PROTEINS AND USES THEREOF

(75) Inventors: Hung Q. Nguyen, Camarillo, CA (US); Fen-Fen Lin, Carlsbad, CA (US); Xiao-juan Bi, Newbury Park, CA (US); Helen J. McBride, Woodland Hills, CA (US); Shaw-Fen Sylvia Hu, Thousand Oaks, CA (US)

(73) Assignee: Amgen Inc., Thousands Oaks, CA (US)

(21) Appl. No.: 13/510,926

(22) PCT Filed: Nov. 19, 2010
(86) PCT No.: PCT/US10/57496

§ 371 Date: May 25, 2012

Related U.S. Application Data
(60) Provisional application No. 61/263,176, filed on Nov. 20, 2009.
(60) Provisional application No. 61/406,054, filed on Oct. 22, 2010.

(51) Int. Cl. A61K 39/395; C12N 15/13; C12N 15/63; C12N 5/10; C12P 21/02; C12N 5/16; A61P 37/00; A61P 3/10; A61P 19/02; A61P 1/00; A61P 29/00; A61P 37/08; A61P 1/16; A61P 35/00; A61P 31/04; A61P 9/10; C12N 1/21; C12N 1/19; C12N 1/15; C07K 16/18

(52) U.S. Cl. 424/139.1; 530/387.9; 530/387.3; 536/23.53; 435/320.1; 435/69.6; 435/331; 435/70.2; 435/252.33; 435/254.21; 435/254.11

(57) ABSTRACT

Disclosed is an isolated antigen binding proteins, such as but not limited to, an antibody or antibody fragment, that specifically bind to SEQ ID NO: 4, the amino acid sequence of extracellular loop 2 (ECL2) of human Orai1. Also disclosed are pharmaceutical compositions and medicaments comprising the antigen binding protein, isolated nucleic acid encoding it, vectors and host cells useful in methods of making it, and methods of using it in treating disorders or diseases in patients.

The instant application contains an ASCII “txt” compliant sequence listing, which serves as both the computer readable form (CRF) and the paper copy, and is hereby incorporated by reference in its entirety. The name of the “txt” file created on Nov. 18, 2010, is: A-1466-WO-PCT-111810rev ST25.txt, and is 514 kb in size.

Throughout this application various publications are referenced within parentheses or brackets. The disclosures of these publications in their entireties are hereby incorporated by reference in this application in order to more fully describe the state of the art to which this invention pertains.

### BACKGROUND OF THE INVENTION

1. Field of the Invention

This invention relates to anti-Orai1 antigen binding proteins for treating disorders and diseases and more particularly to anti-Orai1 antibodies and antibody fragments.

2. Discussion of the Related Art

The plasma membrane presents a barrier that separates the intracellular from the extracellular compartments preventing the movement of ions from one compartment to the other. Ion channels are a diverse group of membrane embedded proteins that form a tunnel to allow small inorganic ions to traverse across the membrane. They include sodium, potassium and calcium cation channels and chloride anion channels that are typically classified into two main groups, voltage-gated and ligand-gated ion channels. The latter group of channels consists of extracellular and intracellular ligand-gated channels.

Calcium channels, regulating the concentration of intracellular calcium, play an important role in many cellular processes ranging from short-term responses such as contraction and secretion to longer-term regulation of cell growth and proliferation. The regulation of intracellular calcium is an important feature of the transduction of signals into and within cells. Intracellular calcium serves as a secondary messenger important in the regulation of gene expression, cell differentiation, cytokine secretion and calcium homeostasis. (Parekh and Putney, Store-operated calcium channels, Physiology Review, 85:757-810 (2005)). Virtually all cell types depend in some manner upon the generation of cytoplasmic Ca²⁺ signals to regulate cell function, or to trigger specific responses to growth factors, neurotransmitters, hormones and a variety of other signal molecules.

Usually, these Ca²⁺-mediated signals involve some combination of release of Ca²⁺ from intracellular stores, such as the endoplasmic reticulum (ER), and influx of Ca²⁺ across the plasma membrane. The majority of intracellular calcium is in the endoplasmic reticular (ER) stores that are distributed throughout the cytoplasm from around the nucleus to the cell periphery.

In one example, cell activation begins with an agonist binding to a surface membrane receptor, coupled to the activation of phospholipase C(PLC) through a G-protein mechanism. Activated PLC, in turn hydrolyses a chemical messenger phosphatidylinositol-4,5-biphosphate into inositol-1,4,5-triphosphate (IP3) and diaglycerol. The second messenger IP3 then binds to IP3 receptor that resides on the ER membrane causing calcium dication to be released from the ER stores (Hogan et al., Transcriptional regulation by calcium, calcineurin and NFAT. Gene Dev. 17:2205-2232 (2003)). The fall in ER Ca²⁺ then signals to plasma membrane store-operated calcium (SOC) channels.

Store-operated calcium influx, or entry, is a process in cellular physiology that controls such diverse functions such as, but not limited to, refilling of intracellular Ca²⁺ stores (Putney, A model for receptor-regulated calcium entry, Cell Calcium, 7:1-12 (1986); Putney et al. Cell, 75, 199-201 (1993)), activation of enzymatic activity (Fagan et al., J. Biol. Chem. 275:26530-26537 (2000)), gene transcription (Lewis, Annu Rev. Immunol. 19:497-521 (2001)), cell proliferation (Nunez et al., J. Physiol. 571.1, 57-73 (2006)), and release of cytokines (Winslow et al., Curr. Opin. Immunol. 15:299-307 (2003)). In some “nonexcitable cells”, e.g., blood cells, hematopoietic cells, and on most cells of the immune system, including monocytes and macrophages, mast cells, natural killer cells, dendritic cells and T lymphocytes, SOC influx occurs through calcium release-activated calcium (CRAC) channels, a type of SOC channel. The CRAC current (“I_{CRAC}” or “ICRAC”) displays an activation kinetics that is delayed by tens of seconds and inwardly rectifying characteristics that decay over a period of minutes. In addition, the channel has high specificity for calcium. (Hoth and Penner, Calcium release-activated calcium current in rat mast cells, J. Physiol., 465:359-386 (1993); Hoth and Penner, Depletion of intracellular calcium stores activates a calcium current in mast cells, Nature 355:353-355 (1992)).

CRAC-mediated calcium regulation in T lymphocytes can be categorized according to (i) short-termed and (ii) long-termed effects. Short-termed effects are cell motility and the formation of an immunological synapse, i.e., an interface where an antigen presenting cell presents antigen to CD4 positive T lymphocyte. The inhibition of the intracellular rise in calcium level has been shown to effectively neutralize the stable formation of an immunological synapse. The long-termed effects are tied to the transcriptional regulation of cytokine expression that influences lymphocyte effector functions, states of unresponsiveness, the differentiation of naïve T cells into T helper 1 or 2, T cells and the development of immature T cells (Feske, Calcium signaling in lymphocyte activation and disease, Nature Rev. Immunol. 7:690-702 (2007)). Impaired calcium signaling in T (and B cells) has been linked to a number of inherited immunodeficiency diseases and has tremendously contributed to our understanding of the role of calcium regulation in the immune response. Autoreactive T cells play an important role in the development of several autoimmune diseases including rheumatoid arthritis, inflammatory bowel disease (IBD), multiple sclerosis, and type-1 diabetes; autoreactive B cells are involved in systemic lupus erythematosus (SLE).

Activation of the SOC entry (SOCE) pathway via CRAC involves stromal interaction molecule 1 (STIM1), localized to the endoplasmic reticulum (ER), and calcium channel subunit (Orai1, also known as calcium release-activated calcium modulator 1 (CRACM1) or Transmembrane Protein 142A (TMEM142A)), localized to the plasma membrane. With the advent of high-throughput RNA interference screening technology to knock down the expression of proteins by eliminating the messenger RNA that encodes them, STIM1 was discovered to play a role in CRAC channel activation in Drosophila S2 insect cells (Roos et al., STIM1 an essential and conserved component of store-operated Ca channel function, J. Cell Biol. 169:435-445 (2005)). Through a similar study, it was discovered that inhibiting the expression of STIM1 or STIM2 in HeLa cells suppressed CRAC activity. (Liou et al., STIM1 is a Ca²⁺ sensor essential for Ca²⁺ store depletion triggered Ca²⁺ influx, Current Biol. 15:1235-1241 (2005)). STIM1 encodes a single pass transmembrane protein that resides mainly in the ER with the C-terminus in the cytoplasm and the N-terminus in the ER lumen. The N-terminal region specifically the helix-loop-helix (EF-hand) containing glutamate and aspartate amino acids and sterile alpha motif (SAM) domains are responsible for binding calcium. (Stathopulos et al., Stored Ca depletion-induced oligomerization of STIM via EF-SAM region: An initiation mechanism for capacitative Ca entry, J. Biol. Chem. 281:35855-35862 (2006)). When the calcium ER store is replete, calcium-bound STIM1 is distributed throughout the ER, but when the calcium store is depleted then the unbound STIM1 forms oligomers that are distributed in subregions of the ER located in proximity to the plasma membrane to form discrete puncta structures. (Zhang et al., STIM1 is a Ca sensor that activates CRAC channels and migrates from the Ca store to the plasma membrane, Nature 437:902-905 (2005)). The carboxy-terminal region of STIM1 is responsible for the activation of the CRAC channel since expression of peptide fragments corresponding to a domain in this region was shown to bind to and open the CRAC channel without calcium store depletion. (Yuan et al., SOAR and the polybasic STIM1 domain gate and regulate Orai channels, Nature Cell Biology 11:337-343 (2009); Park et al., STIM1 clusters and activates CRAC channels via direct binding of a cytosolic domain to Orai1, Cell 136:876-890 (2009)). It is also possible that other diffusible factors, such as calcium inducible factor (CIF), may be involved in STIM1 activating the CRAC channel. (Csutora et al., Novel role for STIM1 as a trigger for calcium influx factor production, J. Biol. Chem. 283:14524-31 (2008)).

Calloway et al. described molecular clustering of STIM1 in the ER with Orai1/CRACM1 at the plasma membrane, dependent dynamically on depletion of Ca²⁺ stores and on electrostatic interactions. ((Calloway et al., Molecular clustering of STIM1 with Orai1/CRACM1 at the plasma membrane depends dynamically on depletion of Ca²⁺ stores and on electrostatic interactions, Mol Biol Cell. 20(1):389-99. (2009 January; Epub 2008 Nov. 5)).

A form of hereditary severe combined immune deficiency (SCID) in human patients has been linked to abrogation of CRAC channel function that resulted from a missense mutation in Orai1. (Feske et al., A severe defect in CRAC Ca²⁺ channel activation and altered of K⁺ channel gating in T cells from immunodeficient patients, J. Exptl. Med. 202(5):651-62 (2005); Feske et al., A mutation in Orai1 causes immune deficiency by abrogating CRAC channel function, Nature 441:179-85 (2006)). Two other groups independently identified the same gene using high-throughput RNA interference with Drosophila S2 cells for genes that play a role in CRAC activity. (Vig et al., CRACM1 is a plasma membrane protein essential for store-operated Ca entry, Science 312:1220-1223 (2006); Zhang et al., Genome-wide RNAi screen of Ca influx identifies genes that regulate Ca release-activated Ca channel activity, PNAS103:9357-9362 (2006)). Although, there is only one Orai1 gene in Drosophila, there are two other homologues in mammals called Orai2 and Orai3. The Orai1 gene encodes for a four transmembrane protein residing on the plasma membrane with the amino-terminus and carboxy-terminus located in the cytoplasm and two short extracellular loops. Mutagenesis studies using electrophysiology concluded that Orai1 is the bona fide CRAC channel by demonstrating that certain mutants negatively affected the selectivity of the channel to calcium. (Yeromin et al., Molecular identification of the CRAC channel b altered ion selectivity in a mutant of Orai1, Nature 433:226-229 (2006); Prakirya et al., Orai1 is an essential pore subunit of the CRAC channel, Nature 443:230-233 (2006); Vig et al., CRACM1 multimers form the ion-selective pore of the CRAC channel, Curr. Biol. 16:2073-2079 (2006)). The CRAC channel is generally thought to be composed of a homotetramer of Orai1 protein, however the possibility still exists that in some cases heterotetramers may form containing Orai1 together with Orai2 and/or Orai3 proteins.

Rao et al. cloned the human Orai1 sequence. (Rao et al., Regulators of NFAT, WO 2007/081804 A2). A set of conserved acidic amino acids in trans membrane domains I and III and in the I-II loop of Orai1 (E106, E190, D110, D112, D114) were identified that are reportedly essential for the CRAC channel's high Ca²⁺ selectivity; Yamashita et al. found that alteration of those acidic residues can lower Ca²⁺ selectivity and resulted in increased Cs⁺ permeation. (Yamashita et al., Orai1 mutations alter ion permeation and Ca2+-dependent fast inactivation of CRAC channels: evidence for coupling of permeation and gating, J Gen Physiol 130 (5): 525. (2007)). Further structure-function analysis of the Orai1 protein revealed the presence of intrinsic gating of the CRAC channel; a mutation of Orai1 (V102I) close to the selectivity filter modified CRAC channel sensitivity to membrane depolarization and resulted in slow gating of the CRAC channel at negative potentials. (Spassova et al., Voltage gating at the selectivity filter of the Ca2+ release-activated Ca2+ channel induced by mutation of the Orai1 protein, J. Biol. Chem. 283(22):14938-45 (2008)).

The cascading signaling events that result in a sustained calcium influx via CRAC channels leads to the activation of several transcription factors and the best characterized is nuclear factor of activated T cells (NFAT). Calmodulin is one of many calcium binding proteins that can sense the level of calcium in the cytoplasm and transmit the calcium signal and to orchestrate the cellular response. Calmodulin when bound to calcium activates calcineurin, a serine and threonine phosphatase that then dephosphorylates NFAT. Phosphorylated NFAT exposes nuclear export sequences and binds to exportin protein resulting in cytoplasmic localization. The dephosphorylated NFAT exposes nuclear localization sequences resulting in binding to importins and translocation to the nucleus. (Okamura et al., Concerted dephosphorylation of the transcription factor NFAT induces a conformational switch that regulates transcriptional activity, Mol. Cell. 6:539-550 (2000)). In the nucleus, NFAT activates the transcription of variety of genes encoding for cytokines such as interleukin-2 (IL2) and interferon gamma (IFNγ) that are crucial for T cell activation. (Feske et al., Ca/calcineurin signaling in cells of the immune system, Biochem. Biophys. Res. Comm. 311:1117-1132 (2003)). Cyclosporin A (Neoral®, Sandlmmune) and FK506 (Tacrolimus; PROGRAF®) are small molecules designed to inhibit calcineurin and are used for the treatment of severe immune disorders including rejection following solid organ transplant. Neoral® has been approved for treating severe rheumatoid arthritis and psoriasis. Other inflammatory diseases that have been suggested for calcineurin inhibitors from preclinical data include inflammatory bowel disease and multiple sclerosis. Lupus may be another indication that may benefit from intervening in the calcineurin pathway. Although calcineurin plays a critical role in the regulation of NFAT activity in T cells, it is expressed in all tissues in the body, including kidney. This expression profile renders cyclosporine and FK506 a narrow safety margin due to on-target-based toxicity, such as hypertension and renal toxicity. Despite cyclosporine and FK506 being efficacious in blocking the calcineurin pathway, these drugs are mainly reserved for treating severe immune diseases due to their toxicity.

Consequently, other therapeutic drugs have been sought for treating human disorders and diseases, e.g., immune disorders or disorders related to venous or arterial thrombus formation, that target CRAC, and Orai1, in particular. (E.g., Normant et al., Methods and Compositions for Screening ICRAC Modulators, U.S. Pat. No. 6,696,267; Cahalan et al., CRAC channel and modulator screening methods, US 2008/039392 A1; Stauderman et al., Calcium Channel Proteins and Uses thereof, WO 2008/148108 A1 and US 2008/0293092 A1; Velcelebi et al., Compounds that Modulate Intracellular Calcium, WO 2009/035818 A1; Roos et al., Methods of Modulating and Identifying Agents that Modulate Intracellular Calcium, WO 2004/078995 A2; Fleig et al., CRAC Modulators and Use of Same for Drug Discovery, WO 2007/121186 A2; Xie et al., Compounds for Inflammation and Immune-related Uses, US 2005/0107436 A1; Xie et al., Method for Modulating Calcium-Ion-Release-Activated Calcium Ion Channels, US 2005/0148633 A1; Vo et al., Fused Ring Compounds for Inflammation and Immune-related Uses, WO 2008/039520 A2 and US 2008/0132513 A1; Bohnert et al., Cyclohexenyl-Aryl Compounds for Inflammation and Immune-related Uses, WO 2008/063504 A2 and US 2008/0207641 A1; Chen, Pyridine Compounds for Inflammation and Immune-related Uses, WO 2009/017819 A1; Chen, Heterocycle-Aryl Compounds for Inflammation and Immune-related Uses, WO 2009/017818 A1; Braun et al., The calcium sensor STIM1 and the platelet SOC channel Orai1 (CRACM1) are essential for pathological thrombus formation, WO 2009/115609 A1; Braun et al., The calcium sensor STIM1 is essential for pathological thrombus formation, EP 2103311 A1).

The present invention provides potent and selective blocking antibodies directed to Orai1.

### SUMMARY OF THE INVENTION

The invention relates to isolated antigen binding proteins, including antibodies or antibody fragments, that specifically bind to SEQ ID NO:4 (i.e., amino acid residues 198-233 of SEQ ID NO:2), which is the amino acid sequence of the putative extracellular loop (ECL) 2 of native human Orai1. In particular embodiments, the antigen binding proteins specifically bind to a subset of amino acid residues 204-223 of SEQ ID NO:2; or to a subset of amino acid residues 204-217 of SEQ ID NO:2; or to a subset of amino acid residues 207 to 213; or to a subset of amino acid residues 213 to 217 of SEQ ID NO:2.

In some embodiments, the inventive antigen binding protein, including an antibody or antibody fragment, specifically binds to a human Orai1 polypeptide, wherein:

(a) the antigen binding protein specifically binds to a polypeptide having an amino acid sequence consisting of:

(i) SEQ ID NO:210 [hOrai1-mOrai1 ECL2 (KQPGQPRPTSKPPASGAA), as described in Example 8]; or

(ii) SEQ ID NO:204 [hOrai1-mOrai1 ECL2 (KQPGQPRPTSKPPA), as described in Example 8]; or

(iii) SEQ ID NO:192 [hOrai1-mOrai1 ECL2 (RPTSKPPASGAA), as described in Example 8]; or

(iv) SEQ ID NO:129 [hOrai1-mOrai1 ECL2 (RPTSKPPA), as described in Example 8]; or

(v) SEQ ID NO:103 [hOrai1-mOrai1 ECL2 (SKPPA), as described in Example 8]; and

(b) the antigen binding protein does not specifically bind to a polypeptide having an amino acid sequence consisting of (vi) SEQ ID NO:91 [hOrai1-mOrai1 ECL2, as described in Example 8]; or

(vii) SEQ ID NO:198 [hOrai1-mOrai1 ECL2 (PASGAAANVST), as described in Example 8]; or

(viii) (SEQ ID NO:113) [hOrai1-mOrai1 ECL2 (PASGAA), as described in Example 8]; or

(ix) SEQ ID NO:123 [hOrai1-mOrai1 ECL2 (AANVST), as described in Example 8]; or

(x) SEQ ID NO:107 [hOrai1-mOrai1 ECL2 (GAA), as described in Example 8]; or

(xi) SEQ ID NO:117 [hOrai1-mOrai1 ECL2 (VST), as described in Example 8].

In some embodiments, the inventive antigen binding protein inhibits human calcium response-activated calcium (CRAC) channel activity. In other embodiments, the inventive antigen binding protein inhibits NFAT-mediated expression and/or inhibits release of IL-2, IFN-gamma, or both, in thapsigargin-treated human whole blood.

The invention also provides materials and methods for producing such inventive antigen binding proteins, including isolated nucleic acids that encode them, vectors and isolated host cells and hybridomas. Also provided are isolated nucleic acids encoding any of the immunoglobulin heavy and/or light chain sequences and/or VH and/or VL sequences and/or CDR sequences disclosed herein. In a related embodiment, an expression vector comprising any of the aforementioned nucleic acids is provided. In still another embodiment, a host cell is provided comprising any of the aforementioned nucleic acids or expression vectors.

The inventive isolated antigen binding protein, including antibody and antibody fragment embodiments, can be used in the manufacture of a pharmaceutical composition or medicament. The inventive pharmaceutical composition or medicament comprises the antigen binding protein and a pharmaceutically acceptable diluent, carrier or excipient. Exemplary embodiments of the invention include a pharmaceutical composition or medicament useful to treat an immune disorder or disease in a human. Other exemplary embodiments of the invention include a pharmaceutical composition or medicament to useful to treat a disorder related to venous or arterial thrombus formation.

The invention further provides methods of using any of the inventive antigen binding proteins, or medicaments containing them, to treat or prevent an immune disorder or disease in a patient, comprising administering an effective amount of the antigen binding protein to the patient wherein the immune disorder is selected from T cell-mediated autoimmunity, transplant rejection (e.g., allograft rejection), graft versus host disease (GVHD), rheumatoid arthritis, multiple sclerosis, type-1 diabetes, systemic lupus erythematosus, psoriasis, inflammatory bowel disease (IBD), asthma, allergic rhinitis, eosinophilic disease, autoimmune central nervous system (CNS) inflammation, inflammation-induced liver injury. (See, e.g., Ma et al., T-cell-specific deletion of STIM1 and STIM2 protects mice from EAE by impairing the effector functions of Th1 and Th17 cells, Eur J Immunol. 2010 November; 40(11):3028-42. doi: 10.1002/eji.201040614. Epub 2010 Oct. 27; Zitt et al., Potent inhibition of Ca2+ release-activated Ca2+ channels and T-lymphocyte activation by the pyrazole derivative BTP2, J Biol Chem. 279(13):12427-37 (2004); Yoshino et al., YM-58483, a selective CRAC channel inhibitor, prevents antigen-induced airway eosinophilia and late phase asthmatic responses via Th2 cytokine inhibition in animal models, Eur J. Pharmacol. 560(2-3):225-33. (2007); Ohga et al., Characterization of YM-58483/BTP2, a novel store-operated Ca2+ entry blocker, on T cell-mediated immune responses in vivo, Int. Immunopharmacol. 8(13-14):1787-92 (2008); Schuhmann et al., Stromal interaction molecules 1 and 2 are key regulators of autoreactive T cell activation in murine autoimmune central nervous system inflammation, J. Immunol. 2010 Feb. 1; 184(3):1536-42. Epub 2009 Dec. 18; Vig et al., Defective mast cell effector functions in mice lacking the CRACM1 pore subunit of store-operated calcium release-activated calcium channels, Nat. Immunol. 9(1):89-96 (2008); Di Sabatino et al., Targeting gut T cell Ca2+ release-activated Ca2+ channels inhibits T cell cytokine production and T-box transcription factor T-bet in inflammatory bowel disease, J. Immunol. 2009 Sep. 1; 183(5):3454-62. Epub 2009 Jul. 31; Djuric et al., 3,5-Bis(trifluoromethyl)pyrazoles: a novel class of NFAT transcription factor regulator, J. Med. Chem. 43(16):2975-81 (2000); Lin et al., Up-regulation of Orai1 in murine allergic rhinitis, Histochem Cell Biol, 2010 Jul, 134(1):93-102. Epub 2010 Jun. 16; Lin et al., 2-Aminoethoxydiphenyl borate administration into the nostril alleviates murine allergic rhinitis, Am J Otolaryngol. 2010 Sep. 9. [Epub ahead of print]; McCarl et al., Store-operated Ca2+ entry through ORAI1 is critical for T cell-mediated autoimmunity and allograft rejection, J. Immunol. 2010 Nov. 15; 185(10):5845-58, Epub 2010 Oct. 18; Yonetoku et al., Novel potent and selective calcium-release-activated calcium (CRAC) channel inhibitors. Part 2: Synthesis and inhibitory activity of aryl-3-trifluoromethylpyrazoles, Bioorg Med Chem. 14(15):5370-83 (2006); Yonetoku et al., Novel potent and selective Ca2+ release-activated Ca2+ (CRAC) channel inhibitors. Part 3: synthesis and CRAC channel inhibitory activity of 4′-[(trifluoromethyl)pyrazol-1-yl]carboxanilides, Bioorg Med Chem. 16(21):9457-66 (2008)).

The invention also provides methods of using any of the inventive antigen binding proteins, or medicaments containing them, to treat or prevent a disorder related to venous or arterial thrombus formation in a patient, comprising administering an effective amount of the antigen binding protein to the patient, wherein the disorder is selected from arterial thrombosis, myocardial infarction, stroke, ischemic reperfusion injury, ischemic brain infarction, inflammation, complement activation, fibrinolysis, angiogenesis related to FXII-induced kinin formation, hereditary angioedema, bacterial infection of the lung, trypanosome infection, hypotensitive shock, pancreatitis, chagas disease, thrombocytopenia and articular gout. (See, e.g., Varga-Szabo et al., The calcium sensor STIM1 is an essential mediator of arterial thrombosis and ischemic brain infarction, J Exp Med. 205(7):1583-91 (2008); Braun et al., Orai1 (CRACM1) is the platelet SOC channel and essential for pathological thrombus formation, Blood 113(9):2056-63 (2009); Varga-Szabo et al., Calcium signaling in platelets, J Thromb Haemost. 7(7):1057-66 (2009); Bergmeier et al., R93W mutation in Orai1 causes impaired calcium influx in platelets, Blood 113(3):675-78 (2009)).

The invention also provides methods of using any of the inventive antigen binding proteins, or medicaments containing them, to treat breast cancer or prevent tumorogenesis, tumor cell migration and/or metastasis, particularly of estrogen receptor-negative (ER—) breast cancer cells. (See, e.g., Yang et al., Orai1 and STIM1 are critical for breast tumor cell migration and metastasis, Cancer Cell 15(2):124-34 (2009); Motiani et al., A novel native store-operated calcium channel encoded by Orai3: selective requirement of Orai3 versus Orai1 in estrogen receptor-positive versus estrogen receptor-negative breast cancer cells, J Biol. Chem. 2010 Jun. 18; 285(25):19173-83. Epub 2010 Apr. 15; Feng et al., Store-independent activation of Orai1 by SPCA2 in mammary tumors, Cell. 2010 Oct. 1; 143(1):84-98).

Numerous methods are contemplated in the present invention. For example, a method is provided involving culturing the aforementioned host cell comprising the expression vector of the invention such that the encoded antigen binding protein is expressed. A method is also provided involving culturing the aforementioned hybridoma in a culture medium under conditions permitting expression of the antigen binding protein by the hybridoma. Such methods can also comprise the step of recovering the antigen binding protein from the host cell culture. In a related embodiment, an isolated antigen binding protein produced by the aforementioned method is provided.

The foregoing summary is not intended to define every aspect of the invention, and additional aspects are described in other sections, such as the Detailed Description of Embodiments. The entire document is intended to be related as a unified disclosure, and it should be understood that all combinations of features described herein are contemplated, even if the combination of features are not found together in the same sentence, or paragraph, or section of this document.

In addition to the foregoing, the invention includes, as an additional aspect, all embodiments of the invention narrower in scope in any way than the variations defined by specific paragraphs above. For example, certain aspects of the invention that are described as a genus, and it should be understood that every member of a genus is, individually, an aspect of the invention. Also, aspects described as a genus or selecting a member of a genus, should be understood to embrace combinations of two or more members of the genus. Although the applicant(s) invented the full scope of the invention described herein, the applicants do not intend to claim subject matter described in the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWINGS

FIG. 1 shows functional binding by hybridoma supernatants to human Orai1, as assessed by inhibition of cytokine release (IL-2, circles; IFN-gamma, squares) from thapsigargin-treated human whole blood. Positive Orai1-binding supernatants along with a negative control hybridoma supernatant were used at 25% (volume/volume) to assess inhibition of cytokine secretion expressed as a percent of control.

FIG. 2A-D demonstrates dose-dependent inhibition by purified monoclonal antibodies of cytokine release (IL-2, FIGS. 2A-B; and IFN-gamma, FIGS. 2C-D) from thapsigargin-treated human whole blood collected from two separate human donors (Donor A: FIG. 2A and FIG. 2C; Donor B: FIG. 2B and FIG. 2D).

FIG. 3A-B shows purified recombinant anti-human Orai1 monoclonal antibodies electrophoresed on a 1.0 mm 4-20% Tris Glycine SDS PAGE gel (Invitrogen) under non-reducing (FIG. 3A) and reducing conditions (FIG. 3B). From left (2 μg protein/well): Lane 1, Mark 12 MW markers; Lane 2, mAb 2D2.1; Lane 3, mAb 2C1.1; Lane 4, blank; Lane 5, mAb 2B7.1; Lane 6, mAb 2A2.2-1; Lane 7, mAb 2A2.2-2; Lane 8, mAb 2B4.1.

FIG. 4 shows FACS analysis demonstrating binding of recombinant monoclonal antibodies to human Orai1 expressed on the surface of AM1-CHO cells. AM1-CHO parental and AM1-CHO/Orai1/STIM1-YFP were stained first with recombinant monoclonal antibodies then counter-stained with a secondary phycoerythrin labeled goat anti-human IgG F(ab′)₂ antibody fragment, and were visualized using FACS.

FIG. 5A-D shows that recombinant monoclonal antibodies, mAb2D2.1, mAb2C1.1 and mAb2B7.1 (but not mAb2B4.1, mAb84.5 and mAb133.4), inhibited interleuklin-2 and interferon-gamma secretion (IL-2, FIGS. 5A-B; and IFN-gamma, FIGS. 5C-D) in a dose-dependent manner from thapsigargin-treated human whole blood from two separate human donors (Donor A: FIG. 5A and FIG. 5C; Donor B: FIG. 5B and FIG. 5D).

FIG. 5E-H shows that purified monoclonal antibodies mAb 5A1.1, mAb 5A4.2, mAb 5B1.1, mAb 5B5.2, mAb 5C1.1, mAb 5F2.1, and mAb 5F7.1 inhibited interleuklin-2 and interferon-gamma secretion (IL-2, FIGS. 5E-F; and IFN-gamma, FIGS. 5G-H) in a dose-dependent manner from thapsigargin-treated human whole blood from two separate human donors (Donor A: FIG. 5E and FIG. 5G; Donor B: FIG. 5F and FIG. 5H).

FIG. 6A shows a plot of calcium entry into HEK-293 cells as represented by the ratio of 395 nm/485 nm emitted light on the y-axis over time (seconds) on the x-axis. The first minute of recording represents the baseline before any treatment with the low ratio representing low calcium level inside the cells. Thapsigargin was added after one minute to induce the internal stored calcium to be released. At about 6 minutes, when the internal calcium level had returned to baseline, external calcium dication was added to 2 mM final concentration resulting in an immediate and sharper rise in the 395 nm/485 nm ratio representing an even higher level of calcium inside the cells caused by the calcium influx via the Orai1 (CRAC) channel.

FIG. 6B-C show representative data illustrating that inventive anti-Orai1 mAbs dose-dependently inhibited luciferase activity in HEK-293 cells expressing human Orai1 and human STIM1 along with an NFAT driven luciferase reporter gene. While mAb 2C1.1 (circle), mAb 2D2.1 (square) and mAb 2B7.1 (diamond) display a dose-dependent blocking of luciferase activity, the Negative Control mAb (triangle) showed a slight dose-dependent increase in activity. In FIG. 6C, the mAb 2B4.1 (triangle) shows a slight dose-dependent inhibition and a much weaker IC50 relative to mAb 2C1.1, mAb 2D2.1, or mAb 2B7.1.

FIG. 7A-C shows that CRAC currents were inhibited by an anti-hOrai1 antibody of the present invention, mAb 2B7.1, but not by anti-dinitrophenol (DNP) mAb (Neg. Control mAb). Cells were held at a holding potential of 0 mV. The membrane potential was stepped to −100 mV for 25 ms and a 100 ms voltage ramp going from −100 to 100 mV was applied to obtain I-V relationships (FIG. 7A). Representative I-V relationships for fully activated ICRAC show that pretreatment with a Negative Control monoclonal antibody (1 μM) had little or no effect on ICRAC as compared to control curves (FIG. 7B). Representative I-V relationships for fully activated ICRAC shows that mAb 2B7.1 (1 μM) inhibited ICRAC compared to control curves (FIG. 7C).

FIGS. 8A-F demonstrate that anti-hOrai1 antibodies (1 μM) of the present invention inhibited ICRAC. The initial leak currents were subtracted from the maximal currents. Average current amplitudes measured at −100 mV were significantly different for cells treated with anti-hOrai1 antibodies mAb 2B7.1 (FIG. 8B), mAb 2D2.1 (FIG. 8C), mAb 2C1.1 (FIG. 8D), mAb 2B4.1 (FIG. 8F), mouse anti-hOrai1 antibodies mAb 133.4 and mAb 84.5 (FIG. 8E), compared to the buffer solution control (10 mM Sodium Acetate, pH5.0 plus 9% sucrose buffer; “A5SU”), which did not significantly alter ICRAC (FIG. 8E), or compared to control cells or cells treated with a negative control mAb (FIG. 8A), which had little or no effect on ICRAC. Data are shown as mean±S.E.M.

FIG. 9 shows an alignment of the amino acid sequences of human Orai1 (SEQ ID NO:2), human Orai2 (SEQ ID NO:61), and human Orai3 (SEQ ID NO:63) proteins. Amino acid residues in putative extracellular loops ECL1 (double underlined) and ECL2 (single underlined) are represented, as predicted using the TMpred program from ch.EMBnet (www.ch.embnet.org/index.html).

FIG. 10A-B shows an alignment of the amino acid sequences of Orai1 proteins from chimpanzee (SEQ ID NO:80), human (SEQ ID NO:2), cynomolgus monkey (N-terminally truncated partial sequence; SEQ ID NO:82), dog (SEQ ID NO:84), mouse (SEQ ID NO:72), and rat (SEQ ID NO:76). Amino acid residues in predicted extracellular loops ECL1 (double underlined) and ECL2 (single underlined) are represented, as predicted using the TMpred program from ch.EMBnet (www.ch.embnet.org/index.html). There is a 100% conservation of amino acid sequence in ECL1 between the different Orai1 proteins from dog and non-human primates compared to human, but only 87.5% conservation between rodents and human. The TMpred program also predicted the ECL2 region (single underlined amino acid residues). Unlike ECL1, the ECL2 varies in length and the conservation is mainly at the ends.

FIG. 11A-B shows FACS binding data demonstrating specific binding to human Orai1 by mAbs of the present invention. Purified monoclonal antibodies from hybridoma supernantants of the subclones derived from initial hits were assessed for binding to human Orai1, Orai2 and Orai3 expressed on HEK-293-EBNA cells along with vector transfected control parental cells. Cells stained with or without primary mAbs were counter-stained with a secondary phycoerythrin-labeled goat (“Gt”) anti-human (“Hu”) IgG F(ab′)₂ antibody fragment and visualized using FACS. For murines mAbs 84.5 and 133.4, a secondary phycoerythrin-labeled Gt anti-mouse (“Mu”) IgG F(ab′)₂ antibody fragment was used instead.

FIG. 12A-B illustrates the results of FACS analysis showing similar binding to human Orai1 wild-type versus single-polynucleotide variant expressed on the surface of HEK-293-EBNA cells. HEK-293-EBNA cells were transiently transfected with a construct expressing human Orai1 or a human Orai1 variant where the amino acid residue serine at position 218 of SEQ ID NO:2 is replaced with a glycine (S218G). Transfected cells were stained first with recombinant monoclonal antibodies then counter-stained with a secondary phycoerythrin-labeled Gt anti-Hu IgG F(ab′)₂ or Gt anti-Mu IgG F(ab′)₂ antibody fragment, as appropriate, and were visualized using FACS.

FIG. 13A-B illustrates the results of FACS analysis showing binding to human Orai1 expressed on the surface of HEK-293-EBNA cells that were treated with 2 μM Thapsigargin. HEK-293-EBNA cells were transiently transfected with human Orai1 and human STIM1, mouse Orai1 and mouse STIM1, rat Orai1 and rat STIM1 and control empty vector. Transfected cells were stained first with recombinant monoclonal antibodies then counter-stained with a secondary phycoerythrin-labeled Gt anti-Hu IgG F(ab′)₂ or Gt anti-Mu IgG F(ab′)₂ antibody fragment, as appropriate, and visualized using FACS.

FIG. 14 shows an alignment of the amino acid sequences of Orai1 proteins from human (SEQ ID NO:2) and mouse (SEQ ID NO:72). Amino acid residues in predicted extracellular loops ECL1 (double underlined) and ECL2 (single underlined) are represented. The double underlined amino acid residues represent the ECL1 domain that is predicted using the TMpred program from ch.EMBnet (www.ch.embnet.org/index.html). The program makes a prediction of membrane-spanning regions based on the statistical analysis of a database of naturally occurring transmembrane proteins, TMbase, using a combination of several weight-matrices for scoring.

FIG. 15A-B illustrates the results of FACS analysis showing binding to human Orai1 extracellular loop 2-mouse Orai1 mutant expressed on the surface of HEK-293-EBNA cells. HEK-293-EBNA cells were transiently transfected with mouse Orai1 mutant where human Orai1 extracellular loop 2 replaced the mouse Orai1 extracellular loop 2 and human Orai1 mutant where mouse Orai1 extracellular loop 2 replaced human extracellular loop 2. Transfected cells were stained first with recombinant monoclonal antibodies then counter-stained with a secondary phycoerythrin-labeled goat (“Gt”) anti-human (“Hu”) IgG F(ab′)₂ antibody fragment and visualized using FACS.

FIG. 16A-D illustrates the results of FACS analysis showing binding to the indicated mOrai1-hOrai1 ECL2 chimeric mutants expressed on the surface of HEK-293-EBNA cells. Transfected cells were stained first with recombinant monoclonal antibodies then counter-stained with a secondary phycoerythrin-labeled goat (“Gt”) anti-human (“Hu”) IgG F(ab′)₂ antibody fragment and visualized using FACS. In FIG. 16B and FIG. 16D, The values of Relative Fluorescence Intensity as Percent of Control (RFI-POC) are calculated from the relative fluorescence intensity geometric mean (Geo Mean). The geometric mean is an average calculated by multiplying a series of numbers and taking the nth root where n is the number of numbers in the series. It is a statistical average of a set of transformed numbers often used to represent a central tendency in a highly variable data set that minimizes the effects of extreme values. The RFI-POC was calculated using Algorithm II described in Example 8, concerning Table 11A-B. The chimera tested were (i) mOrai1-hOrai1 ECL2 (RQAGQPSPTKPPAE) (SEQ ID NO:226); (ii) mOrai1-hOrai1 ECL2 (SPTKPPAE) (SEQ ID NO:214); (iii) mOrai1-hOrai1 ECL2 (KPPAE) (SEQ ID NO:133); (iv) mOrai1-hOrai1 ECL2 (SPTKPPAESVIV) (SEQ ID NO:218); (v) mOrai1-hOrai1 ECL2 (SPTKPPAESVIVANHSD) (SEQ ID NO:232); (vi) mOrai1-hOrai1 ECL2 (AESVIVANHSD) (SEQ ID NO:222); (vii) mOrai1-hOrai1 ECL2 (AESVIV) (SEQ ID NO:141); (viii) mOrai1-hOrai1 ECL2 (VIV) (SEQ ID NO:137); (ix) mOrai1-hOrai1 ECL2 (HSD) (SEQ ID NO:145); (x) hOrai1-mOrai1 ECL2 (SEQ ID NO:91); and (xi) mOrai-hOrai1 ECL2 (SEQ ID NO:97).

FIG. 17A-D illustrates the results of FACS analysis showing binding to the indicated mOrai1-hOrai1 ECL2 chimeric mutants expressed on the surface of HEK-293-EBNA cells. Transfected cells were stained first with recombinant monoclonal antibodies then counter-stained with a secondary phycoerythrin-labeled goat (“Gt”) anti-human (“Hu”) IgG F(ab′)₂ antibody fragment and visualized using FACS. In FIG. 17B and FIG. 17D, the Relative Fluorescence Intensity Percentage of Control (RFI-POC) was calculated from the relative fluorescence intensity geometric mean (Geo Mean) using the Algorithm I described in Example 8, concerning Table 10A-B. The chimera tested were (i) hOrai1-mOrai1 ECL2 (KQPGQPRPTSKPPASGAA) (SEQ ID NO:210); (ii) hOrai1-mOrai1 ECL2 (KQPGQPRPTSKPPA) (SEQ ID NO:204); (iii) hOrai1-mOrai1 ECL2 (RPTSKPPASGAA) (SEQ ID NO: 192); (iv) hOrai1-mOrai1 ECL2 (RPTSKPPA) (SEQ ID NO:129); (v) hOrai1-mOrai1 ECL2 (SKPPA) (SEQ ID NO:103); (vi) hOrai1-mOrai1 ECL2 (SEQ ID NO:91); (vii) hOrai1-mOrai1 ECL2 (PASGAAANVST) (SEQ ID NO:198); (viii) hOrai1-mOrai1 ECL2 (PASGAA) (SEQ ID NO:113); (ix) hOrai1-mOrai1 ECL2 (AANVST) (SEQ ID NO:123); (x) hOrai1-mOrai1 ECL2 (GAA) (SEQ ID NO:107); (xi) hOrai1-mOrai1 ECL2 (VST) (SEQ ID NO:117); and (xii) mOrai-hOrai1 ECL2 (SEQ ID NO:97).

FIG. 18 illustrates the results of FACS analysis showing binding to human Orai1 expressed on the surface of AM1-CHO cells. AM1-CHO parental and AM1-CHO/Orai1/STIM1-YFP were stained first with recombinant monoclonal antibodies, then were counter-stained with a secondary FITC-labeled antibody fragment and were visualized using FACS. FIG. 18 shows the binding assessment by FACS of all the commercially available antibodies that are raised against extracellular epitope antigens such as peptides representing ECL1 or ECL2 of human Orai1.

FIG. 19 shows representative results of a FLIPR-based calcium influx assay using HEK-293/hOrai1/hSTIM1 BB6.3 cells that were stimulated with 1 μM thapsigargin.

FIG. 20 shows FACS binding data demonstrating binding to cynomolgus Orai1 by mAbs of the present invention. HEK-293-EBNA cells were transiently transfected with a construct expressing cyno Orai1 along with vector transfected control parental cells. Transfected cells were stained first with recombinant monoclonal antibodies then counter-stained with a secondary phycoerythrin-labeled Gt anti-Hu IgG F(ab′)₂ antibody fragment and were visualized using FACS.

FIG. 21 illustrates the results of FACS analysis showing binding to human Orai1 single-polynucleotide variant expressed on the surface of HEK-293-EBNA cells along with vector transfected control parental cells. HEK-293-EBNA cells were transiently transfected with a construct expressing a human Orai1 variant where the amino acid residue asparagine at position 223 of SEQ ID NO:2 is replaced with a serine (N223S; SEQ ID NO:317).

FIG. 22A-B illustrates the results of FACS analysis showing lack of binding of the commercially available polyclonal antibodies to the indicated mOrai1-hOrai1 ECL2 chimeric mutants and hOrai1-mOrai1 ECL2 chimeric mutants expressed on the surface of HEK-293-EBNA cells. Transfected cells were stained first with the commercially available antibodies to human Orai1, then counter-stained with a secondary FITC-labeled antibody fragment and visualized using FACS. The chimera tested were (i) mOrai1-hOrai1 ECL2 (RQAGQPSPTKPPAE) (SEQ ID NO:226); (ii) mOrai1-hOrai1 ECL2 (SPTKPPAE) (SEQ ID NO:214); (iii) mOrai1-hOrai1 ECL2 (KPPAE) (SEQ ID NO:133); (iv) mOrai1-hOrai1 ECL2 (SPTKPPAESVIV) (SEQ ID NO:218); (v) mOrai1-hOrai1 ECL2 (SPTKPPAESVIVANHSD) (SEQ ID NO:232); (vi) mOrai1-hOrai1 ECL2 (AESVIVANHSD) (SEQ ID NO:222); (vii) mOrai1-hOrai1 ECL2 (AESVIV) (SEQ ID NO:141); (viii) mOrai1-hOrai1 ECL2 (VIV) (SEQ ID NO:137); (ix) mOrai1-hOrai1 ECL2 (HSD) (SEQ ID NO:145); (x) hOrai1-mOrai1 ECL2 (KQPGQPRPTSKPPASGAA) (SEQ ID NO:210); (xi) hOrai1-mOrai1 ECL2 (KQPGQPRPTSKPPA) (SEQ ID NO:204); (xii) hOrai1-mOrai1 ECL2 (RPTSKPPASGAA) (SEQ ID NO: 192); (xiii) hOrai1-mOrai1 ECL2 (RPTSKPPA) (SEQ ID NO:129); (xiv) hOrai1-mOrai1 ECL2 (SKPPA) (SEQ ID NO:103); (xv) hOrai1-mOrai1 ECL2 (PASGAAANVST) (SEQ ID NO: 198); (xvi) hOrai1-mOrai1 ECL2 (PASGAA) (SEQ ID NO:113); (xvii) hOrai1-mOrai1 ECL2 (AANVST) (SEQ ID NO:123); (xviii) hOrai1-mOrai1 ECL2 (GAA) (SEQ ID NO:107); (xviiii) hOrai1-mOrai1 ECL2 (VST) (SEQ ID NO:117); (xx) hOrai1-mOrai1 ECL2 (SEQ ID NO:91); and (xxi) mOrai-hOrai1 ECL2 (SEQ ID NO:97). FIG. 22A-B shows the binding assessment by FACS of all the commercially available antibodies in Table 12 (Example 9 herein) that were raised against extracellular epitope antigens such as peptides representing ECL1 or ECL2 of human Orai1.

FIG. 23A-E illustrates the results of Western analysis showing detection of the commercially available antibodies to human Orai1 protein under native conditions with HEK-293, HEK-293/hOrai1/hSTIM1 BB6.3, Jurkat, AM1/CHO and AM1/hOrai1 cell lysates. Cell lysates were probed first with the commercially available antibodies to human Orai1, and then detected with a secondary horseradish peroxidase-conjugated IgG antibody. The proteins were visualized using an enhanced luminescence system. FIG. 23A-E shows the Western assessment of five indicated commercially available polyclonal antibodies that were raised against peptides representing ECL1 or ECL2 of human Orai1, as further described in Table 12 (in Example 9 herein).

FIG. 24A-E illustrates the results of Western analysis showing detection of the commercially available antibodies to human Orai1 protein under reducing and non-reducing conditions with HEK-293, HEK-293/hOrai1/hSTIM1 BB6.3, Jurkat, AM1/CHO and AM1/hOrai1 cell lysates. Cell lysates were probed first with the commercially available antibodies to human Orai1, and then detected with a secondary horseradish peroxidase-conjugated IgG antibody. The proteins were visualized using an enhanced luminescence system. FIG. 24A-E shows the Western assessment of five indicated commercially available polyclonal antibodies that were raised against peptides representing ECL1 or ECL2 of human Orai1, as further described in Table 12 (in Example 9 herein).

FIG. 25A-D shows representative data illustrating that inventive anti-Orai1 mAbs detected human Orai1 proteins under native conditions with HEK-293, HEK-293/hOrai1/hSTIM1 BB6.3, Jurkat, AM1/CHO and AM1/hOrai1 cell lysates. Cell lysates were probed first with the recombinant anti-hOrai1 monoclonal antibodies mAb 2B7.1 (FIG. 25A), mAb 2C1.1 (FIG. 25B), mAb 2D2.1 (FIG. 25C) and mAb 5F7.1 (FIG. 25D), and then were detected with a secondary horseradish peroxidase-conjugated IgG antibody. The proteins were visualized using an enhanced luminescence system.

FIG. 26A-D shows representative data illustrating that inventive anti-Orai1 mAbs detected human Orai1 proteins under reducing and non-reducing conditions with HEK-293, HEK-293/hOrai1/hSTIM1 BB6.3, Jurkat, AM1/CHO and AM1/hOrai1 cell lysates. Cell lysates were probed first with the recombinant anti-hOrai1 monoclonal antibodies mAb 2B7.1 (FIG. 26A), mAb 2C1.1 (FIG. 26B), mAb 2D2.1 (FIG. 26C) and mAb 5F7.1 (FIG. 26D), and then detected with a secondary horseradish peroxidase-conjugated IgG antibody. The proteins were visualized using an enhanced luminescence system.

FIG. 27 shows FACS analysis demonstrating binding of recombinant monoclonal antibodies to human Orai1 expressed on the surface of AM1-CHO cells. AM1-CHO parental and AM1-CHO/Orai1 were stained first with recombinant monoclonal antibodies then counter-stained with a secondary phycoerythrin labeled goat anti-human IgG F(ab′)₂ antibody fragment, and were visualized using FACS. Human Anti-DNP mAb (DNP-3A4-F) was described in Walker et al., WO 2010/108153 A2.

FIG. 28 illustrates the pharmacokinetic profile of anti-hOrai1 mAb2C1.1 in human xeno GVHD mice via intravenous or subcutaneous injection. The level of anti-hOrai1 mAb 2C1.1 in serum samples was measured by ELISA and time concentration data were analyzed using non-compartmental methods with WinNonLin®.

FIG. 29 shows anti-hOrai1 mAb2C1.1 preventing weight loss in human xeno GVHD mice. NSG mice were irradiated with 200 Rads Cs-137 and transferred with 20 million of human PBMCs in 2001 of PBS via tail intravenous injection. A group of irradiated mice which did not receive any treatment or human PBMC transfer as controls without GVHD. Recipients were treated with anti-KLH huIgG2 (described in Walker et al., WO 2010/108153 A2), Orencia® or anti-hOrai1 mAb2C1.1 via intraperitoneal injection on day 0 after irradiation prior to human PBMC transfer and on day 5.

FIG. 30A-D illustrates anti-hOrai1 mAb 2C1.1 attenuating the production of inflammatory cytokines, TNF-α, IFN-γ, IL-5 and IL-10, in human xeno GVHD mice. NSG mice were irradiated with 200 Rads Cs-137 and transferred with 20 million of human PBMCs in 2001 of PBS via tail intravenous injection. A group of irradiated mice which did not receive any treatment or human PBMC transfer as controls without GVHD. Recipients were treated with anti-KLH huIgG2 (described in Walker et al., WO 2010/108153 A2), Orencia® (abatacept; Bristol-Myers Squibb) or anti-hOrai1 mAb 2C1.1 via intraperitoneal injection on day-0 after irradiation prior to human PBMC transfer and on day-5.

FIG. 31A-D shows anti-hOrai1 mAb 2C1.1 preventing engraftment of human T cells in the spleens of in human xeno GVHD mice. NSG mice were irradiated with 200 Rads Cs-137 and transferred with 20 million of human PBMCs in 2001 of PBS via tail intravenous injection. A group of irradiated mice which did not receive any treatment or human PBMC transfer as controls without GVHD. Recipients were treated with anti-KLH huIgG2 (described in Walker et al., WO 2010/108153 A2), Orencia® (abatacept; Bristol-Myers Squibb) or anti-hOrai1 mAb 2C1.1 via intraperitoneal injection on day-0 after irradiation prior to human PBMC transfer and on day-5.

FIG. 32 shows that anti-hOrai1 mAb2C1.1, but not anti-hOrai1 mAb2B4.1, prevented weight loss in human xeno GVHD mice. NSG mice were irradiated with 200 Rads Cs-137 and transferred with 20 million of human PBMCs in 2001 of PBS via tail intravenous injection. A group of irradiated mice which did not receive any treatment or human PBMC transfer as controls without GVHD. Recipients were treated with anti-DNP huIgG2 (DNP-3A4-F-G2; described in Walker et al., WO 2010/108153 A2), anti-hOrai1 mAb2C1.1 or anti-hOrai1 mAb2B4.1 via intraperitoneal injection on day 0 after irradiation prior to human PBMC transfer and on day 5.

FIG. 33A-B show FACS binding data (FIG. 33A) and FACS profile (FIG. 33B) demonstrating binding to endogenous human Orai1 expressed in Jurkat cells by indicated mAbs embodiments of the present invention, recombinant anti-hOrai1 monoclonal antibodies mAb 2C1.1 (upper panel), mAb 2D2.1 (middle panel) and mAb 5F7.1 (lower panel), compared to mAb 2B4.1 and human isotype control anti-DNP mAb (DNP-3A4-F-G2; described in Walker et al., WO 2010/108153 A2), unstained control, and directly labeled secondary antibody fragment negative staining control. Jurkat cells were stained first with recombinant monoclonal antibodies or isotype control mAb then counter-stained with a secondary phycoerythrin-labeled Gt anti-Hu IgG F(ab′)₂ antibody fragment and were visualized using FACS.

FIG. 34 shows the binding of inventive anti-Orai1 mAbs for hOrai1 expressed on AM1-CHO/hOrai1/hSTIM1-YFP cells. 30 μM of each anti-Orai1 mAb was incubated with 3.0×10⁵, 1.0×10⁵ or 3.0×10⁴ cells/mL of cells and allowed to equilibrate. The supernatants of free mAb were measured first by passing through the goat-anti-huFc coated beads, then detected by fluorescent (Cy5) labeled goat anti-hulgG (H+L) antibody using the KinExA machine. The percent of binding signal was calculated from the free mAb value of a particular mAb (mAb 5F7.1, 5H3.1, 2C1.1, 5D7.2, 5F2.1, 5A4.2, 2B7.1, 5B1.1, 5B5.1, 2D2.1, or 2B4.1) binding a particular cell density divided by the free mAb value of that mAb binding no cells.

FIG. 35 shows the inhibitory effect of mAb 2C1.1 on CRAC current, measured at 6 concentrations of mAb 2C1.1 (n=3-6) by whole cell patch clamp.

FIG. 36 shows percent inhibition of ICRAC by 1 μM mAb 2C1.1, compared to 1 μM human isotype control anti-DNP mAb (DNP-3A4-F-G2; described in Walker et al., WO 2010/108153 A2).

### DETAILED DESCRIPTION OF EMBODIMENTS

The section headings used herein are for organizational purposes only and are not to be construed as limiting the subject matter described.

### Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular. Thus, as used in this specification and the appended claims, the singular forms “a”, “an” and “the” include plural referents unless the context clearly indicates otherwise. For example, reference to “a protein” includes a plurality of proteins; reference to “a cell” includes populations of a plurality of cells.

“Orai1” means Orai calcium release-activated calcium modulator 1, also known as calcium release-activated calcium modulator 1; CRACM 1; calcium release-activated calcium channel protein 1; transmembrane protein 142A; and TMEM142A. Human Orai1 (“hOrai1”) has been determined to have the following reference amino acid sequence (SEQ ID NO:2; NCBI Reference Sequence NP 116169.2):

SEQ ID NO: 2

MHPEPAPPPS RSSPELPPSG GSTTSGSRRS RRRSGDGEPP
GAPPPPPSAV TYPDWIGQSY SEVMSLNEHS MQALSWRKLY
LSRAKLKASS RTSALLSGFA MVAMVEVQLD ADHDYPPGLL
IAFSACTTVL VAVHLFALMI STCILPNIEA VSNVHNLNSV
KESPHERMHR HIELAWAFST VIGTLLFLAE VVLLCWVKFL
PLKKQPGQPR PTSKPPASGA AANVSTSGIT PGQAAAIAST
TIMVPFGLIF IVFAVHFYRS LVSHKTDRQF QELNELAEFA
RLQDQLDHRG DHPLTPGSHY A//.

SEQ ID NO: 65

MHPEPAPPPS RSSPELPPSG GSTTSGSRRS RRRSGDGEPP
GAPPPPPSAV TYPDWIGQSY SEVMSLNEHS MQALSWRKLY
LSRAKLKASS RTSALLSGFA MVAMVEVQLD ADHDYPPGLL
IAFSACTTVL VAVHLFALMI STCILPNIEA VSNVHNLNSV
KESPHERMHR HIELAWAFST VIGTLLFLAE VVLLCWVKFL
PLKKQPGQPR PTSKPPAGGA AANVSTSGIT PGQAAAIAST
TIMVPFGLIF IVFAVHFYRS LVSHKTDRQF QELNELAEFA
RLQDQLDHRG DHPLTPGSHY A//.

The putative extracellular loop 1 (“ECL1”) domain of human Orai1 is shown above at amino acid residues 110-117 of SEQ ID NO:2 and 110-117 of SEQ ID NO:65 (both above), which is single underlined in boldface and has the sequence DADHDYPP//SEQ ID NO:3.

The extracellular loop 2 (“ECL2”) domain of human Orai1 is shown at amino acid residues 198-233 of SEQ ID NO:2 or 198-233 of SEQ ID NO:65 (both above), which is single underlined and has the sequence of

SEQ ID NO: 4

KFLPLKKQPGQPRPTSKPPASGAAANVSTSGITPGQ//,

or in variant S218G:

SEQ ID NO: 70

KFLPLKKQPGQPRPTSKPPAGGAAANVSTSGITPGQ//.

Also encompassed within human Orai1 is the natural polymorphism variant N223S (see, NCBI SNP database, rs75603737):

SEQ ID NO: 317

MHPEPAPPPS RSSPELPPSG GSTTSGSRRS RRRSGDGEPP
GAPPPPPSAV TYPDWIGQSY SEVMSLNEHS MQALSWRKLY
LSRAKLKASS RTSALLSGFA MVAMVEVQLD ADHDYPPGLL
IAFSACTTVL VAVHLFALMI STCILPNIEA VSNVHNLNSV
KESPHERMHR HIELAWAFST VIGTLLFLAE VVLLCWVKFL
PLKKQPGQPR PTSKPPASGA AASVSTSGIT PGQAAAIAST
TIMVPFGLIF IVFAVHFYRS LVSHKTDRQF QELNELAEFA
RLQDQLDHRG DHPLTPGSHY A//.

The putative extracellular loop 1 (“ECL1”) domain of human Orai1 is shown above at amino acid residues 110-117 of SEQ ID NO:2 and 110-117 of SEQ ID NO:317 (both above), which is single underlined in boldface and has the sequence

SEQ ID NO: 3
DADHDYPP//.

The extracellular loop 2 (“ECL2”) domain of human Orai1 is shown at amino acid residues 198-233 of SEQ ID NO:2 or 198-233 of SEQ ID NO:317 (both above), which is single underlined and has the sequence of

SEQ ID NO: 4
KFLPLKKQPGQPRPTSKPPASGAAANVSTSGITPGQ//,

or in variant N223S:

SEQ ID NO: 313
KFLPLKKQPGQPRPTSKPPASGAAASVSTSGITPGQ//.

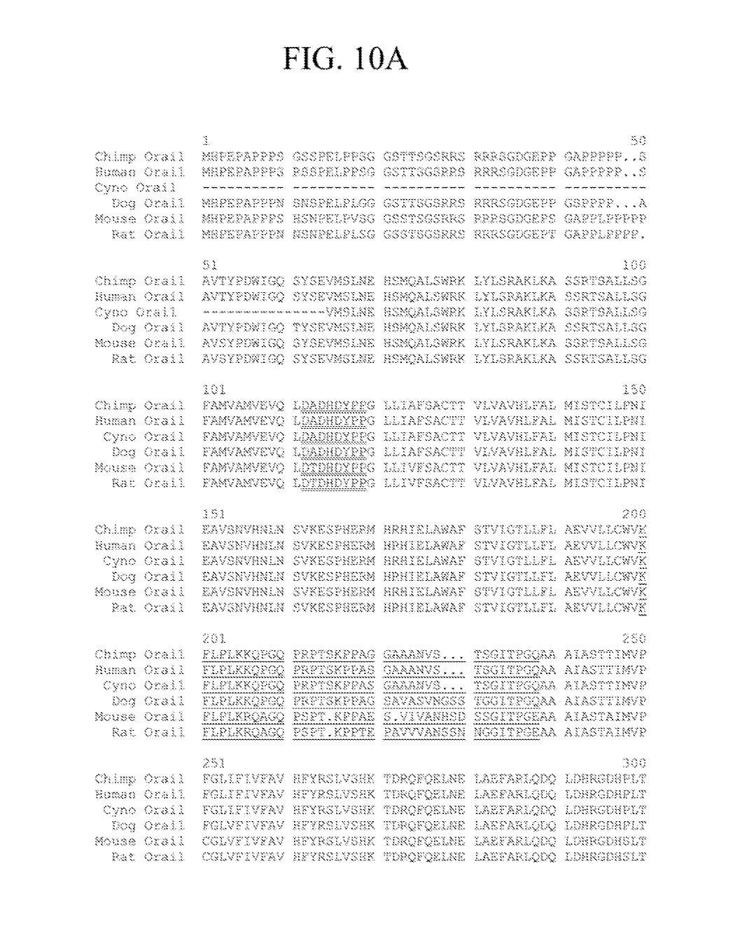

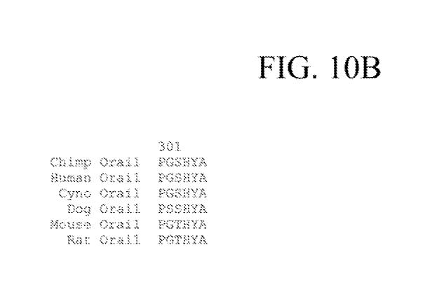

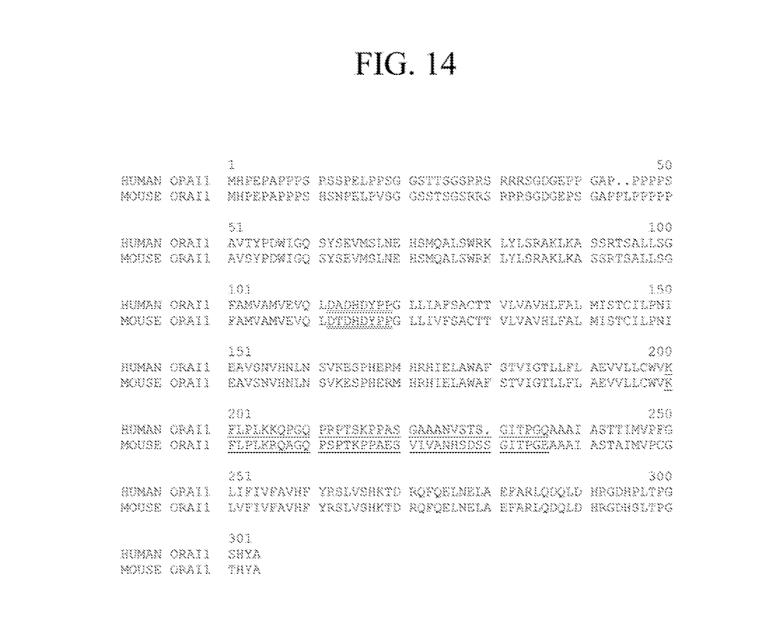

The predicted ECL1 and ECL2 domains at, respectively, positions 110-117 and 198-233 of SEQ ID NO:2, shown above, were determined using the TMpred program (available at www.ch.embnet.org/software/TMPRED_form.html), which makes a prediction of membrane-spanning regions and their orientation. The algorithm in the TMpred software is based on the statistical analysis of TMbase, a database of naturally occurring transmembrane proteins. The prediction is made using a combination of several weight-matrices for scoring. (K. Hofmann & W. Stoffel, TMbase—A database of membrane spanning proteins segments, Biol. Chem. Hoppe-Seyler 374:166 (1993)). Analyzing the human Orai1 amino acid sequence using the TMpred program two structural models resulted: (1) a strongly preferred model having an intracellular N-terminus, four strong transmembrane helices at positions 88-109 of SEQ ID NO:2, 118-136 of SEQ ID NO:2, 172-197 of SEQ ID NO:2, and 234-255 of SEQ ID NO:2; and (2) an alternative less preferred model having three strong transmembrane helices at 118-136 of SEQ ID NO:2, 118-136 of SEQ ID NO:2, 172-197 of SEQ ID NO:2, and 234-255 of SEQ ID NO:2. Other structural models of human Orai1 can be found, based on different algorithms. For example, the UniProtKB/Swiss-Prot Q96D31 prediction also has four transmembrane domains (at 88-105, 120-140, 174-194, and 235-255 of SEQ ID NO:2, respectively), with a cytoplasmic domain at positions 1-87 of SEQ ID NO:2, extracellular domain (ECL1) at 106-119 of SEQ ID NO:2, a cytoplasmic domain at 141-173 of SEQ ID NO:2, an extracellular domain (ECL2) at 195-234 of SEQ ID NO:2, and a cytoplasmic domain at 256-301 of SEQ ID NO:2. Another structural prediction placing the ECL1 at positions 110-125 of SEQ ID NO:2 and ECL2 at positions 197-236 of SEQ ID NO:2 may also be scientifically tenable (see, Vig et al., CRACM1 multimers form the ion-selective pore of the CRAC channel, Curr. Biol. 16:2073-2079 (2006)). As the amino acid sequences for Orai1 tends to be highly conserved among mammalian species at both the N-terminal and C-terminal ends of ECL2 and into the adjoining transmembrane regions (see, e.g., FIG. 10A-B and FIG. 14), we have chosen a reasonable subset 198-233 of SEQ ID NO:2 (i.e., SEQ ID NO:4) to adopt as the putative human Orai1 ECL2 sequence for practical purposes. However, the present invention does not rely on any particular structural model.

“Polypeptide” and “protein” are used interchangeably herein and include a molecular chain of two or more amino acids linked covalently through peptide bonds. The terms do not refer to a specific length of the product. Thus, “peptides,” and “oligopeptides,” are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included within the meaning of polypeptide.

The term “isolated protein” referred means that a subject protein (1) is free of at least some other proteins with which it would normally be found in nature, (2) is essentially free of other proteins from the same source, e.g., from the same species, (3) is expressed recombinantly by a cell of a heterologous species or kind, (4) has been separated from at least about 50 percent of polynucleotides, lipids, carbohydrates, or other materials with which it is associated in nature, (5) is operably associated (by covalent or noncovalent interaction) with a polypeptide with which it is not associated in nature, and/or (6) does not occur in nature. Typically, an “isolated protein” constitutes at least about 5%, at least about 10%, at least about 25%, or at least about 50% of a given sample. Genomic DNA, cDNA, mRNA or other RNA, of synthetic origin, or any combination thereof may encode such an isolated protein. Preferably, the isolated protein is substantially free from proteins or polypeptides or other contaminants that are found in its natural environment that would interfere with its therapeutic, diagnostic, prophylactic, research or other use.

A “variant” of a polypeptide (e.g., an antigen binding protein, or an antibody) comprises an amino acid sequence wherein one or more amino acid residues are inserted into, deleted from and/or substituted into the amino acid sequence relative to another polypeptide sequence. Variants include fusion proteins.

The term “fusion protein” indicates that the protein includes polypeptide components derived from more than one parental protein or polypeptide. Typically, a fusion protein is expressed from a fusion gene in which a nucleotide sequence encoding a polypeptide sequence from one protein is appended in frame with, and optionally separated by a linker from, a nucleotide sequence encoding a polypeptide sequence from a different protein. The fusion gene can then be expressed by a recombinant host cell as a single protein.

A “secreted” protein refers to those proteins capable of being directed to the ER, secretory vesicles, or the extracellular space as a result of a secretory signal peptide sequence, as well as those proteins released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage. In some other embodiments of the inventive composition, the toxin peptide analog can be synthesized by the host cell as a secreted protein, which can then be further purified from the extracellular space and/or medium.

As used herein “soluble” when in reference to a protein produced by recombinant DNA technology in a host cell is a protein that exists in aqueous solution; if the protein contains a twin-arginine signal amino acid sequence the soluble protein is exported to the periplasmic space in gram negative bacterial hosts, or is secreted into the culture medium by eukaryotic host cells capable of secretion, or by bacterial host possessing the appropriate genes (e.g., the kil gene). Thus, a soluble protein is a protein which is not found in an inclusion body inside the host cell. Alternatively, depending on the context, a soluble protein is a protein which is not found integrated in cellular membranes; in contrast, an insoluble protein is one which exists in denatured form inside cytoplasmic granules (called an inclusion body) in the host cell, or again depending on the context, an insoluble protein is one which is present in cell membranes, including but not limited to, cytoplasmic membranes, mitochondrial membranes, chloroplast membranes, endoplasmic reticulum membranes, etc.

The term “recombinant” indicates that the material (e.g., a nucleic acid or a polypeptide) has been artificially or synthetically (i.e., non-naturally) altered by human intervention. The alteration can be performed on the material within, or removed from, its natural environment or state. For example, a “recombinant nucleic acid” is one that is made by recombining nucleic acids, e.g., during cloning, DNA shuffling or other well known molecular biological procedures. Examples of such molecular biological procedures are found in Maniatis et al., Molecular Cloning. A Laboratory Manual. Cold Spring Harbour Laboratory, Cold Spring Harbour, N.Y (1982). A “recombinant DNA molecule,” is comprised of segments of DNA joined together by means of such molecular biological techniques. The term “recombinant protein” or “recombinant polypeptide” as used herein refers to a protein molecule which is expressed using a recombinant DNA molecule. A “recombinant host cell” is a cell that contains and/or expresses a recombinant nucleic acid.

The term “polynucleotide” or “nucleic acid” includes both single-stranded and double-stranded nucleotide polymers containing two or more nucleotide residues. The nucleotide residues comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phosphoraniladate and phosphoroamidate.

The term “oligonucleotide” means a polynucleotide comprising 200 or fewer nucleotide residues. In some embodiments, oligonucleotides are 10 to 60 bases in length. In other embodiments, oligonucleotides are 12, 13, 14, 15, 16, 17, 18, 19, or 20 to 40 nucleotides in length. Oligonucleotides may be single stranded or double stranded, e.g., for use in the construction of a mutant gene. Oligonucleotides may be sense or antisense oligonucleotides. An oligonucleotide can include a label, including a radiolabel, a fluorescent label, a hapten or an antigenic label, for detection assays. Oligonucleotides may be used, for example, as PCR primers, cloning primers or hybridization probes.

A “polynucleotide sequence” or “nucleotide sequence” or “nucleic acid sequence,” as used interchangeably herein, is the primary sequence of nucleotide residues in a polynucleotide, including of an oligonucleotide, a DNA, and RNA, a nucleic acid, or a character string representing the primary sequence of nucleotide residues, depending on context. From any specified polynucleotide sequence, either the given nucleic acid or the complementary polynucleotide sequence can be determined. Included are DNA or RNA of genomic or synthetic origin which may be single- or double-stranded, and represent the sense or antisense strand. Unless specified otherwise, the left-hand end of any single-stranded polynucleotide sequence discussed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences;” sequence regions on the DNA strand having the same sequence as the RNA transcript that are 3′ to the 3′ end of the RNA transcript are referred to as “downstream sequences.”

As used herein, an “isolated nucleic acid molecule” or “isolated nucleic acid sequence” is a nucleic acid molecule that is either (1) identified and separated from at least one contaminant nucleic acid molecule with which it is ordinarily associated in the natural source of the nucleic acid or (2) cloned, amplified, tagged, or otherwise distinguished from background nucleic acids such that the sequence of the nucleic acid of interest can be determined. An isolated nucleic acid molecule is other than in the form or setting in which it is found in nature. However, an isolated nucleic acid molecule includes a nucleic acid molecule contained in cells that ordinarily express the antigen binding protein (e.g., antibody) where, for example, the nucleic acid molecule is in a chromosomal location different from that of natural cells.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of ribonucleotides along the mRNA chain, and also determines the order of amino acids along the polypeptide (protein) chain. The DNA sequence thus codes for the RNA sequence and for the amino acid sequence.

The term “gene” is used broadly to refer to any nucleic acid associated with a biological function. Genes typically include coding sequences and/or the regulatory sequences required for expression of such coding sequences. The term “gene” applies to a specific genomic or recombinant sequence, as well as to a cDNA or mRNA encoded by that sequence. A “fusion gene” contains a coding region that encodes a toxin peptide analog. Genes also include non-expressed nucleic acid segments that, for example, form recognition sequences for other proteins. Non-expressed regulatory sequences including transcriptional control elements to which regulatory proteins, such as transcription factors, bind, resulting in transcription of adjacent or nearby sequences.

“Expression of a gene” or “expression of a nucleic acid” means transcription of DNA into RNA (optionally including modification of the RNA, e.g., splicing), translation of RNA into a polypeptide (possibly including subsequent post-translational modification of the polypeptide), or both transcription and translation, as indicated by the context.

As used herein the term “coding region” or “coding sequence” when used in reference to a structural gene refers to the nucleotide sequences which encode the amino acids found in the nascent polypeptide as a result of translation of an mRNA molecule. The coding region is bounded, in eukaryotes, on the 5′ side by the nucleotide triplet “ATG” which encodes the initiator methionine and on the 3′ side by one of the three triplets which specify stop codons (i.e., TAA, TAG, TGA).

The term “control sequence” or “control signal” refers to a polynucleotide sequence that can, in a particular host cell, affect the expression and processing of coding sequences to which it is ligated. The nature of such control sequences may depend upon the host organism. In particular embodiments, control sequences for prokaryotes may include a promoter, a ribosomal binding site, and a transcription termination sequence. Control sequences for eukaryotes may include promoters comprising one or a plurality of recognition sites for transcription factors, transcription enhancer sequences or elements, polyadenylation sites, and transcription termination sequences. Control sequences can include leader sequences and/or fusion partner sequences. Promoters and enhancers consist of short arrays of DNA that interact specifically with cellular proteins involved in transcription (Maniatis, et al., Science 236:1237 (1987)). Promoter and enhancer elements have been isolated from a variety of eukaryotic sources including genes in yeast, insect and mammalian cells and viruses (analogous control elements, i.e., promoters, are also found in prokaryotes). The selection of a particular promoter and enhancer depends on what cell type is to be used to express the protein of interest. Some eukaryotic promoters and enhancers have a broad host range while others are functional in a limited subset of cell types (for review see Voss, et al., Trends Biochem. Sci., 11:287 (1986) and Maniatis, et al., Science 236:1237 (1987)).

The term “vector” means any molecule or entity (e.g., nucleic acid, plasmid, bacteriophage or virus) used to transfer protein coding information into a host cell.

The term “expression vector” or “expression construct” as used herein refers to a recombinant DNA molecule containing a desired coding sequence and appropriate nucleic acid control sequences necessary for the expression of the operably linked coding sequence in a particular host cell. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired. Such techniques are well known in the art. (E.g., Goodey, Andrew R.; et al., Peptide and DNA sequences, U.S. Pat. No. 5,302,697; Weiner et al., Compositions and methods for protein secretion, U.S. Pat. No. 6,022,952 and U.S. Pat. No. 6,335,178; Uemura et al., Protein expression vector and utilization thereof, U.S. Pat. No. 7,029,909; Ruben et al., 27 human secreted proteins, US 2003/0104400 A1).

The terms “in operable combination”, “in operable order” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced. For example, a control sequence in a vector that is “operably linked” to a protein coding sequence is ligated thereto so that expression of the protein coding sequence is achieved under conditions compatible with the transcriptional activity of the control sequences.

The term “host cell” means a cell that has been transformed, or is capable of being transformed, with a nucleic acid and thereby expresses a gene of interest. The term includes the progeny of the parent cell, whether or not the progeny is identical in morphology or in genetic make-up to the original parent cell, so long as the gene of interest is present. Any of a large number of available and well-known host cells may be used in the practice of this invention. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all hosts may be equally effective for the expression of a particular DNA sequence. Within these general guidelines, useful microbial host cells in culture include bacteria (such as Escherichia coli sp.), yeast (such as Saccharomyces sp.) and other fungal cells, insect cells, plant cells, mammalian (including human) cells, e.g., CHO cells and HEK-293 cells. Modifications can be made at the DNA level, as well. The peptide-encoding DNA sequence may be changed to codons more compatible with the chosen host cell. For E. coli, optimized codons are known in the art. Codons can be substituted to eliminate restriction sites or to include silent restriction sites, which may aid in processing of the DNA in the selected host cell. Next, the transformed host is cultured and purified. Host cells may be cultured under conventional fermentation conditions so that the desired compounds are expressed. Such fermentation conditions are well known in the art.

The term “transfection” means the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al., 1981, Gene 13:197. Such techniques can be used to introduce one or more exogenous DNA moieties into suitable host cells.

The term “transformation” refers to a change in a cell's genetic characteristics, and a cell has been transformed when it has been modified to contain new DNA or RNA. For example, a cell is transformed where it is genetically modified from its native state by introducing new genetic material via transfection, transduction, or other techniques. Following transfection or transduction, the transforming DNA may recombine with that of the cell by physically integrating into a chromosome of the cell, or may be maintained transiently as an episomal element without being replicated, or may replicate independently as a plasmid. A cell is considered to have been “stably transformed” when the transforming DNA is replicated with the division of the cell.

By “physiologically acceptable salt” of a composition of matter, for example a salt of the antigen binding protein, such as an antibody, is meant any salt or salts that are known or later discovered to be pharmaceutically acceptable. Some non-limiting examples of pharmaceutically acceptable salts are: acetate; trifluoroacetate; hydrohalides, such as hydrochloride and hydrobromide; sulfate; citrate; maleate; tartrate; glycolate; gluconate; succinate; mesylate; besylate; salts of gallic acid esters (gallic acid is also known as 3,4,5 trihydroxybenzoic acid) such as PentaGalloylGlucose (PGG) and epigallocatechin gallate (EGCG), salts of cholesteryl sulfate, pamoate, tannate and oxalate salts.

A “domain” or “region” (used interchangeably herein) of a protein is any portion of the entire protein, up to and including the complete protein, but typically comprising less than the complete protein. A domain can, but need not, fold independently of the rest of the protein chain and/or be correlated with a particular biological, biochemical, or structural function or location (e.g., a ligand binding domain, or a cytosolic, transmembrane or extracellular domain).

“Treatment” or “treating” is an intervention performed with the intention of preventing the development or altering the pathology of a disorder. Accordingly, “treatment” refers to both therapeutic treatment and prophylactic or preventative measures. Those in need of treatment include those already with the disorder as well as those in which the disorder is to be prevented. “Treatment” includes any indicia of success in the amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, self-reporting by a patient, neuropsychiatric exams, and/or a psychiatric evaluation.

An “effective amount” is generally an amount sufficient to reduce the severity and/or frequency of symptoms, eliminate the symptoms and/or underlying cause, prevent the occurrence of symptoms and/or their underlying cause, and/or improve or remediate the damage that results from or is associated with migraine headache. In some embodiments, the effective amount is a therapeutically effective amount or a prophylactically effective amount. A “therapeutically effective amount” is an amount sufficient to remedy a disease state (e.g., transplant rejection or GVHD) or symptom(s), particularly a state or symptom(s) associated with the disease state, or otherwise prevent, hinder, retard or reverse the progression of the disease state or any other undesirable symptom associated with the disease in any way whatsoever (i.e. that provides “therapeutic efficacy”). A “prophylactically effective amount” is an amount of a pharmaceutical composition that, when administered to a subject, will have the intended prophylactic effect, e.g., preventing or delaying the onset (or reoccurrence) of migraine headache, or reducing the likelihood of the onset (or reoccurrence) of migraine headache or migraine headache symptoms. The full therapeutic or prophylactic effect does not necessarily occur by administration of one dose, and may occur only after administration of a series of doses. Thus, a therapeutically or prophylactically effective amount may be administered in one or more administrations.

“Mammal” for purposes of treatment refers to any animal classified as a mammal, including humans, domestic and farm animals, and zoo, sports, or pet animals, such as dogs, horses, cats, cows, rats, mice, monkeys, etc. Preferably, the mammal is human.

The term “naturally occurring” as used throughout the specification in connection with biological materials such as polypeptides, nucleic acids, host cells, and the like, refers to materials which are found in nature.

The term “antibody” is used in the broadest sense and includes fully assembled antibodies, monoclonal antibodies (including human, humanized or chimeric antibodies), polyclonal antibodies, multispecific antibodies (e.g., bispecific antibodies), and antibody fragments that can bind antigen (e.g., Fab, Fab′, F(ab′)₂, Fv, single chain antibodies, diabodies), comprising complementarity determining regions (CDRs) of the foregoing as long as they exhibit the desired biological activity. Multimers or aggregates of intact molecules and/or fragments, including chemically derivatized antibodies, are contemplated. Antibodies of any isotype class or subclass, including IgG, IgM, IgD, IgA, and IgE, IgG1, IgG2, IgG3, IgG4, IgA1 and IgA2, or any allotype, are contemplated. Different isotypes have different effector functions; for example, IgG1 and IgG3 isotypes typically have antibody-dependent cellular cytotoxicity (ADCC) activity. Glycosylated and unglycosylated antibodies are included within the term “antibody”.

The term “antigen binding protein” (ABP) includes antibodies or antibody fragments, as defined above, and recombinant peptides or other compounds that contain sequences derived from CDRs having the desired antigen-binding properties such that they specifically bind a target antigen of interest.

In general, an antigen binding protein, e.g., an antibody or antibody fragment, “specifically binds” to an antigen when it has a significantly higher binding affinity for, and consequently is capable of distinguishing, that antigen, compared to its affinity for other unrelated proteins, under similar binding assay conditions. Typically, an antigen binding protein is said to “specifically bind” its target antigen when the equilibrium dissociation constant (K_{d}) is <10⁻⁸ M. The antibody specifically binds antigen with “high affinity” when the K_{d} is <5×10⁻⁹ M, and with “very high affinity” when the K_{d} is <5×10⁻¹⁰ M. In one embodiment, the antibodies will bind to human Orai1 with a K_{d} of between about 10⁻⁸ M and 10⁻¹⁰ M, and in yet another embodiment the antibodies will bind with a K_{d}<5×10⁻⁹. In particular embodiments of the inventive antigen binding protein, the isolated antigen binding protein specifically binds to SEQ ID NO:2 expressed by a mammalian cell (e.g., CHO, HEK 293, Jurkat), with a K_{d} of 500 μM (5.0×10⁻¹⁰ M) or less, 200 μM (2.0×10⁻¹⁰ M) or less, 150 pM (1.50×10⁻¹⁰ M) or less, 125 pM (1.25×10⁻¹⁰ M) or less, 105 pM (1.05×10⁻¹⁰ M) or less, 50 pM (5.0×10⁻¹¹ M) or less, or 20 pM (2.0×10⁻¹¹ M) or less, as determined by a Kinetic Exclusion Assay, conducted by the method of Rathanaswami et al. (2008) (Rathanaswami et al., High affinity binding measurements of antibodies to cell-surface-expressed antigens, Analytical Biochemistry 373:52-60 (2008; see, e.g., Example 15 herein).

An antigen binding protein of the present invention, e.g., an antibody or antibody fragment, is “specifically binding” or “specifically binds” to a human Orai1 polypeptide (SEQ ID NO:2), and/or “specifically binds” to SEQ ID NO:4 (amino acid residues 198-233 of SEQ ID NO:2), and/or “specifically binds” to a polypeptide having an amino acid sequence consisting of: (i) SEQ ID NO:210; or (ii) SEQ ID NO:204; or (iii) SEQ ID NO:192; or (iv) SEQ ID NO:129; or (v) SEQ ID NO:103, in a fluorescently-activated cell sorting (FACS) assay system. First, a mammalian host cell, such as a CHO (e.g., AM-1-CHO), HEK-293 (e.g., HEK-293-EBNA or HEK-293T), U20S, or other transfectable mammalian cell type, is transfected (stably or transiently) with an expression vector (e.g., pcDNA5/TO or pcDNA3.1) including a recombinant DNA molecule containing an operably linked coding sequence encoding one of the target amino acid sequences enumerated in this paragraph and appropriate nucleic acid control sequences necessary for the expression of the operably linked coding sequence in the particular host cell such that the target sequence is expressed on the surface of the host cell. Within 48 hours of transfection, the host cells are harvested and washed once with ice-cold 1× Dulbecco's Phosphate-Buffered Saline (D-PBS; 0.901 mM Calcium Chloride (CaCl₂) (anhyd.), 0.493 mM Magnesium Chloride (MgCl₂-6H₂O), 2.67 mM Potassium Chloride (KCl), 1.47 mM Potassium Phosphate monobasic (KH₂PO₄), 137.93 Sodium Chloride (NaCl) and 8.06 mM Sodium Phosphate dibasic (Na₂HPO₄-7H₂O)), pH 7.2, and resuspended in ice-cold FACS buffer (1×D-PBS+2% goat serum) to a concentration of 2×10⁵ cells in 1001. All of the following antibody incubation steps are performed on ice for 1 hour: (a) Cells are first incubated with 1 g of unlabeled test antigen binding protein (ABP; e.g., an antibody or antibody fragment) followed by a wash with 200 μL of FACS buffer; (b) the unlabelled test ABP (e.g., an antibody or antibody fragment) in (a) is then detected using a labeled secondary antibody suitable for detecting the test ABP, such as goat F(ab′)₂ anti-mouse IgG-phycoerythrin (PE) (or fluorescein isothiocyanate [FITC]-labeled) or anti-human IgG-phycoerythrin (or —FITC labeled), followed by a wash with 200 μL of ice-cold FACS buffer before flow cytometry analysis within two hours using any suitable FACS machine. If the ABP is not of an immunoglobulin type (e.g., mouse IgG or human IgG) for which secondary labeling antibodies are readily available, then a sandwich assay can be employed wherein 1-hour incubation with a secondary antibody (or antibody fragment; e.g., goat anti-ABP antibody) that specifically binds the ABP is followed by an additional wash with ice cold FACS buffer before the final 1-hour incubation with the secondary (now tertiary) labeling antibody, followed by an additional wash with ice cold FACS buffer to remove unbound labeling antibody, resuspension of the cells in fresh ice cold FACS buffer, and fluorescense detection with a suitable FACS-capable instrument (e.g., BD FACSCalibur™, BD FACSCanto™ II, BD LSR II, BD LSRFortessa™[BD Biosciences]; or Cytomics FC 500 [Beckman Coulter]). The following negative controls are also processed (1) Unstained Cells (incubated with FACS buffer not containing secondary labeling antibody); (2) cells stained with detecting antibodies (i.e., incubated with secondary labeling antibody after 1-hour incubation with FACS buffer not containing test ABP); and (3) host cells transfected with the expression vector system employed, but without the target coding sequence, and otherwise incubated with test ABP and secondary/tertiary labeling antibody. Additional negative controls can be employed as appropriate. A positive control employs the same type of mammalian host cell used above transfected to express on its surface the recombinant protein having amino acid sequence consisting of SEQ ID NO:97 encoded by an operably linked DNA contained by the expression vector, and a recombinant mAb2D2.1, having two immunoglobulin heavy chains with amino acid sequence consisting of SEQ ID NO:33 and two immunonoglobulin light chains with amino acid sequence consisting of SEQ ID NO:31, is made and purified by well known recombinant techniques as described herein (e.g., Example 4 and Cabilly et al., Methods of producing immunoglobulins, vectors and transformed host cells for use therein, U.S. Pat. No. 6,331,415). The values of relative fluorescence intensity (RFI) are calculated using FCS Express (De Novo Software), or another software package suitable for FACS analysis, and mean values are calculated using log-transformed data (geometric mean). The RFI as a percent of control (RFI-POC) value is calculated from the relative fluorescence intensity geometric mean (Geo Mean) using the algorithm (Algorithm I in Example 8 herein) of Geo Mean of a particular mAb binding a particular sample chimera minus the average Geo Mean of Unstained Cells and directly labeled secondary antibody only (negative controls) divided by the Geo Mean of that mAb binding the mOrai1-hOrai1 ECL2 chimera, multiplied by 100 to give percent. Binding with RFI-POC equal to or greater than 1% of the positive control is considered “specifically binding”.

In some embodiments the inventive antigen binding protein (e.g., antibody or anbody fragment) has a RFI-POC value 1% to less than 5%. In other embodiments the inventive antigen binding protein has a RFI-POC value 5% to less than 40%. In still other embodiments, the inventive antigen binding protein has a RFI-POC value 40% or greater.

For example, the antigen binding proteins of the present invention specifically bind to SEQ ID NO:4 (human Orai1 ECL2 having the amino acid sequence of 198-233 of SEQ ID NO:2) in a polypeptide consisting of the amino acid sequence of SEQ ID NO:2, but do not cross-react significantly with mouse or rat Orai1, or with human Orai2 or human Orai3. In some embodiments the antigen binding protein will cross-react with Orai1 of other mammalian species, such as primate, e.g., Orai1 of cynomolgus monkey; or Orai1 of dog, while in other embodiments, the antigen binding proteins bind only to human or primate Orai1 and not significantly to other mammalian Orai1s.

“Antigen binding region” or “antigen binding site” means a portion of a protein, that specifically binds a specified antigen. For example, that portion of an antigen binding protein that contains the amino acid residues that interact with an antigen and confer on the antigen binding protein its specificity and affinity for the antigen is referred to as “antigen binding region.” An antigen binding region typically includes one or more “complementary binding regions” (“CDRs”). Certain antigen binding regions also include one or more “framework” regions (“FRs”). A “CDR” is an amino acid sequence that contributes to antigen binding specificity and affinity. “Framework” regions can aid in maintaining the proper conformation of the CDRs to promote binding between the antigen binding region and an antigen.

An “isolated” antigen binding protein or antibody is one that has been identified and separated from one or more components of its natural environment or of a culture medium in which it has been secreted by a producing cell. “Contaminant” components of its natural environment or medium are materials that would interfere with diagnostic or therapeutic uses for the antibody, and may include enzymes, hormones, and other proteinaceous or nonproteinaceous solutes. In some embodiments, the antibody will be purified (1) to greater than 95% by weight of antibody, and most preferably more than 99% by weight, or (2) to homogeneity by SDS-PAGE under reducing or nonreducing conditions, optionally using a stain, e.g., Coomassie blue or silver stain. Isolated naturally occurring antibody includes the antibody in situ within recombinant cells since at least one component of the antibody's natural environment will not be present. Typically, however, isolated antibody will be prepared by at least one purification step.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against an individual antigenic site or epitope, in contrast to polyclonal antibody preparations that typically include different antibodies directed against different epitopes. Nonlimiting examples of monoclonal antibodies include murine, rabbit, rat, chicken, chimeric, humanized, or human antibodies, fully assembled antibodies, multispecific antibodies (including bispecific antibodies), antibody fragments that can bind an antigen (including, Fab, Fab′, F(ab′)₂, Fv, single chain antibodies, diabodies), maxibodies, nanobodies, and recombinant peptides comprising CDRs of the foregoing as long as they exhibit the desired biological activity, or variants or derivatives thereof.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 [1975], or may be made by recombinant DNA methods (see, e.g., U.S. Pat. No. 4,816,567). The “monoclonal antibodies” may also be isolated from phage antibody libraries using the techniques described in Clackson et al., Nature, 352:624-628[1991] and Marks et al., J. Mol. Biol., 222:581-597 (1991), for example.

A “multispecific” binding agent or antigen binding protein or antibody is one that targets more than one antigen or epitope.

A “bispecific,” “dual-specific” or “bifunctional” binding agent or antigen binding protein or antibody is a hybrid having two different antigen binding sites. Biantigen binding proteins, antigen binding proteins and antibodies are a species of multiantigen binding protein, antigen binding protein or multispecific antibody and may be produced by a variety of methods including, but not limited to, fusion of hybridomas or linking of Fab′ fragments. See, e.g., Songsivilai and Lachmann, 1990, Clin. Exp. Immunol. 79:315-321; Kostelny et al., 1992, J. Immunol. 148:1547-1553. The two binding sites of a bispecific antigen binding protein or antibody will bind to two different epitopes, which may reside on the same or different protein targets.

An “immunoglobulin” or “native antibody” is a tetrameric glycoprotein. In a naturally-occurring immunoglobulin, each tetramer is composed of two identical pairs of polypeptide chains, each pair having one “light” chain of about 220 amino acids (about 25 kDa) and one “heavy” chain of about 440 amino acids (about 50-70 kDa). The amino-terminal portion of each chain includes a “variable” (“V”) region of about 100 to 110 or more amino acids primarily responsible for antigen recognition. The carboxy-terminal portion of each chain defines a constant region primarily responsible for effector function. The variable region differs among different antibodies, the constant region is the same among different antibodies. Within the variable region of each heavy or light chain, there are three hypervariable subregions that help determine the antibody's specificity for antigen. The variable domain residues between the hypervariable regions are called the framework residues and generally are somewhat homologous among different antibodies. Immunoglobulins can be assigned to different classes depending on the amino acid sequence of the constant domain of their heavy chains. Human light chains are classified as kappa (κ) and lambda (λ) light chains. Within light and heavy chains, the variable and constant regions are joined by a “J” region of about 12 or more amino acids, with the heavy chain also including a “D” region of about 10 more amino acids. See generally, Fundamental Immunology, Ch. 7 (Paul, W., ed., 2nd ed. Raven Press, N.Y. (1989)).

The term “light chain” or “immunoglobulin light chain” includes a full-length light chain and fragments thereof having sufficient variable region sequence to confer binding specificity. A full-length light chain includes a variable region domain, V_{L}, and a constant region domain, C_{L}. The variable region domain of the light chain is at the amino-terminus of the polypeptide. Light chains include kappa chains and lambda chains.

The term “heavy chain” or “immunoglobulin heavy chain” includes a full-length heavy chain and fragments thereof having sufficient variable region sequence to confer binding specificity. A full-length heavy chain includes a variable region domain, V_{H}, and three constant region domains, C_{H}1, C_{H}2, and C_{H}3. The V_{H} domain is at the amino-terminus of the polypeptide, and the C_{H} domains are at the carboxyl-terminus, with the C_{H}3 being closest to the carboxy-terminus of the polypeptide. Heavy chains are classified as mu (μ), delta (Δ), gamma (γ), alpha (α), and epsilon (ε), and define the antibody's isotype as IgM, IgD, IgG, IgA, and IgE, respectively. In separate embodiments of the invention, heavy chains may be of any isotype, including IgG (including IgG1, IgG2, IgG3 and IgG4 subtypes), IgA (including IgA1 and IgA2 subtypes), IgM and IgE. Several of these may be further divided into subclasses or isotypes, e.g. IgG1, IgG2, IgG3, IgG4, IgA1 and IgA2. Different IgG isotypes may have different effector functions (mediated by the Fc region), such as antibody-dependent cellular cytotoxicity (ADCC) and complement-dependent cytotoxicity (CDC). In ADCC, the Fc region of an antibody binds to Fc receptors (FcγR5) on the surface of immune effector cells such as natural killers and macrophages, leading to the phagocytosis or lysis of the targeted cells. In CDC, the antibodies kill the targeted cells by triggering the complement cascade at the cell surface.

An “Fc region” contains two heavy chain fragments comprising the C_{H}1 and C_{H}2 domains of an antibody. The two heavy chain fragments are held together by two or more disulfide bonds and by hydrophobic interactions of the C_{H}3 domains.

The term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

“Allotypes” are variations in antibody sequence, often in the constant region, that can be immunogenic and are encoded by specific alleles in humans. Allotypes have been identified for five of the human IGHC genes, the IGHG 1, IGHG2, IGHG3, IGHA2 and IGHE genes, and are designated as G1m, G2m, G3m, A2m, and Em allotypes, respectively. At least 18 Gm allotypes are known: nGlm(1), nGlm(2), Glm (1, 2, 3, 17) or G1m (a, x, f, z), G2m (23) or G2m (n), G3m (5, 6, 10, 11, 13, 14, 15, 16, 21, 24, 26, 27, 28) or G3m (b1, c3, b5, b0, b3, b4, s, t, g1, c5, u, v, g5). There are two A2m allotypes A2m(1) and A2m(2).

For a detailed description of the structure and generation of antibodies, see Roth, D. B., and Craig, N. L., Cell, 94:411-414 (1998), herein incorporated by reference in its entirety. Briefly, the process for generating DNA encoding the heavy and light chain immunoglobulin sequences occurs primarily in developing B-cells. Prior to the rearranging and joining of various immunoglobulin gene segments, the V, D, J and constant (C) gene segments are found generally in relatively close proximity on a single chromosome. During B-cell-differentiation, one of each of the appropriate family members of the V, D, J (or only V and J in the case of light chain genes) gene segments are recombined to form functionally rearranged variable regions of the heavy and light immunoglobulin genes. This gene segment rearrangement process appears to be sequential. First, heavy chain D-to-J joints are made, followed by heavy chain V-to-DJ joints and light chain V-to-J joints. In addition to the rearrangement of V, D and J segments, further diversity is generated in the primary repertoire of immunoglobulin heavy and light chains by way of variable recombination at the locations where the V and J segments in the light chain are joined and where the D and J segments of the heavy chain are joined. Such variation in the light chain typically occurs within the last codon of the V gene segment and the first codon of the J segment. Similar imprecision in joining occurs on the heavy chain chromosome between the D and J_{H} segments and may extend over as many as 10 nucleotides. Furthermore, several nucleotides may be inserted between the D and J_{H} and between the V_{H} and D gene segments which are not encoded by genomic DNA. The addition of these nucleotides is known as N-region diversity. The net effect of such rearrangements in the variable region gene segments and the variable recombination which may occur during such joining is the production of a primary antibody repertoire.

The term “hypervariable” region refers to the amino acid residues of an antibody which are responsible for antigen-binding. The hypervariable region comprises amino acid residues from a complementarity determining region or CDR [i.e., residues 24-34 (L1), 50-56 (L2) and 89-97 (L3) in the light chain variable domain and 31-35 (H1), 50-65 (H2) and 95-102 (H3) in the heavy chain variable domain as described by Kabat et al., Sequences of Proteins of Immunological Interest, 5^{th} Ed. Public Health Service, National Institutes of Health, Bethesda, Md. (1991)]. Even a single CDR may recognize and bind antigen, although with a lower affinity than the entire antigen binding site containing all of the CDRs.

An alternative definition of residues from a hypervariable “loop” is described by Chothia et al., J. Mol. Biol. 196: 901-917 (1987) as residues 26-32 (L1), 50-52 (L2) and 91-96 (L3) in the light chain variable domain and 26-32 (H1), 53-55 (H2) and 96-101 (H3) in the heavy chain variable domain.

“Framework” or “FR” residues are those variable region residues other than the hypervariable region residues.

“Antibody fragments” comprise a portion of an intact full length antibody, preferably the antigen binding or variable region of the intact antibody. Examples of antibody fragments include Fab, Fab′, F(ab′)₂, and Fv fragments; diabodies; linear antibodies (Zapata et al., Protein Eng., 8(10):1057-1062 (1995)); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment which contains the constant region. The Fab fragment contains all of the variable domain, as well as the constant domain of the light chain and the first constant domain (CH1) of the heavy chain. The Fc fragment displays carbohydrates and is responsible for many antibody effector functions (such as binding complement and cell receptors), that distinguish one class of antibody from another.

Pepsin treatment yields an F(ab′)₂ fragment that has two “Single-chain Fv” or “scFv” antibody fragments comprising the VH and VL domains of antibody, wherein these domains are present in a single polypeptide chain. Fab fragments differ from Fab′ fragments by the inclusion of a few additional residues at the carboxy terminus of the heavy chain CH1 domain including one or more cysteines from the antibody hinge region. Preferably, the Fv polypeptide further comprises a polypeptide linker between the VH and VL domains that enables the Fv to form the desired structure for antigen binding. For a review of scFv see Pluckthun in The Pharmacology of Monoclonal Antibodies, vol. 1 13, Rosenburg and Moore eds., Springer-Verlag, New York, pp. 269-315 (1994).

A “Fab fragment” is comprised of one light chain and the C_{H}1 and variable regions of one heavy chain. The heavy chain of a Fab molecule cannot form a disulfide bond with another heavy chain molecule.

A “Fab′ fragment” contains one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form an F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

“Fv” is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen binding site on the surface of the VH VL dimer. A single variable domain (or half of an Fv comprising only three CDRs specific for an antigen) has the ability to recognize and bind antigen, although at a lower affinity than the entire binding site.

“Single-chain antibodies” are Fv molecules in which the heavy and light chain variable regions have been connected by a flexible linker to form a single polypeptide chain, which forms an antigen-binding region. Single chain antibodies are discussed in detail in International Patent Application Publication No. WO 88/01649 and U.S. Pat. No. 4,946,778 and No. 5,260,203, the disclosures of which are incorporated by reference in their entireties.

“Single-chain Fv” or “scFv” antibody fragments comprise the V_{H} and V_{L} domains of antibody, wherein these domains are present in a single polypeptide chain, and optionally comprising a polypeptide linker between the V_{H} and V_{L} domains that enables the Fv to form the desired structure for antigen binding (Bird et al., Science 242:423-426, 1988, and Huston et al., Proc. Natl. Acad. Sci. USA 85:5879-5883, 1988). An “Fd” fragment consists of the V_{H} and C_{H}1 domains.

The term “diabodies” refers to small antibody fragments with two antigen-binding sites, which fragments comprise a heavy-chain variable domain (VH) connected to a light-chain variable domain (VL) in the same polypeptide chain (VH VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

The term “compete” when used in the context of antigen binding proteins (e.g., neutralizing antigen binding proteins or neutralizing antibodies) that compete for the same epitope means competition between antigen binding proteins is determined by an assay in which the antigen binding protein (e.g., antibody or immunologically functional fragment thereof) under test prevents or inhibits specific binding of a reference antigen binding protein (e.g., a ligand, or a reference antibody) to a common antigen (e.g., hOrai1 or a fragment thereof). Numerous types of competitive binding assays can be used, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using I-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabelled test antigen binding protein and a labeled reference antigen binding protein. Competitive inhibition is measured by determining the amount of label bound to the solid surface or cells in the presence of the test antigen binding protein. Usually the test antigen binding protein is present in excess. Antigen binding proteins identified by competition assay (competing antigen binding proteins) include antigen binding proteins binding to the same epitope as the reference antigen binding proteins and antigen binding proteins binding to an adjacent epitope sufficiently proximal to the epitope bound by the reference antigen binding protein for steric hindrance to occur. Additional details regarding methods for determining competitive binding are provided in the examples herein. Usually, when a competing antigen binding protein is present in excess, it will inhibit specific binding of a reference antigen binding protein to a common antigen by at least 40%, 45%, 50%, 55%, 60%, 65%, 70% or 75%. In some instance, binding is inhibited by at least 80%, 85%, 90%, 95%, or 97% or more.

The term “antigen” refers to a molecule or a portion of a molecule capable of being bound by a selective binding agent, such as an antigen binding protein (including, e.g., an antibody or immunological functional fragment thereof), and additionally capable of being used in an animal to produce antibodies capable of binding to that antigen. An antigen may possess one or more epitopes that are capable of interacting with different antigen binding proteins, e.g., antibodies.

The term “epitope” is the portion of a molecule that is bound by an antigen binding protein (for example, an antibody). The term includes any determinant capable of specifically binding to an antigen binding protein, such as an antibody or to a T-cell receptor. An epitope can be contiguous or non-contiguous (e.g., in a single-chain polypeptide, amino acid residues that are not contiguous to one another in the polypeptide sequence but that within the context of the molecule are bound by the antigen binding protein). In certain embodiments, epitopes may be mimetic in that they comprise a three dimensional structure that is similar to an epitope used to generate the antigen binding protein, yet comprise none or only some of the amino acid residues found in that epitope used to generate the antigen binding protein. Most often, epitopes reside on proteins, but in some instances may reside on other kinds of molecules, such as nucleic acids. Epitope determinants may include chemically active surface groupings of molecules such as amino acids, sugar side chains, phosphoryl or sulfonyl groups, and may have specific three dimensional structural characteristics, and/or specific charge characteristics. Generally, antibodies specific for a particular target antigen will preferentially recognize an epitope on the target antigen in a complex mixture of proteins and/or macromolecules.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) must be addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073. For example, sequence identity can be determined by standard methods that are commonly used to compare the similarity in position of the amino acids of two polypeptides. Using a computer program such as BLAST or FASTA, two polypeptide or two polynucleotide sequences are aligned for optimal matching of their respective residues (either along the full length of one or both sequences, or along a pre-determined portion of one or both sequences). The programs provide a default opening penalty and a default gap penalty, and a scoring matrix such as PAM 250 [a standard scoring matrix; see Dayhoff et al., in Atlas of Protein Sequence and Structure, vol. 5, supp. 3 (1978)] can be used in conjunction with the computer program. For example, the percent identity can then be calculated as: the total number of identical matches multiplied by 100 and then divided by the sum of the length of the longer sequence within the matched span and the number of gaps introduced into the longer sequences in order to align the two sequences. In calculating percent identity, the sequences being compared are aligned in a way that gives the largest match between the sequences.

The GCG program package is a computer program that can be used to determine percent identity, which package includes GAP (Devereux et al., 1984, Nucl. Acid Res. 12:387; Genetics Computer Group, University of Wisconsin, Madison, Wis.). The computer algorithm GAP is used to align the two polypeptides or two polynucleotides for which the percent sequence identity is to be determined. The sequences are aligned for optimal matching of their respective amino acid or nucleotide (the “matched span”, as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

Recommended parameters for determining percent identity for polypeptides or nucleotide sequences using the GAP program include the following:

Algorithm: Needleman et al., 1970, J. Mol. Biol. 48:443-453;

Comparison matrix: BLOSUM 62 from Henikoff et al., 1992, supra;

Gap Penalty: 12 (but with no penalty for end gaps)

Gap Length Penalty: 4

Threshold of Similarity: 0

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least 50 contiguous amino acids of the target polypeptide.

The term “modification” when used in connection with antigen binding proteins, including antibodies and antibody fragments, of the invention, include, but are not limited to, one or more amino acid changes (including substitutions, insertions or deletions); chemical modifications; covalent modification by conjugation to therapeutic or diagnostic agents; labeling (e.g., with radionuclides or various enzymes); covalent polymer attachment such as PEGylation (derivatization with polyethylene glycol) and insertion or substitution by chemical synthesis of non-natural amino acids. Modified antigen binding proteins of the invention will retain the binding properties of unmodified molecules of the invention.

The term “derivative” when used in connection with antigen binding proteins (including antibodies and antibody fragments) of the invention refers to antigen binding proteins that are covalently modified by conjugation to therapeutic or diagnostic agents, labeling (e.g., with radionuclides or various enzymes), covalent polymer attachment such as PEGylation (derivatization with polyethylene glycol) and insertion or substitution by chemical synthesis of non-natural amino acids. Derivatives of the invention will retain the binding properties of underivatized molecules of the invention.

An embodiment of the isolated antigen binding protein that “inhibits human calcium response-activated calcium (CRAC) channel activity” is one that in a test sample (i) reduces, decreases, or eliminates CRAC current (IC_{RAC} or ICRAC), as measured using well known electrophysiological techniques and suitable equipment, for example those employed in Example 6 herein; and/or (ii) reduces, decreases, or eliminates calcium influx through CRAC channels as measured using well known FLIPR techniques and fluorescence detection/imaging equipment (e.g., Example 6 herein) or well known ratiometric calcium influx assay techniques (e.g., Example 5 herein). The inhibition of CRAC channel activity is detected and recorded relative to CRAC channel activity in the same test sample before exposure to the antigen binding protein, or in a different, comparable test sample not exposed to the antigen binding protein.

An embodiment of the isolated antigen binding protein that “inhibits release of IL-2, IFN-gamma, or both, in thapsigargin-treated human whole blood” is one that in a test sample in the human whole blood ex vivo assay, disclosed in Example 4 herein, reduces, decreases, or eliminates release of IL-2, IFN-gamma, or both. The inhibition of release of IL-2, IFN-gamma, or both, is detected relative to release of IL-2, IFN-gamma, or both, in comparable test samples not exposed to the antigen binding protein.

An embodiment of the isolated antigen binding protein that “inhibits NFAT-mediated expression” is one that in a test sample of the NFAT-Luciferase Reporter assay, disclosed in Example 5 herein, reduces, decreases, or eliminates detectable expression of the luciferase reporter gene relative to a comparable test sample not exposed to the antigen binding protein.

Immunoglobulin Embodiments of Antigen Binding Proteins

In full-length immunoglobulin light and heavy chains, the variable and constant regions are joined by a “J” region of about twelve or more amino acids, with the heavy chain also including a “D” region of about ten more amino acids. See, e.g., Fundamental Immunology, 2nd ed., Ch. 7 (Paul, W., ed.) 1989, New York: Raven Press (hereby incorporated by reference in its entirety for all purposes). The variable regions of each light/heavy chain pair typically form the antigen binding site.

One example of a human IgG2 heavy chain (HC) constant domain has the amino acid sequence:

SEQ. ID NO: 22
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDP
EVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDIAVEWESNGQPENNYKTTPPMLDSDGSFFLYSKLTVDKSRWQQGN
VFSCSVMHEALHNHYTQKSLSLSPGK//.

Constant region sequences of other IgG isotypes are known in the art for making recombinant versions of the inventive antigen binding protein having an IgG1, IgG2, IgG3, or IgG4 immunoglobulin isotype, if desired. In general, human IgG2 can be used for targets where effector functions are not desired, and human IgG1 in situations where such effector functions (e.g., antibody-dependent cytotoxicity (ADCC)) are desired. Human IgG3 has a relatively short half life and human IgG4 forms antibody “half-molecules.” There are four known allotypes of human IgG1. The preferred allotype is referred to as “hIgG1z”, also known as the “KEEM” allotype. Human IgG1 allotypes “hIgG1za” (KDEL), “hIgG1f” (REEM), and “hIgG1fa” are also useful; all appear to have ADCC effector function.

Human hIgG1z heavy chain (HC) constant domain has the amino acid sequence:

SEQ ID NO: 243
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK//.

Human hIgG1za heavy chain (HC) constant domain has the amino acid sequence:

SEQ ID NO: 244
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK//.

Human hIgG1f heavy chain (HC) constant domain has the amino acid sequence:

SEQ ID NO: 245
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK//.

Human hIgG1fa heavy chain (HC) constant domain has the amino acid sequence:

SEQ ID NO: 246
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK//.

One example of a human immunoglobulin light chain (LC) constant region sequence is the following (designated “CL-1”):

SEQ ID NO: 14
GQPKANPTVTLFPPSSEELQANKATLVCLISDFYPGAVTVAWKADGSPVK
AGVETTKPSKQSNNKYAASSYLSLTPEQWKSHRSYSCQVTHEGSTVEKTV
APTECS//.

CL-1 is useful to increase the pI of antibodies and is convenient. There PGP-34DNA are three other human immunoglobulin light chain constant regions, designated “CL-2”, “CL-3” and “CL-7”, which can also be used within the scope of the present invention. CL-2 and CL-3 are more common in the human population.

CL-2 human light chain (LC) constant domain has the amino acid sequence:

SEQ ID NO: 247
GQPKAAPSVTLFPPSSEELQANKATLVCLISDFYPGAVTVAWKADSSPVK
AGVETTTPSKQSNNKYAASSYLSLTPEQWKSHRSYSCQVTHEGSTVEKTV
APTECS//.

CL-3 human LC constant domain has the amino acid sequence:

SEQ ID NO: 248
GQPKAAPSVTLFPPSSEELQANKATLVCLISDFYPGAVTVAWKADSSPVK
AGVETTTPSKQSNNKYAASSYLSLTPEQWKSHKSYSCQVTHEGSTVEKTV
APTECS//.

CL-7 human LC constant domain has the amino acid sequence:

SEQ ID NO: 249
GQPKAAPSVTLFPPSSEELQANKATLVCLVSDFYPGAVTVAWKADGSPVK
VGVETTKPSKQSNNKYAASSYLSLTPEQWKSHRSYSCRVTHEGSTVEKTV
APAECS//.

Variable regions of immunoglobulin chains generally exhibit the same overall structure, comprising relatively conserved framework regions (FR) joined by three hypervariable regions, more often called “complementarity determining regions” or CDRs. The CDRs from the two chains of each heavy chain/light chain pair mentioned above typically are aligned by the framework regions to form a structure that binds specifically with a specific epitope or domain on the target protein (e.g., hOrai1). From N-terminal to C-terminal, naturally-occurring light and heavy chain variable regions both typically conform with the following order of these elements: FR1, CDR1, FR2, CDR2, FR3, CDR3 and FR4. A numbering system has been devised for assigning numbers to amino acids that occupy positions in each of these domains. This numbering system is defined in Kabat Sequences of Proteins of Immunological Interest (1987 and 1991, NIH, Bethesda, Md.), or Chothia & Lesk, 1987, J. Mol. Biol. 196:901-917; Chothia et al., 1989, Nature 342:878-883.

Specific examples of some of the full length light and heavy chains of the antibodies that are provided and their corresponding amino acid sequences are summarized in Table 1A and Table 1B below. Table 1A shows exemplary light chain sequences, all of which have a common constant region lambda constant region 1 (CL-1; SEQ ID NO:14) for all lambda light chains. Table 1B shows exemplary heavy chain sequences, all of which include constant region human IgG2 (SEQ ID NO:22). However, encompassed within the present invention are immunoglobulins with sequence changes in the constant or framework regions of those listed in Table 1A and/or Table 1B (e.g. IgG4 vs IgG2, CL2 vs CL1). Also, the signal peptide (SP) sequences for the L1-L3 sequence in Table 1A and H1-H4 sequences in Table 1B are the same, i.e., the VK-1 SP (MDMRVPAQLLGLLLLWLRGARC// SEQ ID NO:234; single underlined) that is used in the high throughput cloning process, but any other suitable signal peptide sequence may be employed within the scope of the invention. Some examples of useful signal peptide sequences include:

(SEQ ID NO: 329)
MEAPAQLLFLLLLWLPDTTG,
(SEQ ID NO: 330)
MEWTWRVLFLVAAATGAHS,
(SEQ ID NO: 331)
METPAQLLFLLLLWLPDTTG,
and
(SEQ ID NO: 332)
MKHLWFFLLLVAAPRWVLS.

Another example of a useful signal peptide sequence is VH21 SP MEWSWVFLFFLSVTTGVHS (SEQ ID NO:332).

TABLE 1A
Immunoglobulin Light Chain Sequences. Signal peptide sequences,
when present, are indicated by a single underline,
CDR regions are indicated by double underline,
and framework and constant regions are not underlined.

| SEQ | | Contained | |
|---|---|---|---|
| ID | Desig- | in | |
| NO: | nation | Clone(s) | Sequence |
| 31 | L1 | 2D2.1 2C1.1 2G1.1 | 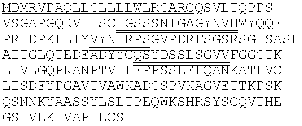 |
| 322 | L4 | 2D2.1 2C1.1 2G1.1 | 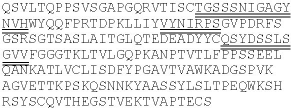 |
| 32 | L2 | 2B7.1 | 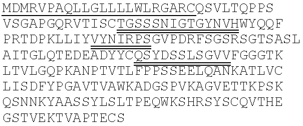 |
| 323 | L5 | 2B7.1 | 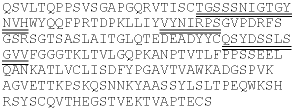 |
| 30 | L3 | 2B4.1 | 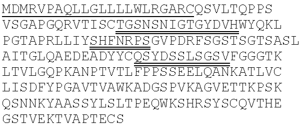 |
| 324 | L6 | 2B4.1 | 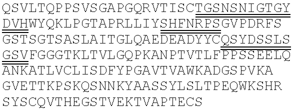 |
| 333 | L7 | 5F7.1 | QSVLTQPPSVSGAPGQRVTISCTGSSSNIGAGFD |
| | | | VHWYQQLPVTAPKLLIYGNRNRPSGVPARFSG |
| | | | SKSGTSASLAITGLQAEDEAVYYCQSYDSSLTV |
| | | | FGGGTKLTVLGQPKANPTVTLFPPSSEELQANK |
| | | | ATLVCLISDFYPGAVTVAWKADGSPVKAGVET |
| | | | TKPSKQSNNKYAASSYLSLTPEQWKSHRSYSC |
| | | | QVTHEGSTVEKTVAPTECS |
| 334 | L8 | 5F7.1 | SCELTQSPSVSVSPGQTARITCSGDALPKKYAC |
| | | | CYQQKSGQAPVLVVYDDHKRPSGIPERFSGSSS |
| | | | GTLATLIISGAQVEDEADSYCYSTDSSGNHSWV |
| | | | FGGGTKLTVLGQPKANPTVTLFPPSSEELQANK |
| | | | ATLVCLISDFYPGAVTVAWKADGSPVKAGVET |
| | | | TKPSKQSNNKYAASSYLSLTPEQWKSHRSYSC |
| | | | QVTHEGSTVEKTVAPTECS |
| 335 | L9 | 5C1.1 | QSVLTQPPSVSGAPGQRVTISCTGSSSNIGAGYD |
| | | 5H3.1 | VHWYQQLPGTAPKLLIYGNSNRPSGVPDRFSG |
| | | | SKSGTSASLAITGLQAEDEADYYCQSYDNRLSD |
| | | | SVVIGGGTKLAVQGQPKANPTVTLFPPSSEELQ |
| | | | ANKATLVCLISDFYPGAVTVAWKADGSPVKA |
| | | | GVETTKPSKQSNNKYAASSYLSLTPEQWKSHR |
| | | | SYSCQVTHEGSTVEKTVAPTECS |
| 336 | L10 | 5C1.1 | QSALTQPPSASGSPGQSVTSSCTGTSSDVGGYN |
| | | 5H3.1 | YVSWYQQQPGKAPKLMIYEVSKRPSGVPDRFS |
| | | | GSKSGNTASLTVSGLQAEDEADYYFSSYAGSN |
| | | | NFDVFGTGTKVTVLGQPKANPTVTLFPPSSEEL |
| | | | QANKATLVCLISDFYPGAVTVAWKADGSPVK |
| | | | AGVETTKPSKQSNNKYAASSYLSLTPEQWKSH |
| | | | RSYSCQVTHEGSTVEKTVAPTECS |
| 337 | L11 | 5D7.2 | QSVLTQPPSVSGAPGQRVTISCTGSSSNIGAGYD |
| | | | VHWYQQSPGTAPKLLIYGNSNRPSGVPDRFSGS |
| | | | KSGTSASLAITGLQAEDEADYYCQSYDNRLSDS |
| | | | VVIGGGTKLTVQKQPKANPTVTLFPPSSEELQA |
| | | | NKATLVCLISDFYPGAVTVAWKADGSPVKAG |
| | | | VETTKPSKQSNNKYAASSYLSLTPEQWKSHRS |
| | | | YSCQVTHEGSTVEKTVAPTECS |
| 338 | L12 | 5F2.1 | QSVLTQPPSVSGAPGQRVTISCTGSRSNIGAGY |
| | | | DVHWYQQLPRTAPKLLIYDNSNRPSGVPDRFS |
| | | | GSKSGSSASLAITGLQAEDEADYYCQSYDNSLS |
| | | | DSVLIGGGTKLTVLGQPKANPTVTLFPPSSEEL |
| | | | QANKATLVCLISDFYPGAVTVAWKADGSPVK |
| | | | AGVETTKPSKQSNNKYAASSYLSLTPEQWKSH |
| | | | RSYSCQVTHEGSTVEKTVAPTECS |
| 339 | L13 | 5F2.1 | QSALTQPPSASGSPGQSVTSSCTGTSSDVGGYN |
| | | | YVSWYQQHPGKAPKLMIYEVSKRPSGVPDWFS |
| | | | GSKSGNTASLTVSGLQAEDEADYYYNSYSGSN |
| | | | NFDVFGTGTKVTVLGQPKANPTVTLFPPSSEEL |
| | | | QANKATLVCLISDFYPGAVTVAWKADGSPVK |
| | | | AGVETTKPSKQSNNKYAASSYLSLTPEQWKSH |
| | | | RSYSCQVTHEGSTVEKTVAPTECS |
| 340 | L14 | 5A4.2 | QSVLTQPPSVSGAPGQRVTISCTGSSSNIGAGYD |
| | | | VHWYQQLPGTAPKLLIYGNSNRPSGVPDRFSG |
| | | | SKSGTSASLAITGLQAEDEADYYCQSYDNRLSD |
| | | | SVVIGGGTKLTVQGQPKANPTVTLFPPSSEELQ |
| | | | ANKATLVCLISDFYPGAVTVAWKADGSPVKA |
| | | | GVETTKPSKQSNNKYAASSYLSLTPEQWKSHR |
| | | | SYSCQVTHEGSTVEKTVAPTECS |
| 341 | L15 | 5B1.1 | QSVLTQPPSVSGAPGQRVTISCTGSSSNIGAGYD |
| | | | VHWYQQLPGTAPKLLIYGNSNRPSGVPDRFSG |
| | | | SKSGTSASLAITKFQAEDEAVYYCQSYGSGLSG |
| | | | VVFGGGTKLTVLGQPKANPTVTLFPPSSEELQA |
| | | | NKATLVCLISDFYPGAVTVAWKADGSPVKAG |
| | | | VETTKPSKQSNNKYAASSYLSLTPEQWKSHRS |
| | | | YSCQVTHEGSTVEKTVAPTECS |
| 342 | L16 | 5B5.1 | QSVLTQPPSVSGAPGQRVTISCTGSNSNIGAGFD |
| | | 5B5.2 | VHWYQQLPGTVPKLLIYGNNNRPSGVPDRFSG |
| | | | SKSGTSASLAITGLQAEDEADYYCQSYDSRLTV |
| | | | FGGGTKLTVLGQPKANPTVTLFPPSSEELQANK |
| | | | ATLVCLISDFYPGAVTVAWKADGSPVKAGVET |
| | | | TKPSKQSNNKYAASSYLSLTPEQWKSHRSYSC |
| | | | QVTHEGSTVEKTVAPTECS |

```
TABLE 1B
  Immunoglobulin Heavy Chain Sequences. Signal peptide sequences,
when present, are indicated by a single underline, CDR
regions are indicated by double underline,
    and framework and constant regions are not underlined.
```

| SEQ | Desig- | Contained | |
|---|---|---|---|
| ID NO: | nation | in Clone | Sequence |
| 33 | H1 | 2D2.1 | 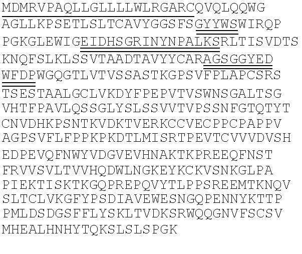 |
| 325 | H5 | 2D2.1 | 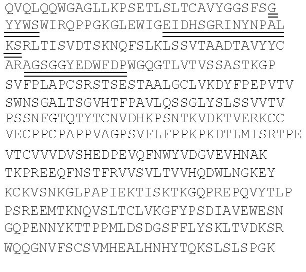 |
| 34 | H2 | 2C1.1 | 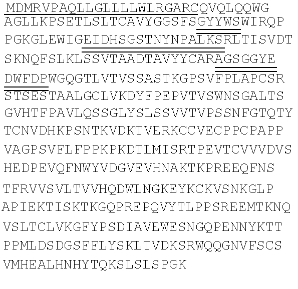 |
| 326 | H6 | 2C1.1 | 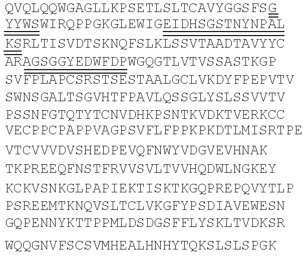 |
| 35 | H3 | 2G1.1 | 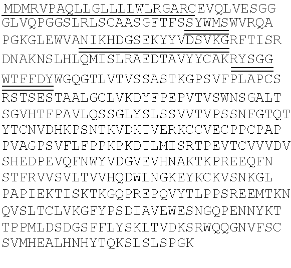 |
| 327 | H7 | 2G1.1 | 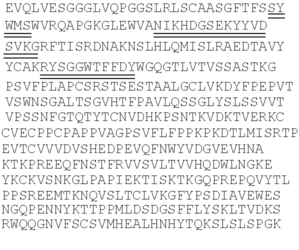 |
| 29 | H4 | 2B4.1 | 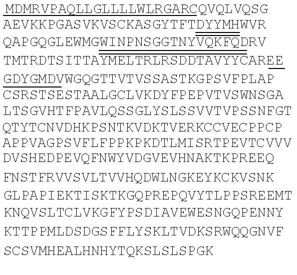 |
| 328 | H8 | 2B4.1 | 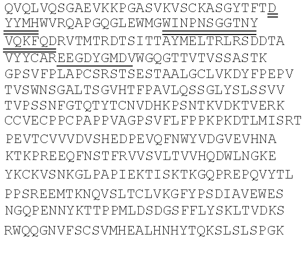 |
| 343 | H9 | 5F7.1 5A1.1 | 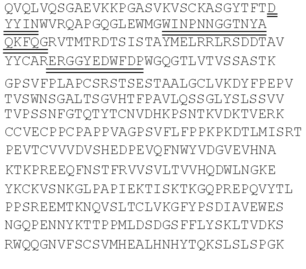 |
| 344 | H10 | 5C1.1 5H3.1 5D7.2 | 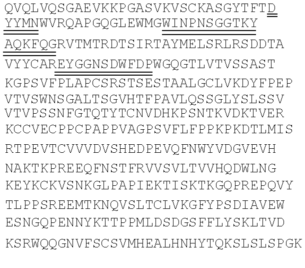 |
| 345 | H11 | 5F2.1 | 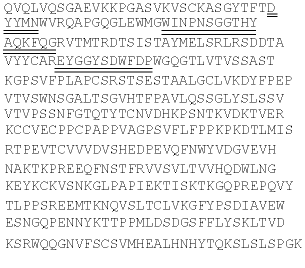 |
| 346 | H12 | 5A4.2 | 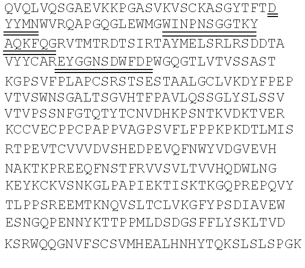 |
| 347 | H13 | 5B1.1 | 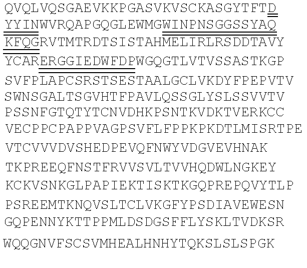 |
| 348 | H14 | 5B5.1 5B5.2 | 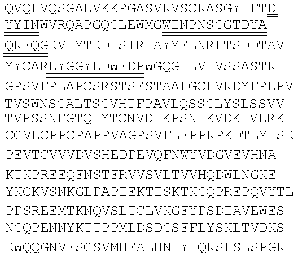 |

Some embodiments of the isolated antigen binding protein comprising an antibody or antibody fragment, comprise:

(a) an immunoglobulin heavy chain having the amino acid sequence of SEQ ID NO: 29, SEQ ID NO:33, SEQ ID NO:34, or SEQ ID NO:35, or comprising the foregoing sequence from which one, two, three, four or five amino acid residues are lacking from the N-terminal or C-terminal, or both; or

(b) an immunoglobulin light chain having the amino acid sequence of SEQ ID NO: 30, SEQ ID NO:31, or SEQ ID NO:32, or comprising the foregoing sequence from which one, two, three, four or five amino acid residues are lacking from the N-terminal or C-terminal, or both; or

(c) the immunoglobulin heavy chain of (a) and the immunoglobulin light chain of (b).

Some other embodiments of the isolated antigen binding protein comprising an antibody or antibody fragment, in which the signal peptide sequences are absent from the heavy chain and light chain, comprise:

(a) an immunoglobulin heavy chain comprising the amino acid sequence of SEQ ID NO: 325, SEQ ID NO:326, SEQ ID NO:327, SEQ ID NO:328, SEQ ID NO:343, SEQ ID NO:344, SEQ ID NO:345, SEQ ID NO:346, SEQ ID NO:347, or SEQ ID NO:348, or comprising the foregoing sequence from which one, two, three, four or five amino acid residues are lacking from the N-terminal or C-terminal, or both; or

(b) an immunoglobulin light chain comprising the amino acid sequence of SEQ ID NO: 322, SEQ ID NO:323, SEQ ID NO:324, SEQ ID NO:333, SEQ ID NO:334, SEQ ID NO:335, SEQ ID NO:336, SEQ ID NO:337, SEQ ID NO:338, SEQ ID NO:339, SEQ ID NO:340, SEQ ID NO:341, or SEQ ID NO:342, or comprising the foregoing sequence from which one, two, three, four or five amino acid residues are lacking from the N-terminal or C-terminal, or both; or

(c) the immunoglobulin heavy chain of (a) and the immunoglobulin light chain of (b).

Again, each of the exemplary heavy chains (H1, H2, H3, H4, H5, H6, H7, H8, H9, H10, H11, H12, H13, or H14) listed in Table 1B can be combined with any of the exemplary light chains shown in Table 1A to form an antibody. Examples of such combinations include H1 combined with any of L1 through L16; H2 combined with any of L1 through L16; H3 combined with any of L1 through L16, H4 combined with any of L1 through L16, and so on. In some instances, the antibodies include at least one heavy chain and one light chain from those listed in Table 1A and 1B. In some instances, the antibodies comprise two different heavy chains and two different light chains listed in Table 1A and Table 1B. In other instances, the antibodies contain two identical light chains and two identical heavy chains. As an example, an antibody or immunologically functional fragment may include two H1 heavy chains and two L1 light chains, or two H2 heavy chains and two L2 light chains, or two H3 heavy chains and two L3 light chains and other similar combinations of pairs of light chains and pairs of heavy chains as listed in Table 1A and Table iB.

Other antigen binding proteins that are provided are variants of antibodies formed by combination of the heavy and light chains shown in Tables iA and Table 1B and comprise light and/or heavy chains that each have at least 70%, at least 75%, at least 80%, at least 85%, at least 90%, at least 95%, at least 97% or at least 99% identity to the amino acid sequences of these chains. In some instances, such antibodies include at least one heavy chain and one light chain, whereas in other instances the variant forms contain two identical light chains and two identical heavy chains.

It is within the scope of the invention that the heavy chain(s) and/or light chain(s) may have one, two, three, four or five amino acid residues lacking from the N-terminal or C-terminal, or both, in relation to any one of the heavy and light chains set forth in Tables iA and Table 1B, e.g., due to post-translational modifications. For example, CHO cells typically cleave off a C-terminal lysine.

Variable Domains of Antibodies

The various heavy chain and light chain variable regions provided herein are depicted in Table 2. Each of these variable regions may be attached to the above heavy and light chain constant regions to form a complete antibody heavy and light chain, respectively. Further, each of the so generated heavy and light chain sequences may be combined to form a complete antibody structure. It should be understood that the heavy chain and light chain variable regions provided herein can also be attached to other constant domains having different sequences than the exemplary sequences listed above.

Also provided are antigen binding proteins, including antibodies or antibody fragments, that contain or include at least one immunoglobulin heavy chain variable region selected from V_{H}1, V_{H}2, V_{H}3, V_{H}4, V_{H}5, V_{H}6, V_{H}7, V_{H}8, V_{H}9, and V_{H}10 and/or at least one immunoglobulin light chain variable region selected from V_{L}1, V_{L}2, V_{L}3, V_{L}4, V_{L}5, V_{L}6, V_{L}7, V_{L}8, V_{L}9, V_{L}10, V_{L}11, V_{L}12, and V_{L}13, as shown in Table 2 below, and immunologically functional fragments, derivatives, muteins and variants of these light chain and heavy chain variable regions.

Antigen binding proteins of this type can generally be designated by the formula “V_{H}x/V_{L}y,” where “x” corresponds to the number of heavy chain variable regions included in the antigen binding protein and “y” corresponds to the number of the light chain variable regions included in the antigen binding protein (in general, x and y are each 1 or 2).

```
TABLE 2
  Exemplary V_{H} and V_{L} Chains: CDR regions are indicated by
double underline, and framework regions are not underlined.
Optional N-terminal signal sequences are not shown
    (See, SEQ ID NOS:15-20 and 23-28).
```

| Contained | Desig- | | |
|---|---|---|---|
| in Clone | nation | SEQ ID NO | Amino Acid Sequence |
| 2D2.1 | VL1 | 36 | QSVLTQPPSVSGAPGQRVTISCTGSSSNIGTGYNVHWY |
| 2C1.1 | | | QQFPRTDPKLLIYVYNIRPSGVPDRFSGSRSGTSASLAIT |
| 2G1.1 | | | GLQTEDEADYYCQSYDSSLSGVVFGGGTKLTVL |
| 2B7.1 | VL2 | 37 | QSVLTQPPSVSGAPGQRVTISCTGSSSNIGTGYNVHWY |
| | | | QQFPRTDPKLLIYVYNIRPSGVPDRFSGSRSGTSASLAIT |
| | | | GLQTEDEADYYCCQSYDSSLSGVVFGGGTKLTVL |
| 2B4.1 | VL3 | 38 | QSVLTQPPSVSGAPGQRVTISCTGSNSNIGTGYDVHWY |
| | | | QKLPGTAPRLLIYSHFNRPSGVPDRFSGSTSGTSASLAIT |
| | | | GLQAEDEADYYCCQSYDSSLSGSVFGGGTKLTVL |
| 5F7.1 | VL4 | 256 | QSVLTQPPSVSGAPGQRVTISCTGSSSNIGAGFDVHWY |
| 5A1.1 | | | QQLPVTAPKLLIYGNRNRPSGVPARFSGSKSGTSASLAI |
| | | | TGLQAEDEAVYYCCQSYDSSLTVFGGGTKLTVL |
| 5B1.1 | VL5 | 257 | QSVLTQPPSVSGAPGQRVTISCTGSSSNIGAGYDVHWY |
| | | | QQLPGTAPKLLIYGNSNRPSGVPDRFSGSKSGTSASLAI |
| | | | TKFQAEDEAVYYCCQSYGSGLSGVVFGGGTKLTVL |
| 5F7.1 | VL6 | 258 | SCELTQSPSVSVSPGQTARITCSGDALPKKYACCYQQK |
| 5A1.1 | | | SGQAPVLVVYDDHKRPSGIPERFSGSSSGTLATLIISGA |
| | | | QVEDEADSYCYSTDSSGNHSWVFGGGTKLTVL |
| 5B5.1 | VL7 | 259 | QSVLTQPPSVSGAPGQRVTISCTGSNSNIGAGFDVHWY |
| 5B5.2 | | | QQLPGTVPKLLIYGNNNRPSGVPDRFSGSKSGTSASLAI |
| | | | TGLQAEDEADYYCCQSYDSRLTVFGGGTKLTVL |
| 5F2.1 | VL8 | 260 | QSVLTQPPSVSGAPGQRVTISCTGSRSNIGAGYDVHWY |
| | | | QQLPRTAPKLLIYDNSNRPSGVPDRFSGSKSGSSASLAI |
| | | | TGLQAEDEADYYCCQSYDNSLSDSVLIGGGTKLTVL |
| 5F2.1 | VL9 | 261 | QSALTQPPSASGSPGQSVTSSCTGTSSDVGGYNYVSWY |
| | | | QQHPGKAPKLMIYEVSKRPSGVPDWFSGSKSGNTASL |
| | | | TVSGLQAEDEADYYYNSYSGSNNFDVFGTGTKVTVL |
| 5H3.1 | VL10 | 262 | QSVLTQPPSVSGAPGQRVTISCTGSSSNIGAGYDVHWY |
| 5C1.1 | | | QQLPGTAPKLLIYGNSNRPSGVPDRFSGSKSGTSASLAI |
| | | | TGLQAEDEADYYCCQSYDNRLSDSVVIGGGTKLAVQ |
| 5H3.1 | VL11 | 263 | QSALTQPPSASGSPGQSVTSSCTGTSSDVGGYNYVSWY |
| 5C1.1 | | | QQQPGKAPKLMIYEVSKRPSGVPDRFSGSKSGNTASLT |
| | | | VSGLQAEDEADYYFSSYAGSNNFDVFGTGTKVTVL |
| 5D7.2 | VL12 | 264 | QSVLTQPPSVSGAPGQRVTISCTGSSSNIGAGYDVHWY |
| | | | QQSPGTAPKLLIYGNSNRPSGVPDRFSGSKSGTSASLAI |
| | | | TGLQAEDEADYYCQSYDNRLSDSVVIGGGTKLTVQ |
| 5A4.2 | VL13 | 265 | QSVLTQPPSVSGAPGQRVTISCTGSSSNIGAGYDVHWY |
| | | | QQLPGTAPKLLIYGNSNRPSGVPDRFSGSKSGTSASLAI |
| | | | TGLQAEDEADYYCQSYDNRLSDSVVIGGGTKLTVQ |
| 2D2.1 | VH1 | 39 | 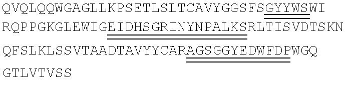 |
| 2C1.1 2B7.1 | VH2 | 40 | 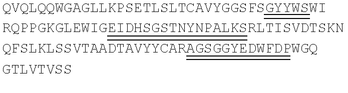 |
| 2G1.1 | VH3 | 41 | 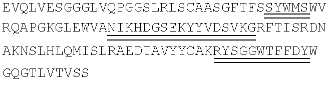 |
| 2B4.1 | VH4 | 42 | QVQLVQSGAEVKKPGASVKVSCKASGYTFTDYYMHW |
| | | | VRQAPGQGLEWMGWINPNSGGTNYVQKFQDRVTMT |
| | | | RDTSITTAYMELTRLRSDDTAVYYCAREEGDYGMDV |
| | | | WGQGTTVTVSS |
| 5F7.1 | VH5 | 250 | QVQLVQSGAEVKKPGASVKVPCKASGYTFTDYYINW |
| 5A1.1 | | | VRQAPGQGLEWMGWINPNNGGTNYAQKFQGRVTMT |
| | | | RDTSISTAYMELRRLRSDDTAVYYCARERGGYEDWFD |
| | | | PWGQGTLVTVSS |
| 5B1.1 | VH6 | 251 | QVQLVQSGAEVKKPGASVKVSCKASGYTFTDYYINW |
| | | | VRQAPGQGLEWMGWINPNSGGSSYAQKFQGRVTMTR |
| | | | DTSISTAHMELIRLRSDDTAVYYCARERGGIEDWFDPW |
| | | | GQGTLVTVSS |
| 5B5.1 | VH7 | 252 | QVQLVQSGAEVKKPGASVKVSCKASGYTFTDYYINW |
| 5B5.2 | | | VRQAPGQGLEWMGWINPNSGGTDYAQKFQGRVTMT |
| | | | RDTSIRTAYMELNRLTSDDTAVYYCAREYGGYEDWFD |
| | | | PWGQGTLVTVSS |
| 5F2.1 | VH8 | 253 | QVQLVQSGAEVKKPGASVKVSCKASGYTFTDYYMNW |
| | | | VRQAPGQGLEWMGWINPNSGGTHYAQKFQGRVTMT |
| | | | RDTSISTAYMELSRLRSDDTAVYYCAREYGGNSDWFD |
| | | | PWGQGTLVTVSS |
| 5H3.1 | VH9 | 254 | QVQLVQSGAEVKKPGASVKVSCKASGYTFTDYYMNW |
| 5D7.2 | | | VRQAPGQGLEWMGWINPNSGGTHYAQKFQGRVTMT |
| 5C1.1 | | | RDTSIRTAYMELSRLRSDDTAVYYCAREYGGNSDWFD |
| | | | PWGQGTLVTVSS |
| 5A4.2 | VH10 | 255 | QVQLVQSGAEVKKGASVKVSCKASGYTFTDYYMNW |
| | | | VRQAPGQGLEWMGWINPNSGGTKYAQKFQGRVTMT |
| | | | RDTSIRTAYMELSRLRSDDTAVYYCSREYGGNSDWFD |
| | | | EWGQGTLVTVSS |

Some embodiments of the isolated antigen binding protein that comprises an antibody or antibody fragment, comprising an immunoglobulin heavy chain variable region and an immunoglobulin light chain variable region:

(a) the heavy chain variable region comprises an amino acid sequence at least 95% identical to SEQ ID NO:39, SEQ ID NO:40, SEQ ID NO:41, SEQ ID NO:42, SEQ ID NO:250, SEQ ID NO:251; SEQ ID NO:252, SEQ ID NO:253, SEQ ID NO:254, or SEQ ID NO:255; or

(b) the light chain variable region comprises an amino acid sequence at least 95% identical to SEQ ID NO:36, SEQ ID NO:37, SEQ ID NO:38; SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, or SEQ ID NO:265; or

(c) the heavy chain variable region of (a) and the light chain variable region of (b).

Each of the heavy chain variable regions listed in Table 2, whether or not it is included in a larger heavy chain, may be combined with any of the light chain variable regions shown in Table 2 to form an antigen binding protein. Examples of such combinations include V_{H}1 combined with any of V_{L}1, V_{L}2, or V_{L}3; V_{H}2 combined with any of V_{L}1, V_{L}2, or V_{L}3; V_{H}3 combined with any of V_{L}1, V_{L}2, or V_{L}3; V_{H}4 combined with any of V_{L}1, V_{L}2, or V_{L}3, and so on.

In some instances, the antigen binding protein includes at least one heavy chain variable region and/or one light chain variable region from those listed in Table 2. In some instances, the antigen binding protein includes at least two different heavy chain variable regions and/or light chain variable regions from those listed in Table 2. An example of such an antigen binding protein comprises (a) one V_{H}1, and (b) one of V_{H}2, V_{H}3, or V_{H}4. Another example comprises (a) one V_{H}2, and (b) one of V_{H}1, V_{H}3, or V_{H}4. Again another example comprises (a) one V_{H}3, and (b) one of V_{H}1, V_{H}2, or V_{H}4. Again another example comprises (a) one V_{H}4, and (b) one of V_{H}1, V_{H}2, or V_{H}3, etc.

Again another example of such an antigen binding protein comprises (a) one V_{L}1, and (b) one of V_{L}2 or V_{L}3. Again another example of such an antigen binding protein comprises (a) one V_{L}2, and (b) one of V_{L}1 or V_{L}3. Again another example of such an antigen binding protein comprises (a) one V_{L}3, and (b) one of V_{L}1 or V_{L}2, etc.

The various combinations of heavy chain variable regions may be combined with any of the various combinations of light chain variable regions.

In other instances, the antigen binding protein contains two identical light chain variable regions and/or two identical heavy chain variable regions. As an example, the antigen binding protein may be an antibody or immunologically functional fragment that includes two light chain variable regions and two heavy chain variable regions in combinations of pairs of light chain variable regions and pairs of heavy chain variable regions as listed in Table 2.

Some antigen binding proteins that are provided comprise a heavy chain variable domain comprising a sequence of amino acids that differs from the sequence of a heavy chain variable domain selected from V_{H}1, V_{H}2, V_{H}3, V_{H}4, V_{H}5, V_{H}6, V_{H}7, V_{H}8, V_{H}9, and V_{H}10 at only 1, 2, 3, 4, 5, 6, 7, 8, 9, 10, 11, 12, 13, 14 or 15 amino acid residues, wherein each such sequence difference is independently either a deletion, insertion or substitution of one amino acid, with the deletions, insertions and/or substitutions resulting in no more than 15 amino acid changes relative to the foregoing variable domain sequences. The heavy chain variable region in some antigen binding proteins comprises a sequence of amino acids that has at least 70%, at least 75%, at least 80%, at least 85%, at least 90%, at least 95%, at least 97% or at least 99% sequence identity to the amino acid sequences of the heavy chain variable region of V_{H}1, V_{H}2, V_{H}3, or V_{H}4.

Certain antigen binding proteins comprise a light chain variable domain comprising a sequence of amino acids that differs from the sequence of a light chain variable domain selected from V_{L}1, V_{L}2, V_{L}3, V_{L}4, V_{L} 5, V_{L}6, V_{L}7, V_{L}8, V_{L}9, V_{L}10, V_{L}11, V_{L}12, and V_{L}13 at only 1, 2, 3, 4, 5, 6, 7, 8, 9, 10, 11, 12, 13, 14 or 15 amino acid residues, wherein each such sequence difference is independently either a deletion, insertion or substitution of one amino acid, with the deletions, insertions and/or substitutions resulting in no more than 15 amino acid changes relative to the foregoing variable domain sequences. The light chain variable region in some antigen binding proteins comprises a sequence of amino acids that has at least 70%, at least 75%, at least 80%, at least 85%, at least 90%, at least 95%, at least 97% or at least 99% sequence identity to the amino acid sequences of the light chain variable region of V_{L}1, V_{L} 2, or V_{L} 3.

Still other antigen binding proteins, e.g., antibodies or immunologically functional fragments, include variant forms of a variant heavy chain and a variant light chain as described herein.

CDRs

The antigen binding proteins disclosed herein are polypeptides into which one or more CDRs are grafted, inserted and/or joined. An antigen binding protein can have 1, 2, 3, 4, 5 or 6 CDRs. An antigen binding protein thus can have, for example, one heavy chain CDR1 (“CDRH1”), and/or one heavy chain CDR2 (“CDRH2”), and/or one heavy chain CDR3 (“CDRH3”), and/or one light chain CDR1 (“CDRL1”), and/or one light chain CDR2 (“CDRL2”), and/or one light chain CDR3 (“CDRL3”). Some antigen binding proteins include both a CDRH3 and a CDRL3. Specific heavy and light chain CDRs are identified in Table 3A and Table 3B, respectively.

Complementarity determining regions (CDRs) and framework regions (FR) of a given antibody may be identified using the system described by Kabat et al. in Sequences of Proteins of Immunological Interest, 5th Ed., US Dept. of Health and Human Services, PHS, NIH, NIH Publication no. 91-3242, 1991. Certain antibodies that are disclosed herein comprise one or more amino acid sequences that are identical or have substantial sequence identity to the amino acid sequences of one or more of the CDRs presented in Table 3A (CDRHs) and Table 3B (CDRLs).

TABLE 3A
Exemplary CDRH Sequences

| Contained in | | | SEQ ID |
|---|---|---|---|
| Reference | Designation | Sequence | NO: |
| 2D2.1 VHCDR1 | CDRH 1-1 | GYYWS | 43 |
| 2C1.1 VHCDR1 | | | |
| 2G1.1 VHCDR1 | CDRH 1-2 | SYWMS | 44 |
| 2B4.1 VHCDR1 | CDRH 1-3 | DYYMH | 45 |
| 5F7.1 VHCDR1 | CDRH 1-4 | DYYIN | 266 |
| 5A1.1 VHCDR1 | | | |
| 5B1.1 VHCDR1 | | | |
| 5B5.1 VHCDR1 | | | |
| 5B5.2 VHCDR1 | | | |
| 5F2.1 VHCDR1 | CDRH 1-5 | DYYMN | 267 |
| 5H3.1 VHCDR1 | | | |
| 5C1.1 VHCDR1 | | | |
| 5A4.2 VHCDR1 | | | |
| 2D2.1 VHCDR2 | CDRH 2-1 | EIDHSGRINYNPALKS | 46 |
| 2C1.1 VHCDR2 | CDRH 2-2 | EIDHSGSTNYNPALKS | 47 |
| 2G1.1 VHCDR2 | CDRH 2-3 | NIKHDGSEKYYVDSVKG | 48 |
| 2B4.1 VHCDR2 | CDRH 2-4 | WINPNSGGTNYVQKFQD | 49 |
| 5F7.1 VHCDR2 | CDRH 2-5 | WINPNNGGTNYAQKFQG | 268 |
| 5A1.1 VHCDR2 | | | |
| 5B1.1 VHCDR2 | CDRH 2-6 | WINPNSGGSSYAQKFQG | 269 |
| 5B5.1 VHCDR2 | CDRH 2-7 | WINPNSGGTDYAQKFQG | 270 |
| 5B5.2 VHCDR2 | | | |
| 5F2.1 VHCDR2 | CDRH 2-8 | WINPNSGGTHYAQKFQG | 271 |
| 5H3.1 VHCDR2 | CDRH 2-9 | WINPNSGGTKYAQKFQG | 272 |
| 5C1.1 VHCDR2 | | | |
| 5A4.2 VHCDR2 | | | |
| 2C1.1 VHCDR3 | CDRH 3-1 | AGSGGYEDWFDP | 50 |
| 2D2.1 VHCDR3 | | | |
| 2G1.1 VHCDR3 | CDRH 3-2 | RYSGGWTFFDY | 51 |
| 2B4.1 VHCDR3 | CDRH 3-3 | EEGDYGMDV | 52 |
| 5A1.1 VHCDR3 | CDRH 3-4 | ERGGYEDWFDP | 273 |
| 5F7.1 VHCDR3 | | | |
| 5B1.1 VHCDR3 | CDRH 3-5 | ERGGIEDWFDP | 274 |
| 5B5.1 VHCDR3 | CDRH 3-6 | EYGGYEDWFDP | 275 |
| 5B5.2 VHCDR3 | | | |
| 5F2.1 VHCDR3 | CDRH 3-7 | EYGGYSDWFDP | 276 |
| 5A4.2 VHCDR3 | CDRH 3-8 | EYGGNSDWFDP | 277 |
| 5C1.1 VHCDR3 | | | |
| 5H3.1 VHCDR3 | | | |

TABLE 3B
Exemplary CDRL Sequences

| Contained in | | | |
|---|---|---|---|
| Reference | Designation | Sequence | SEQ ID NO: |
| 2D2.1 VLCDR1 | CDRL 1-1 | TGSSSNIGAGYNVH | 53 |
| 2B.7.1 VLCDR1 | CDRL 1-2 | TGSSSNIGTGYNVH | 54 |
| 2B4.1 VLCDR1 | CDRL 1-3 | TGSNSNIGTGYDVH | 55 |
| 5A1.1 VLCDR1 | CDRL 1-4 | TGSSSNIGAGFDVH | 278 |
| 5F7.1 VLCDR1 | | | |
| 5A4.2 VLCDR1 | CDRL 1-5 | TGSSSNIGAGYDVH | 279 |
| 5B1.1 VLCDR1 | | | |
| 5C1.1 VLCDR1 | | | |
| 5D7.2 VLCDR1 | | | |
| 5H3.1 VLCDR1 | | | |
| 5A1.1 VLCDR1 | CDRL 1-6 | SGDALPKKYAC | 280 |
| 5F7.1 VLCDR1 | | | |
| 5B5.1 VLCDR1 | CDRL 1-7 | TGSNSNIGAGFDVH | 281 |
| 5B5.2 VLCDR1 | | | |
| 5F2.1 VLCDR1 | CDRL 1-8 | TGSRSNIGAGYDVH | 282 |
| 5C1.1 VLCDR1 | CDRL 1-9 | TGTSSDVGGYNYVS | 283 |
| 5F2.1 VLCDR1 | | | |
| 5H3.1 VLCDR1 | | | |
| 2B.7.1 VLCDR2 | CDRL 2-1 | VYNIRPS | 56 |
| 2D2.1 VLCDR2 | | | |
| 2B4.1 VLCDR2 | CDRL 2-2 | SHFNRPS | 57 |
| 5A1.1 VLCDR2 | CDRL 2-3 | GNRNRPS | 284 |
| 5F7.1 VLCDR2 | | | |
| 5A4.2 VLCDR2 | CDRL 2-4 | GNSNRPS | 285 |
| 5B1.1 VLCDR2 | | | |
| 5C1.1 VLCDR2 | | | |
| 5D7.2 VLCDR2 | | | |
| 5H3.1 VLCDR2 | | | |
| 5F7.1 VLCDR2 | CDRL 2-5 | DDHKRPS | 286 |
| 5A1.1 VLCDR2 | | | |
| 5B5.1 VLCDR2 | CDRL 2-6 | GNNNRPS | 287 |
| 5B5.2 VLCDR2 | | | |
| 5F2.1 VLCDR2 | CDRL 2-7 | DNSNRPS | 288 |
| 5C1.1 VLCDR2 | CDRL 2-8 | EVSKRPS | 289 |
| 5F2.1 VLCDR2 | | | |
| 5H3.1 VLCDR2 | | | |
| 2D2.1 VLCDR3 | CDRL 3-1 | QSYDSSLSGVV | 58 |
| 2B.7.1 VLCDR3 | | | |
| 2B4.1 VLCDR3 | CDRL 3-2 | QSYDSSLSGSV | 59 |
| 5F7.1 VLCDR3 | CDRL 3-3 | QSYDSSLTV | 290 |
| 5A1.1 VLCDR3 | | | |
| 5B1.1 VLCDR3 | CDRL 3-4 | QSYGSGLSGVV | 291 |
| 5F7.1 VLCDR3 | CDRL 3-5 | YSTDSSGNHSWV | 292 |
| 5A1.1 VLCDR3 | | | |
| 5B5.1 VLCDR3 | CDRL 3-6 | QSYDSRLTV | 293 |
| 5B5.2 VLCDR3 | | | |
| 5F2.1 VLCDR3 | CDRL 3-7 | QSYDNSLSDSVL | 294 |
| 5F2.1 VLCDR3 | CDRL 3-8 | NSYSGSNNFDV | 295 |
| 5A4.2 VLCDR3 | CDRL 3-9 | QSYDNRLSDSVV | 296 |
| 5C1.1 VLCDR3 | | | |
| 5D7.2 VLCDR3 | | | |
| 5H3.1 VLCDR3 | | | |
| 5H3.1 VLCDR3 | CDRL 3-10 | SSYAGSNNFDV | 297 |
| 5C1.1 VLCDR3 | | | |

The structure and properties of CDRs within a naturally occurring antibody have been described, supra. Briefly, in a traditional antibody, the CDRs are embedded within a framework in the heavy and light chain variable region where they constitute the regions responsible for antigen binding and recognition. A variable region comprises at least three heavy or light chain CDRs, see, supra (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991, supra; see also Chothia and Lesk, 1987, supra). The CDRs provided herein, however, may not only be used to define the antigen binding domain of a traditional antibody structure, but may be embedded in a variety of other polypeptide structures, as described herein.

Some embodiments of the isolated antigen binding protein comprise an antibody or antibody fragment, comprising an immunoglobulin heavy chain variable region and an immunoglobulin light chain variable region. The heavy chain variable region comprise three complementarity determining regions designated CDRH1, CDRH2 and CDRH3, and/or the light chain variable region comprises three CDRs designated CDRL1, CDRL2 and CDRL3, wherein:

(a) CDRH1 has the amino acid sequence of SEQ ID NO:43, SEQ ID NO:44, SEQ ID NO:45, SEQ ID NO:266, or SEQ ID NO:267; and/or

(b) CDRH2 has the amino acid sequence of SEQ ID NO:46, SEQ ID NO:47, SEQ ID NO:48, SEQ ID NO:49; SEQ ID NO:268, SEQ ID NO:269, SEQ ID NO:270, SEQ ID NO:271, or SEQ ID NO:272; and/or

(c) CDRH3 has the amino acid sequence of SEQ ID NO:50, SEQ ID NO:51, SEQ ID NO:52, SEQ ID NO:273, SEQ ID NO:274, SEQ ID NO:275, SEQ ID NO:276, or SEQ ID NO:277; and/or

(d) CDRL1 has the amino acid sequence of SEQ ID NO:53, SEQ ID NO:54, SEQ ID NO:55, SEQ ID NO:278, SEQ ID NO:279, SEQ ID NO:280, SEQ ID NO:281, SEQ ID NO:282, or SEQ ID NO:283; and/or

(e) CDRL2 has the amino acid sequence of SEQ ID NO:56, SEQ ID NO:57, SEQ ID NO:284, SEQ ID NO:285, SEQ ID NO:286, SEQ ID NO:287, SEQ ID NO:288, or SEQ ID NO:289; and/or

(f) CDRL3 has the amino acid sequence of SEQ ID NO:58, SEQ ID NO:59, SEQ ID NO:290, SEQ ID NO:291, SEQ ID NO:292, SEQ ID NO:293, SEQ ID NO:294, SEQ ID NO:295, SEQ ID NO:296, or SEQ ID NO:297.

In other aspects, the CDRs provided are (A) a CDRH selected from (i) a CDRH1 selected from SEQ ID NOS:43, 44, 45, 266, and 267; (ii) a CDRH2 selected from SEQ ID NOS:46, 47, 48, 49, 268, 269, 270, 271, and 272; (iii) a CDRH3 selected from SEQ ID NOS:50, 51, 52, 273, 274, 275, 276, and 277; and (iv) a CDRH of (i), (ii) and (iii) that contains one or more amino acid substitutions, deletions or insertions of no more than five, four, three, two, or one amino acids; (B) a CDRL selected from (i) a CDRL1 selected from SEQ ID NOS:53, 54, 55, 278, 279, 280, 281, 282, and 283; (ii) a CDRL2 selected from SEQ ID NO:56, 57, 284, 285, 286, 287, 288, and 289; (iii) a CDRL3 selected from SEQ ID NO:58, 59:290, 291, 292, 293, 294, 295, 296, and 297; and (iv) a CDRL of (i), (ii) and (iii) that contains one or more amino acid substitutions, deletions or insertions of no more than five, four, three, two, or one amino acids amino acids.

In another aspect, an antigen binding protein includes 1, 2, 3, 4, 5, or 6 variant forms of the CDRs listed in Table 3A and Table 3B, each having at least 80%, at least 85%, at least 90% or at least 95% sequence identity to a CDR sequence listed in Table 3A and Table 3B. Some antigen binding proteins include 1, 2, 3, 4, 5, or 6 of the CDRs listed in Table 3A and Table 3B, each differing by no more than 1, 2, 3, 4 or 5 amino acids from the CDRs listed in these tables.

In yet another aspect, the CDRs disclosed herein include consensus sequences derived from groups of related monoclonal antibodies. As described herein, a “consensus sequence” refers to amino acid sequences having conserved amino acids common among a number of sequences and variable amino acids that vary within a given amino acid sequences. The CDR consensus sequences provided include CDRs corresponding to each of CDRH1, CDRH2, CDRH3, CDRL1, CDRL2 and CDRL3.

In still another embodiment, the antigen binding protein comprises an immunoglobulin heavy chain variable region comprising three complementarity determining regions designated CDRH1, CDRH2 and CDRH3, wherein:

(a) CDRH1 has the amino acid sequence of

| | |
|---|---|
| a¹ Y a³ a⁴ a⁵//, | SEQ ID NO: 298 |

wherein
a¹ is a glycine, serine or aspartate residue; and
a³ is a tyrosine or tryptophan residue; and
a⁴ is a tryptophan, methionine, or isoleucine residue; and
a⁵ is a serine, histidine, or asparagine residue; and

(b) CDRH2 has the amino acid sequence of

SEQ ID NO: 299

b¹ I b³ b⁴ b⁵ b⁶ b⁷ b⁸ b⁹ b¹⁰ b¹¹
b¹² b¹³ b¹⁴ b¹⁵ b¹⁶ b¹⁷//,

wherein
b¹ is a tryptophan, glutamate, or asparagine residue; and
b³ is an asparagine, aspartate, or lysine residue; and
b⁴ is a proline or histidine residue; and
b⁵ is an asparagine, aspartate, or serine residue; and
b⁶ is an asparagine, serine, or glycine residue; and
b⁷ is a glycine, serine, or arginine residue; and
b⁸ is a glycine, threonine, isoleucine, or glutamate residue; and
b⁹ is a threonine, serine, asparagine, or lysine residue; and
b¹⁰ is a tyrosine, serine, asparagine, aspartate, histidine, or lysine residue; and
b¹¹ is a tyrosine or asparagine residue; and
b¹² is an alanine, valine, or proline residue; and
b¹³ is a glutamine, alanine, or aspartate residue; and
b¹⁴ is a lysine, leucine, or serine residue; and
b¹⁵ is a phenylalanine, lysine, or valine residue; and
b¹⁶ is a glutamine, serine, or lysine residue; and
b¹⁷ is a glycine or aspartate residue, or absent; and

(c) CDRH3 has an amino acid sequence of

SEQ ID NO: 300

c¹ c² c³ G c⁵ c⁶ c⁷ c⁸ c⁹ c¹⁰ c¹¹ c¹² c¹³//,

wherein
c¹ is an alanine or arginine residue, or absent; and
c² is a glutamate, glycine, or tyrosine residue; and
c³ is a glutamate, serine, arginine, or tyrosine residue; and
c⁵ is a glycine or aspartate residue; and
c⁶ is a tyrosine, tryptophan, isoleucine, or asparagine residue; and
c⁷ is a glutamate, serine, or glycine residue, or absent; and
c⁸ is a methionine residue, or absent; and
c⁹ is a threonine or aspartate residue; and
c¹⁰ is a phenylalanine, tryptophan, or valine residue; and
c¹¹ is a phenylalanine residue, or absent; and
c¹² is an aspartate residue, or absent; and
c¹³ is a proline or tyrosine residue, or absent.

In still another embodiment of the antigen binding protein, which may optionally include the immunoglobulin heavy chain variable region described in the previous paragraph, the antigen binding protein comprises an immunoglobulin light chain variable region, the light chain variable region comprising three CDRs designated CDRL1, CDRL2 and CDRL3, wherein:

(a) CDRL1 has the amino acid sequence of

SEQ ID NO: 301

d¹ G d³ d⁴ d⁵ d⁶ d⁷ d⁸ d⁹ d¹⁰ d¹¹ d¹² d¹³ d¹⁴//,

wherein
d¹ is a threonine or serine residue; and
d³ is a serine, threonine, or aspartate residue; and
d⁴ is an asparagine, arginine, alanine, or serine residue; and
d⁵ is a leucine or serine residue; and
d⁶ is an asparagine, aspartate, or proline residue; and
d⁷ is an isoleucine, valine, or lysine residue; and
d⁸ is a glycine or lysine residue; and
d⁹ is an alanine, threonine, glycine, or tyrosine residue; and
d¹⁰ is an alanine, glycine, or tyrosine residue; and
d¹¹ is a tyrosine, phenylalanine, cysteine, or asparagine residue; and
d¹² is an asparagine, aspartate, or tyrosine residue, or absent; and
d¹³ is a valine residue, or absent; and
d¹⁴ is a histidine or serine residue, or absent; and

(b) CDRL2 has the amino acid sequence of

| | |
|---|---|
| e¹ e² e³ e⁴ R P S//, | SEQ ID NO: 302 |

wherein
e¹ is a valine, serine, aspartate, glutamate, or glycine residue; and
e² is a histidine, aspartate, asparagine, valine, or tyrosine residue; and
e³ is a histidine, phenylalanine, arginine, serine, or asparagine residue; and
e⁴ is an isoleucine, asparagine, or lysine residue; and

(c) CDRL3 has the amino acid sequence of

SEQ ID NO: 303

f¹S f³ f⁴ f⁵ f⁶ f⁷ f⁸ f⁹ f¹⁰ f¹¹ f¹²//,

wherein
f¹ is a glutamine, serine, tyrosine, or asparagine residue; and
f³ is a tyrosine or threonine residue; and
f⁴ is a serine, aspartate, glycine, or alanine residue; and
f⁵ is a serine, glycine, or asparagine residue; and
f⁶ is a serine, glycine, or arginine residue; and
f⁷ is a leucine, glycine, or asparagine residue; and
f⁸ is a serine or asparagine residue, or absent; and
f⁹ is a histidine or aspartate residue, or absent; and f¹⁰ is a serine, glycine, or phenylalanine residue, or absent; and
f¹¹ is a valine, serine, tryptophan, aspartate, or threonine residue; and
f¹² is a leucine or valine residue.

In a subset of these embodiments:
d¹ is a threonine residue; and
d³ is a serine or threonine residue; and
d⁴ is an asparagine, arginine, or serine residue; and
d⁵ is a serine residue; and
d⁶ is an asparagine or aspartate residue; and
d⁷ is an isoleucine or valine residue; and
d⁸ is a glycine residue; and

d⁹ is an alanine, threonine, or glycine residue; and
d¹⁰ is a glycine or tyrosine residue; and
d¹¹ is a tyrosine, phenylalanine, or asparagine residue; and
d¹² is an asparagine, aspartate, or tyrosine residue; and
d¹³ is a valine residue; and

d¹⁴ is a histidine or serine residue.

In general, antibody-antigen interactions can be characterized by the association rate constant in M⁻¹ s⁻¹ (kₐ or Kₒₙ), or the dissociation rate constant in s⁻¹ (k_{d} or K_{off}), or alternatively the equilibrium dissociation constant in M (K_{d}), which is a measure of binding affinity that can be determined by a Kinetic Exclusion Assay (KinExA) using general procedures outlined by the manufacturer or other methods known in the art. (See, e.g., Rathanaswami et al., High affinity binding measurements of antibodies to cell-surface-expressed antigens, Analytical Biochemistry 373:52-60 (2008). If KinExA technology is used one can measure the following:

K_{d} (M), the equilibrium dissociation constant and
Kₒₙ (M⁻¹ s⁻¹), the association rate constant. From these values, the dissociation rate constant K_{off} (s⁻¹) can be calculated from K_{d}×Kₒₙ.

Binding affinity can also be characterized by equilibrium constant (K_{D}), which can be determined using surface plasmon resonance (e.g., BIAcore®; e.g., Fischer et al., A peptide-immunoglobulin-conjugate, WO 2007/045463 A1). If a Biacore® instrument is used, then one can measure the following:

kₐ (M⁻¹ s⁻¹), the association rate constant and
k_{d} (s⁻¹), the dissociation rate constant. From theses values, the equilibrium constant K_{D} (M) can be calculated which is the ratio of the kinetic rate constants, k_{d}/kₐ.

The present invention provides a variety of antigen binding proteins, including but not limited to antibodies that specifically bind human Orai1, that exhibit desirable characteristics such as binding affinity as measured by K_{d} or K_{D} for hOrai1 in the range of 10⁻⁹ M or lower, ranging down to 10⁻¹² M or lower, or avidity as measured by k_{d} (dissociation rate constant) for hOrai1 in the range of 10⁻⁴ s⁻¹ or lower, or ranging down to 10⁻¹⁰ s⁻¹ or lower.

In some embodiments, the antigen binding proteins (e.g., antibodies or antibody fragments) exhibit desirable characteristics such as binding avidity as measured by k_{d} (dissociation rate constant) for hOrai1 of about 10⁻², 10⁻³, 10⁻⁴, 10⁻⁵, 10⁻⁶, 10⁻⁷, 10⁻⁸, 10⁻⁹, 10⁻¹⁰ s⁻¹ or lower (lower values indicating higher binding avidity), and/or binding affinity as measured by K_{d} (equilibrium dissociation constant) or K_{D} (equilibrium constant) for hOrai1 of about 10⁻⁹, 10⁻¹⁰, 10⁻¹¹, 10⁻¹², 10⁻¹³, 10⁻¹⁴, 10⁻¹⁵, 10⁻¹⁶ M or lower (lower values indicating higher binding affinity). Association rate constants, dissociation rate constants, or equilibrium constants may be readily determined using kinetic analysis techniques such as surface plasmon resonance (BIAcore®; e.g., Fischer et al., A peptide-immunoglobulin-conjugate, WO 2007/045463 A1, Example 10, which is incorporated herein by reference in its entirety), or equilibrium dissociation constant may be determined using Kinetic Exclusion Assay (KinExA) using general procedures outlined by the manufacturer or other methods known in the art. (See, Rathanaswami et al., High affinity binding measurements of antibodies to cell-surface-expressed antigens, Analytical Biochemistry 373:52-60 (2008), which is incorporated herein by reference in its entirety). The kinetic data obtained by BIAcore® or KinExA may be analyzed by methods described by the manufacturers.

In some embodiments, the antibody comprises all three light chain CDRs, all three heavy chain CDRs, or all six CDRs. In some exemplary embodiments, two light chain CDRs from an antibody may be combined with a third light chain CDR from a different antibody. Alternatively, a CDRL1 from one antibody can be combined with a CDRL2 from a different antibody and a CDRL3 from yet another antibody, particularly where the CDRs are highly homologous. Similarly, two heavy chain CDRs from an antibody may be combined with a third heavy chain CDR from a different antibody; or a CDRH1 from one antibody can be combined with a CDRH2 from a different antibody and a CDRH3 from yet another antibody, particularly where the CDRs are highly homologous.

Thus, the invention provides a variety of compositions comprising one, two, and/or three CDRs of a heavy chain variable region and/or a light chain variable region of an antibody including modifications or derivatives thereof. Such compositions may be generated by techniques described herein or known in the art.

As provided herein, the inventive antigen binding proteins (including antibodies and antibody fragments) and methods of treating immune disorders or disorders related to venous or arterial thrombus formation may utilize one or more anti-hOrai1 antigen binding proteins used singularly or in combination with other therapeutics to achieve the desired effects. Useful preclinical animal models are known in the art for use in validating a drug in a therapeutic indication (e.g., an adoptive-transfer model of periodontal disease by Valverde et al., J. Bone Mineral Res. 19:155 (2004); an ultrasonic perivascular Doppler flow meter-based animal model of arterial thrombosis in Gruner et al., Blood 105:1492-99 (2005); pulmonary thromboembolism model, aorta occlusion model, and murine stroke model in Braun et al., WO 2009/115609 A1). For example, an adoptive transfer experimental autoimmune encephalomyelitis (AT-EAE) model of multiple sclerosis has been described for investigations concerning immune diseases, such as multiple sclerosis (Beeton et al., J. Immunol. 166:936 (2001); Beeton et al., PNAS 98:13942 (2001); Sullivan et al., Example 45 of WO 2008/088422 A2, incorporated herein by reference in its entirety). In the AT-EAE model, significantly reduced disease severity and increased survival are expected for animals treated with an effective amount of the inventive pharmaceutical composition, while untreated animals are expected to develop severe disease and/or mortality. For running the AT-EAE model, the encephalomyelogenic CD4+ rat T cell line, PAS, specific for myelin-basic protein (MBP) originated from Dr. Evelyne Beraud. The maintenance of these cells in vitro and their use in the AT-EAE model has been described earlier [Beeton et al. (2001) PNAS 98, 13942]. PAS T cells are maintained in vitro by alternating rounds of antigen stimulation or activation with MBP and irradiated thymocytes (2 days), and propagation with T cell growth factors (5 days). Activation of PAS T cells (3×10⁵/ml) involves incubating the cells for 2 days with 10 μg/ml MBP and 15×10⁶/ml syngeneic irradiated (3500 rad) thymocytes. On day 2 after in vitro activation, 10−15×10⁶ viable PAS T cells are injected into 6-12 week old female Lewis rats (Charles River Laboratories) by tail IV. Daily subcutaneous injections of vehicle (2% Lewis rat serum in PBS) or test pharmaceutical composition are given from days −1 to 3, where day −1 represent 1 day prior to injection of PAS T cells (day 0). In vehicle treated rats, acute EAE is expected to develop 4 to 5 days after injection of PAS T cells. Typically, serum is collected by tail vein bleeding at day 4 and by cardiac puncture at day 8 (end of the study) for analysis of levels of inhibitor. Rats are typically weighed on days −1, 4, 6, and 8. Animals may be scored blinded once a day from the day of cell transfer (day 0) to day 3, and twice a day from day 4 to day 8. Clinical signs are evaluated as the total score of the degree of paresis of each limb and tail. Clinical scoring: 0=No signs, 0.5=distal limp tail, 1.0=limp tail, 2.0=mild paraparesis, ataxia, 3.0=moderate paraparesis, 3.5=one hind leg paralysis, 4.0=complete hind leg paralysis, 5.0=complete hind leg paralysis and incontinence, 5.5=tetraplegia, 6.0=moribund state or death. Rats reaching a score of 5.0 are typically euthanized.

A useful peptide induced-experimental autoimmune encephalomyelitis (EAE) model has also been described as a model of autoimmune CNS inflammation (Schuhmann et al., Stromal interaction molecules 1 and 2 are key regulators of autoreactive T cell activation in murine autoimmune central nervous system inflammation, J. Immunol. 2010 Feb. 1; 184(3):1536-42. Epub 2009 Dec. 18, incorporated herein by reference in its entirety).

Production of Antibody Embodiments of the Antigen Binding Proteins

Polyclonal Antibodies.

Polyclonal antibodies are preferably raised in animals by multiple subcutaneous (sc) or intraperitoneal (ip) injections of the relevant antigen and an adjuvant. Alternatively, antigen may be injected directly into the animal's lymph node (see Kilpatrick et al., Hybridoma, 16:381-389, 1997). An improved antibody response may be obtained by conjugating the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride or other agents known in the art.

Animals are immunized against the antigen, immunogenic conjugates, or derivatives by combining, e.g., 100 μg of the protein or conjugate (for mice) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. At 7-14 days post-booster injection, the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal Antibodies.

The inventive antigen binding proteins or antigen binding proteins that are provided include monoclonal antibodies that bind to hOrai1. Monoclonal antibodies may be produced using any technique known in the art, e.g., by immortalizing spleen cells harvested from the transgenic animal after completion of the immunization schedule. The spleen cells can be immortalized using any technique known in the art, e.g., by fusing them with myeloma cells to produce hybridomas. For example, monoclonal antibodies may be made using the hybridoma method first described by Kohler et al., Nature, 256:495 (1975), or may be made by recombinant DNA methods (e.g., Cabilly et al., Methods of producing immunoglobulins, vectors and transformed host cells for use therein, U.S. Pat. No. 6,331,415), including methods, such as the “split DHFR” method, that facilitate the generally equimolar production of light and heavy chains, optionally using mammalian cell lines (e.g., CHO cells) that can glycosylate the antibody (See, e.g., Page, Antibody production, EP0481790 A2 and U.S. Pat. No. 5,545,403).

In the hybridoma method, a mouse or other appropriate host mammal, such as rats, hamster or macaque monkey, is immunized as herein described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

In some instances, a hybridoma cell line is produced by immunizing a transgenic animal having human immunoglobulin sequences with a hOrai1 immunogen; harvesting spleen cells from the immunized animal; fusing the harvested spleen cells to a myeloma cell line, thereby generating hybridoma cells; establishing hybridoma cell lines from the hybridoma cells, and identifying a hybridoma cell line that produces an antibody that binds hOrai1. Such hybridoma cell lines, and anti-Orai1 monoclonal antibodies produced by them, are aspects of the present invention.

The present invention also encompasses a hybridoma that produces the inventive antigen binding protein that is a monoclonal antibody. Accordingly, the present invention is also directed to a method, comprising:

(a) culturing the hybridoma in a culture medium under conditions permitting expression of the antigen binding protein by thehybridoma; and

(b) recovering the antigen binding protein from the culture medium, which can be accomplished by known antibody purification techniques, such as but not limited to, monoclonal antibody purification techniques disclosed in Example 4 herein.

The hybridoma cells, once prepared, are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium. Human myeloma and mouse-human heteromyeloma cell lines also have been described for the production of human monoclonal antibodies (Kozbor, J. Immunol., 133: 3001 (1984); Brodeur et al., Monoclonal Antibody Production Techniques and Applications, pp. 51-63 (Marcel Dekker, Inc., New York, 1987)). Myeloma cells for use in hybridoma-producing fusion procedures preferably are non-antibody-producing, have high fusion efficiency, and enzyme deficiencies that render them incapable of growing in certain selective media which support the growth of only the desired fused cells (hybridomas). Examples of suitable cell lines for use in mouse fusions include Sp-20, P3-X63/Ag8, P3-X63-Ag8.653, NS1/1.Ag 4 1, Sp210-Ag14, FO, NSO/U, MPC-11, MPC11-X45-GTG 1.7 and S194/5XXO Bul; examples of cell lines used in rat fusions include R210.RCY3, Y3-Ag 1.2.3, IR983F and 4B210. Other cell lines useful for cell fusions are U-266, GM1500-GRG2, LICR-LON-HMy2 and UC729-6.

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA). The binding affinity of the monoclonal antibody can, for example, be determined by BIAcore® or Scatchard analysis (Munson et al., Anal. Biochem., 107:220 (1980); Fischer et al., A peptide-immunoglobulin-conjugate, WO 2007/045463 A1, Example 10, which is incorporated herein by reference in its entirety).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

Hybridomas or mAbs may be further screened to identify mAbs with particular properties, such as the ability to inhibit Ca2+ flux though CRAC channels. Examples of such screens are provided in the examples below. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, affinity chromatography, or any other suitable purification technique known in the art.

Recombinant Production of Antibodies.

The invention provides isolated nucleic acids encoding any of the antibodies (polyclonal and monoclonal), including antibody fragments, of the invention described herein, optionally operably linked to control sequences recognized by a host cell, vectors and host cells comprising the nucleic acids, and recombinant techniques for the production of the antibodies, which may comprise culturing the host cell so that the nucleic acid is expressed and, optionally, recovering the antibody from the host cell culture or culture medium. Similar materials and methods apply to production of polypeptide-based antigen binding proteins.

Relevant amino acid sequences from an immunoglobulin or polypeptide of interest may be determined by direct protein sequencing, and suitable encoding nucleotide sequences can be designed according to a universal codon table. Alternatively, genomic or cDNA encoding the monoclonal antibodies may be isolated and sequenced from cells producing such antibodies using conventional procedures (e.g., by using oligonucleotide probes that are capable of binding specifically to genes encoding the heavy and light chains of the monoclonal antibodies).

Cloning of DNA is carried out using standard techniques (see, e.g., Sambrook et al. (1989) Molecular Cloning: A Laboratory Guide, Vols 1-3, Cold Spring Harbor Press, which is incorporated herein by reference). For example, a cDNA library may be constructed by reverse transcription of polyA+mRNA, preferably membrane-associated mRNA, and the library screened using probes specific for human immunoglobulin polypeptide gene sequences. In one embodiment, however, the polymerase chain reaction (PCR) is used to amplify cDNAs (or portions of full-length cDNAs) encoding an immunoglobulin gene segment of interest (e.g., a light or heavy chain variable segment). The amplified sequences can be readily cloned into any suitable vector, e.g., expression vectors, minigene vectors, or phage display vectors. It will be appreciated that the particular method of cloning used is not critical, so long as it is possible to determine the sequence of some portion of the immunoglobulin polypeptide of interest.

One source for antibody nucleic acids is a hybridoma produced by obtaining a B cell from an animal immunized with the antigen of interest and fusing it to an immortal cell. Alternatively, nucleic acid can be isolated from B cells (or whole spleen) of the immunized animal. Yet another source of nucleic acids encoding antibodies is a library of such nucleic acids generated, for example, through phage display technology. Polynucleotides encoding peptides of interest, e.g., variable region peptides with desired binding characteristics, can be identified by standard techniques such as panning.

The sequence encoding an entire variable region of the immunoglobulin polypeptide may be determined; however, it will sometimes be adequate to sequence only a portion of a variable region, for example, the CDR-encoding portion. Sequencing is carried out using standard techniques (see, e.g., Sambrook et al. (1989) Molecular Cloning: A Laboratory Guide, Vols 1-3, Cold Spring Harbor Press, and Sanger, F. et al. (1977) Proc. Natl. Acad. Sci. USA 74: 5463-5467, which is incorporated herein by reference). By comparing the sequence of the cloned nucleic acid with published sequences of human immunoglobulin genes and cDNAs, one of skill will readily be able to determine, depending on the region sequenced, (i) the germline segment usage of the hybridoma immunoglobulin polypeptide (including the isotype of the heavy chain) and (ii) the sequence of the heavy and light chain variable regions, including sequences resulting from N-region addition and the process of somatic mutation. One source of immunoglobulin gene sequence information is the National Center for Biotechnology Information, National Library of Medicine, National Institutes of Health, Bethesda, Md.

Isolated DNA can be operably linked to control sequences or placed into expression vectors, which are then transfected into host cells that do not otherwise produce immunoglobulin protein, to direct the synthesis of monoclonal antibodies in the recombinant host cells. Recombinant production of antibodies is well known in the art.

Nucleic acid is operably linked when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, operably linked means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

Many vectors are known in the art. Vector components may include one or more of the following: a signal sequence (that may, for example, direct secretion of the antibody; e.g., ATGGACATGAGGGTGCCCGCTCAGCTCCTGGGGCTCCTGCTGCTGTGGCT GAGAGGTGCGCGCTGT// SEQ ID NO:233, which encodes the VK-1 signal peptide sequence MDMRVPAQLLGLLLLWLRGARC// SEQ ID NO:234, an origin of replication, one or more selective marker genes (that may, for example, confer antibiotic or other drug resistance, complement auxotrophic deficiencies, or supply critical nutrients not available in the media), an enhancer element, a promoter, and a transcription termination sequence, all of which are well known in the art.

Cell, cell line, and cell culture are often used interchangeably and all such designations herein include progeny. Transformants and transformed cells include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included.

Exemplary host cells include prokaryote, yeast, or higher eukaryote cells. Prokaryotic host cells include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacillus such as B. subtilis and B. licheniformis, Pseudomonas, and Streptomyces. Eukaryotic microbes such as filamentous fungi or yeast are suitable cloning or expression hosts for recombinant polypeptides or antibodies. Saccharomyces cerevisiae, or common baker's yeast, is the most commonly used among lower eukaryotic host microorganisms. However, a number of other genera, species, and strains are commonly available and useful herein, such as Pichia, e.g. P. pastoris, Schizosaccharomyces pombe; Kluyveromces, Yarrowia; Candida; Trichoderma reesia; Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Host cells for the expression of glycosylated antigen binding protein, including antibody, can be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection of such cells are publicly available, e.g., the L-1 variant of Autographa californica NPV and the Bm-5 strain of Bombyx mori NPV.

Vertebrate host cells are also suitable hosts, and recombinant production of antigen binding protein (including antibody) from such cells has become routine procedure. Examples of useful mammalian host cell lines are Chinese hamster ovary cells, including CHOK1 cells (ATCC CCL61), DXB-11, DG-44, and Chinese hamster ovary cells/−DHFR(CHO, Urlaub et al., Proc. Natl. Acad. Sci. USA 77: 4216 (1980)); monkey kidney CV1 line transformed by SV40 (COS-7, ATCC CRL 1651); human embryonic kidney line (293 or 293 cells subcloned for growth in suspension culture, [Graham et al., J. Gen Virol. 36: 59 (1977)]; baby hamster kidney cells (BHK, ATCC CCL 10); mouse sertoli cells (TM4, Mather, Biol. Reprod. 23: 243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TR1 cells (Mather et al., Annals N.Y. Acad. Sci. 383: 44-68 (1982)); MRC 5 cells or FS4 cells; or mammalian myeloma cells.

Host cells are transformed or transfected with the above-described nucleic acids or vectors for production antigen binding proteins and are cultured in conventional nutrient media modified as appropriate for inducing promoters, selecting transformants, or amplifying the genes encoding the desired sequences. In addition, novel vectors and transfected cell lines with multiple copies of transcription units separated by a selective marker are particularly useful for the expression of antigen binding proteins.

The host cells used to produce the antigen binding proteins of the invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58: 44 (1979), Barnes et al., Anal. Biochem. 102: 255 (1980), U.S. Pat. No. 4,767,704; 4,657,866; 4,927,762; 4,560,655; or 5,122,469; WO90103430; WO 87/00195; or U.S. Pat. Re. No. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Upon culturing the host cells, the antigen binding protein can be produced intracellularly, in the periplasmic space, or directly secreted into the medium. If the antigen binding protein is produced intracellularly, as a first step, the particulate debris, either host cells or lysed fragments, is removed, for example, by centrifugation or ultrafiltration.

The antigen binding protein (e.g., an antibody or antibody fragment) can be purified using, for example, hydroxylapatite chromatography, cation or anion exchange chromatography, or preferably affinity chromatography, using the antigen of interest or protein A or protein G as an affinity ligand. Protein A can be used to purify proteins that include polypeptides are based on human γ1, γ2, or γ⁴ heavy chains (Lindmark et al., J. Immunol. Meth. 62: 1-13 (1983)). Protein G is recommended for all mouse isotypes and for human y3 (Guss et al., EMBO J. 5: 15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the protein comprises a C_{H}3 domain, the Bakerbond ABX™ resin (J. T. Baker, Phillipsburg, N.J.) is useful for purification. Other techniques for protein purification such as ethanol precipitation, Reverse Phase HPLC, chromatofocusing, SDS-PAGE, and ammonium sulfate precipitation are also possible depending on the antibody to be recovered.

Chimeric, Humanized and Human Engineered™ Monoclonal Antibodies.

Chimeric monoclonal antibodies, in which the variable Ig domains of a rodent monoclonal antibody are fused to human constant Ig domains, can be generated using standard procedures known in the art (See Morrison, S. L., et al. (1984) Chimeric Human Antibody Molecules; Mouse Antigen Binding Domains with Human Constant Region Domains, Proc. Natl. Acad. Sci. USA 81, 6841-6855; and, Boulianne, G. L., et al, Nature 312, 643-646 (1984)). A number of techniques have been described for humanizing or modifying antibody sequence to be more human-like, for example, by (1) grafting the non-human complementarity determining regions (CDRs) onto a human framework and constant region (a process referred to in the art as humanizing through “CDR grafting”) or (2) transplanting the entire non-human variable domains, but “cloaking” them with a human-like surface by replacement of surface residues (a process referred to in the art as “veneering”) or (3) modifying selected non-human amino acid residues to be more human, based on each residue's likelihood of participating in antigen-binding or antibody structure and its likelihood for immunogenicity. See, e.g., Jones et al., Nature 321:522 525 (1986); Morrison et al., Proc. Natl. Acad. Sci., U.S.A., 81:6851 6855 (1984); Morrison and Oi, Adv. Immunol., 44:65 92 (1988); Verhoeyer et al., Science 239:1534 1536 (1988); Padlan, Molec. Immun. 28:489 498 (1991); Padlan, Molec. Immunol. 31(3):169 217 (1994); and Kettleborough, C. A. et al., Protein Eng. 4(7):773 83 (1991); Co, M. S., et al. (1994), J. Immunol. 152, 2968-2976); Studnicka et al. Protein Engineering 7: 805-814 (1994); each of which is incorporated herein by reference in its entirety.

A number of techniques have been described for humanizing or modifying antibody sequence to be more human-like, for example, by (1) grafting the non-human complementarity determining regions (CDRs) onto a human framework and constant region (a process referred to in the art as humanizing through “CDR grafting”) or (2) transplanting the entire non-human variable domains, but “cloaking” them with a human-like surface by replacement of surface residues (a process referred to in the art as “veneering”) or (3) modifying selected non-human amino acid residues to be more human, based on each residue's likelihood of participating in antigen-binding or antibody structure and its likelihood for immunogenicity. See, e.g., Jones et al., Nature 321:522 525 (1986); Morrison et al., Proc. Natl. Acad. Sci., U.S.A., 81:6851 6855 (1984); Morrison and Oi, Adv. Immunol., 44:65 92 (1988); Verhoeyer et al., Science 239:1534 1536 (1988); Padlan, Molec. Immun. 28:489 498 (1991); Padlan, Molec. Immunol. 31(3):169 217 (1994); and Kettleborough, C. A. et al., Protein Eng. 4(7):773 83 (1991); Co, M. S., et al. (1994), J. Immunol. 152, 2968-2976); Studnicka et al. Protein Engineering 7: 805-814 (1994); each of which is incorporated herein by reference in its entirety.

In one aspect, the CDRs of the light and heavy chain variable regions of the antibodies provided herein (see, Table 2) are grafted to framework regions (FRs) from antibodies from the same, or a different, phylogenetic species. For example, the CDRs of the heavy chain variable regions (e.g., V_{H}1, V_{H}2, V_{H}3, V_{H}4, V_{H}5, V_{H}6, V_{H}7, V_{H}8, V_{H}9, or V_{H}10) and/or light chain variable regions (e.g., V_{L}1, V_{L}2, V_{L}3, V_{L}4, V_{L}5, V_{L}6, V_{L}7, V_{L}8, V_{L}9, V_{L}10, V_{L}11, V_{L}12, or V_{L}13) can be grafted to consensus human FRs. To create consensus human FRs, FRs from several human heavy chain or light chain amino acid sequences may be aligned to identify a consensus amino acid sequence. In other embodiments, the FRs of a heavy chain or light chain disclosed herein are replaced with the FRs from a different heavy chain or light chain. In one aspect, rare amino acids in the FRs of the heavy and light chains of anti-hOrai1 ECL2 antibody are not replaced, while the rest of the FR amino acids are replaced. A “rare amino acid” is a specific amino acid that is in a position in which this particular amino acid is not usually found in an FR. Alternatively, the grafted variable regions from the one heavy or light chain may be used with a constant region that is different from the constant region of that particular heavy or light chain as disclosed herein. In other embodiments, the grafted variable regions are part of a single chain Fv antibody.

Antibodies to hOrai1 ECL2 can also be produced using transgenic animals that have no endogenous immunoglobulin production and are engineered to contain human immunoglobulin loci. For example, WO 98/24893 discloses transgenic animals having a human Ig locus wherein the animals do not produce functional endogenous immunoglobulins due to the inactivation of endogenous heavy and light chain loci. WO 91/10741 also discloses transgenic non-primate mammalian hosts capable of mounting an immune response to an immunogen, wherein the antibodies have primate constant and/or variable regions, and wherein the endogenous immunoglobulin encoding loci are substituted or inactivated. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy chains, and express an exogenous immunoglobulin locus comprising one or more xenogeneic constant regions.

Using a transgenic animal described above, an immune response can be produced to a selected antigenic molecule, and antibody producing cells can be removed from the animal and used to produce hybridomas that secrete human-derived monoclonal antibodies. Immunization protocols, adjuvants, and the like are known in the art, and are used in immunization of, for example, a transgenic mouse as described in WO 96/33735. The monoclonal antibodies can be tested for the ability to inhibit or neutralize the biological activity or physiological effect of the corresponding protein. See also Jakobovits et al., Proc. Natl. Acad. Sci. USA, 90:2551 (1993); Jakobovits et al., Nature, 362:255-258 (1993); Bruggermann et al., Year in Immuno., 7:33 (1993); Mendez et al., Nat. Genet. 15:146-156 (1997); and U.S. Pat. No. 5,591,669, U.S. Pat. No. 5,589,369, U.S. Pat. No. 5,545,807; and U.S Patent Application No. 20020199213. U.S. patent application No. and 20030092125 describes methods for biasing the immune response of an animal to the desired epitope. Human antibodies may also be generated by in vitro activated B cells (see U.S. Pat. Nos. 5,567,610 and 5,229,275).

Antibody Production by Phage Display Techniques

The development of technologies for making repertoires of recombinant human antibody genes, and the display of the encoded antibody fragments on the surface of filamentous bacteriophage, has provided another means for generating human-derived antibodies. Phage display is described in e.g., Dower et al., WO 91/17271, McCafferty et al., WO 92/01047, and Caton and Koprowski, Proc. Natl. Acad. Sci. USA, 87:6450-6454 (1990), each of which is incorporated herein by reference in its entirety. The antibodies produced by phage technology are usually produced as antigen binding fragments, e.g. Fv or Fab fragments, in bacteria and thus lack effector functions. Effector functions can be introduced by one of two strategies: The fragments can be engineered either into complete antibodies for expression in mammalian cells, or into bispecific antibody fragments with a second binding site capable of triggering an effector function.

Typically, the Fd fragment (V_{H}-C_{H}1) and light chain (V_{L}-C_{L}) of antibodies are separately cloned by PCR and recombined randomly in combinatorial phage display libraries, which can then be selected for binding to a particular antigen. The antibody fragments are expressed on the phage surface, and selection of Fv or Fab (and therefore the phage containing the DNA encoding the antibody fragment) by antigen binding is accomplished through several rounds of antigen binding and re-amplification, a procedure termed panning. Antibody fragments specific for the antigen are enriched and finally isolated.

Phage display techniques can also be used in an approach for the humanization of rodent monoclonal antibodies, called “guided selection” (see Jespers, L. S., et al., Bio/Technology 12, 899-903 (1994)). For this, the Fd fragment of the mouse monoclonal antibody can be displayed in combination with a human light chain library, and the resulting hybrid Fab library may then be selected with antigen. The mouse Fd fragment thereby provides a template to guide the selection. Subsequently, the selected human light chains are combined with a human Fd fragment library. Selection of the resulting library yields entirely human Fab.

A variety of procedures have been described for deriving human antibodies from phage-display libraries (See, for example, Hoogenboom et al., J. Mol. Biol., 227:381 (1991); Marks et al., J. Mol. Biol, 222:581-597 (1991); U.S. Pat. Nos. 5,565,332 and 5,573,905; Clackson, T., and Wells, J. A., TIBTECH 12, 173-184 (1994)). In particular, in vitro selection and evolution of antibodies derived from phage display libraries has become a powerful tool (See Burton, D. R., and Barbas III, C. F., Adv. Immunol. 57, 191-280 (1994); and, Winter, G., et al., Annu. Rev. Immunol. 12, 433-455 (1994); U.S. patent application no. 20020004215 and WO92/01047; U.S. patent application no. 20030190317 published Oct. 9, 2003 and U.S. Pat. No. 6,054,287; U.S. Pat. No. 5,877,293.

Watkins, “Screening of Phage-Expressed Antibody Libraries by Capture Lift,” Methods in Molecular Biology, Antibody Phage Display: Methods and Protocols 178: 187-193, and U.S. Patent Application Publication No. 20030044772 published Mar. 6, 2003 describes methods for screening phage-expressed antibody libraries or other binding molecules by capture lift, a method involving immobilization of the candidate binding molecules on a solid support.

Other Embodiments of Antigen binding proteins: Antibody Fragments

As noted above, antibody fragments comprise a portion of an intact full length antibody, preferably an antigen binding or variable region of the intact antibody, and include linear antibodies and multispecific antibodies formed from antibody fragments. Nonlimiting examples of antibody fragments include Fab, Fab′, F(ab′)2, Fv, Fd, domain antibody (dAb), complementarity determining region (CDR) fragments, single-chain antibodies (scFv), single chain antibody fragments, maxibodies, diabodies, triabodies, tetrabodies, minibodies, linear antibodies, chelating recombinant antibodies, tribodies or bibodies, intrabodies, nanobodies, small modular immunopharmaceuticals (SMIPs), an antigen-binding-domain immunoglobulin fusion protein, a camelized antibody, a VHH containing antibody, or muteins or derivatives thereof, and polypeptides that contain at least a portion of an immunoglobulin that is sufficient to confer specific antigen binding to the polypeptide, such as a CDR sequence, as long as the antibody retains the desired biological activity. Such antigen fragments may be produced by the modification of whole antibodies or synthesized de novo using recombinant DNA technologies or peptide synthesis.

Additional antibody fragments include a domain antibody (dAb) fragment (Ward et al., Nature 341:544-546, 1989) which consists of a V_{H} domain.

“Linear antibodies” comprise a pair of tandem Fd segments (V_{H}-C_{H}1-V_{H}-C_{H}1) which form a pair of antigen binding regions. Linear antibodies can be bispecific or monospecific (Zapata et al. Protein Eng. 8:1057-62 (1995)).

A “minibody” consisting of scFv fused to CH3 via a peptide linker (hingeless) or via an IgG hinge has been described in Olafsen, et al., Protein Eng Des Sel. 2004 Apr.; 17(4):315-23.

The term “maxibody” refers to bivalent scFvs covalently attached to the Fc region of an immunoglobulin, see, for example, Fredericks et al, Protein Engineering, Design & Selection, 17:95-106 (2004) and Powers et al., Journal of Immunological Methods, 251:123-135 (2001).

Functional heavy-chain antibodies devoid of light chains are naturally occurring in certain species of animals, such as nurse sharks, wobbegong sharks and Camelidae, such as camels, dromedaries, alpacas and llamas. The antigen-binding site is reduced to a single domain, the VH_{H} domain, in these animals. These antibodies form antigen-binding regions using only heavy chain variable region, i.e., these functional antibodies are homodimers of heavy chains only having the structure H₂L₂ (referred to as “heavy-chain antibodies” or “HCAbs”). Camelized V_{HH} reportedly recombines with IgG2 and IgG3 constant regions that contain hinge, CH2, and CH3 domains and lack a CH1 domain. Classical V_{H}-only fragments are difficult to produce in soluble form, but improvements in solubility and specific binding can be obtained when framework residues are altered to be more VH_{H}-like. (See, e.g., Reichman, et al., J Immunol Methods 1999, 231:25-38.) Camelized V_{HH} domains have been found to bind to antigen with high affinity (Desmyter et al., J. Biol. Chem. 276:26285-90, 2001) and possess high stability in solution (Ewert et al., Biochemistry 41:3628-36, 2002). Methods for generating antibodies having camelized heavy chains are described in, for example, in U.S. Patent Publication Nos. 2005/0136049 and 2005/0037421. Alternative scaffolds can be made from human variable-like domains that more closely match the shark V-NAR scaffold and may provide a framework for a long penetrating loop structure.

Because the variable domain of the heavy-chain antibodies is the smallest fully functional antigen-binding fragment with a molecular mass of only 15 kDa, this entity is referred to as a nanobody (Cortez-Retamozo et al., Cancer Research 64:2853-57, 2004). A nanobody library may be generated from an immunized dromedary as described in Conrath et al., (Antimicrob Agents Chemother 45: 2807-12, 2001).

Intrabodies are single chain antibodies which demonstrate intracellular expression and can manipulate intracellular protein function (Biocca, et al., EMBO J. 9:101-108, 1990; Colby et al., Proc Natl Acad Sci USA. 101:17616-21, 2004). Intrabodies, which comprise cell signal sequences which retain the antibody contruct in intracellular regions, may be produced as described in Mhashilkar et al (EMBO J. 14:1542-51, 1995) and Wheeler et al. (FASEB J. 17:1733-5. 2003). Transbodies are cell-permeable antibodies in which a protein transduction domains (PTD) is fused with single chain variable fragment (scFv) antibodies Heng et al., (Med. Hypotheses. 64:1105-8, 2005).

Further encompassed by the invention are antibodies that are SMIPs or binding domain immunoglobulin fusion proteins specific for target protein. These constructs are single-chain polypeptides comprising antigen binding domains fused to immunoglobulin domains necessary to carry out antibody effector functions. See e.g., WO03/041600, U.S. Patent publication 20030133939 and US Patent Publication 20030118592.

Various techniques have been developed for the production of antibody fragments. Traditionally, these fragments were derived via proteolytic digestion of intact antibodies, but can also be produced directly by recombinant host cells. See, for example, Better et al., Science 240: 1041-1043 (1988); Skerra et al. Science 240: 1038-1041 (1988); Carter et al., Bio/Technology 10:163-167 (1992).

Other Embodiments of Antigen Binding Proteins: Multivalent Antibodies

In some embodiments, it may be desirable to generate multivalent or even a multispecific (e.g. bispecific, trispecific, etc.) monoclonal antibody. Such antibody may have binding specificities for at least two different epitopes of the target antigen, or alternatively it may bind to two different molecules, e.g. to the target antigen and to a cell surface protein or receptor. For example, a bispecific antibody may include an arm that binds to the target and another arm that binds to a triggering molecule on a leukocyte such as a T-cell receptor molecule (e.g., CD2 or CD3), or Fc receptors for IgG (FcγR), such as FcγRI (CD64), FcγRII (CD32) and FcγRIII (CD16) so as to focus cellular defense mechanisms to the target-expressing cell. As another example, bispecific antibodies may be used to localize cytotoxic agents to cells which express target antigen. These antibodies possess a target-binding arm and an arm which binds the cytotoxic agent (e.g., saporin, anti-interferon-60, vinca alkaloid, ricin A chain, methotrexate or radioactive isotope hapten). Multispecific antibodies can be prepared as full length antibodies or antibody fragments.

Additionally, the anti-Aβ antibodies of the present invention can also be constructed to fold into multivalent forms, which may improve binding affinity, specificity and/or increased half-life in blood. Multivalent forms of anti-Aβ antibodies can be prepared by techniques known in the art.

Bispecific or multispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods. Suitable cross-linking agents are well known in the art, and are disclosed in U.S. Pat. No. 4,676,980, along with a number of cross-linking techniques. Another method is designed to make tetramers by adding a streptavidin-coding sequence at the C-terminus of the scFv. Streptavidin is composed of four subunits, so when the scFv-streptavidin is folded, four subunits associate to form a tetramer (Kipriyanov et al., Hum Antibodies Hybridomas 6(3): 93-101 (1995), the disclosure of which is incorporated herein by reference in its entirety).

According to another approach for making bispecific antibodies, the interface between a pair of antibody molecules can be engineered to maximize the percentage of heterodimers which are recovered from recombinant cell culture. One interface comprises at least a part of the C_{H}3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. See WO 96/27011 published Sep. 6, 1996.

Techniques for generating bispecific or multispecific antibodies from antibody fragments have also been described in the literature. For example, bispecific or trispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes. Better et al., Science 240: 1041-1043 (1988) disclose secretion of functional antibody fragments from bacteria (see, e.g., Better et al., Skerra et al. Science 240: 1038-1041 (1988)). For example, Fab′-SH fragments can be directly recovered from E. coli and chemically coupled to form bispecific antibodies (Carter et al., Bio/Technology 10:163-167 (1992); Shalaby et al., J. Exp. Med. 175:217-225 (1992)).

Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecfic antibody.

Various techniques for making and isolating bispecific or multispecific antibody fragments directly from recombinant cell culture have also been described. For example, bispecific antibodies have been produced using leucine zippers, e.g. GCN4. (See generally Kostelny et al., J. Immunol. 148(5):1547-1553 (1992).) The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers.

Diabodies, described above, are one example of a bispecific antibody. See, for example, Hollinger et al., Proc. Natl. Acad. Sci. USA, 90:6444-6448 (1993). Bivalent diabodies can be stabilized by disulfide linkage.

Stable monospecific or bispecific Fv tetramers can also be generated by noncovalent association in (scFv₂)₂ configuration or as bis-tetrabodies. Alternatively, two different scFvs can be joined in tandem to form a bis-scFv.

Another strategy for making bispecific antibody fragments by the use of single-chain Fv (sFv) dimers has also been reported. See Gruber et al., J. Immunol. 152: 5368 (1994). One approach has been to link two scFv antibodies with linkers or disulfide bonds (Mallender and Voss, J. Biol. Chem. 269:199-2061994, WO 94/13806, and U.S. Pat. No. 5,989,830, the disclosures of which are incorporated herein by reference in their entireties).

Alternatively, the bispecific antibody may be a “linear antibody” produced as described in Zapata et al. Protein Eng. 8(10):1057-1062 (1995). Briefly, these antibodies comprise a pair of tandem Fd segments (V_{H}-C_{H}1-V_{H}-C_{H}1) which form a pair of antigen binding regions. Linear antibodies can be bispecific or monospecific.

Antibodies with more than two valencies are also contemplated. For example, trispecific antibodies can be prepared. (Tutt et al., J. Immunol. 147:60 (1991)).

A “chelating recombinant antibody” is a bispecific antibody that recognizes adjacent and non-overlapping epitopes of the target antigen, and is flexible enough to bind to both epitopes simultaneously (Neri et al., J Mol. Biol. 246:367-73, 1995).

Production ofbispecific Fab-scFv (“bibody”) and trispecific Fab-(scFv)(2) (“tribody”) are described in Schoonjans et al. (J Immunol. 165:7050-57, 2000) and Willems et al. (J Chromatogr B Analyt Technol Biomed Life Sci. 786:161-76, 2003). For bibodies or tribodies, a scFv molecule is fused to one or both of the VL-CL (L) and VH-CH₁ (Fd) chains, e.g., to produce a tribody two scFvs are fused to C-term of Fab while in a bibody one scFv is fused to C-term of Fab.

In yet another method, dimers, trimers, and tetramers are produced after a free cysteine is introduced in the parental protein. A peptide-based cross linker with variable numbers (two to four) of maleimide groups was used to cross link the protein of interest to the free cysteines (Cochran et al., Immunity 12(3): 241-50 (2000), the disclosure of which is incorporated herein in its entirety).

Other Embodiments of Antigen Binding Proteins

Other antigen binding proteins can be prepared, for example, based on CDRs from an antibody or by screening libraries of diverse peptides or organic chemical compounds for peptides or compounds that exhibit the desired binding properties for hOrai1 ECL2. Human Orai1 ECL2-antigen binding proteins include peptides containing amino acid sequences that are at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more identical to one or more of the CDR sequences as set forth in Table 3A and Table 3B herein.

Inventive antigen binding proteins also include peptibodies. The term “peptibody” refers to a molecule comprising an antibody Fc domain attached to at least one peptide. The production of peptibodies is generally described in PCT publication WO 00/24782, published May 4, 2000. Any of these peptides may be linked in tandem (i.e., sequentially), with or without linkers. Peptides containing a cysteinyl residue may be cross-linked with another Cys-containing peptide, either or both of which may be linked to a vehicle. Any peptide having more than one Cys residue may form an intrapeptide disulfide bond, as well. Any of these peptides may be derivatized, for example the carboxyl terminus may be capped with an amino group, cysteines may be cappe, or amino acid residues may substituted by moieties other than amino acid residues (see, e.g., Bhatnagar et al., J. Med. Chem. 39: 3814-9 (1996), and Cuthbertson et al., J. Med. Chem. 40: 2876-82 (1997), which are incorporated by reference herein in their entirety). The peptide sequences may be optimized, analogous to affinity maturation for antibodies, or otherwise altered by alanine scanning or random or directed mutagenesis followed by screening to identify the best binders. Lowman, Ann. Rev. Biophys. Biomol. Struct. 26: 401-24 (1997). Various molecules can be inserted into the antigen binding protein structure, e.g., within the peptide portion itself or between the peptide and vehicle portions of the antigen binding proteins, while retaining the desired activity of antigen binding protein. One can readily insert, for example, molecules such as an Fc domain or fragment thereof, polyethylene glycol or other related molecules such as dextran, a fatty acid, a lipid, a cholesterol group, a small carbohydrate, a peptide, a detectable moiety as described herein (including fluorescent agents, radiolabels such as radioisotopes), an oligosaccharide, oligonucleotide, a polynucleotide, interference (or other) RNA, enzymes, hormones, or the like. Other molecules suitable for insertion in this fashion will be appreciated by those skilled in the art, and are encompassed within the scope of the invention. This includes insertion of, for example, a desired molecule in between two consecutive amino acids, optionally joined by a suitable linker.

Linkers. A “linker” or “linker moiety”, as used interchangeably herein, refers to a biologically acceptable peptidyl or non-peptidyl organic group that is covalently bound to an amino acid residue of a polypeptide chain (e.g., an immunoglobulin HC or immunoglobulin LC or immunoglobulin Fc domain) contained in the inventive composition, which linker moiety covalently joins or conjugates the polypeptide chain to another peptide or polypeptide chain in the molecule, or to a therapeutic moiety, such as a biologically active small molecule or oligopeptide, or to a half-life extending moiety, e.g., see, Sullivan et al., Toxin Peptide Therapeutic Agents, US2007/0071764; Sullivan et al., Toxin Peptide Therapeutic Agents, PCT/US2007/022831, published as WO 2008/088422; and U.S. Provisional Application Ser. No. 61/210,594, filed Mar. 20, 2009, which are all incorporated herein by reference in their entireties.

The presence of any linker moiety in the antigen binding proteins of the present invention is optional. When present, the linker's chemical structure is not critical, since it serves primarily as a spacer to position, join, connect, or optimize presentation or position of one functional moiety in relation to one or more other functional moieties of a molecule of the inventive antigen binding protein. The presence of a linker moiety can be useful in optimizing pharamcologial activity of some embodiments of the inventive antigen binding protein (including antibodies and antibody fragments). The linker is preferably made up of amino acids linked together by peptide bonds. The linker moiety, if present, can be independently the same or different from any other linker, or linkers, that may be present in the inventive antigen binding protein.

As stated above, the linker moiety, if present (whether within the primary amino acid sequence of the antigen binding protein, or as a linker for attaching a therapeutic moiety or half-life extending moiety to the inventive antigen binding protein), can be “peptidyl” in nature (i.e., made up of amino acids linked together by peptide bonds) and made up in length, preferably, of from 1 up to about 40 amino acid residues, more preferably, of from 1 up to about 20 amino acid residues, and most preferably of from 1 to about 10 amino acid residues. Preferably, but not necessarily, the amino acid residues in the linker are from among the twenty canonical amino acids, more preferably, cysteine, glycine, alanine, proline, asparagine, glutamine, and/or serine. Even more preferably, a peptidyl linker is made up of a majority of amino acids that are sterically unhindered, such as glycine, serine, and alanine linked by a peptide bond. It is also desirable that, if present, a peptidyl linker be selected that avoids rapid proteolytic turnover in circulation in vivo. Some of these amino acids may be glycosylated, as is well understood by those in the art. For example, a useful linker sequence constituting a sialylation site is X₁X₂NX₄XsG (SEQ ID NO: 148), wherein X₁, X₂, X₄ and X₅ are each independently any amino acid residue.

In other embodiments, the 1 to 40 amino acids of the peptidyl linker moiety are selected from glycine, alanine, proline, asparagine, glutamine, and lysine. Preferably, a linker is made up of a majority of amino acids that are sterically unhindered, such as glycine and alanine. Thus, preferred linkers include polyglycines, polyserines, and polyalanines, or combinations of any of these. Some exemplary peptidyl linkers are poly(Gly)₁₋₈, particularly (Gly)₃, (Gly)₄ (SEQ ID NO:149), (Gly)₅ (SEQ ID NO:150) and (Gly)₇ (SEQ ID NO:151), as well as, poly(Gly)₄ Ser (SEQ ID NO: 152), poly(Gly-Ala)₂₋₄ and poly(Ala)₁₋₈. Other specific examples of peptidyl linkers include (Gly)₅Lys (SEQ ID NO: 154), and (Gly)₅LysArg (SEQ ID NO:155). Other specific examples of linkers are: Other examples of useful peptidyl linkers are:

| | |
|---|---|
| (Gly)₃Lys(Gly)₄; | (SEQ ID NO: 159) |
| (Gly)₃AsnGlySer(Gly)₂; | (SEQ ID NO: 156) |
| (Gly)₃Cys(Gly)₄; | (SEQ ID NO: 157) |
| and | |
| GlyProAsnGlyGly. | (SEQ ID NO: 158) |

To explain the above nomenclature, for example, (Gly)₃ Lys(Gly)₄ means Gly-Gly-Gly-Lys-Gly-Gly-Gly-Gly (SEQ ID NO:159). Other combinations of Gly and Ala are also useful.

Commonly used linkers include those which may be identified herein as “L5” (GGGGS; or “G₄S”; SEQ ID NO:152), “L10” (GGGGSGGGGS; SEQ ID NO:153), “L25” (GGGGSGGGGSGGGGSGGGGSGGGGS; SEQ ID NO:146) and any linkers used in the working examples hereinafter.

In some embodiments of the compositions of this invention, which comprise a peptide linker moiety, acidic residues, for example, glutamate or aspartate residues, are placed in the amino acid sequence of the linker moiety. Examples include the following peptide linker sequences:

| | |
|---|---|
| GGEGGG; | (SEQ ID NO: 160) |
| GGEEEGGG; | (SEQ ID NO: 161) |
| GEEEG; | (SEQ ID NO: 162) |
| GEEE; | (SEQ ID NO: 163) |
| GGDGGG; | (SEQ ID NO: 164) |
| GGDDDGG; | (SEQ ID NO: 165) |
| GDDDG; | (SEQ ID NO: 166) |
| GDDD; | (SEQ ID NO: 167) |
| GGGGSDDSDEGSDGEDGGGGS; | (SEQ ID NO: 168) |
| WEWEW; | (SEQ ID NO: 169) |
| FEFEF; | (SEQ ID NO: 170) |
| EEEWWW; | (SEQ ID NO: 171) |
| EEEFFF; | (SEQ ID NO: 172) |
| WWEEEWW; | (SEQ ID NO: 173) |
| or | |
| FFEEEFF. | (SEQ ID NO: 174) |

In other embodiments, the linker constitutes a phosphorylation site, e.g., X₁X₂YX₄X₅G (SEQ ID NO:175), wherein X₁, X₂, X₄, and X₅ are each independently any amino acid residue; X₁X₂SX₄XsG (SEQ ID NO:176), wherein X₁, X₂, X₄ and X₅ are each independently any amino acid residue; or X₁X₂TX₄XsG (SEQ ID NO:177), wherein X₁, X₂, X₄ and X₅ are each independently any amino acid residue.

The linkers shown here are exemplary; peptidyl linkers within the scope of this invention may be much longer and may include other residues. A peptidyl linker can contain, e.g., a cysteine, another thiol, or nucleophile for conjugation with a half-life extending moiety. In another embodiment, the linker contains a cysteine or homocysteine residue, or other 2-amino-ethanethiol or 3-amino-propanethiol moiety for conjugation to maleimide, iodoacetaamide or thioester, functionalized half-life extending moiety.

Another useful peptidyl linker is a large, flexible linker comprising a random Gly/Ser/Thr sequence, for example: GSGSATGGSGSTASSGSGSATH (SEQ ID NO:178) or HGSGSATGGSGSTASSGSGSAT (SEQ ID NO:179), that is estimated to be about the size of a 1 kDa PEG molecule. Alternatively, a useful peptidyl linker may be comprised of amino acid sequences known in the art to form rigid helical structures (e.g., Rigid linker: -AEAAAKEAAAKEAAAKAGG-) (SEQ ID NO: 180). Additionally, a peptidyl linker can also comprise a non-peptidyl segment such as a 6 carbon aliphatic molecule of the formula —CH₂—CH₂—CH₂—CH₂—CH₂—CH₂—. The peptidyl linkers can be altered to form derivatives as described herein.

Optionally, a non-peptidyl linker moiety is also useful for conjugating the half-life extending moiety to the peptide portion of the half-life extending moiety-conjugated toxin peptide analog. For example, alkyl linkers such as —NH—(CH₂)ₛ— C(O)—, wherein s=2-20 can be used. These alkyl linkers may further be substituted by any non-sterically hindering group such as lower alkyl (e.g., C₁-C₆) lower acyl, halogen (e.g., Cl, Br), CN, NH₂, phenyl, etc. Exemplary non-peptidyl linkers are polyethylene glycol (PEG) linkers (e.g., shown below):

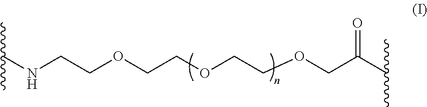

wherein n is such that the linker has a molecular weight of about 100 to about 5000 Daltons (Da), preferably about 100 to about 500 Da.

In one embodiment, the non-peptidyl linker is aryl. The linkers may be altered to form derivatives in the same manner as described in the art, e.g., in Sullivan et al., Toxin Peptide Therapeutic Agents, US2007/0071764; Sullivan et al., Toxin Peptide Therapeutic Agents, PCT/US2007/022831, published as WO 2008/088422; and U.S. Provisional Application Ser. No. 61/210,594, filed Mar. 20, 2009, which are all incorporated herein by reference in their entireties.

In addition, PEG moieties may be attached to the N-terminal amine or selected side chain amines by either reductive alkylation using PEG aldehydes or acylation using hydroxysuccinimido or carbonate esters of PEG, or by thiol conjugation.

“Aryl” is phenyl or phenyl vicinally-fused with a saturated, partially-saturated, or unsaturated 3-, 4-, or 5 membered carbon bridge, the phenyl or bridge being substituted by 0, 1, 2 or 3 substituents selected from C₁₋₈ alkyl, C₁₋₄ haloalkyl or halo.

“Heteroaryl” is an unsaturated 5, 6 or 7 membered monocyclic or partially-saturated or unsaturated 6-, 7-, 8-, 9-, 10- or 11 membered bicyclic ring, wherein at least one ring is unsaturated, the monocyclic and the bicyclic rings containing 1, 2, 3 or 4 atoms selected from N, O and S, wherein the ring is substituted by 0, 1, 2 or 3 substituents selected from C₁₋₈ alkyl, C₁₋₄haloalkyl and halo.

Non-peptide portions of the inventive composition of matter, such as non-peptidyl linkers or non-peptide half-life extending moieties can be synthesized by conventional organic chemistry reactions.

The above is merely illustrative and not an exhaustive treatment of the kinds of linkers that can optionally be employed in accordance with the present invention.

Production of Antigen Binding Protein Variants.

As noted above, recombinant DNA- and/or RNA-mediated protein expression and protein engineering techniques, or any other methods of preparing peptides, are applicable to the making of the inventive compositions. For example, polypeptides can be made in transformed host cells. Briefly, a recombinant DNA molecule, or construct, coding for the peptide is prepared. Methods of preparing such DNA molecules are well known in the art. For instance, sequences encoding the peptides can be excised from DNA using suitable restriction enzymes. Any of a large number of available and well-known host cells may be used in the practice of this invention. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all hosts may be equally effective for the expression of a particular DNA sequence. Within these general guidelines, useful microbial host cells in culture include bacteria (such as Escherichia coli sp.), yeast (such as Saccharomyces sp.) and other fungal cells, insect cells, plant cells, mammalian (including human) cells, e.g., CHO cells and HEK-293 cells, and others noted herein or otherwise known in the art. Modifications can be made at the DNA level, as well. The peptide-encoding DNA sequence may be changed to codons more compatible with the chosen host cell. For E. coli, optimized codons are known in the art. Codons can be substituted to eliminate restriction sites or to include silent restriction sites, which may aid in processing of the DNA in the selected host cell. Next, the transformed host is cultured and purified. Host cells may be cultured under conventional fermentation conditions so that the desired compounds are expressed. Such fermentation conditions are well known in the art. In addition, the DNA optionally further encodes, 5′ to the coding region of a fusion protein, a signal peptide sequence (e.g., a secretory signal peptide) operably linked to the expressed specific binding agent or antigen binding protein, e.g., an immunoglobulin protein. For further examples of appropriate recombinant methods and exemplary DNA constructs useful for recombinant expression of the inventive compositions by mammalian cells, including dimeric Fc fusion proteins (“peptibodies”) or chimeric immunoglobulin (light chain+heavy chain)-Fc heterotrimers (“hemibodies”), conjugated to specific binding agents of the invention, see, e.g., Sullivan et al., Toxin Peptide Therapeutic Agents, US2007/0071764; Sullivan et al., Toxin Peptide Therapeutic Agents, PCT/US2007/022831, published as WO 2008/088422; and U.S. Provisional Application Ser. No. 61/210,594, filed Mar. 20, 2009, which are all incorporated herein by reference in their entireties.

Amino acid sequence variants of the desired antigen binding protein may be prepared by introducing appropriate nucleotide changes into the encoding DNA, or by peptide synthesis. Such variants include, for example, deletions and/or insertions and/or substitutions of residues within the amino acid sequences of the antigen binding proteins or antibodies. Any combination of deletion, insertion, and substitution is made to arrive at the final construct, provided that the final construct possesses the desired characteristics. The amino acid changes also may alter post-translational processes of the antigen binding protein, such as changing the number or position of glycosylation sites. In certain instances, antigen binding protein variants are prepared with the intent to modify those amino acid residues which are directly involved in epitope binding. In other embodiments, modification of residues which are not directly involved in epitope binding or residues not involved in epitope binding in any way, is desirable, for purposes discussed herein. Mutagenesis within any of the CDR regions and/or framework regions is contemplated. Covariance analysis techniques can be employed by the skilled artisan to design useful modifications in the amino acid sequence of the antigen binding protein, including an antibody or antibody fragment. (E.g., Choulier, et al., Covariance Analysis of Protein Families The Case of the Variable Domains of Antibodies, Proteins: Structure, Function, and Genetics 41:475-484 (2000); Demarest et al., Optimization of the Antibody C_{H}3 Domain by Residue Frequency Analysis of IgG Sequences, J. Mol. Biol. 335:41-48 (2004); Hugo et al., VL position 34 is a key determinant for the engineering of stable antibodies with fast dissociation rates, Protein Engineering 16(5):381-86 (2003); Aurora et al., Sequence covariance networks, methods and uses thereof, US 2008/0318207 A1; Glaser et al., Stabilized polypeptide compositions, US 2009/0048122 A1; Urech et al., Sequence based engineering and optimization of single chain antibodies, WO 2008/110348 A1; Borras et al., Methods of modifying antibodies, and modified antibodies with improved functional properties, WO 2009/000099 A2). Such modifications determined by covariance analysis can improve potency, pharmacokinetic, pharmacodynamic, and/or manufacturability characteristics of an antigen binding protein.

Nucleic acid molecules encoding amino acid sequence variants of the antigen binding protein or antibody are prepared by a variety of methods known in the art. Such methods include oligonucleotide-mediated (or site-directed) mutagenesis, PCR mutagenesis, and cassette mutagenesis of an earlier prepared variant or a non-variant version of the antigen binding protein.

Substitutional mutagenesis within any of the hypervariable or CDR regions or framework regions is contemplated. A useful method for identification of certain residues or regions of the antigen binding protein that are preferred locations for mutagenesis is called “alanine scanning mutagenesis,” as described by Cunningham and Wells Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed variants are screened for the desired activity.

Some embodiments of the antigen binding proteins of the present invention can also be made by synthetic methods. Solid phase synthesis is the preferred technique of making individual peptides since it is the most cost-effective method of making small peptides. For example, well known solid phase synthesis techniques include the use of protecting groups, linkers, and solid phase supports, as well as specific protection and deprotection reaction conditions, linker cleavage conditions, use of scavengers, and other aspects of solid phase peptide synthesis. Suitable techniques are well known in the art. (E.g., Merrifield (1973), Chem. Polypeptides, pp. 335-61 (Katsoyannis and Panayotis eds.); Merrifield (1963), J. Am. Chem. Soc. 85: 2149; Davis et al. (1985), Biochem. Intl. 10: 394-414; Stewart and Young (1969), Solid Phase Peptide Synthesis; U.S. Pat. No. 3,941,763; Finn et al. (1976), The Proteins (3rd ed.) 2: 105-253; and Erickson et al. (1976), The Proteins (3rd ed.) 2: 257-527; “Protecting Groups in Organic Synthesis,” 3rd Edition, T. W. Greene and P. G. M. Wuts, Eds., John Wiley & Sons, Inc., 1999; NovaBiochem Catalog, 2000; “Synthetic Peptides, A User's Guide,” G. A. Grant, Ed., W.H. Freeman & Company, New York, N.Y., 1992; “Advanced Chemtech Handbook of Combinatorial & Solid Phase Organic Chemistry,” W. D. Bennet, J. W. Christensen, L. K. Hamaker, M. L. Peterson, M. R. Rhodes, and H. H. Saneii, Eds., Advanced Chemtech, 1998; “Principles of Peptide Synthesis, 2nd ed.,” M. Bodanszky, Ed., Springer-Verlag, 1993; “The Practice of Peptide Synthesis, 2nd ed.,” M. Bodanszky and A. Bodanszky, Eds., Springer-Verlag, 1994; “Protecting Groups,” P. J. Kocienski, Ed., Georg Thieme Verlag, Stuttgart, Germany, 1994; “Fmoc Solid Phase Peptide Synthesis, A Practical Approach,” W. C. Chan and P. D. White, Eds., Oxford Press, 2000, G. B. Fields et al., Synthetic Peptides: A User's Guide, 1990, 77-183). For further examples of synthetic and purification methods known in the art, which are applicable to making the inventive compositions of matter, see, e.g., Sullivan et al., Toxin Peptide Therapeutic Agents, US2007/0071764 and Sullivan et al., Toxin Peptide Therapeutic Agents, PCT/US2007/022831, published as WO 2008/088422 A2, which are both incorporated herein by reference in their entireties.

In further describing any of the antigen binding proteins herein, as well as variants, a one-letter abbreviation system is frequently applied to designate the identities of the twenty “canonical” amino acid residues generally incorporated into naturally occurring peptides and proteins (Table 4). Such one-letter abbreviations are entirely interchangeable in meaning with three-letter abbreviations, or non-abbreviated amino acid names. Within the one-letter abbreviation system used herein, an upper case letter indicates a L-amino acid, and a lower case letter indicates a D-amino acid. For example, the abbreviation “R” designates L-arginine and the abbreviation “r” designates D-arginine.

TABLE 4
One-letter abbreviations for the canonical amino acids.
Three-letter abbreviations are in parentheses.

| | |
|---|---|
| Alanine (Ala) | A |
| Glutamine (Gln) | Q |
| Leucine (Leu) | L |
| Serine (Ser) | S |
| Arginine (Arg) | R |
| Glutamic Acid (Glu) | E |
| Lysine (Lys) | K |
| Threonine (Thr) | T |
| Asparagine (Asn) | N |
| Glycine (Gly) | G |
| Methionine (Met) | M |
| Tryptophan (Trp) | W |
| Aspartic Acid (Asp) | D |
| Histidine (His) | H |
| Phenylalanine (Phe) | F |
| Tyrosine (Tyr) | Y |
| Cysteine (Cys) | C |
| Isoleucine (Ile) | I |
| Proline (Pro) | P |
| Valine (Val) | V |

An amino acid substitution in an amino acid sequence is typically designated herein with a one-letter abbreviation for the amino acid residue in a particular position, followed by the numerical amino acid position relative to an original sequence of interest, which is then followed by the one-letter symbol for the amino acid residue substituted in. For example, “T30D” symbolizes a substitution of a threonine residue by an aspartate residue at amino acid position 30, relative to the original sequence of interest. Another example, “S218G” symbolizes a substitution of a serine residue by a glycine residue at amino acid position 218, relative to the original sequence of interest, e.g., SEQ ID NO:2.

Non-canonical amino acid residues can be incorporated into a polypeptide within the scope of the invention by employing known techniques of protein engineering that use recombinantly expressing cells. (See, e.g., Link et al., Non-canonical amino acids in protein engineering, Current Opinion in Biotechnology, 14(6):603-609 (2003)). The term “non-canonical amino acid residue” refers to amino acid residues in D- or L-form that are not among the 20 canonical amino acids generally incorporated into naturally occurring proteins, for example, β-amino acids, homoamino acids, cyclic amino acids and amino acids with derivatized side chains. Examples include (in the L-form or D-form) β-alanine, β-aminopropionic acid, piperidinic acid, aminocaprioic acid, aminoheptanoic acid, aminopimelic acid, desmosine, diaminopimelic acid, N^{α}-ethylglycine, N^{α}-ethylaspargine, hydroxylysine, allo-hydroxylysine, isodesmosine, allo-isoleucine, ω-methylarginine, N^{α}-methylglycine, N^{α}-methylisoleucine, N^{α}-methylvaline, γ-carboxyglutamate, ε-N,N,N-trimethyllysine, ε-N-acetyllysine, O-phosphoserine, N^{α}-acetylserine, N^{α}-formylmethionine, 3-methylhistidine, 5-hydroxylysine, and other similar amino acids, and those listed in Table 5 below, and derivatized forms of any of these as described herein. Table 5 contains some exemplary non-canonical amino acid residues that are useful in accordance with the present invention and associated abbreviations as typically used herein, although the skilled practitioner will understand that different abbreviations and nomenclatures may be applicable to the same substance and appear interchangeably herein.

TABLE 5
Useful non-canonical amino acids for amino acid addition, insertion, or
substitution into peptide sequences in accordance with the present
invention. In the event an abbreviation listed in Table 5 differs from
another abbreviation for the same substance disclosed elsewhere
herein, both abbreviations are understood to be applicable.
The amino acids listed in Table 5 can be in the L-form or D-form.

| Amino Acid | Abbreviation(s) |
|---|---|
| Acetamidomethyl | Acm |
| Acetylarginine | acetylarg |
| α-aminoadipic acid | Aad |
| aminobutyric acid | Abu |
| 6-aminohexanoic acid | Ahx; εAhx |
| 3-amino-6-hydroxy-2-piperidone | Ahp |
| 2-aminoindane-2-carboxylic acid | Aic |
| α-amino-isobutyric acid | Aib |
| 3-amino-2-naphthoic acid | Anc |
| 2-aminotetraline-2-carboxylic acid | Atc |
| Aminophenylalanine | Aminophe; Amino-Phe |
| 4-amino-phenylalanine | 4AmP |
| 4-amidino-phenylalanine | 4AmPhe |
| 2-amino-2-(1-carbamimidoylpiperidin-4- | 4AmPig |
| yl)acetic acid | |
| Arg ψ(CH₂NH)-reduced amide bond | rArg |
| β-homoarginine | bhArg |
| β-homolysine | bhomoK |
| β-homo Tic | BhTic |
| β-homophenylalanine | BhPhe |
| β-homoproline | BhPro |
| β-homotryptophan | BhTrp |
| 4,4′-biphenylalanine | Bip |
| β,β-diphenyl-alanine | BiPhA |
| β-phenylalanine | BPhe |
| p-carboxyl-phenylalanine | Cpa |
| Citrulline | Cit |
| Cyclohexylalanine | Cha |
| Cyclohexylglycine | Chg |
| Cyclopentylglycine | Cpg |
| 2-amino-3-guanidinopropanoic acid | 3G-Dpr |
| α,γ-diaminobutyric acid | Dab |
| 2,4-diaminobutyric acid | Dbu |
| diaminopropionic acid | Dap |
| α,β-diaminopropionoic acid (or 2,3- | Dpr |
| diaminopropionic acid | |
| 3,3-diphenylalanine | Dip |
| 4-guanidino phenylalanine | Guf |
| 4-guanidino proline | 4GuaPr |
| Homoarginine | hArg; hR |
| Homocitrulline | hCit |
| Homoglutamine | hQ |
| Homolysine | hLys; hK; homoLys |
| Homophenylalanine | hPhe; homoPhe |
| 4-hydroxyproline (or hydroxyproline) | Hyp |
| 2-indanylglycine (or indanylglycine) | IgI |
| indoline-2-carboxylic acid | Idc |
| Iodotyrosine | I-Tyr |
| Lys ψ(CH₂NH)-reduced amide bond | rLys |
| methinine oxide | Met[O] |
| methionine sulfone | Met[O]₂ |
| N^{α}-methylarginine | NMeR |
| Nα-[(CH₂)₃NHCH(NH)NH₂] substituted | N-Arg |
| glycine | |
| N^{α}-methylcitrulline | NMeCit |
| N^{α}-methylglutamine | NMeQ |
| N^{α}-methylhomocitrulline | N^{α}-MeHoCit |
| N^{α}-methylhomolysine | NMeHoK |
| N^{α}-methylleucine | N^{α}-MeL; NMeL; NMeLeu; |
| | NMe-Leu |
| N^{α}-methyllysine | NMe-Lys |
| Nε-methyl-lysine | N-eMe-K |
| Nε-ethyl-lysine | N-eEt-K |
| Nε-isopropyl-lysine | N-eIPr-K |
| N^{α}-methylnorleucine | NMeNle; NMe-Nle |
| N^{α}-methylornithine | N^{α}-MeOrn; NMeOrn |
| N^{α}-methylphenylalanine | NMe-Phe |
| 4-methyl-phenylalanine | MePhe |
| α-methylphenyalanine | AMeF |
| N^{α}-methylthreonine | NMe-Thr; NMeThr |
| N^{α}-methylvaline | NMeVal; NMe-Val |
| Nε-(O-(aminoethyl)-O′-(2-propanoyl)- | K(NPeg11) |
| undecaethyleneglycol)-Lysine | |
| Nε-(O-(aminoethyl)-O′-(2-propanoyl)- | K(NPeg27) |
| (ethyleneglycol)27-Lysine | |
| 3-(1-naphthyl)alanine | 1-Nal; 1Nal |
| 3-(2-naphthyl)alanine | 2-Nal; 2Nal |
| nipecotic acid | Nip |
| Nitrophenylalanine | nitrophe |
| norleucine | Nle |
| norvaline | Nva or Nvl |
| O-methyltyrosine | Ome-Tyr |
| octahydroindole-2-carboxylic acid | Oic |
| Ornithine | Orn |
| Orn ψ(CH2NH)-reduced amide bond | rOrn |
| 4-piperidinylalanine | 4PipA |
| 4-pyridinylalanine | 4Pal |
| 3-pyridinylalanine | 3Pal |
| 2-pyridinylalanine | 2Pal |
| para-aminophenylalanine | 4AmP; 4-Amino-Phe |
| para-iodophenylalanine (or 4- | pI-Phe |
| iodophenylalanine) | |
| Phenylglycine | Phg |
| 4-phenyl-phenylalanine (or | 4Bip |
| biphenylalanine) | |
| 4,4′-biphenyl alanine | Bip |
| pipecolic acid | Pip |
| 4-amino-1-piperidine-4-carboxylic acid | 4Pip |
| Sarcosine | Sar |
| 1,2,3,4-tetrahydroisoquinoline | Tic |
| 1,2,3,4-tetrahydroisoquinoline-1- | Tiq |
| carboxylic acid | |
| 1,2,3,4-tetrahydroisoquinoline-7- | Hydroxyl-Tic |
| hydroxy-3-carboxylic acid | |
| 1,2,3,4-tetrahydronorharman-3- | Tpi |
| carboxylic acid | |
| thiazolidine-4-carboxylic acid | Thz |
| 3-thienylalanine | Thi |

Nomenclature and Symbolism for Amino Acids and Peptides by the UPAC-IUB Joint Commission on Biochemical Nomenclature (JCBN) have been published in the following documents: Biochem. J., 1984, 219, 345-373; Eur. J. Biochem., 1984, 138, 9-37; 1985, 152, 1; 1993, 213, 2; Internat. J. Pept. Prot. Res., 1984, 24, following p 84; J. Biol. Chem., 1985, 260, 14-42; Pure Appl. Chem., 1984, 56, 595-624; Amino Acids and Peptides, 1985, 16, 387-410; Biochemical Nomenclature and Related Documents, 2nd edition, Portland Press, 1992, pages 39-69.

The one or more useful modifications to peptide domains of the inventive antigen binding protein can include amino acid additions or insertions, amino acid deletions, peptide truncations, amino acid substitutions, and/or chemical derivatization of amino acid residues, accomplished by known chemical techniques. For example, the thusly modified amino acid sequence includes at least one amino acid residue inserted or substituted therein, relative to the amino acid sequence of the native sequence of interest, in which the inserted or substituted amino acid residue has a side chain comprising a nucleophilic or electrophilic reactive functional group by which the peptide is conjugated to a linker and/or half-life extending moiety. In accordance with the invention, useful examples of such a nucleophilic or electrophilic reactive functional group include, but are not limited to, a thiol, a primary amine, a seleno, a hydrazide, an aldehyde, a carboxylic acid, a ketone, an aminooxy, a masked (protected) aldehyde, or a masked (protected) keto functional group. Examples of amino acid residues having a side chain comprising a nucleophilic reactive functional group include, but are not limited to, a lysine residue, a homolysine, an α,β-diaminopropionic acid residue, an α,γ-diaminobutyric acid residue, an ornithine residue, a cysteine, a homocysteine, a glutamic acid residue, an aspartic acid residue, or a selenocysteine residue.

Amino acid residues are commonly categorized according to different chemical and/or physical characteristics. The term “acidic amino acid residue” refers to amino acid residues in D- or L-form having side chains comprising acidic groups. Exemplary acidic residues include aspartatic acid and glutamatic acid residues. The term “alkyl amino acid residue” refers to amino acid residues in D- or L-form having C₁₋₆ alkyl side chains which may be linear, branched, or cyclized, including to the amino acid amine as in proline, wherein the C₁₋₆ alkyl is substituted by 0, 1, 2 or 3 substituents selected from C₁₋₄ haloalkyl, halo, cyano, nitro, —C(═O)R^{b}, —C(═O)OR^{a}, —C(═O)NR^{a}R^{a}, —C(═NR^{a})NR^{a}R^{a}, —NR^{a}C(═NR^{a})NR^{a}R^{a}, —OR^{a}, —OC(═O)R^{b}, —OC(═O)NR^{a}R^{a}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}; wherein R^{a} is independently, at each instance, H or R^{b}; and R^{b} is independently, at each instance C₁₋₆ alkyl substituted by 0, 1, 2 or 3 substituents selected from halo, C₁₋₄alk, C₁₋₃ haloalk, —OC₁₋₄alk, —NH₂, —NHC₁₋₄alk, and —N(C₁₋₄ alk)Cl₁₋₄ alk; or any protonated form thereof, including alanine, valine, leucine, isoleucine, proline, serine, threonine, lysine, arginine, histidine, aspartate, glutamate, asparagine, glutamine, cysteine, methionine, hydroxyproline, but which residues do not contain an aryl or aromatic group. The term “aromatic amino acid residue” refers to amino acid residues in D- or L-form having side chains comprising aromatic groups. Exemplary aromatic residues include tryptophan, tyrosine, 3-(1-naphthyl)alanine, or phenylalanine residues. The term “basic amino acid residue” refers to amino acid residues in D- or L-form having side chains comprising basic groups. Exemplary basic amino acid residues include histidine, lysine, homolysine, ornithine, arginine, N-methyl-arginine, o-aminoarginine, o-methyl-arginine, 1-methyl-histidine, 3-methyl-histidine, and homoarginine (hR) residues. The term “hydrophilic amino acid residue” refers to amino acid residues in D- or L-form having side chains comprising polar groups. Exemplary hydrophilic residues include cysteine, serine, threonine, histidine, lysine, asparagine, aspartate, glutamate, glutamine, and citrulline (Cit) residues. The terms “lipophilic amino acid residue” refers to amino acid residues in D- or L-form having sidechains comprising uncharged, aliphatic or aromatic groups. Exemplary lipophilic sidechains include phenylalanine, isoleucine, leucine, methionine, valine, tryptophan, and tyrosine. Alanine (A) is amphiphilic—it is capable of acting as a hydrophilic or lipophilic residue. Alanine, therefore, is included within the definition of both “lipophilic residue” and “hydrophilic residue.” The term “nonfunctional amino acid residue” refers to amino acid residues in D- or L-form having side chains that lack acidic, basic, or aromatic groups. Exemplary neutral amino acid residues include methionine, glycine, alanine, valine, isoleucine, leucine, and norleucine (Nle) residues.

Additional useful embodiments of can result from conservative modifications of the amino acid sequences of the polypeptides disclosed herein. Conservative modifications will produce half-life extending moiety-conjugated peptides having functional, physical, and chemical characteristics similar to those of the conjugated (e.g., PEG-conjugated) peptide from which such modifications are made. Such conservatively modified forms of the conjugated polypeptides disclosed herein are also contemplated as being an embodiment of the present invention.

In contrast, substantial modifications in the functional and/or chemical characteristics of peptides may be accomplished by selecting substitutions in the amino acid sequence that differ significantly in their effect on maintaining (a) the structure of the molecular backbone in the region of the substitution, for example, as an α-helical conformation, (b) the charge or hydrophobicity of the molecule at the target site, or (c) the size of the molecule.

For example, a “conservative amino acid substitution” may involve a substitution of a native amino acid residue with a normative residue such that there is little or no effect on the polarity or charge of the amino acid residue at that position. Furthermore, any native residue in the polypeptide may also be substituted with alanine, as has been previously described for “alanine scanning mutagenesis” (see, for example, MacLennan et al., Acta Physiol. Scand. Suppl., 643:55-67 (1998); Sasaki et al., 1998, Adv. Biophys. 35:1-24 (1998), which discuss alanine scanning mutagenesis).

Desired amino acid substitutions (whether conservative or non-conservative) can be determined by those skilled in the art at the time such substitutions are desired. For example, amino acid substitutions can be used to identify important residues of the peptide sequence, or to increase or decrease the affinity of the peptide or vehicle-conjugated peptide molecules described herein.

Naturally occurring residues may be divided into classes based on common side chain properties:

1) hydrophobic: norleucine (Nor or Nle), Met, Ala, Val, Leu, Ile;

2) neutral hydrophilic: Cys, Ser, Thr, Asn, Gln;

3) acidic: Asp, Glu;

4) basic: H is, Lys, Arg;

5) residues that influence chain orientation: Gly, Pro; and

6) aromatic: Trp, Tyr, Phe.

Conservative amino acid substitutions may involve exchange of a member of one of these classes with another member of the same class. Conservative amino acid substitutions may encompass non-naturally occurring amino acid residues, which are typically incorporated by chemical peptide synthesis rather than by synthesis in biological systems. These include peptidomimetics and other reversed or inverted forms of amino acid moieties.

Non-conservative substitutions may involve the exchange of a member of one of these classes for a member from another class. Such substituted residues may be introduced into regions of the toxin peptide analog.

In making such changes, according to certain embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art (see, for example, Kyte et al., 1982, J. Mol. Biol. 157:105-131). It is known that certain amino acids may be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those that are within ±1 are included, and in certain embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as disclosed herein. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0+1); glutamate (+3.0+1); serine (+0.3); asparagine (+0.2); glutamine (+0.2); glycine (0); threonine (−0.4); proline (−0.5+1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those that are within ±1 are included, and in certain embodiments, those within ±0.5 are included. One may also identify epitopes from primary amino acid sequences on the basis of hydrophilicity. These regions are also referred to as “epitopic core regions.”

Examples of conservative substitutions include the substitution of one non-polar (hydrophobic) amino acid residue such as isoleucine, valine, leucine norleucine, alanine, or methionine for another, the substitution of one polar (hydrophilic) amino acid residue for another such as between arginine and lysine, between glutamine and asparagine, between glycine and serine, the substitution of one basic amino acid residue such as lysine, arginine or histidine for another, or the substitution of one acidic residue, such as aspartic acid or glutamic acid for another. The phrase “conservative amino acid substitution” also includes the use of a chemically derivatized residue in place of a non-derivatized residue, provided that such polypeptide displays the requisite bioactivity. Other exemplary amino acid substitutions that can be useful in accordance with the present invention are set forth in Table 6 below.

TABLE 6
Some Useful Amino Acid Substitutions.

| Original | Exemplary |
|---|---|
| Residues | Substitutions |
| Ala | Val, Leu, Ile |
| Arg | Lys, Gln, Asn |
| Asn | Gln |
| Asp | Glu |
| Cys | Ser, Ala |
| Gln | Asn |
| Glu | Asp |
| Gly | Pro, Ala |
| His | Asn, Gln, Lys, Arg |
| Ile | Leu, Val, Met, Ala, |
| | Phe, Norleucine |
| Leu | Norleucine, Ile, |
| | Val, Met, Ala, Phe |
| Lys | Arg, 1,4-Diamino- |
| | butyric Acid, Gln, |
| | Asn |
| Met | Leu, Phe, Ile |
| Phe | Leu, Val, Ile, Ala, |
| | Tyr |
| Pro | Ala |
| Ser | Thr, Ala, Cys |
| Thr | Ser |
| Trp | Tyr, Phe |
| Tyr | Trp, Phe, Thr, Ser |
| Val | Ile, Met, Leu, Phe, |
| | Ala, Norleucine |

Ordinarily, amino acid sequence variants of the antigen binding protein will have an amino acid sequence having at least 60% amino acid sequence identity with the original antigen binding protein or antibody amino acid sequences of either the heavy or the light chain variable region, or at least 65%, or at least 70%, or at least 75% or at least 80% identity, more preferably at least 85% identity, even more preferably at least 90% identity, and most preferably at least 95% identity, including for example, 80%, 81%, 82%, 83%, 84%, 85%, 86%, 87%, 88%, 89%, 90%, 91%, 92%, 93%, 94%, 95%, 96%, 97%, 98%, 99%, and 100%. Identity or homology with respect to this sequence is defined herein as the percentage of amino acid residues in the candidate sequence that are identical with the original sequence, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antigen binding protein or antibody sequence shall be construed as affecting sequence identity or homology.

Amino acid sequence insertions include amino- and/or carboxyl-terminal fusions ranging in length from one residue to polypeptides containing a hundred or more residues, as well as intra-sequence insertions of single or multiple amino acid residues. Examples of terminal insertions include an antigen binding protein with an N-terminal methionyl residue or the antigen binding protein (including antibody or antibody fragment) fused to an epitope tag or a salvage receptor binding epitope. Other insertional variants of the antigen binding protein or antibody molecule include the fusion to a polypeptide which increases the serum half-life of the antigen binding protein, e.g. at the N-terminus or C-terminus.

Examples of epitope tags include the flu HA tag polypeptide and its antibody 12CA5 [Field et al., Mol. Cell. Biol. 8: 2159-2165 (1988)]; the c-myc tag and the 8F9, 3C7, 6E10, G4, B7 and 9E10 antibodies thereto [Evan et al., Mol. Cell. Biol. 5(12): 3610-3616 (1985)]; and the Herpes Simplex virus glycoprotein D (gD) tag and its antibody [Paborsky et al., Protein Engineering 3(6): 547-553 (1990)]. Other exemplary tags are a poly-histidine sequence, generally around six histidine residues, that permits isolation of a compound so labeled using nickel chelation. Other labels and tags, such as the FLAG®tag (Eastman Kodak, Rochester, N.Y.) are well known and routinely used in the art.

Some particular, non-limiting, embodiments of amino acid substitution variants of the inventive antigen binding proteins, including antibodies and antibody fragments are exemplified below.

Any cysteine residue not involved in maintaining the proper conformation of the antigen binding protein also may be substituted, generally with serine, to improve the oxidative stability of the molecule and prevent aberrant crosslinking. Conversely, cysteine bond(s) may be added to the antigen binding protein to improve its stability (particularly where the antigen binding protein is an antibody fragment such as an Fv fragment).

In certain instances, antigen binding protein variants are prepared with the intent to modify those amino acid residues which are directly involved in epitope binding. In other embodiments, modification of residues which are not directly involved in epitope binding or residues not involved in epitope binding in any way, is desirable, for purposes discussed herein. Mutagenesis within any of the CDR regions and/or framework regions is contemplated.

In order to determine which antigen binding protein amino acid residues are important for epitope recognition and binding, alanine scanning mutagenesis can be performed to produce substitution variants. See, for example, Cunningham et al., Science, 244:1081-1085 (1989), the disclosure of which is incorporated herein by reference in its entirety. In this method, individual amino acid residues are replaced one-at-a-time with an alanine residue and the resulting anti-Aβ antigen binding protein is screened for its ability to bind its specific epitope relative to the unmodified polypeptide. Modified antigen binding proteins with reduced binding capacity are sequenced to determine which residue was changed, indicating its significance in binding or biological properties.

Substitution variants of antigen binding proteins can be prepared by affinity maturation wherein random amino acid changes are introduced into the parent polypeptide sequence. See, for example, Ouwehand et al., Vox Sang 74 (Suppl 2):223-232, 1998; Rader et al., Proc. Natl. Acad. Sci. USA 95:8910-8915, 1998; Dall' Acqua et al., Curr. Opin. Struct. Biol. 8:443-450, 1998, the disclosures of which are incorporated herein by reference in their entireties. Affinity maturation involves preparing and screening the anti-hOrai1 antigen binding proteins, or variants thereof and selecting from the resulting variants those that have modified biological properties, such as increased binding affinity relative to the parent anti-Aβ antigen binding protein. A convenient way for generating substitutional variants is affinity maturation using phage display. Briefly, several hypervariable region sites are mutated to generate all possible amino substitutions at each site. The variants thus generated are expressed in a monovalent fashion on the surface of filamentous phage particles as fusions to the gene III product of M13 packaged within each particle. The phage-displayed variants are then screened for their biological activity (e.g., binding affinity). See e.g., WO 92/01047, WO 93/112366, WO 95/15388 and WO 93/19172.

Current antibody affinity maturation methods belong to two mutagenesis categories: stochastic and nonstochastic. Error prone PCR, mutator bacterial strains (Low et al., J. Mol. Biol. 260, 359-68, 1996), and saturation mutagenesis (Nishimiya et al., J. Biol. Chem. 275:12813-20, 2000; Chowdhury, P. S. Methods Mol. Biol. 178, 269-85, 2002) are typical examples of stochastic mutagenesis methods (Rajpal et al., Proc Natl Acad Sci US A. 102:8466-71, 2005). Nonstochastic techniques often use alanine-scanning or site-directed mutagenesis to generate limited collections of specific muteins. Some methods are described in further detail below.

Affinity maturation via panning methods-Affinity maturation of recombinant antibodies is commonly performed through several rounds of panning of candidate antibodies in the presence of decreasing amounts of antigen. Decreasing the amount of antigen per round selects the antibodies with the highest affinity to the antigen thereby yielding antibodies of high affinity from a large pool of starting material. Affinity maturation via panning is well known in the art and is described, for example, in Huls et al. (Cancer Immunol Immunother. 50:163-71, 2001). Methods of affinity maturation using phage display technologies are described elsewhere herein and known in the art (see e.g., Daugherty et al., Proc Natl Acad Sci USA. 97:2029-34, 2000).

Look-through mutagenesis-Look-through mutagenesis (LTM) (Rajpal et al., Proc Natl Acad Sci USA. 102:8466-71, 2005) provides a method for rapidly mapping the antibody-binding site. For L™, nine amino acids, representative of the major side-chain chemistries provided by the 20 natural amino acids, are selected to dissect the functional side-chain contributions to binding at every position in all six CDRs of an antibody. LTM generates a positional series of single mutations within a CDR where each “wild type” residue is systematically substituted by one of nine selected amino acids. Mutated CDRs are combined to generate combinatorial single-chain variable fragment (scFv) libraries of increasing complexity and size without becoming prohibitive to the quantitative display of all muteins. After positive selection, clones with improved binding are sequenced, and beneficial mutations are mapped.

Error-prone PCR—Error-prone PCR involves the randomization of nucleic acids between different selection rounds. The randomization occurs at a low rate by the intrinsic error rate of the polymerase used but can be enhanced by error-prone PCR (Zaccolo et al., J. Mol. Biol. 285:775-783, 1999) using a polymerase having a high intrinsic error rate during transcription (Hawkins et al., J Mol. Biol. 226:889-96, 1992). After the mutation cycles, clones with improved affinity for the antigen are selected using routine methods in the art.

Techniques utilizing gene shuffling and directed evolution may also be used to prepare and screen anti-hOrai1 ECL2 antigen binding proteins, or variants thereof, for desired activity. For example, Jermutus et al., Proc Natl Acad Sci U S A., 98(1):75-80 (2001) showed that tailored in vitro selection strategies based on ribosome display were combined with in vitro diversification by DNA shuffling to evolve either the off-rate or thermodynamic stability of scFvs; Fermer et al., Tumour Biol. 2004 Jan.-Apr.; 25(1-2):7-13 reported that use of phage display in combination with DNA shuffling raised affinity by almost three orders of magnitude. Dougherty et al., Proc Natl Acad Sci USA. 2000 Feb. 29; 97(5):2029-2034 reported that (i) functional clones occur at an unexpectedly high frequency in hypermutated libraries, (ii) gain-of-function mutants are well represented in such libraries, and (iii) the majority of the scFv mutations leading to higher affinity correspond to residues distant from the binding site.

Alternatively, or in addition, it may be beneficial to analyze a crystal structure of the antigen-antibody complex to identify contact points between the antibody and antigen, or to use computer software to model such contact points. Such contact residues and neighboring residues are candidates for substitution according to the techniques elaborated herein. Once such variants are generated, they are subjected to screening as described herein and antibodies with superior properties in one or more relevant assays may be selected for further development.

Antigen Binding Proteins with Modified Carbohydrate

Antigen binding protein variants can also be produced that have a modified glycosylation pattern relative to the parent polypeptide, for example, adding or deleting one or more of the carbohydrate moieties bound to the antigen binding protein, and/or adding or deleting one or more glycosylation sites in the antigen binding protein.

Glycosylation of polypeptides, including antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. The presence of either of these tripeptide sequences in a polypeptide creates a potential glycosylation site. Thus, N-linked glycosylation sites may be added to a antigen binding protein by altering the amino acid sequence such that it contains one or more of these tripeptide sequences. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. O-linked glycosylation sites may be added to a antigen binding protein by inserting or substituting one or more serine or threonine residues to the sequence of the original antigen binding protein or antibody.

Altered Effector Function

Cysteine residue(s) may be removed or introduced in the Fc region of an antibody or Fc-containing polypeptide, thereby eliminating or increasing interchain disulfide bond formation in this region. A homodimeric antigen binding protein thus generated may have improved internalization capability and/or increased complement-mediated cell killing and antibody-dependent cellular cytotoxicity (ADCC). See Caron et al., J. Exp Med. 176: 1191-1195 (1992) and Shopes, B. J. Immunol. 148: 2918-2922 (1992). Homodimeric antigen binding proteins or antibodies may also be prepared using heterobifunctional cross-linkers as described in Wolff et al., Cancer Research 53: 2560-2565 (1993). Alternatively, a antigen binding protein can be engineered which has dual Fc regions and may thereby have enhanced complement lysis and ADCC capabilities. See Stevenson et al., Anti-CancerDrug Design 3: 219-230 (1989).

It has been shown that sequences within the CDR can cause an antibody to bind to MHC Class II and trigger an unwanted helper T-cell response. A conservative substitution can allow the antigen binding protein to retain binding activity yet reduce its ability to trigger an unwanted T-cell response. It is also contemplated that one or more of the N-terminal 20 amino acids of the heavy or light chain are removed.

Modifications to increase serum half-life also may desirable, for example, by incorporation of or addition of a salvage receptor binding epitope (e.g., by mutation of the appropriate region or by incorporating the epitope into a peptide tag that is then fused to the antigen binding protein at either end or in the middle, e.g., by DNA or peptide synthesis) (see, e.g., WO96/32478) or adding molecules such as PEG or other water soluble polymers, including polysaccharide polymers.

The salvage receptor binding epitope preferably constitutes a region wherein any one or more amino acid residues from one or two loops of a Fc domain are transferred to an analogous position of the antigen binding protein or fragment. Even more preferably, three or more residues from one or two loops of the Fc domain are transferred. Still more preferred, the epitope is taken from the CH2 domain of the Fc region (e.g., of an IgG) and transferred to the CH1, CH3, or VH region, or more than one such region, of the antigen binding protein or antibody. Alternatively, the epitope is taken from the CH2 domain of the Fc region and transferred to the C_{L} region or V_{L} region, or both, of the antigen binding protein fragment. See also International applications WO 97/34631 and WO 96/32478 which describe Fc variants and their interaction with the salvage receptor.

Other sites and amino acid residue(s) of the constant region have been identified that are responsible for complement dependent cytotoxicity (CDC), such as the C1q binding site, and/or the antibody-dependent cellular cytotoxicity (ADCC) [see, e.g., Molec. Immunol. 29 (5): 633-9 (1992); Shields et al., J. Biol. Chem., 276(9):6591-6604 (2001); Lazar et al., Proc. Nat'l. Acad. Sci. 103(11): 4005 (2006) which describe the effect of mutations at specific positions, each of which is incorporated by reference herein in its entirety]. Mutation of residues within Fc receptor binding sites can result in altered (i.e. increased or decreased) effector function, such as altered affinity for Fc receptors, altered ADCC or CDC activity, or altered half-life. As described above, potential mutations include insertion, deletion or substitution of one or more residues, including substitution with alanine, a conservative substitution, a non-conservative substitution, or replacement with a corresponding amino acid residue at the same position from a different subclass (e.g. replacing an IgG1 residue with a corresponding IgG2 residue at that position).

The invention also encompasses production of antigen binding protein molecules, including antibodies and antibody fragments, with altered carbohydrate structure resulting in altered effector activity, including antibody molecules with absent or reduced fucosylation that exhibit improved ADCC activity. A variety of ways are known in the art to accomplish this. For example, ADCC effector activity is mediated by binding of the antibody molecule to the FcγRIII receptor, which has been shown to be dependent on the carbohydrate structure of the N-linked glycosylation at the Asn-297 of the CH2 domain. Non-fucosylated antibodies bind this receptor with increased affinity and trigger FcγRIII-mediated effector functions more efficiently than native, fucosylated antibodies. For example, recombinant production of non-fucosylated antibody in CHO cells in which the alpha-1,6-fucosyl transferase enzyme has been knocked out results in antibody with 100-fold increased ADCC activity (Yamane-Ohnuki et al., Biotechnol Bioeng. 2004 Sep. 5; 87(5):614-22). Similar effects can be accomplished through decreasing the activity of this or other enzymes in the fucosylation pathway, e.g., through siRNA or antisense RNA treatment, engineering cell lines to knockout the enzyme(s), or culturing with selective glycosylation inhibitors (Rothman et al., Mol Immunol. 1989 Dec.; 26(12):1113-23). Some host cell strains, e.g. Lec13 or rat hybridoma YB2/0 cell line naturally produce antibodies with lower fucosylation levels. Shields et al., J Biol Chem. 2002 Jul. 26; 277(30):26733-40; Shinkawa et al., J Biol Chem. 2003 Jan. 31; 278(5):3466-73. An increase in the level of bisected carbohydrate, e.g. through recombinantly producing antibody in cells that overexpress GnTIII enzyme, has also been determined to increase ADCC activity. Umana et al., Nat. Biotechnol. 1999 Feb.; 17(2):176-80. It has been predicted that the absence of only one of the two fucose residues may be sufficient to increase ADCC activity. (Ferrara et al., J Biol Chem. 2005 Dec. 5).

Other Covalent Modifications of Antigen Binding Proteins

Other particular covalent modifications of the anti-hOrai1 ECL2 antigen binding protein, are also included within the scope of this invention. They may be made by chemical synthesis or by enzymatic or chemical cleavage of the antigen binding protein or antibody, if applicable. Other types of covalent modifications can be introduced by reacting targeted amino acid residues with an organic derivatizing agent that is capable of reacting with selected side chains or the N- or C-terminal residues.

Cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, .alpha.-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1 M sodium cacodylate at pH 6.0.

Lysinyl and amino-terminal residues are reacted with succinic or other carboxylic acid anhydrides. Derivatization with these agents has the effect of reversing the charge of the lysinyl residues. Other suitable reagents for derivatizing .alpha.-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pK, of the guanidine functional group. Furthermore, these reagents may react with the groups of lysine as well as the arginine epsilon-amino group.

The specific modification oftyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using ¹²⁵I or ¹³¹I to prepare labeled proteins for use in radioimmunoassay.

Carboxyl side groups (aspartyl or glutamyl) are selectively modified by reaction with carbodiimides (R—N.dbd.C.dbd.N—R′), where R and R′ are different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues, respectively. These residues are deamidated under neutral or basic conditions. The deamidated form of these residues falls within the scope of this invention.

Other modifications include hydroxylation ofproline and lysine, phosphorylation of hydroxyl groups of seryl or threonyl residues, methylation of the .alpha.-amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group.

Another type of covalent modification involves chemically or enzymatically coupling glycosides to the antigen binding protein (e.g., antibody or antibody fragment). These procedures are advantageous in that they do not require production of the antigen binding protein in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO87/05330 published 11 Sep. 1987, and in Aplin and Wriston, CRC Crit. Rev. Biochem., pp. 259-306 (1981).

Removal of any carbohydrate moieties present on the antigen binding protein may be accomplished chemically or enzymatically. Chemical deglycosylation requires exposure of the antigen binding protein to the compound trifluoromethanesulfonic acid, or an equivalent compound. This treatment results in the cleavage of most or all sugars except the linking sugar (N-acetylglucosamine or N-acetylgalactosamine), while leaving the antigen binding protein intact. Chemical deglycosylation is described by Hakimuddin, et al. Arch. Biochem. Biophys. 259: 52 (1987) and by Edge et al. Anal. Biochem., 118: 131 (1981). Enzymatic cleavage of carbohydrate moieties on a antigen binding protein can be achieved by the use of a variety of endo- and exo-glycosidases as described by Thotakura et al. Meth. Enzymol. 138: 350 (1987).

Another type of covalent modification of the antigen binding proteins of the invention (including antibodies and antibody fragments) comprises linking the antigen binding protein to one of a variety of nonproteinaceous polymers, e.g., polyethylene glycol, polypropylene glycol, polyoxyethylated polyols, polyoxyethylated sorbitol, polyoxyethylated glucose, polyoxyethylated glycerol, polyoxyalkylenes, or polysaccharide polymers such as dextran. Such methods are known in the art, see, e.g. U.S. Pat. Nos. 4,640,835; 4,496,689; 4,301,144; 4,670,417; 4,791,192, 4,179,337, 4,766,106, 4,179,337, 4,495,285, 4,609,546 or EP 315 456.

Isolated Nucleic Acids

Another aspect of the present invention is an isolated nucleic acid that encodes an antigen binding protein of the invention, such as, but not limited to, an isolated nucleic acid that encodes an antibody or antibody fragment of the invention. Such nucleic acids are made by recombinant techniques known in the art and/or disclosed herein.

For example, the isolated nucleic acid can encode an antigen binding protein comprising an immunoglobulin heavy chain variable region comprising an amino acid sequence at least 95% identical to SEQ ID NO:39, SEQ ID NO:40, SEQ ID NO:41, or SEQ ID NO:42.

In other exemplary embodiments, the isolated nucleic acid encodes an immunoglobulin heavy chain variable region and N-terminal signal sequence, the nucleic acid having SEQ ID NO:23, SEQ ID NO:25, or SEQ ID NO:27.

In other embodiments, the isolated nucleic acid encodes an antigen binding protein comprising an immunoglobulin light chain variable region comprising an amino acid sequence at least 95% identical to SEQ ID NO:36, SEQ ID NO:37, or SEQ ID NO:38.

Some other embodiments involve the isolated nucleic acid encoding an immunoglobulin light chain variable region and N-terminal signal sequence, the nucleic acid having SEQ ID NO:15, SEQ ID NO:17, or SEQ ID NO:19.

Other examples of the isolated nucleic acid include such that encodes an immunoglobulin heavy chain variable region, wherein the isolated nucleic acid comprises coding sequences for three complementarity determining regions, designated CDRH1, CDRH2 and CDRH3, and wherein:

(a) CDRH1 has the amino acid sequence of SEQ ID NO:43, SEQ ID NO:44, or SEQ ID NO:45;

(b) CDRH2 has the amino acid sequence of SEQ ID NO:46, SEQ ID NO:47, SEQ ID NO:48, or SEQ ID NO:49; and

(c) CDRH3 has the amino acid sequence of SEQ ID NO:50, SEQ ID NO:51, or SEQ ID NO:52.

Still other examples of the isolated nucleic acid include such that encodes an immunoglobulin light chain variable region, wherein the isolated nucleic acid comprises coding sequences for three complementarity determining regions, designated CDRL1, CDRL2 and CDRL3, and wherein:

(a) CDRL1 has the amino acid sequence of SEQ ID NO:53, SEQ ID NO:54, or SEQ ID NO:55;

(b) CDRL2 has the amino acid sequence of SEQ ID NO:56 or SEQ ID NO:57; and

(c) CDRL3 has the amino acid sequence of SEQ ID NO:58 or SEQ ID NO:59.

In other embodiments the isolated nucleic acid encodes an antigen binding protein comprising an immunoglobulin heavy chain comprising the amino acid sequence of SEQ ID NO: 29, SEQ ID NO:33, SEQ ID NO:34, or SEQ ID NO:35.

And in some embodiments the isolated nucleic acid encodes an antigen binding protein comprising an immunoglobulin light chain comprising the amino acid sequence of SEQ ID NO: 30, SEQ ID NO:31, or SEQ ID NO:32.

The present invention is also directed to vectors, including expression vectors, that comprise any of the inventive isolated nucleic acids. An isolated host cell that comprises the expression vector is also encompassed by the present invention, which is made by molecular biological techniques known in the art and/or disclosed herein. The invention is also directed to a method involving:

(a) culturing the host cell in a culture medium under conditions permitting expression of the antigen binding protein encoded by the expression vector; and

(b) recovering the antigen binding protein from the culture medium. Recovering the antigen binding protein is accomplished by known methods of antibody purification, such as but not limited to, antibody purification techniques disclosed in Example 4 and elsewhere herein.

Gene Therapy

Delivery of a therapeutic antigen binding protein to appropriate cells can be effected via gene therapy ex vivo, in situ, or in vivo by use of any suitable approach known in the art. For example, for in vivo therapy, a nucleic acid encoding the desired antigen binding protein or antibody, either alone or in conjunction with a vector, liposome, or precipitate may be injected directly into the subject, and in some embodiments, may be injected at the site where the expression of the antigen binding protein compound is desired. For ex vivo treatment, the subject's cells are removed, the nucleic acid is introduced into these cells, and the modified cells are returned to the subject either directly or, for example, encapsulated within porous membranes which are implanted into the patient. See, e.g. U.S. Pat. Nos. 4,892,538 and 5,283,187.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, chemical treatments, DEAE-dextran, and calcium phosphate precipitation. Other in vivo nucleic acid transfer techniques include transfection with viral vectors (such as adenovirus, Herpes simplex I virus, adeno-associated virus or retrovirus) and lipid-based systems. The nucleic acid and transfection agent are optionally associated with a microparticle. Exemplary transfection agents include calcium phosphate or calcium chloride co-precipitation, DEAE-dextran-mediated transfection, quaternary ammonium amphiphile DOTMA ((dioleoyloxypropyl) trimethylammonium bromide, commercialized as Lipofectin by GIBCO-BRL))(Felgner et al, (1987) Proc. Natl. Acad. Sci. USA 84, 7413-7417; Malone et al. (1989) Proc. Natl. Acad. Sci. USA 86 6077-6081); lipophilic glutamate diesters with pendent trimethylammonium heads (Ito et al. (1990) Biochem. Biophys. Acta 1023, 124-132); the metabolizable parent lipids such as the cationic lipid dioctadecylamido glycylspermine (DOGS, Transfectam, Promega) and dipalmitoylphosphatidyl ethanolamylspermine (DPPES)(J. P. Behr (1986) Tetrahedron Lett. 27, 5861-5864; J. P. Behr et al. (1989) Proc. Natl. Acad. Sci. USA 86, 6982-6986); metabolizable quaternary ammonium salts (DOTB, N-(1-[2,3-d]oleoyloxy]propyl)-N,N,N-trimethylammonium methylsulfate (DOTAP)(Boehringer Mannheim), polyethyleneimine (PEI), dioleoyl esters, ChoTB, ChoSC, DOSC)(Leventis et al. (1990) Biochim. Inter. 22, 235-241); 3beta[N—(N′,N′-dimethylaminoethane)-carbamoyl]cholesterol (DC-Chol), dioleoylphosphatidyl ethanolamine (DOPE)/3beta[N—(N′,N′-dimethylaminoethane)-carbamoyl]cholesterolDC-Chol in one to one mixtures (Gao et al., (1991) Biochim. Biophys. Acta 1065, 8-14), spermine, spermidine, lipopolyamines (Behr et al., Bioconjugate Chem, 1994, 5: 382-389), lipophilic polylysines (LPLL) (Zhou et al., (1991) Biochim. Biophys. Acta 939, 8-18), [[(1,1,3,3-tetramethylbutyl)cre-soxy]ethoxy]ethyl]dimethylbenzylammonium hydroxide (DEBDA hydroxide) with excess phosphatidylcholine/cholesterol (Ballas et al., (1988) Biochim. Biophys. Acta 939, 8-18), cetyltrimethylammonium bromide (CTAB)/DOPE mixtures (Pinnaduwage et al, (1989) Biochim. Biophys. Acta 985, 33-37), lipophilic diester of glutamic acid (TMAG) with DOPE, CTAB, DEBDA, didodecylammonium bromide (DDAB), and stearylamine in admixture with phosphatidylethanolamine (Rose et al., (1991) Biotechnique 10, 520-525), DDAB/DOPE (TransfectACE, GIBCO BRL), and oligogalactose bearing lipids. Exemplary transfection enhancer agents that increase the efficiency of transfer include, for example, DEAE-dextran, polybrene, lysosome-disruptive peptide (Ohmori N I et al, Biochem Biophys Res Commun Jun. 27, 1997; 235(3):726-9), chondroitan-based proteoglycans, sulfated proteoglycans, polyethylenimine, polylysine (Pollard H et al. J Biol Chem, 1998 273 (13):7507-11), integrin-binding peptide CYGGRGDTP (SEQ ID NO:235), linear dextran nonasaccharide, glycerol, cholesteryl groups tethered at the 3′-terminal internucleoside link of an oligonucleotide (Letsinger, R. L. 1989 Proc Natl Acad Sci USA 86: (17):6553-6), lysophosphatide, lysophosphatidylcholine, lysophosphatidylethanolamine, and 1-oleoyl lysophosphatidylcholine.

In some situations it may be desirable to deliver the nucleic acid with an agent that directs the nucleic acid-containing vector to target cells. Such “targeting” molecules include antigen binding proteins specific for a cell-surface membrane protein on the target cell, or a ligand for a receptor on the target cell. Where liposomes are employed, proteins which bind to a cell-surface membrane protein associated with endocytosis may be used for targeting and/or to facilitate uptake. Examples of such proteins include capsid proteins and fragments thereof tropic for a particular cell type, antigen binding proteins for proteins which undergo internalization in cycling, and proteins that target intracellular localization and enhance intracellular half-life. In other embodiments, receptor-mediated endocytosis can be used. Such methods are described, for example, in Wu et al., 1987 or Wagner et al., 1990. For review of the currently known gene marking and gene therapy protocols, see Anderson 1992. See also WO 93/25673 and the references cited therein. For additional reviews of gene therapy technology, see Friedmann, Science, 244: 1275-1281 (1989); Anderson, Nature, supplement to vol. 392, no 6679, pp. 25-(1998); Verma, Scientific American: 68-84 (1990); and Miller, Nature, 357: 455460 (1992).

Administration and Preparation of Pharmaceutical Formulations

The anti-hOrai1 antigen binding proteins or antibodies used in the practice of a method of the invention may be formulated into pharmaceutical compositions and medicaments comprising a carrier suitable for the desired delivery method. Suitable carriers include any material which, when combined with the anti-hOrai1 antigen binding protein or antibody, retains the high-affinity binding of hOrai1 and is nonreactive with the subject's immune systems. Examples include, but are not limited to, any of a number of standard pharmaceutical carriers such as sterile phosphate buffered saline solutions, bacteriostatic water, and the like. A variety of aqueous carriers may be used, e.g., water, buffered water, 0.4% saline, 0.3% glycine and the like, and may include other proteins for enhanced stability, such as albumin, lipoprotein, globulin, etc., subjected to mild chemical modifications or the like.

Exemplary antigen binding protein concentrations in the formulation may range from about 0.1 mg/ml to about 180 mg/ml or from about 0.1 mg/mL to about 50 mg/mL, or from about 0.5 mg/mL to about 25 mg/mL, or alternatively from about 2 mg/mL to about 10 mg/mL. An aqueous formulation of the antigen binding protein may be prepared in a pH-buffered solution, for example, at pH ranging from about 4.5 to about 6.5, or from about 4.8 to about 5.5, or alternatively about 5.0. Examples of buffers that are suitable for a pH within this range include acetate (e.g. sodium acetate), succinate (such as sodium succinate), gluconate, histidine, citrate and other organic acid buffers. The buffer concentration can be from about 1 mM to about 200 mM, or from about 10 mM to about 60 mM, depending, for example, on the buffer and the desired isotonicity of the formulation.

A tonicity agent, which may also stabilize the antigen binding protein, may be included in the formulation. Exemplary tonicity agents include polyols, such as mannitol, sucrose or trehalose. Preferably the aqueous formulation is isotonic, although hypertonic or hypotonic solutions may be suitable. Exemplary concentrations of the polyol in the formulation may range from about 1% to about 15% w/v.

A surfactant may also be added to the antigen binding protein formulation to reduce aggregation of the formulated antigen binding protein and/or minimize the formation of particulates in the formulation and/or reduce adsorption. Exemplary surfactants include nonionic surfactants such as polysorbates (e.g. polysorbate 20, or polysorbate 80) or poloxamers (e.g. poloxamer 188). Exemplary concentrations of surfactant may range from about 0.001% to about 0.5%, or from about 0.005% to about 0.2%, or alternatively from about 0.004% to about 0.01% w/v.

In one embodiment, the formulation contains the above-identified agents (i.e. antigen binding protein, buffer, polyol and surfactant) and is essentially free of one or more preservatives, such as benzyl alcohol, phenol, m-cresol, chlorobutanol and benzethonium Cl. In another embodiment, a preservative may be included in the formulation, e.g., at concentrations ranging from about 0.1% to about 2%, or alternatively from about 0.5% to about 1%. One or more other pharmaceutically acceptable carriers, excipients or stabilizers such as those described in Remington's Pharmaceutical Sciences 16th edition, Osol, A. Ed. (1980) may be included in the formulation provided that they do not adversely affect the desired characteristics of the formulation. Acceptable carriers, excipients or stabilizers are nontoxic to recipients at the dosages and concentrations employed and include; additional buffering agents; co-solvents; antoxidants including ascorbic acid and methionine; chelating agents such as EDTA; metal complexes (e.g. Zn-protein complexes); biodegradable polymers such as polyesters; and/or salt-forming counterions such as sodium.

Therapeutic formulations of the antigen binding protein are prepared for storage by mixing the antigen binding protein having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, maltose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In one embodiment, a suitable formulation of the claimed invention contains an isotonic buffer such as a phosphate, acetate, or TRIS buffer in combination with a tonicity agent such as a polyol, Sorbitol, sucrose or sodium chloride which tonicifies and stabilizes. One example of such a tonicity agent is 5% Sorbitol or sucrose. In addition, the formulation could optionally include a surfactant such as to prevent aggregation and for stabilization at 0.01 to 0.02% wt/vol. The pH of the formulation may range from 4.5-6.5 or 4.5 to 5.5. Other exemplary descriptions of pharmaceutical formulations for antibodies may be found in US 2003/0113316 and U.S. Pat. No. 6,171,586, each incorporated herein by reference in its entirety.

The formulation herein may also contain more than one active compound as necessary for the particular indication being treated, preferably those with complementary activities that do not adversely affect each other. For example, it may be desirable to further provide an immunosuppressive agent. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Suspensions and crystal forms of antigen binding proteins are also contemplated. Methods to make suspensions and crystal forms are known to one of skill in the art.

The formulations to be used for in vivo administration must be sterile. The compositions of the invention may be sterilized by conventional, well known sterilization techniques. For example, sterilization is readily accomplished by filtration through sterile filtration membranes. The resulting solutions may be packaged for use or filtered under aseptic conditions and lyophilized, the lyophilized preparation being combined with a sterile solution prior to administration.

The process of freeze-drying is often employed to stabilize polypeptides for long-term storage, particularly when the polypeptide is relatively unstable in liquid compositions. A lyophilization cycle is usually composed of three steps: freezing, primary drying, and secondary drying; Williams and Polli, Journal of Parenteral Science and Technology, Volume 38, Number 2, pages 48-59 (1984). In the freezing step, the solution is cooled until it is adequately frozen. Bulk water in the solution forms ice at this stage. The ice sublimes in the primary drying stage, which is conducted by reducing chamber pressure below the vapor pressure of the ice, using a vacuum. Finally, sorbed or bound water is removed at the secondary drying stage under reduced chamber pressure and an elevated shelf temperature. The process produces a material known as a lyophilized cake. Thereafter the cake can be reconstituted prior to use.

The standard reconstitution practice for lyophilized material is to add back a volume of pure water (typically equivalent to the volume removed during lyophilization), although dilute solutions of antibacterial agents are sometimes used in the production of pharmaceuticals for parenteral administration; Chen, Drug Development and Industrial Pharmacy, Volume 18, Numbers 11 and 12, pages 1311-1354 (1992).

Excipients have been noted in some cases to act as stabilizers for freeze-dried products; Carpenter et al., Developments in Biological Standardization, Volume 74, pages 225-239 (1991). For example, known excipients include polyols (including mannitol, sorbitol and glycerol); sugars (including glucose and sucrose); and amino acids (including alanine, glycine and glutamic acid).

In addition, polyols and sugars are also often used to protect polypeptides from freezing and drying-induced damage and to enhance the stability during storage in the dried state. In general, sugars, in particular disaccharides, are effective in both the freeze-drying process and during storage. Other classes of molecules, including mono- and di-saccharides and polymers such as PVP, have also been reported as stabilizers of lyophilized products.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Sustained-release preparations may be prepared. Suitable examples of sustained-release preparations include semipermeable matrices of solid hydrophobic polymers containing the antigen binding protein, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated polypeptides remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The formulations of the invention may be designed to be short-acting, fast-releasing, long-acting, or sustained-releasing as described herein. Thus, the pharmaceutical formulations may also be formulated for controlled release or for slow release.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant invention.

The antigen binding protein is administered by any suitable means, including parenteral, subcutaneous, intraperitoneal, intrapulmonary, and intranasal, and, if desired for local treatment, intralesional administration. Parenteral infusions include intravenous, intraarterial, intraperitoneal, intramuscular, intradermal or subcutaneous administration. In addition, the antigen binding protein is suitably administered by pulse infusion, particularly with declining doses of the antigen binding protein or antibody. Preferably the dosing is given by injections, most preferably intravenous or subcutaneous injections, depending in part on whether the administration is brief or chronic. Other administration methods are contemplated, including topical, particularly transdermal, transmucosal, rectal, oral or local administration e.g. through a catheter placed close to the desired site. Most preferably, the antigen binding protein of the invention is administered intravenously in a physiological solution at a dose ranging between 0.01 mg/kg to 100 mg/kg at a frequency ranging from daily to weekly to monthly (e.g. every day, every other day, every third day, or 2, 3, 4, 5, or 6 times per week), preferably a dose ranging from 0.1 to 45 mg/kg, 0.1 to 15 mg/kg or 0.1 to 10 mg/kg at a frequency of 2 or 3 times per week, or up to 45 mg/kg once a month.

The invention is illustrated by the following examples, which are not intended to be limiting in any way.

### EXAMPLES

### Example 1

### Generation of Orai1 Channel as Antigens

Molecular Cloning of Human Orai1 and Human STIM1

The human Orai1 (hOrai1; SEQ ID NO:2), encoded by the following cDNA sequence (NCBI Reference Sequence NM_{—}032790):

SEQ ID NO: 1

| | |
|---|---|
| 1 | ATGCATCCGG AGCCCGCCCC GCCCCCGAGC CGCAGCAGTC |
| | CCGAGCTTCC |
| 51 | CCCAAGCGGC GGCAGCACCA CCAGCGGCAG CCGCCGGAGC |
| | CGCCGCCGCA |
| 101 | GCGGGGACGG GGAGCCCCCG GGGGCCCCGC CACCGCCGCC |
| | GTCCGCCGTC |
| 151 | ACCTACCCGG ACTGGATCGG CCAGAGTTAC TCCGAGGTGA |
| | TGAGCCTCAA |
| 201 | CGAGCACTCC ATGCAGGCGC TGTCCTGGCG CAAGCTCTAC |
| | TTGAGCCGCG |
| 251 | CCAAGCTTAA AGCCTCCAGC CGGACCTCGG CTCTGCTCTC |
| | CGGCTTCGCC |
| 301 | ATGGTGGCAA TGGTGGAGGT GCAGCTGGAC GCTGACCACG |
| | ACTACCCACC |
| 351 | GGGGCTGCTC ATCGCCTTCA GTGCCTGCAC CACAGTGCTG |
| | GTGGCTGTGC |
| 401 | ACCTGTTTGC GCTCATGATC AGCACCTGCA TCCTGCCCAA |
| | CATCGAGGCG |
| 451 | GTGAGCAACG TGCACAATCT CAACTCGGTC AAGGAGTCCC |
| | CCCATGAGCG |
| 501 | CATGCACCGC CACATCGAGC TGGCCTGGGC CTTCTCCACC |
| | GTCATCGGCA |
| 551 | CGCTGCTCTT CCTAGCTGAG GTGGTGCTGC TCTGCTGGGT |
| | CAAGTTCTTG |
| 601 | CCCCTCAAGA AGCAGCCAGG CCAGCCAAGG CCCACCAGCA |
| | AGCCCCCCGC |
| 651 | CAGTGGCGCA GCAGCCAACG TCAGCACCAG CGGCATCACC |
| | CCGGGCCAGG |
| 701 | CAGCTGCCAT CGCCTCGACC ACCATCATGG TGCCCTTCGG |
| | CCTGATCTTT |
| 751 | ATCGTCTTCG CCGTCCACTT CTACCGCTCA CTGGTTAGCC |
| | ATAAGACTGA |
| 801 | CCGACAGTTC CAGGAGCTCA ACGAGCTGGC GGAGTTTGCC |
| | CGCTTACAGG |
| 851 | ACCAGCTGGA CCACAGAGGG GACCACCCCC TGACGCCCGG |
| | CAGCCACTAT |
| 901 | GCCTAG// |

and cDNA of human STIM1 (NCBI Reference Sequence NM_{—}003156):

SEQ ID NO: 5

| | |
|---|---|
| 1 | ATGGATGTAT GCGTCCGTCT TGCCCTGTGG CTCCTCTGGG |
| | GACTCCTCCT |
| 51 | GCACCAGGGC CAGAGCCTCA GCCATAGTCA CAGTGAGAAG |
| | GCGACAGGAA |
| 101 | CCAGCTCGGG GGCCAACTCT GAGGAGTCCA CTGCAGCAGA |
| | GTTTTGCCGA |
| 151 | ATTGACAAGC CCCTGTGTCA CAGTGAGGAT GAGAAACTCA |
| | GCTTCGAGGC |
| 201 | AGTCCGTAAC ATCCACAAAC TGATGGACGA TGATGCCAAT |
| | GGTGATGTGG |
| 251 | ATGTGGAAGA AAGTGATGAG TTCCTGAGGG AAGACCTCAA |
| | TTACCATGAC |
| 301 | CCAACAGTGA AACACAGCAC CTTCCATGGT GAGGATAAGC |
| | TCATCAGCGT |
| 351 | GGAGGACCTG TGGAAGGCAT GGAAGTCATC AGAAGTATAC |
| | AATTGGACCG |
| 401 | TGGATGAGGT GGTACAGTGG CTGATCACAT ATGTGGAGCT |
| | GCCTCAGTAT |
| 451 | GAGGAGACCT TCCGGAAGCT GCAGCTCAGT GGCCATGCCA |
| | TGCCAAGGCT |
| 501 | GGCTGTCACC AACACCACCA TGACAGGGAC TGTGCTGAAG |
| | ATGACAGACC |
| 551 | GGAGTCATCG GCAGAAGCTG CAGCTGAAGG CTCTGGATAC |
| | AGTGCTCTTT |
| 601 | GGGCCTCCTC TCTTGACTCG CCATAATCAC CTCAAGGACT |
| | TCATGCTGGT |
| 651 | GGTGTCTATC GTTATTGGTG TGGGCGGCTG CTGGTTTGCC |
| | TATATCCAGA |
| 701 | ACCGTTACTC CAAGGAGCAC ATGAAGAAGA TGATGAAGGA |
| | CTTGGAGGGG |
| 751 | TTACACCGAG CTGAGCAGAG TCTGCATGAC CTTCAGGAAA |
| | GGCTGCACAA |
| 801 | GGCCCAGGAG GAGCACCGCA CAGTGGAGGT GGAGAAGGTC |
| | CATCTGGAAA |
| 851 | AGAAGCTGCG CGATGAGATC AACCTTGCTA AGCAGGAAGC |
| | CCAGCGGCTG |
| 901 | AAGGAGCTGC GGGAGGGTAC TGAGAATGAG CGGAGCCGCC |
| | AAAAATATGC |
| 951 | TGAGGAGGAG TTGGAGCAGG TTCGGGAGGC CTTGAGGAAA |
| | GCAGAGAAGG |
| 1001 | AGCTAGAATC TCACAGCTCA TGGTATGCTC CAGAGGCCCT |
| | TCAGAAGTGG |
| 1051 | CTGCAGCTGA CACATGAGGT GGAGGTGCAA TATTACAACA |
| | TCAAGAAGCA |
| 1101 | AAATGCTGAG AAGCAGCTGC TGGTGGCCAA GGAGGGGGCT |
| | GAGAAGATAA |
| 1151 | AAAAGAAGAG AAACACACTC TTTGGCACCT TCCACGTGGC |
| | CCACAGCTCT |
| 1201 | TCCCTGGATG ATGTAGATCA TAAAATTCTA ACAGCTAAGC |
| | AAGCACTGAG |
| 1251 | CGAGGTGACA GCAGCATTGC GGGAGCGCCT GCACCGCTGG |
| | CAACAGATCG |
| 1301 | AGATCCTCTG TGGCTTCCAG ATTGTCAACA ACCCTGGCAT |
| | CCACTCACTG |
| 1351 | GTGGCTGCCC TCAACATAGA CCCCAGCTGG ATGGGCAGTA |
| | CACGCCCCAA |
| 1401 | CCCTGCTCAC TTCATCATGA CTGACGACGT GGATGACATG |
| | GATGAGGAGA |
| 1451 | TTGTGTCTCC CTTGTCCATG CAGTCCCCTA GCCTGCAGAG |
| | CAGTGTTCGG |
| 1501 | CAGCGCCTGA CGGAGCCACA GCATGGCCTG GGATCTCAGA |
| | GGGATTTGAC |
| 1551 | CCATTCCGAT TCGGAGTCCT CCCTCCACAT GAGTGACCGC |
| | CAGCGTGTGG |
| 1601 | CCCCCAAACC TCCTCAGATG AGCCGTGCTG CAGACGAGGC |
| | TCTCAATGCC |
| 1651 | ATGACTTCCA ATGGCAGCCA CCGGCTGATC GAGGGGGTCC |
| | ACCCAGGGTC |
| 1701 | TCTGGTGGAG AAACTGCCTG ACAGCCCTGC CCTGGCCAAG |
| | AAGGCATTAC |
| 1751 | TGGCGCTGAA CCATGGGCTG GACAAGGCCC ACAGCCTGAT |
| | GGAGCTGAGC |
| 1801 | CCCTCAGCCC CACCTGGTGG CTCTCCACAT TTGGATTCTT |
| | CCCGTTCTCA |
| 1851 | CAGCCCCAGC TCCCCAGACC CAGACACACC ATCTCCAGTT |
| | GGGGACAGCC |
| 1901 | GAGCCCTGCA AGCCAGCCGA AACACACGCA TTCCCCACCT |
| | GGCTGGCAAG |
| 1951 | AAGGCTGTGG CTGAGGAGGA TAATGGCTCT ATTGGCGAGG |
| | AAACAGACTC |
| 2001 | CAGCCCAGGC CGGAAGAAGT TTCCCCTCAA AATCTTTAAG |
| | AAGCCTCTTA |
| 2051 | AGAAGTAG// |

were cloned into the pcDNA3.1/Neomycin (Invitrogen, Carlsbad, Calif.) and pcDNA3.1/Zeocin (Invitrogen), respectively for expression in mammalian cells. In addition, the human Orai1 was cloned into a CMV-based mammalian expression vector pTT14 (Amgen vector (an Amgen vector containing a CMV promoter, Poly A tail and a Puromycin resistance gene).

Briefly, to generate human Orai1, two oligonucleotide primers with the sequences depicted below (SEQ ID NO:11 and SEQ ID NO₁₂) were used in a Polymerase Chain Reaction (PCR) method using human brain cDNA from Biochain Inc. as a template.

Forward primer:

(SEQ ID NO: 11)

5′-CGGATCCTGAACCACCATGCATCCGGAGCCCGCCCCGCC-3′
and
Reverse primer:

(SEQ ID NO: 12)

5′-GCGGCCGCCTAGGCATAGTGGCTGCCGGGCG-3′.

The resulting 930-bp PCR product was purified and digested with BamHI and Not1 restriction enzymes. The pcDNA3.1/Neomycin vector was also digested with BamHI and Not1 restriction enzymes. The digested PCR product and vector were ligated to create a pcDNA3.1/Neomycin-hOrai1 vector. The insert was sequenced and determined to be 100% identical to the human Orai1 cDNA coding sequence (SEQ ID NO:1 Human Orai1 cDNA NCBI Reference Sequence NM_{—}032790, encoding SEQ ID NO:2). Human STIM1 in pcDNA3.1/Zeocin was generated similarly using PCR methodology and referred to as pcDNA3.1/Zeocin-hSTIM1 vector. The insert was sequenced and determined to be 100% identical to human STIM1 (SEQ ID NO:5; Human STIM1 cDNA NCBI Reference Sequence NM_{—}003156, encoding the human STIM1 protein sequence SEQ ID NO:6):

SEQ ID NO: 6

| | |
|---|---|
| 1 | MDVCVRLALW LLWGLLLHQG QSLSHSHSEK ATGTSSGANS |
| | EESTAAEFCR |
| 51 | IDKPLCHSED EKLSFEAVRN IHKLMDDDAN GDVDVEESDE |
| | FLREDLNYHD |
| 101 | PTVKHSTFHG EDKLISVEDL WKAWKSSEVY NWTVDEVVQW |
| | LITYVELPQY |
| 151 | EETFRKLQLS GHAMPRLAVT NTTMTGTVLK MTDRSHRQKL |
| | QLKALDTVLF |
| 201 | GPPLLTRHNH LKDFMLVVSI VIGVGGCWFA YIQNRYSKEH |
| | MKKMMKDLEG |
| 251 | LHRAEQSLHD LQERLHKAQE EHRTVEVEKV HLEKKLRDEI |
| | NLAKQEAQRL |
| 301 | KELREGTENE RSRQKYAEEE LEQVREALRK AEKELESHSS |
| | WYAPEALQKW |
| 351 | LQLTHEVEVQ YYNIKKQNAE KQLLVAKEGA EKIKKKRNTL |
| | FGTFHVAHSS |
| 401 | SLDDVDHKIL TAKQALSEVT AALRERLHRW QQIEILCGFQ |
| | IVNNPGIHSL |
| 451 | VAALNIDPSW MGSTRPNPAH FIMTDDVDDM DEEIVSPLSM |
| | QSPSLQSSVR |
| 501 | QRLTEPQHGL GSQRDLTHSD SESSLHMSDR QRVAPKPPQM |
| | SRAADEALNA |
| 551 | MTSNGSHRLI EGVHPGSLVE KLPDSPALAK KALLALNHGL |
| | DKAHSLMELS |
| 601 | PSAPPGGSPH LDSSRSHSPS SPDPDTPSPV GDSRALQASR |
| | NTRIPHLAGK |
| 651 | KAVAEEDNGS IGEETDSSPG RKKFPLKIFK KPLKK//. |

The human STIM1 cDNA fused to yellow fluorescent protein (YFP) cDNA (SEQ ID NO:7, encoding YFP SEQ ID NO:8), and referred to as hSTIM1-YFP (SEQ ID NO:9, encoding Human Stiml-YFP protein SEQ ID NO:10), was constructed using PCR technology. This construct was generated in two parts with the first part joining the first 39 amino acid residues of the hSTIM1 to YFP. For the second part, the hSTIM1 without the first 39 amino acids was generated by PCR with appropriate restriction enzyme sites at the ends. The joining of a DNA fragment encoding the first 39 amino acids ofhSTIM1 to the YFP gene without its stop codon was accomplished using the three forward primers (A1, A2 and A3 and one reverse primer (A4) with the sequences indicated below in a PCR reaction with YFP gene as the template:

Forward primer A1:

(SEQ ID NO: 181)

5′-GCTAGCTGAACCACCATGGATGTATGCGTCCGTCTTG-3′;
Forward primer A2:

(SEQ ID NO: 182)

5′-GGGACTCCTCCTGCACCAGGGCCAGAGCCTCAGCCATAGTCACA
GTGAGAAG-3′;
Forward primer A3:

(SEQ ID NO: 183)

5′-CAGCCATAGTCACAGTGAGAAGGCGACAGGAACCAGCTCGGGAGCCA
ACATGGTGAGCAAGGGCGAGGAG-3′;
Reverse primer A4:

(SEQ ID NO: 184)

5′-CGGCATGGACGAGCTGTACAAGTCTGAGGAGTCGACTGCAGCAG-3′

The resulting PCR product of 870-bp was visually confirmed on a 0.8% agarose gel and restriction enzyme digested with NheI and SalI restriction enzymes (Roche) after purifification using PCR Purification Kit (Qiagen). For the second part, the hSTIM1 fragment lacking the first 117-bp was generated by PCR using the forward (B1) and reverse (B2) primers with the sequences indicated below and the hSTIM1 as a template:

Forward primer B1:

(SEQ ID NO: 185)

5′-GGAGTCGACTGCAGCAGAGTTTTGCCG-3′;
and
Reverse primer B2:

(SEQ ID NO: 186)

5′-CTTTAAGAAGCCTCTTAAGAAGTAGGCGGCCGC-3′.

The resulting PCR product was visualized on a 0.8% agarose gel and restriction enzyme digested with SalI and Not1 restriction enzymes (Roche) after purification using the PCR Purification Kit (Qiagen).

The pcDNA3.1/Zeocin expression vector vector was digested with NheI and Not1 restriction enzymes and the large fragment was resolved and excised on a 0.8% agarose gel and purified by Gel Extraction Kit (Qiagen). The restriction enzyme digested PCR products were resolved and excised on a 0.8% agarose gel and purified by Gel Extraction Kit (Qiagen). The gel purified large fragment of the pcDNA3.1/Zeocin vector and restriction enzyme disgested PCR products were ligated and the transformed into One Shot® Top 10 (Invitrogen) to create a pcDNA3.1/Zeocin-hSTIM1-YFP. The DNA from hSTIM-YFP in pcDNA3.1/Zeocin vector was sequenced to confirm the hSTIM-YFP regions and the sequence was 100% identical to (SEQ ID NO:9, Human Stiml-YFP cDNA and SEQ ID NO:10, Human Stiml-YFP protein seqeunce).

Cell Line Development.

The pcDNA3.1/Neomycin-hOrai1 vector was transfected into U20S, a human osteosarcoma cell line (ATCC HTB-96) using FuGENE 6 in growth medium according to the manufacturer's protocol (Roche). Two days after transfection, cells were dislodged from plate surface using 0.5% trypsin (Gibco) and re-plated into growth medium containing 500 g/ml of Geneticin (Gibco). The human Orai1 expressing U20S cells were selected for binding to monoclonal antibody 84.5, a mouse anti-human Orai1 antibody (mAb84.5). Briefly, cells were incubated with mAb84.5 at 4° C. for 30 minutes, followed by staining with goat anti-mouse immunoglobulin gamma antibody fragment that is directly labeled with phycoerythrin at 4° C. for 30 minutes and then subjected to fluorescently-activated cell sorting (FACS). After two times FACS with mAb84.5, the cell line was referred to as U20S/hOrai1.

The pcDNA3.1/Neomycin-hOrai1 vector described above was also transfected into AM-1 CHO cells (a serum-free growth media-adapted variant from the CHO DHFR-deficient cell line described in Urlaub and Chasin, Proc. Natl. Acad. Sci. 77, 4216 (1980)) using FuGENE 6 in growth medium according to the manufacturer's protocol (Roche). Two days after transfection, the cells were dislodged from plate surface using 0.5% trypsin (Gibco) and re-plated into growth medium containing 700 g/ml geneticin (Gibco). The human Orai1 expressing cells were sorted two times by FACS with anti-human Orai1 mAb84.5. Subsequently, the sorted pool was then transfected with pcDNA3.1/Zeocin-hSTIM1-YFP using FuGENE 6 in growth medium according to the manufacturer's protocol (Roche). Two days after transfection, cells were dislodged from plate surface using 0.5% trypsin (Gibco) and plated into growth medium containing 500 μg/ml of Geneticin and 200 μg/ml of Zeocin (Invitrogen). Cells stably co-expressing human Orai1 and hSTIM1-YFP proteins were selected by FACS with anti-hOrai1 mAb84.5 and by detection of Yellow Fluorescent Protein, and were referred to as AM1-CHO/hOrai 1/hSTIM1-YFP.

### Example 2

### Generation of Antibodies to Human Orai1

Immunization

In two separate campaigns, designated “Campaign 1” and “Campaign 2”, Xenomouse® XMG2KL, XMG4KL, XMG1KL and XMG2k strains of mice were generated generally as described previously in Mendez et al., Nat. Genet. 15:146-156 (1997) and immunized, in Campaign 1, with AM1-CHO/hOrai1/hSTIM1-YFP or U20S/hOrai1/hSTIM1 cells using a dose of 4.0×10⁶ cells per mouse and with subsequent boosts of the same type antigen at 2.0×10⁶ cells/mouse. In Campaign 2, thapsigargin-treated U20S/hOrai1/hSTIM1 cells were used to immunize the mice at 4.0×10⁶ cells per mouse, with subsequent boosts of 2.0×10⁶ thapsigargin-treated U20S/hOrai1/hSTIM1 cells/mouse. Thapsigargin treated U20S/Orai1 cells were prepared by diluting cells to 3 million cells per mL, in complete growth media containing 2 mM thapsigargin (Thapsigargin, Sigma cat#T9033), cells were incubated for 30 minutes at 37° C., washed twice with 1× phosphate buffered saline and then immediately used for immunization. Injection sites used were combinations of subcutaneous base-of-tail and intraperitoneal. Immunizations were performed in accordance with methods disclosed in U.S. Pat. No. 7,064,244, the disclosure of which is hereby incorporated by reference in its entirety. Adjuvant Alum (E.M. Sergent Pulp and Chemical Co., Clifton, N.J., cat. #1452-250) was prepared according to the manufacturers' instructions and mixed in a 1:1 ratio of adjuvant emulsion to antigen solution. To monitor titers raised against human Orai1, sera were collected 4 to 6 weeks after the first injection, and specific titers were determined by FACs staining using either thapsigargin-untreated U20S/hOrai1 or AM1-CHO/hOrai1/hSTIM1-YFP cells. Immunized mice were boosted with a range of 11 to 17 immunizations over a period of approximately one to three and one-half months. Mice with the highest sera titer were identified and prepared for hybridoma generation. The immunizations were performed in groups of multiple mice, typically ten. Mesenteric, inguinal, and peri-aortic lymph nodes and spleen tissues were typically pooled from each group for generating hybridoma fusions.

Preparation of Monoclonal Antibodies.

Mice exhibiting suitable titers were identified, and lymphocytes were obtained from draining lymph nodes and, if necessary, pooled for each cohort. Lymphocytes were dissociated from lymphoid tissue in a suitable medium (for example, Dulbecco's Modified Eagle Medium; DMEM; obtainable from Invitrogen, Carlsbad, Calif.) to release the cells from the tissues, and suspended in DMEM. B cells were selected and/or expanded using a suitable method, and fused with suitable fusion partner, for example, nonsecretory myeloma P3X₆₃Ag8.653 cells (American Type Culture Collection CRL 1580; Keamey et al, J. Immunol. 123, 1979, 1548-1550).

Lymphocytes were mixed with fusion partner cells at a ratio of 1:4. The cell mixture was gently pelleted by centrifugation at 400×g for 4 minutes, the supernatant was decanted, and the cell mixture was gently mixed by using a 1 ml pipette. Fusion was induced with PEG/DMSO (polyethylene glycol/dimethyl sulfoxide; obtained from Sigma-Aldrich, St. Louis Mo.; 1 ml per million of lymphocytes). PEG/DMSO was slowly added with gentle agitation over one minute followed, by one minute of mixing. IDMEM (DMEM without glutamine; 2 ml per million of B cells), was then added over 2 minutes with gentle agitation, followed by additional IDMEM (8 ml per million B-cells) which was added over 3 minutes.

The fused cells were gently pelleted (400×g 6 minutes) and resuspended in 20 ml Selection medium (for example, DMEM containing Azaserine and Hypoxanthine [HA] and other supplemental materials as necessary) per million B-cells. Cells were incubated for 20-30 minutes at 37° C. and then were resuspended in 200 ml Selection medium and cultured for three to four days in T175 flasks prior to 96-well plating.

Cells were distributed into 96-well plates using standard techniques to maximize clonality of the resulting colonies. After several days of culture, the hybridoma supernatants were collected and subjected to screening assays as detailed in the examples below, including confirmation of binding to human Orai1 channel. Positive cells were further selected and subjected to standard cloning and subcloning techniques. Clonal lines were expanded in vitro, and the secreted human antibodies obtained for analysis.

### Example 3

### Identification of Orai1-Specific Monoclonal Antibodies

Selection of Orai1-specific binding antibodies by FMAT. After 14 days of culture, hybridoma supernatants were screened for human Orai1-specific monoclonal antibodies by Fluorometric Microvolume Assay Technology (FMAT) (Applied Biosystems, Foster City, Calif.). The supernatants were screened against the AM1-CHO/hOrai1/hSTIM1-YFP cells and counter-screened against parental AM1-CHO cells for hybridomas supernatants derived from immunization with U20S/hOrai1 cells (prepared as described in Example 1). Conversely, for hydridomas derived from the immunization with AM1-CHO/hOrai1/hSTIM1-YFP cells, binding screen was performed with U20S/Orai1 and counter-screened with parental U20S cells.

Briefly, the cells in Freestyle™ medium (Invitrogen, Carlsbad, Calif.) were seeded into 384-well FMAT plates a mixture of approximately 4000 AM1-CHO/hOrai1/hSTIM1-YFP or U20S/hOrai1 cells/well and approximately 16,000 corresponding parental cells/well in a total volume of 50 μL/well, and cells were incubated overnight at 37° C. Then, 10 μL/well of supernatant was added and plates were incubated for approximately one hour at 4° C., after which 10 μL/well of anti-human IgG-Cy5 secondary antibody (Jackson Immunoresearch, West Grove, Pa.) was added at a concentration of 2.8 μg/ml (400 ng/ml final concentration). Plates were then incubated for one hour at 4° C., and fluorescence was read using an FMAT macroconfocal scanner (Applied Biosystems, Foster City, Calif.). For counter screens, the parental AM-1 CHO cells or U20S cells were seeded at approximately 16,000 cells/well in a total volume of 50 μL/well and cells were incubated overnight at 37° C. The FMAT counter screen was performed similarly and in parallel to the binding screen to differentiate and eliminate hybridomas binding to cellular proteins, but not to the Orai1 channel.

Selection of Orai1-Specific Binding Antibodies by FACS.

The hybridoma supernatants that were scored as positives in the FMAT binding assay along with a few negatives binders were assessed for Orai1 binding by FACS analysis as described herein. The hybridoma supernatants derived from the immunization with AM1-CHO/hOrai1/hSTIM1-YFP cells were screened against U20S/Orai1 cells and counter-screened against parental U20S cells. The hybridoma supernatants derived from the immunization with U20S/hOrai1 cells were screened against AM1-CHO/hOrai1/hSTIM1-YFP cells and counter-screened against parental AM1-CHO cells. Exemplary data from the hybridoma supernatant binding screen by FACS are shown in Table 7 below.

TABLE 7
Exemplary FACS binding screen data with hybridoma supernatants shown
as relative fluorescence intensity geometric mean to parental AM1-CHO,
AM1-CHO/hOrai1/hSTIM1-YFP, a ratio of relative fluorescence
intensity geometric mean of AM1-CHO/hOrai1/hSTIM1-YFP
over AM1-CHO and binder score of Yes (ratio of greater or equal to 5)
or No (ratio of less than 5).

| | | AM1-CHO/ | Orai1/parent | |
|---|---|---|---|---|
| | AM1-CHO parental | Orai1/STIM1-YFP | Geometric | |
| Name | Geometric Mean | Geometric Mean | Mean Ratio | Binder |
| 2A1 | 34 | 1592 | 47 | Yes |
| 2B1 | 39 | 2227 | 57 | Yes |
| 2C1 | 46 | 2749 | 60 | Yes |
| 2D1 | 46 | 2583 | 56 | Yes |
| 2E1 | 43 | 2189 | 51 | Yes |
| 2F1 | 42 | 2594 | 62 | Yes |
| 2G1 | 36 | 1969 | 55 | Yes |
| 2H1 | 10 | 89 | 9 | Yes |
| 2A2 | 46 | 2802 | 61 | Yes |
| 2B2 | 11 | 122 | 11 | Yes |
| 2C2 | 45 | 2863 | 64 | Yes |
| 2D2 | 45 | 2750 | 62 | Yes |
| 2E2 | 17 | 280 | 16 | Yes |
| 2G2 | 47 | 2618 | 56 | Yes |
| 2H2 | 9 | 89 | 10 | Yes |
| 2A3 | 18 | 374 | 21 | Yes |
| 2B3 | 43 | 2563 | 60 | Yes |
| 2C3 | 27 | 941 | 34 | Yes |
| 2D3 | 16 | 262 | 16 | Yes |
| 2E3 | 19 | 320 | 17 | Yes |
| 2F3 | 49 | 2487 | 50 | Yes |
| 2G3 | 208 | 253 | 1 | No |
| 2H3 | 42 | 2108 | 50 | Yes |

### Example 4

### Functional Assessment of Anti-Human Orai1 Monoclonal Antibodies in Inhibiting Cytokine Release from Thapsigargin-Treated Human Whole Blood

Ex vivo assay to examine impact of hOrai1 inhibitors on secretion of Interleukin-2 (IL-2) and Interferon (IFN)-gamma. The human Orai1 binding antibodies resulting from Campaign 1 and Campaign 2 were tested for their ability to inhibit T cell activation in human whole blood using an ex vivo assay that has been described earlier (see Example 46 of WO 2008/088422 A2, incorporated herein by reference in its entirety). In brief, 50% human whole blood is stimulated with thapsigargin (a sarcoplasmic recticulum calcium ATPase [SERCA] pump inhibitor) to induce store depletion, calcium mobilization and cytokine secretion. To assess the potency of molecules in blocking T cell cytokine secretion, various concentrations of the anti-Orai1 monoclonal antibodies were pre-incubated with the human whole blood sample for 30-60 min prior to addition of the thapsigargin stimulus. After 48 hours at 37° C., 5% CO₂, conditioned media was collected, and the level of cytokine secretion was determined using a 4-spot electrochemilluminescent immunoassay from MesoScale Discovery. Using the thapsigargin stimulus, the cytokines IL-2 and IFN-gamma were secreted robustly from blood isolated from multiple donors. Positive hybridoma supernatants were selected on their ability to block atleast 80% of both IL-2 and IFN-gamma release. At least one representative subclone from each hit was selected to generate exhaustive supernatants from which the corresponding monoclonal antibodies were purified.

In more detail, human whole blood was obtained from healthy, non-medicated donors in a heparin vacutainer. DMEM complete media was Iscoves DMEM (with L-glutamine and 25 mM Hepes buffer) containg 0.1% human albumin (Gemini, #800-120), 55 μM 2-mercaptoethanol (Gibco), and 1× Pen-Strep-Gln (PSG, Gibco, Cat#10378-016). Thapsigargin was obtained from Alomone Labs (Israel). A 10 mM stock solution of thapsigargin in 100% DMSO was diluted with DMEM complete media to a 40 μM (4× solution) to provide the 4× thapsigargin stimulus for calcium mobilization. Controls: The Kv1.3 inhibitor peptide ShK (Stichodactyla helianthus toxin, Cat#H-2358, Bachem) was used as a positive control in a N-terminally PEGylated form (e.g., according to Example 34 of WO 2008/088422 A2); Charybdotoxin (Cat#H-9595m, Bachem) was also used as a positive control; Fc-L10-ShK[2-35] was another positive control that was used, made and purified as described in Example 2 of WO 2008/088422 A2; Maurotoxin (Alomone RTM-340, Alomone Labs, Jerusalem, Israel) was used as a negative control. These polypeptides controls were used at 100 nM final concn in the assay. Other, or additional, positive and/or negative controls can be employed as long as at least one positive and at least one negative control is employed for the assay; for example, the calcineurin inhibitor cyclosporin A can also be used as a positive control and is available commercially from a variety of vendors.

Ten 3-fold serial dilutions of inhibitors were prepared in DMEM complete media at 4× final concentration and 501 of each were added to wells of a 96-well Falcon 3075 flat-bottom microtiter plate. Whereas columns 1-5 and 7-11 of the microtiter plate contained inhibitors (each row with a separate inhibitor dilution series), 501 of DMEM complete media alone was added to the 8 wells in column 6 and 1001 of DMEM complete media alone was added to the 8 wells in column 12. To initiate the experiment, 1001 of whole blood was added to each well of the microtiter plate. The plate was then incubated at 37° C., 5% CO₂ for one hour. After one hour, the plate was removed and 501 of the 4× thapsigargin stimulus (10 M thapsigargin final concn) was added to all wells of the plate, except the 8 wells in column 12. The plates were placed back at 37° C., 5% CO₂ for 48 hours. To determine the amount of IL-2 and IFN-gamma secreted in whole blood, 100 μl of the supernatant (conditioned media) from each well of the 96-well plate was transferred to a storage plate. For MSD electrochemilluminesence analysis of cytokine production, 25 μl of the supernatants (conditioned medium) were added to MSD Multi-Spot Custom Coated plates (www.meso-scale.com). The working electrodes on these plates were coated with four Capture Antibodies (hIL-5, hIL-2, hIFNg and hIL-4) in advance. After addition of 25 μl of conditioned medium to the MSD plate, 130 μl of a cocktail of Detection Antibodies and P4 Buffer were added to each well. The 130 μl cocktail contained 20 μl of four Detection Antibodies (hIL-5, hIL-2, hIFNg and hIL-4) at 1 μg/ml each and 1101 of 2×P4 Buffer. The plates were covered and placed on a shaking platform overnight (in the dark). The next morning the plates were read on the MSD Sector Imager. Since the 8 wells in column 6 of each plate received only the thapsigargin stimulus and no inhibitor, the average MSD response here was used to calculate the “High” value for a plate. The calculate “Low” value for the plate was derived from the average MSD response from the 8 wells in column 12 which contained no thapsigargin stimulus and no inhibitor. Percent of control (POC) is a measure of the response relative to the unstimulated versus stimulated controls, where 100 POC is equivalent to the average response of thapsigargin stimulus alone or the “High” value. Therefore, 100 POC represents 0% inhibition of the response. In contrast, 0 POC represents 100% inhibition of the response and would be equivalent to the response where no stimulus is given or the “Low” value. To calculate percent of control (POC), the following formula is used: [(MSD response of well)−(“Low”)]/[(“High”)−(“Low”)]×100. The potency of the molecules in whole blood was calculated after curve fitting from the inhibition curve (IC) and IC50 was derived using standard curve fitting software. Although we describe here measurement of cytokine production using a high throughput MSD electrochemillumenescence assay, one of skill in the art can readily envision lower throughput ELISA assays are equally applicable for measuring cytokine production.

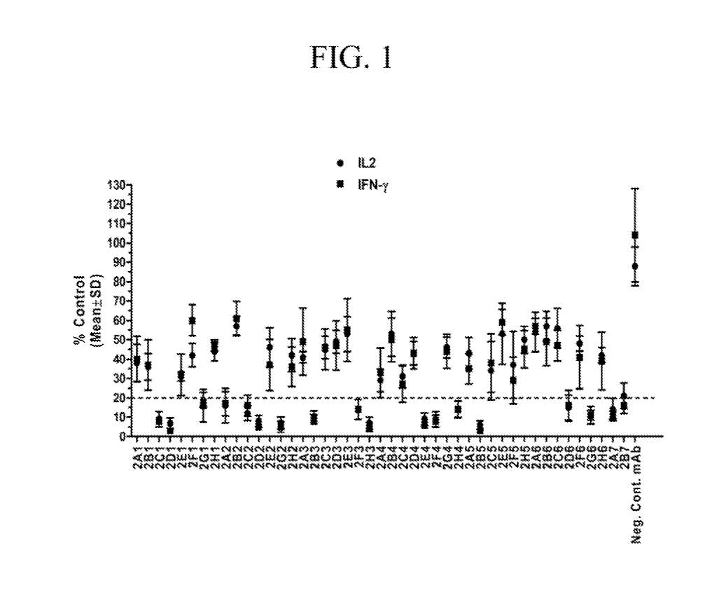

The hits identified in Campaign 1 (Table 7) and Campaign 2 as specific binders to human Orai1 as determined by FACS analysis were assessed in a cytokine-release human whole blood assay where Interleukin-2(IL-2) and Interferon-gamma (IFN-g) released from human whole blood that has been stimulated with thapsigargin was monitored, as described above. The assay was performed on two different human donor blood samples and the results of IL-2 and IFN-g release are expressed as a percent of control where no inhibitor is added. Representative results are shown in FIG. 1. While 25% (volume/volume) of the hybridoma supernatants from the binding hits showed a range of inhibition from greater than 95% to about 40%, the negative control monoclonal antibody only inhibited from less than 15%. In addition, there seems to be a tight correlation between the blocking of IL-2 and IFN-g release. The hits that display greater than 80% inhibition were selected to proceed forward with subcloning and sequencing to obtain purified monoclonal from exhaustive supernatant of subclones of the hits and to determine the sequence of the heavy and light chain antibody sequences.

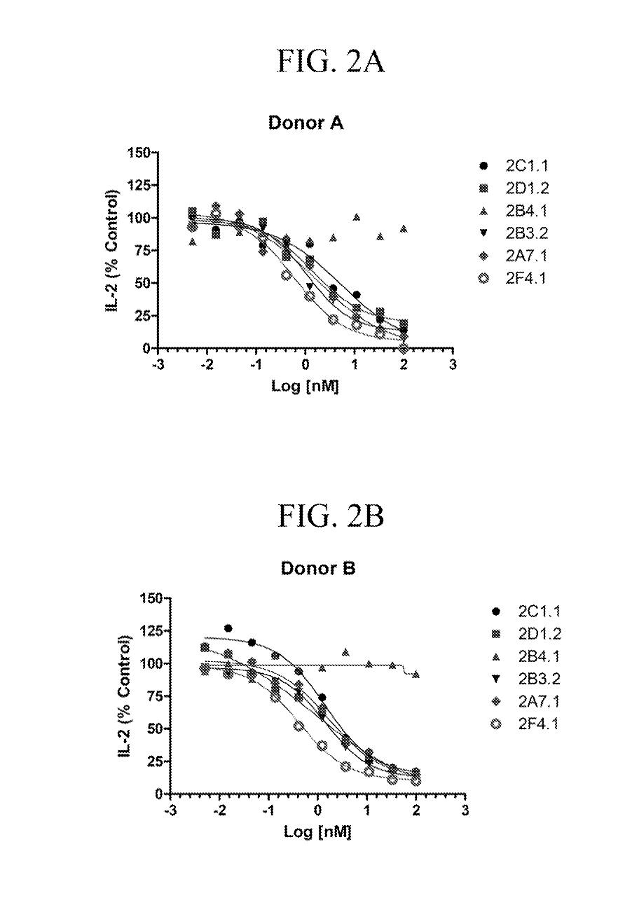

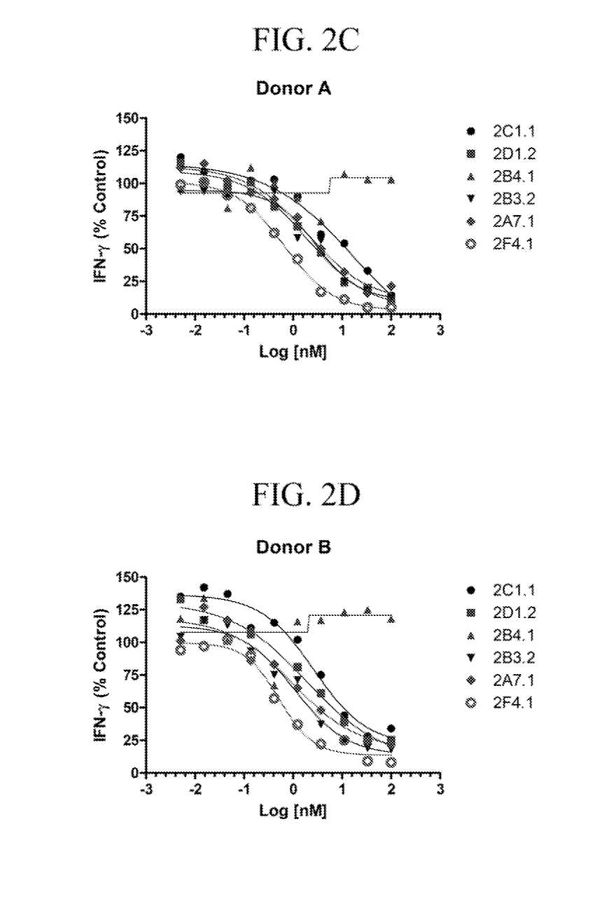

Purified monoclonal antibodies were assessed for binding to human Orai1 by FACS and for their functional activity in inhibiting IL-2 and IFN-gamma cytokine release from thapsgigargin-treated human whole blood (FIG. 2A-D). Exhaustive hybridoma supernatants from the subclones of selected hits were generated so that their monoclonal antibodies (mAb) could be purified after the mAbs were verified for their specific binding to human Orai1. Purified mAbs were assessed for their ability to block cytokine release from human whole blood assay at various concentrations and graphed as percent of control without inhibitor versus different concentrations of the mAbs, as described above. FIGS. 2A and 2B show exemplary results of selected mAbs (2C1.1, 2D1.2, 2B3.2, 2A7.1 and 2F4.1) inhibiting IL-2 release from human whole blood from donor A and B that has been treated with thapsigargin. However, mAb 2B4.1, which was selected as an internal negative control for blocking cytokine release from human whole blood assay but a binder of human Orai1, showed no inhibition of IL-2 release with increasing concentrations. FIGS. 2C and 2E show a similar dose-response inhibition curves for mAbs 2C1.1, 2D1.2, 2B3.2, 2A7.1 and 2F4.1 but not for the binder only control mAb 2B4.1. The inhibition curves for both IL-2 and IFN-g display a complete inhibition of both cytokines' release. The potency assessment displayed a dose-dependent inhibition of both IL-2 and IFN-gamma release from two different donor whole bloods that were treated with thapsigargin and IC50s in the range of low nanomolar, as shown in Table 8A (Campaign 1) and Table 8B (Campaign 2) below.

Shown in Table 8A are the half-maximal inhibitory concentrations (IC50) of the purified monoclonal antibodies from 16 selected subclones from Campaign 1 in blocking IL-2 and IFN-gamma secretion from thapsigargin-treated human whole blood. In addition to the 16 selected blocking mAbs, two binding-only mAbs 2B4.1 and 2H4.1 were selected as internal negative control mAbs. All of the 16 selected mAbs displayed potent IC50s in the low nanomolar concentration range inhibiting IL-2 and IFN-g release in human blood from two different donors. On the other hand, as expected, no inhibition was observed with mAbs 2B4.1 and 2H4.1. The table also shows the R² coefficient of determination very close to one indicating a very good fit between how well the regression line approximates the real data points.

TABLE 8A
Half-maximal inhibitory concentrations (IC50) of purified monoclonal
antibodies from selected subclones derived from the Campaign 1 hits in FIG. 1 in
blocking IL-2 and IFN-gamma secretion detected in the thapsigargin-treated human
whole blood assay system. Also shown is the R2 coefficient of determination, a
statistical measure of how well the regression line approximates the real data points
with 1.0 indicating that the regression line perfectly fits the data.

| | IL2 | | | | IFN-γ | | | |
|---|---|---|---|---|---|---|---|---|
| Clone | Donor A, | | Donor B, | | Donor A, | | Donor B, | |
| Name | IC₅₀, nM | R² | IC₅₀, nM | R² | IC₅₀, nM | R² | IC₅₀, nM | R² |
| 2C1.1 | 5.23 | 0.96 | 1.44 | 0.99 | 17.10 | 0.97 | 2.67 | 0.97 |
| 2D1.2 | 1.46 | 0.96 | 0.89 | 0.99 | 2.00 | 0.99 | 1.94 | 0.99 |
| 2B4.1 | No Inhibition | | No Inhibition | | No Inhibition | | No Inhibition | |
| 2B3.2 | 1.08 | 0.98 | 1.44 | 0.99 | 2.97 | 0.97 | 1.10 | 0.98 |
| 2A7.1 | 1.65 | 0.97 | 1.70 | 0.98 | 1.96 | 0.99 | 1.01 | 0.95 |
| 2F4.1 | 0.62 | 0.98 | 0.42 | 1.00 | 0.67 | 1.00 | 0.52 | 0.98 |
| 2A2.1 | 1.32 | 0.99 | 2.51 | 0.98 | 6.91 | 0.99 | 1.79 | 0.98 |
| 2F3.2 | 2.98 | 1.00 | 0.86 | 0.99 | 3.92 | 0.98 | 1.00 | 0.94 |
| 2H4.1 | No Inhibition | | No Inhibition | | No Inhibition | | No Inhibition | |
| 2C2.1 | 1.22 | 0.99 | 0.74 | 1.00 | 2.04 | 0.97 | 1.37 | 0.96 |
| 2E4.1 | 1.85 | 0.99 | 1.03 | 0.99 | 1.36 | 0.98 | 1.02 | 0.92 |
| 2B7.1 | 3.50 | 0.91 | 4.67 | 0.96 | 4.60 | 0.93 | 14.04 | 0.99 |
| 2G6.1 | 3.98 | 0.95 | 1.55 | 0.99 | 4.48 | 0.98 | 2.46 | 0.94 |
| 2H3.1 | 5.72 | 0.99 | 1.86 | 0.99 | 4.91 | 0.98 | 1.22 | 0.98 |
| 2G2.1 | 1.84 | 0.97 | 1.43 | 0.98 | 1.33 | 0.96 | 1.25 | 0.88 |
| 2B5.1 | 1.39 | 1.00 | 1.10 | 1.00 | 1.61 | 0.98 | 1.40 | 0.94 |
| 2D2.1 | 1.31 | 0.98 | 1.16 | 0.98 | 1.98 | 0.99 | 2.06 | 0.95 |
| 2F1.2 | 4.76 | 0.99 | 2.86 | 1.00 | 22.91 | 0.94 | 2.18 | 0.95 |

TABLE 8B
Half-maximal inhibitory concentrations (IC50) of purified monoclonal
antibodies from selected subclones derived from the Campaign 2 hits in blocking IL-2
and IFN-gamma secretion detected in the thapsigargin-treated human whole blood
assay system. Potencies were in the low nano-molar IC50 range in blocking IL-2
and IFN-g release from human whole blood assay. Also shown is the R2 coefficient
of determination, a statistical measure of how well the regression line approximates
the real data points with 1.0 indicating that the regression line perfectly fits the data.

| | IL-2 | | | | IFN-γ | | | |
|---|---|---|---|---|---|---|---|---|
| Clone # | Donor A, IC₅₀, nM | R² | Donor B, IC₅₀, nM | R² | Donor A, IC₅₀, nM | R² | Donor B, IC₅₀, nM | R² |
| 5A1.1 | 11.24 | 0.98 | 5.59 | 0.99 | 8.15 | 0.97 | 1.66 | 0.90 |
| 5A4.2 | 1.88 | 1.00 | 1.21 | 0.99 | 4.83 | 0.96 | 2.76 | 0.99 |
| 5B1.1 | 2.73 | 0.94 | 4.45 | 0.95 | 7.40 | 0.91 | 10.72 | 0.89 |
| 5B5.2 | 1.39 | 0.88 | 3.10 | 0.99 | Ambiguous | | 6.58 | 0.85 |
| 5C1.1 | 2.18 | 0.99 | 1.24 | 0.99 | 5.42 | 0.97 | 2.41 | 0.97 |
| 5F2.1 | 4.83 | 0.91 | 3.79 | 1.00 | Ambiguous | | Ambiguous | |
| 5F7.1 | 4.05 | 0.97 | 3.49 | 0.98 | Ambiguous | | 11.85 | 0.98 |

Sequence Analysis of Selected Monoclonal Antibodies.

Eighteen positive hits from the initial functional screen (FIG. 1) were subcloned. The hybridoma supernatants containing the monoclonal antibodies expressed from the subclones of each hit were verifiied for their binding to human Orai1 by FMAT binding screen. Three selected subclones from each hit were chosen to proceed forward to sequencing of the heavy and light antibody chains based on positive binding to human Orai1. To obtain the heavy chain and light chain antibody sequences of the monoclonal antibodies from the three selected sublcones from each hit, messenger RNAs of the heavy chain (HC) and light chain (LC) antibody genes were isolated and sequenced using standard reverse transcription-PCR method. The HC and LC sequences are shown in Table 1A and Table 1B herein above. Several clones from Campaign 1 share the same LC or HC sequence, which indicates that the human antibodies found during the anti-Orai1 Campaign 1 are very closely related.

Transient Expression to Generate Recombinant Monoclonal Antibodies.

HEK 293-6E cells were maintained in 3-L Fernbach Erlenmeyer Flasks between 2×10⁵ and 1.2×10⁶ cells/ml in F 17 medium supplemented with L-Glutamine (6 mM) and Geneticin (25 μg/ml) at 37° C., 5% CO₂, and shaken at 65 RPM. At the time of transfection, cells were diluted to 1.1×10⁶ cells/mL in the F 17 medium mentioned above at 90% of the final culture volume. DNA of a one to one ratio of the heavy and light chain complex was prepared in Freestyle™293 medium (Invitrogen) at 10% of the final culture volume. DNA complex included 500 μg total DNA per liter of culture and 1.5 ml PEImax per liter of culture. DNA complex was briefly shaken once the ingredients were added and incubated at room temperature for 10 to 20 minutes before being added to the cell culture, which was then placed back in the incubator. The day after transfection, Tryptone N1 (5 g/L) was added to the culture from liquid 20% stock. Six days after transfection, culture was centrifuged at 4,000 RPM for 40 minutes to pellet the cells, and the cultured medium was harvested through a 0.45 μm filter.

Purification of Recombinant Monoclonal Antibodies.

The conditioned media were purified by affinity capture binding of the Fc region using rProtein A Sepharose Fast Flow medium (GE Healthcare). The affinity capture system was purified on an AKTA fast protein liquid chromatography (FPLC) Explorer™ automated system. The buffer system used was Buffer A Equilibration Buffer: Dulbecco's PBS without cations (Gibco); Buffer B elution: 100 mM Acetic Acid, pH 3.5 and an optional Buffer C: 100 mM Glycine, pH 2.7. Each sample was loaded onto the affinity media and washed with buffer A, then isocratic 100% step elution Buffer B at not more than 2.5 cm/hour. The protein pool was determined by peak absorbance of the FPLC system at λ=280 nm. If no peak was eluted at λ=280 nm from Buffer B peak, then Buffer C was used to elute any remaining antibody. The eluted fractions were pooled by peak chromatography fractions. The pool was then adjusted to pH 5.0 using 2 M Tris base. The pool sample was filtered using a 0.8 μm/0.2 μm filter (Pall). The samples were directly dialyzed into 10 mM sodium acetate, 9% sucrose, pH 5.0, using a 10 kDa molecular weight cut off membrane (Pierce). If necessary, the samples were concentrated using a 30 kDa molecular weight cut-off (MWCO; Vivaspin) centrifuge concentrator. The protein concentration was determined by analyzing the UV absorbance spectra (λ=260; 280; 340 nm) on a spectrophotometer (Nanodrop).

Characterization of Recombinant Antibodies.

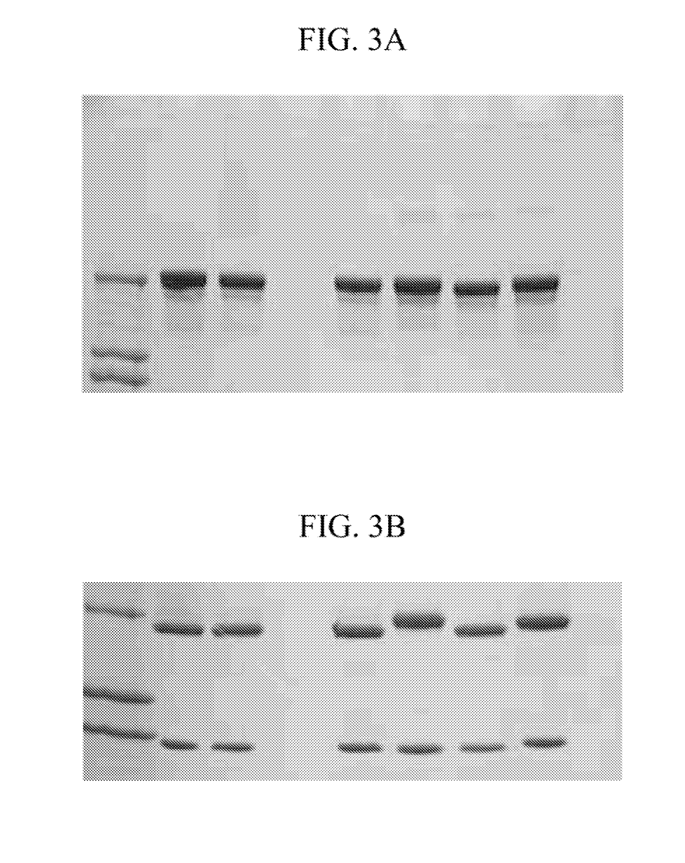

The recombinant antibodies were analyzed by SDS-PAGE gel analysis under reducing (+beta-mercaptoethanol) and non-reducing conditions with Iodoacetamide on a Tris-Glycine SDS-PAGE gel (Invitrogen) and stained with Boston Biologics dye. Sample sterility was confirmed using a limulus amebocyte lysate test cartridge (Charles Rivers Laboratories). After transient expression of the heavy and light chain antibody genes in HEK-293 cells, the recombinant mAbs were purified by affinity capture binding using Protein A. After purification, the recombinant mAbs were visualized on Tris-Glycine SDS-PAGE gel under non-reducing (FIG. 3A) and reducing conditions (FIG. 3B). The photographs of the gels show that the mAbs were purified to high purity.

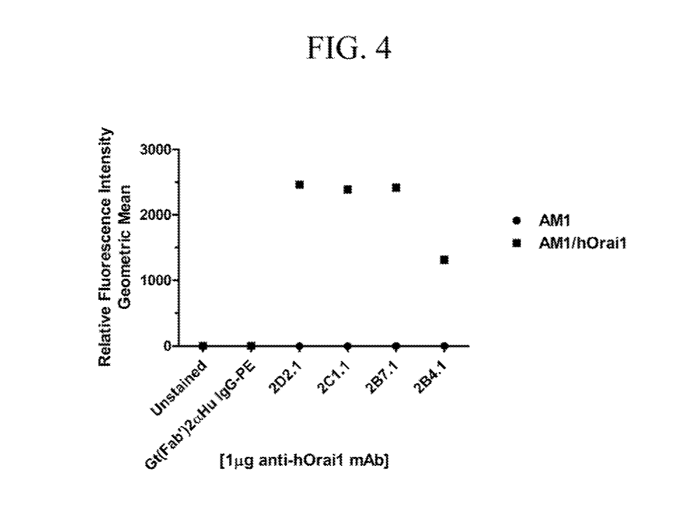

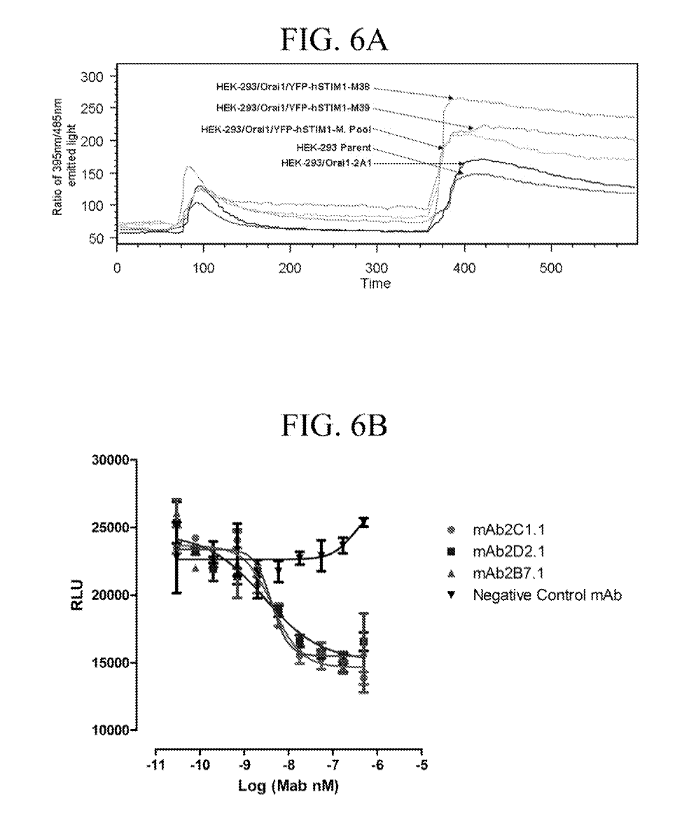

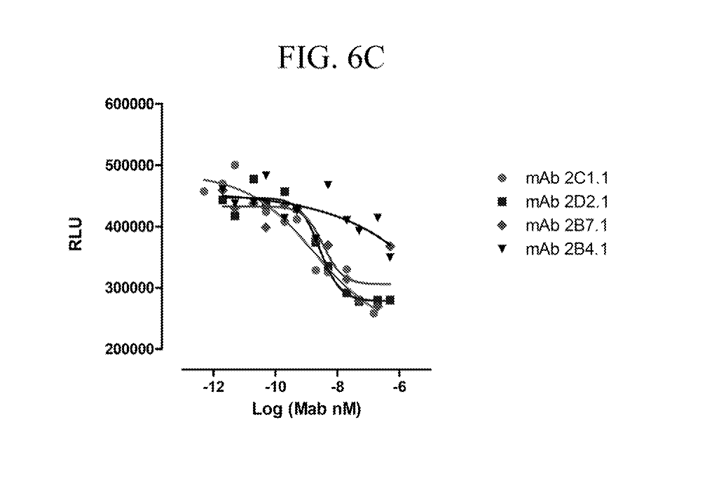

Binding to human Orai1 by recombinant antibodies was assessed by the FACS method described herein. FIG. 4 demonstrates binding to human Orai1 by recombinant monoclonal antibodies 2D2.1, 2C1.1, 2B7.1, and 2B4.1. Out of the 16 mAbs identified for potent inhibition of cytokine release from human whole blood assay, analysis of their corresponding heavy and light chain antibody sequences indicated that there were three unique monoclonal antibodies referred to as mAb 2C1.1, mAb 2D2.1 and mAb 2B7.1. The recombinant mAbs were first assessed to confirm their specific binding to human Orai1 expressed on the surface of AM1/CHO cells. FIG. 4 shows mAb 2C1.1, mAb 2D2.1 and mAb 2B7.1 binding to parental CHO was negligible, with a low relative fluorescence intensity geometric mean (geo mean) value that was comparable to the unstained control and directly labeled secondary reagent-only staining control. The geo mean values for the AM1/hOrai1 were also low for the unstained control and secondary reagent-only. However, there was significant specific binding as indicated by the huge increase in values of the geo mean for mAb 2C1.1, mAb 2D2.1, mAb 2B7.1 and mAb 2B4.1 on AM1/hOrai1. It is noteworthy that the geo mean value for the mAb 2B4.1 was about half the value of the other mAbs and probably indicates a lower binding affinity of mAb 2B4 compared to the other mAbs tested. The mAb 2B4.1 binds AM1/hOrai1, and shows the ability to inhibit CRAC channel activity to some extent (see FIG. 6C), but was ineffective in inhibiting IL-2 and IFN-gamma release in the thapsigargin whole blood assay described above. It is unclear whether mAb 2.B4.1's lower relative binding strength was directly related to its inability to inhibit cytokine release in the whole blood assay.

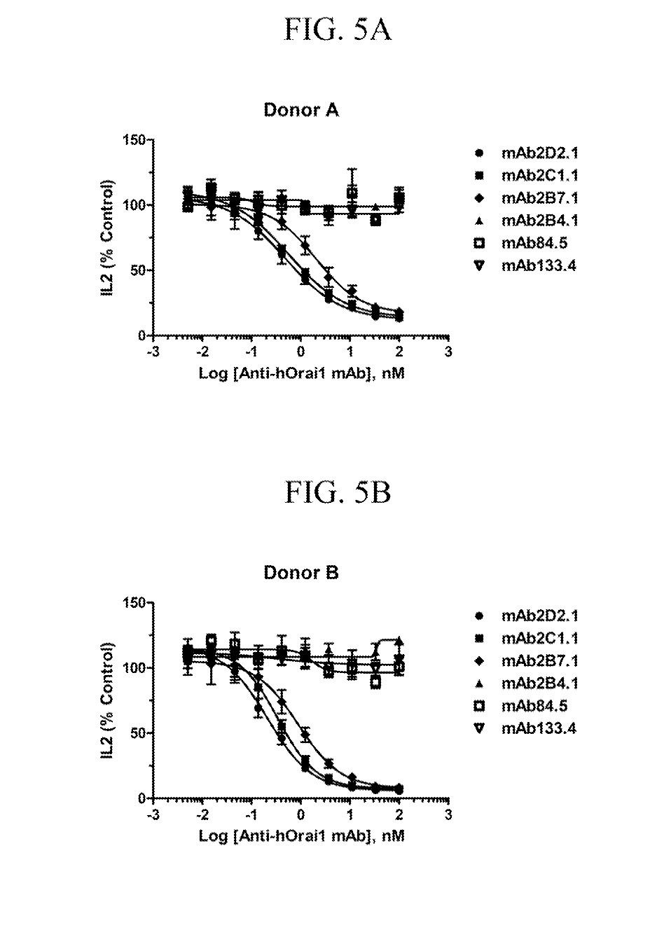

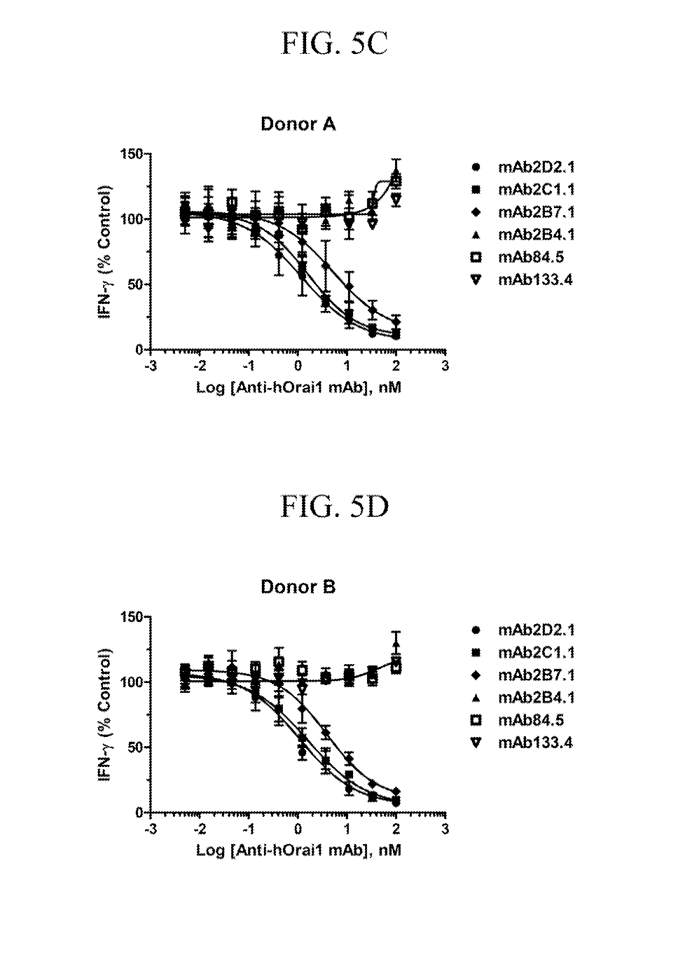

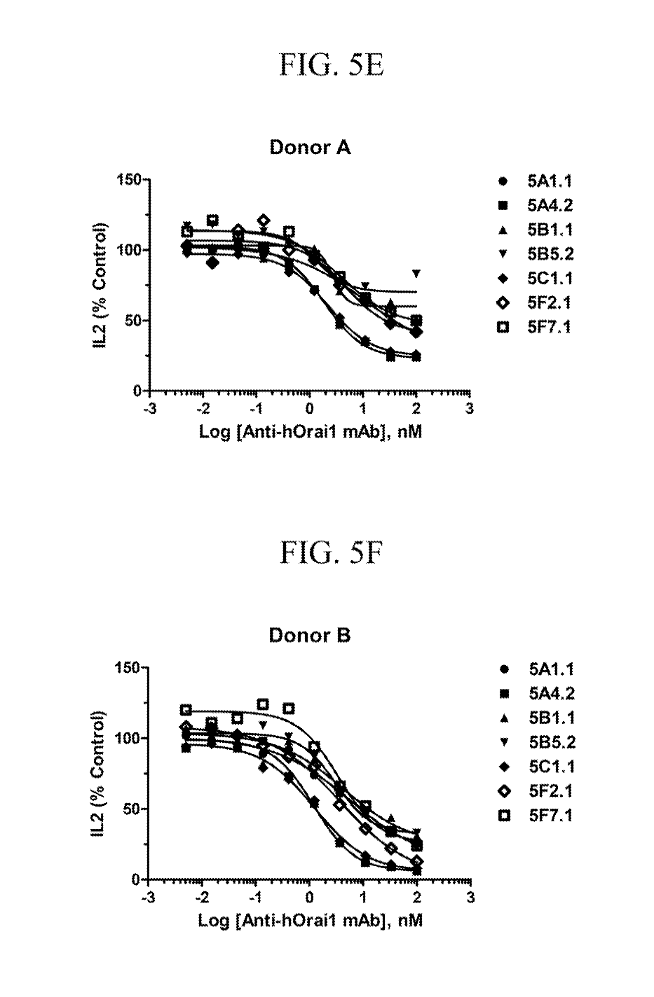

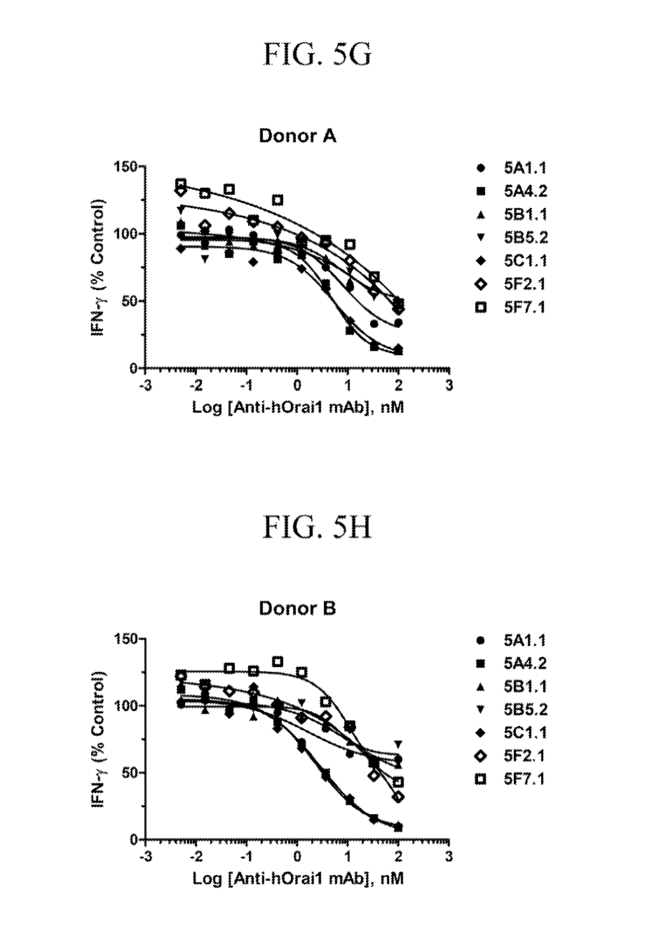

As noted above, recombinant antibody mAb 2B4.1 specifically bound human Orai1 but did not exhibit detectable functional activity in inhibiting cytokine secretion (FIG. 4 and FIG. 5A-D) in the whole blood assay system. FIG. 5A-D illustrates dose-dependent inhibition of IL-2 and IFN-gamma secretion by Campaign 1 monoclonal antibodies mAb 2D2.1, mAb 2C1.1, and mAb 2B7.1 in the whole blood assay. FIG. 5E-H illustrates dose-dependent inhibition of IL-2 and IFN-gamma secretion by Campaign 2 antibodies mAb 5A1.1, mAb 5A4.2, mAb 5B1.1, mAb 5B5.2, mAb 5C1.1, mAb 5F2.1, and mAb 5F7.1 in the whole blood assay. IC50 values for each of the Campaign 1 recombinant antibodies are shown in Table 9A below. Recombinant mAbs were assessed for their ability to block cytokine release from human whole blood assay at various concentrations and graphed as percent of control without inhibitor versus different concentrations of the mAbs. FIG. 5A-H show that while mAb 2C1.1, mAb 2D2.1, mAb 2B7.1, mAb 5A1.1, mAb 5A4.2, mAb 5Bl1.1, mAb 5B5.2, mAb 5C1.1, mAb 5F2.1, and mAb 5F7.1 dose-dependently blocked IL-2 release from human whole blood, the mAb2B4.1, and mAb84.5 and mAb 133.4 did not inhibit IL-2 and IFN-gamma release in human whole blood from two different donors. The results indicate that while all thirteen mAbs bound strongly to human Orai1, only mAb 2C1.1, mAb 2D2.1, mAb 2B7.1, mAb 5A1.1, mAb 5A4.2, mAb 5Bl1.1, mAb 5B5.2, mAb 5C1.1, mAb 5F2.1, and mAb 5F7.1 inhibited both IL-2 and IFN-g release in the human whole blood assay system.

Table 9A (below) shows the half-maximal inhibitory concentrations (IC50) of the recombinant mAbs 2C1.1, 2D2.1 and 2B7.1 in blocking IL-2 and IFN-gamma secretion from thapsigargin-treated human whole blood. Comparing the IC50s of purified mAbs from Table 8A (above) with the IC50s of recombinant mAbs in Table 9A (below), it was observed that the potency increased with lower IC50 values in inhibiting cytokine release from human whole blood assay. It is also interesting that all three recombinant mAbs blocked more potently IL-2 secretion than IFN-g release from human whole blood as indicated by the lower IC50s.

TABLE 9A
IC50s of mAb 2D2.1, mAb 2C1.1 and mAb 2B7.1 in inhibiting
Interleukin-2 (IL-2) and interferon-gamma (IFN-g) release in the
whole blood assay system.

| | IC₅₀ in | | IC₅₀ in | |
|---|---|---|---|---|
| | nM of blocking IL2 | | nM of blocking IFN-g | |
| | Donor A | Donor B | Donor A | Donor B |
| mAb2D2.1 | 0.49 | 0.21 | 1.18 | 1.10 |
| mAb2C1.1 | 0.57 | 0.34 | 1.62 | 1.61 |
| mAb2B7.1 | 2.06 | 0.90 | 5.27 | 3.93 |

### Example 5

### Assessment of anti-Human Orai1 Monoclonal Antibodies in Functional Luciferase Assay

Cell Line Development.

HEK-293T cells were transfected with pTT14/puro-hOrai1 and pcDNA 3.1/zeo-YFP-hSTIM1 using FuGENE 6 in growth medium according to the manufacturer's protocol (Roche). Two days after transfection, cells were dislodged from plate surface using 0.5% trypsin (Gibco) and re-plated into growth medium containing 0.5 μg/mL of Puromycin (BD Biosciences) and 0.5 μg/mL of Zeocin (Invitrogen). The pool was sorted twice by FACS for YFP into low, medium and high pools. The medium pool was subcloned and clones were evaluated using Indo-1 AM (Invitrogen) ratiometric Ca²⁺ flux assay. The cell line generated was then plated at 1×10⁶ cells/well into a 6-well plate and transfected with 2 μg of pGL4.30 (luc2P/NFAT-RE/Hygro) from Promega using FugeneHD (Roche) and was selected with hygromycin (Roche) at 200 μg/mL while maintaining the puromycin and zeomycin selection. This pool was subcloned by single cell limited dilution cloning and the subclones were evaluated based on NFAT-luc activity resulting in the cell line used for the NFAT-Luciferase reporter assays.

Ratiometric Calcium Influx Assay.

HEK-293 cells expressing human Orai1 and hStiml-YFP fusion protein were generated and characterized using an Indo-1 ratiometric calcium influx assay. This assay uses an Indo-1 ratiometric calcium dye (Invitrogen) in a FACS machine to measure the calcium influx into cells by the changes in emission wavelength of Indo-1 in the presence and absence of calcium. To initiate calcium influx through Orai1 channels, internal calcium release from stores was caused by treatment with thapsigargin that triggers the Stiml to translocate to the punctae and activate Orai1 channels. The opening of the Orai1 channel results from an influx of calcium down the concentration gradient into the cells that can be visualized using Indo-1 dye. FIG. 6A shows a plot of calcium entry into cells as represented by the ratio of 395 nm/485 nm emitted light on the y-axis over time (seconds) on the x-axis. The first minute of recording represents the baseline before any treatment with the low ratio representing low calcium level inside the cells. FIG. 6A shows that following stimulation with thapsigargin after one minute to induce the internal stored calcium to be released, the 395 nm/485 nm ratio increased immediately but quickly declined over time to baseline representing the transient increase in calcium inside the cells. When the internal calcium level returned to baseline then external calcium dication was added to 2 mM resulting in an immediate and sharper rise in the 395 nm/485 nm ratio representing an even higher level of calcium inside the cells caused by the calcium influx into the cells by the opening of the Orai1 channel. In FIG. 6A, the first peak representing calcium release from the internal stores, were similar in all the cell lines (albeit somewhat sharper in HEK-293T/hOrai1/Stiml-YFP-M38), including the HEK-293T parental cells. However, the second peak representing the calcium influx through Orai1 channel for the clone HEK-293T/hOrai1/Stiml-YFP-M38 was dramatically higher than all the other cell lines and was chosen as the lead for further experiments, because it demonstrated higher Orai1 activity as measured by this Indo-1 ratiometric calcium influx assay.

NFAT-Luciferase Reporter Assay. The HEK-293T/hOrai1/Stiml-YFP-M38 cell line was engineered to harbor a reporter plasmid containing the luciferase gene under control of the NFAT transcription factor so that thapsigargin stimulated increase in Orai1 activity can be reported with NFAT activated luciferase activity. The NFAT-luciferase assay is based on the fact that thapsigargin treatment will cause sustained influx of calcium through Orai1 channel (see, FIG. 6A). The sustained increase in intracellular calcium level can orchestrate a myriad of cellular responses through many calcium binding proteins such as calmodulin. Calmodulin, when bound to calcium dication activates calcineurin, a serine and threonine phosphatase that, in turn dephosphorylates NFAT resulting in the translocation of NFAT into the nucleus. In the nucleus, NFAT activates the transcription of the luciferase reporter gene encoding for luciferase enzyme the activity of which can be easily measured. The lead cell line HEK-293T/hOrai1/Stiml-YFP-M38-NFAT-Luc-3C12 was chosen based on the criteria that thapsigargin treatment in the presence of external calcium elicits a big increase in relative light unit (RLU), but a negligible increase in RLU when treated with thapsigargin in the absence of external calcium.

In the NFAT-Luciferase reporter assay, transfected HEK-293T cells were plated at 1×10⁵/well (25 μL/well) in calcium free media (DMEM [Gibco] supplemented with 10% FBS+1×NEAA+1× sodium pyruvate+L-glutamine). Anti-Orai1 mAbs were added to the wells starting at 500 nM (25 μL/well) in log dose and incubated for one hour at 37° C. degrees. Thapsigargin was added to each well (25 μL/well) at 10 μM final concentration, and the mixture was incubated at 37° C. degrees for one hour. Two mM final concentration of calcium was then added to each well and the plate was incubated for 5 hours at 37° C. degrees. Steady-Glo® luciferase assay substrate (Promega) was added at 100 μL/well, the plate was incubated at room temperature for 5 minutes and then read on a Wallac EnVision™ plate reader.

FIG. 6B-C show results from two representative assays to assess anti-Orai1 mAbs in blocking NFAT activated transcription of the luciferase reporter gene in response to thapsigargin stimulated calcium influx through Orai1 channel. These results indicate that calcium flux through CRAC was inhibited by mAb 2C1.1 (IC50=4.4 nM), mAb 2D2.1 (IC50=2.8 nM), and mAb 2B7.1 (IC50=4.2 nM) in a dose-dependent manner in the NFAT-mediated luciferase assay; the Negative Control mAb failed to do so (FIG. 6B). In the experiment represented in FIG. 6C, similar IC50 values were determined for mAb 2C1.1 (IC50=1.8 nM), mAb 2D2.1 (IC50=2.7 nM), mAb 2B7.1 (IC50=3.5 nM), and mAb 2B4.1 (IC50=1.8 μM).

The experiment was repeated with mAb 2B4.1, which binds human Orai1 but was inactive in cytokine release human whole blood assay, as noted above. The results in FIG. 6C show that again the mAb 2C1.1, mAb 2D2.1 and mAb 2B7.1 displayed dose-dependent inhibitions of luciferase activity. The mAb 2B4.1 only inhibited slightly at higher concentrations of antibodies with a trend to dose-dependency. However, calculated IC50 for mAb 2B4.1 shows this antibody to be about 1000-fold less potent, with an IC50 in the micro-molar range, whereas IC50 was in the low nano-molar for mAb 2C1.1, mAb 2D2.1 and mAb 2B7.1 (see, previous paragraph). It is interesting that the inhibition, while dose-dependent, by all the mAbs, is only partial at about fifty percent maximal inhibition, indicating the calcium influx in this assay system may be mediated by not only Orai1 but other SOCs that are not blocked by the mAbs.

### Example 6

### Assessment of Anti-Human Orai1 Monoclonal Antibodies by Electrophysiological Assay

Cell Line Development. Human Orai1 cDNA (SEQ ID NO:1) was cloned into the tetracycline-inducible vector pcDNA5/TO (Invitrogen) and referred to as pcDNA5/TO-hOrai1. This inducible vector along with pcDNA6-TR (Invitrogen), an expression vector expressing tetracycline repressor, and the pcDNA3.1/zeocin-hSTIM1 were co-transfected into HEK-293T cells. Two days after transfection, cells were dislodged from plate surface using 0.5% trypsin (Gibco) and were re-plated into growth medium containing 5 μg/mL of Blasticidin (Invitrogen), 200 μg/mL of Hygromycin (Roche) and 100 μg/ml Zeocin (Invitrogen). The selection for transfected cells continued until visible colonies arose. Single colonies were picked into 24-well tissue culture plates and expanded for functional calcium influx assessment. Selected colonies were further subcloned to ensure clonality and the sublcones were similarly assessed by a fluorescence imaging plate reader (FLIPR)-based calcium influx assay system. The cell line referred to as HEK-293/hOrai1/hSTIM1 BB6.3 clone was chosen as the lead for calcium influx and electrophysiology assays.

Functional assessment of cell lines by FLIPR-based calcium influx assay system. BB6.3 cells were plated at 25,000 cells in a 50 μL volume of growth medium per well into a Collagen I Cellware 384-well black/clear plates (BD Bioscience) the day before the assay. A Dye Load buffer containing Calcium Ringer Solution Base (10 mM HEPES, 4 mM MgCl₂, 120 mM NaCl, 5 mM KCl, pH 7.2 and 0.1% bovine serum albumin (Sigma)), 100×PBX signal Enhancer (BD Bioscience) and Calcium Indicator (BD Bioscience) was added immediately before use. The growth medium was carefully decanted from the cell plate; Dye Load buffer was added, followed by incubation for at least 1.5 hours at 29° C. After the Calcium Indicator was loaded, 1 μM Thapsigargin (final concentration) in Calcium Ringer Solution Base was added to the cells. The plate was imaged for 1.5 minutes and data were recorded with an imaging machine with fluorescence detection/plate imaging capability, as described in Rasnow et al., Apparatus and Method for Interleaving Detection of Fluorescence and Luminescence, WO 2008/091425 and US2008/0179539, however any suitable FLIPR machine may be used instead. The plate was then incubated for 30 minutes at 29° C., after which calcium in Calcium Ringer Solution Base was added back. The plate was imaged for 1.5 minutes in the calcium imaging machine, and data were analyzed. As previously noted, thapsigargin-treatment of the cell causes calcium to be released from the stores and the activation and translocation of Stiml, and in the absence of external calcium there should not be any detectable calcium influx. Stiml then activates the opening of Orai1 channel to allow the influx of calcium into the cells when external calcium is added back. The calcium is bound by the dye and read on an imager.

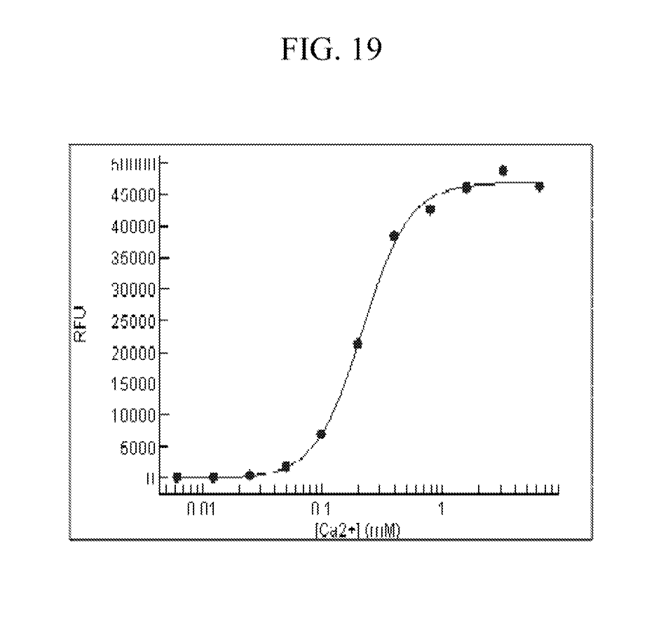

Representative results are shown in FIG. 19 showing RFU that represents the Orai1 activity in response to external calcium dication concentration. The cells showed a dose-dependent increase in relative fluorescence units when calcium was added back to the medium and calcium cation influx into the cells via Orai1 channels, demonstrating functional CRAC channel activity. Although expression of the human Orai1 protein was under control of the tetracycline-inducible vector, there was leakiness of expression of human Orai1. This expression level coupled with the constitutive expression level of human Stiml gave the highest relative fluorescent unit (RFU) in the FLIPR-based calcium influx assay system as compared to tetracylcine-treated cells. The selected clone HEK-293/hOrai1/hSTIM1 BB6.3 was chosen as the lead cell line without induction with tetracycline as the optimal condition for the FLIPR-based calcium influx assay system.

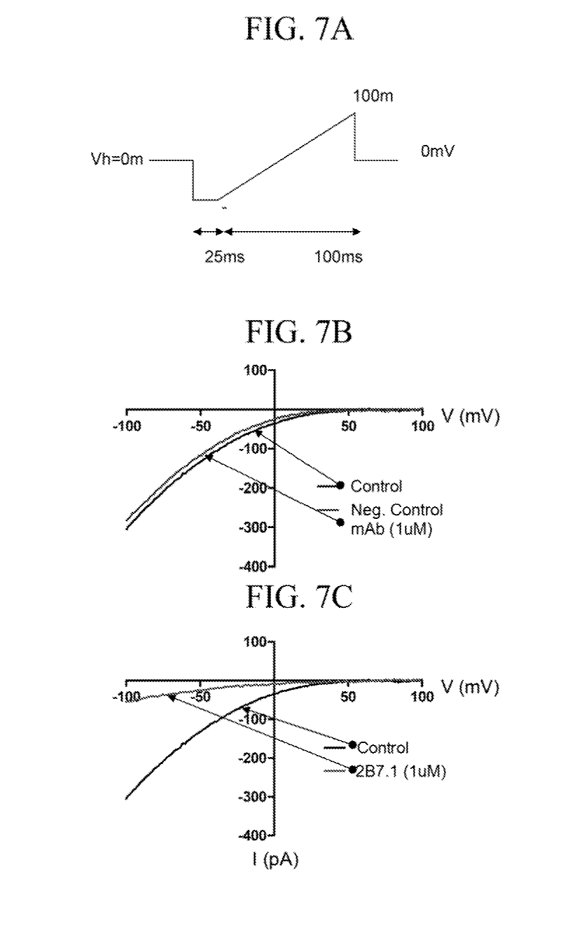

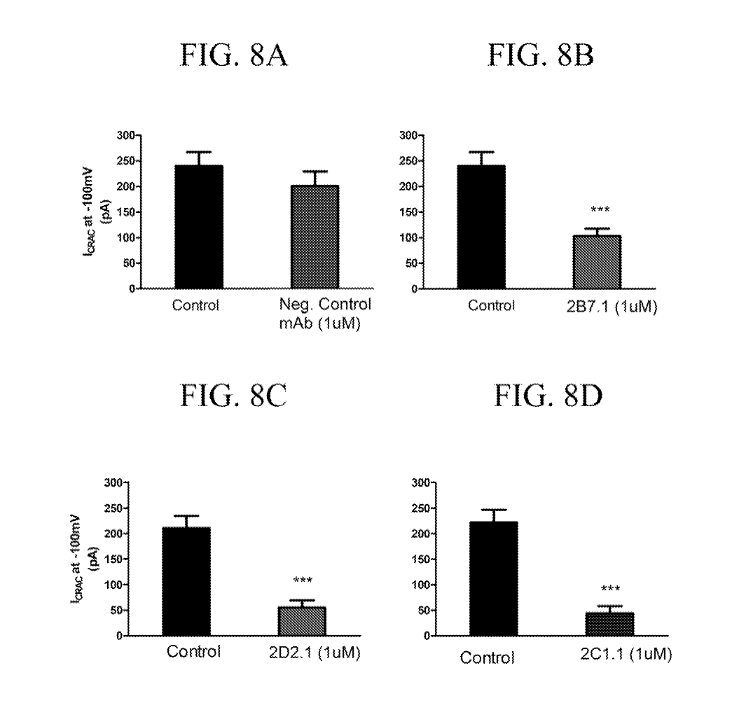

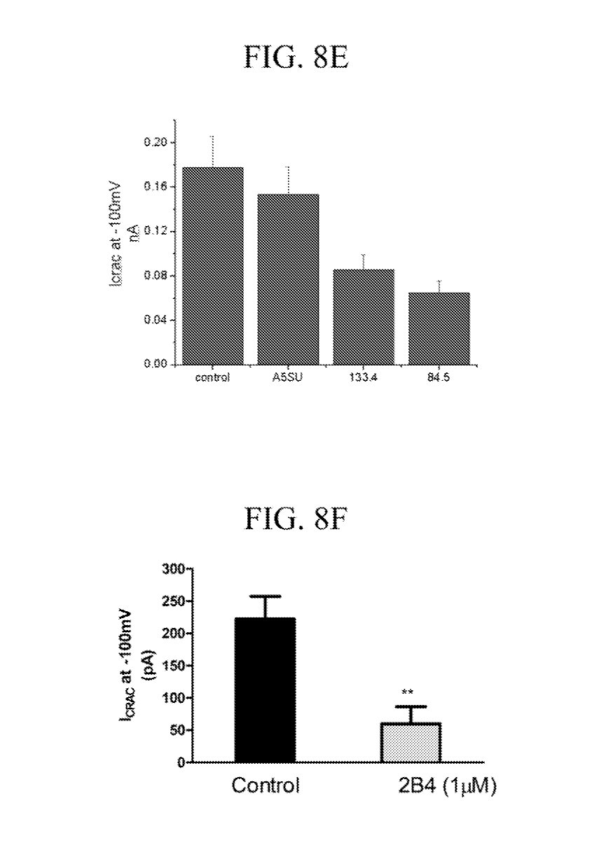

Electrophysioloyv. The HEK-293/hOral/ hSTIM1 BB6.3 cells were pre-treated with 1 μM of antibodies or without antibodies (control) for 1 hour at room temperature. All experiments were carried out with PatchXpress 600™ electrophysiology system from Molecular Devices Corporation (Sunnyvale, Calif.). Cells were bathed in an extracellular solution containing 112 mM NaCl, 2 mM MgCl₂, 10 mM CsC1, 10 mM CaCl₂, 10 mM Glucose, 10 mM HEPES, pH=7.2, 298 mOsm. A resuspended cell in the 1.5 ml Eppendorf tube was inserted into the appropriate slot of the PatchXpress™. Sealchips™ were filled by automation on PatchXpress with an internal solution containing 10 mM BAPTA, 120 mM CsGlutamate, 8 mM NaCl, 8 mM MgCl₂, 10 mM HEPES, pH=7.2, 292 mOsm. On PatchXpress, cells were patched and once a gigaOhm seal was achieved, whole cell conformation was obtained. Currents were visualized using PatchXpress® Commander software. Cells were held at a holding potential of 0 mV. The membrane potential was stepped to −100 mV for 25 ms, and a 100 ms voltage ramp going from −100 to 100 mV with an interval of 15 s was applied. Data were analyzed using GraphPad Prism software. Averages are presented as mean±S.E.M. For statistical analysis, unpaired two-tailed t-test was used. Representative results are shown in FIG. 7A-C and FIG. 8A-F, which illustrates the ability of several anti-hOrai1 antibodies of the present invention to inhibit CRAC current (ICRAC), including mAb 2B7.1, mAb 2D2.1, mAb 2C1.1, mAb 2B4.1, mAb 84.5 and mAb 133.4. The IC50 of mAb 2C1.1 for ICRAC was determined (see, Example 20 herein).

### Example 7

### Assessment of the Binding Specificity of Anti-Human Orai1 Monoclonal Antibodies

Molecular Cloning of Human Orai2 and Human Orai3.

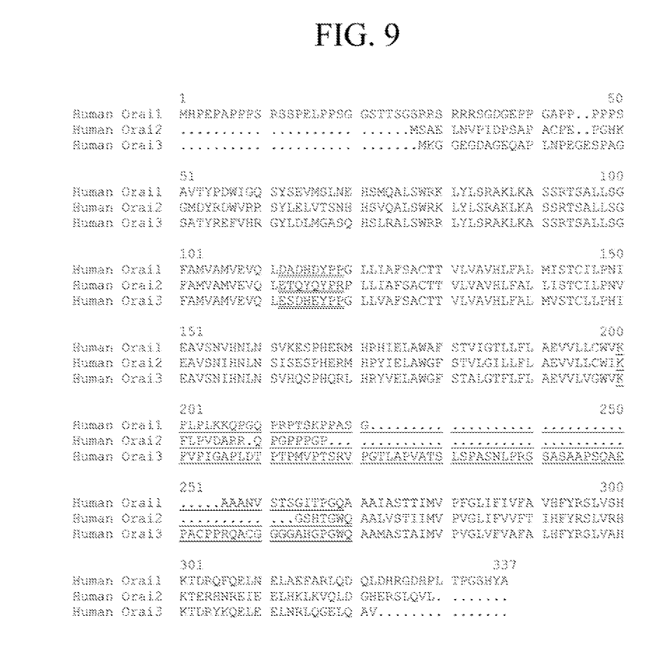

The human Orai2 cDNA (NCBI Reference Sequence NM_{—}032831; SEQ ID NO:60) and human Orai3 cDNA (NCBI Reference Sequence NM_{—}152288; SEQ ID NO:62) were cloned into the pcDNA3.1/Neomycin for expression in mammalian cells. The DNA from human Orai2 in pcDNA3.1/Neomycin vector was sequenced to confirm the human Orai2 cDNA and the sequence was 100% identical to SEQ ID NO:60 and the encoded amino acid sequence of hOrai2 protein (SEQ ID NO:61). The DNA from human Orai3 in pcDNA3.1/Neomycin vector was sequenced to confirm the human Orai3 cDNA and the sequence was 100% identical to the SEQ ID NO:62 and the encoded amino acid sequence of hOrai3 protein (SEQ ID NO:63). FIG. 9 shows an alignment of human Orai1, Orai2 and Orai3 proteins which are predicted to be four transmembrane proteins with cytoplasmic amino-termini and carboxy-termini. Based on the structure prediction, the double underlined amino acids representing the extracellular loop 1 (ECL1) and the single underlined amino acid representing the extracellular loop 2 (ECL2). The alignment among the three human Orais show that while the length of ECL1 is the same for the three proteins, the length of ECL2 varies with Orai3 being the longest. The sequence similarity is about 50% in the ECL1 and much more divergent in the ECL2 of the three different human Orai proteins.

Generating human Orai1 single nucleotide polymorphism variant S218G. To generate human Orai1 (S218G) variant protein (SEQ ID NO:65), two oligonucleotide primers (SEQ ID NO:66 and SEQ ID NO:67), depicted below, were used in a site directed mutagenesis PCR reaction using the QuikChange II XL Site-Directed Mutagenesis Kit (Agilent Technologies, Stratagene Products Division, La Jolla, Calif.), with all PCR amplification conditions as recommended by the manufacturer:

Forward primer:

(SEQ ID NO: 66)

5′-CCACCAGCAAGCCCCCCGCCGGTGGCGCAGCAGCCAACGTCAG-3′
and
Reverse primer:

(SEQ ID NO: 67)

5′-CTGACGTTGGCTGCTGCGCCACCGGCGGGGGGCTTGCTGGTG
G-3′.

The template that was used for the site-directed mutagenesis was the full length human Orai1 wild-type construct (SEQ ID NO:1), which was previously cloned into pcDNA3.1/Hygromycin and the resulting construct is referred to as hOrai1 (S218G) cDNA (SEQ ID NO:64; encoding hOrai1 (S218G) protein (SEQ ID NO:65)).

Molecular Cloning of Mouse and Rat Orai1 and Mouse and Rat STIM1.

The mouse Orai1 (mOrai1) was constructed using standard PCR technology. Briefly, the two following primers:

Forward primer:

(SEQ ID NO: 68)

5′-GGATCCTGAACCACCATGCATCCGGAGCCTGCCCCGCC-3′
and
Reverse primer:

(SEQ ID NO: 69)

5′-GCGGCCGCTTAGGCATAGTGGGTGCCCGGTG-3′

were used in a PCR reaction using mouse brain cDNA, obtained from BioChain Institute, Inc. (Hayward, Calif.) as a template.

The resulting 938-bp PCR products were purified and digested with BamHI and Not1 restriction enzymes. The pcDNA3.1/Neomycin vector was digested with BamHI and Not1 restriction enzymes; then the mOrai1 fragment (SEQ ID NO:71) was ligated to the BamHI and Not1 sites of pcDNA3.1/Neomycin vector to create a pcDNA3.1/Neomycin-mOrai1 vector. The DNA from mOrai1 in pcDNA3.1/Neomycin vector was sequenced to confirm the mouse Orai1 regions and the sequence was 100% identical to SEQ ID NO:71 (Mouse Orai1 cDNA NCBI Reference Sequence NM_{—}175423; encoding mOrai1 protein of SEQ ID NO:72). The mouse STIM1 (Mouse STIM1 cDNA NCBI Reference Sequence NM_{—}009287; SEQ ID NO:73; encoding Mouse STIM1 protein of SEQ ID NO:74) was cloned into the pcDNA3.1/Zeocin expression vector using PCR technology. In addition, rat Orai1 (rOrai1; Rat Orai1 cDNA NCBI Reference Sequence NM_{—}001013982; SEQ ID NO:75; encoding Rat Orai1 protein of SEQ ID NO:76) and rat STIM1 (R. norvegicus STIM1 cDNA NCBI Reference Sequence XM_{—}341896 (SEQ ID NO:77; encoding R. norvegicus STIM1 of SEQ ID NO:78) were cloned into the pcDNA3.1/Neomycin and pcDNA3.1/Zeocin, respectively, for expression in mammalian cells. An alignment of Orai1 protein sequences from different species including human, non-human primates (chimpanzee and cynomolgus monkey), dog, and rodents (mouse and rat) is depicted in FIG. 10A-B. Although the cynomolgus Orai1 protein sequence is incomplete and lacks the first 63 amino acids, the alignment indicates that the Orai1 proteins are conserved between species to a high degree. The double underlined amino acids represent the ECL1 domain that is predicted using the TMpred program from ch.EMBnet (www.ch.embnet.org/index.html). The program makes a prediction of membrane-spanning regions based on the statistical analysis of a database of naturally occurring transmembrane proteins, TMbase, using a combination of several weight-matrices for scoring. There is a 100% conservation of amino acid sequence in ECL1 between the different Orai1 proteins from dog and non-human primates compared to human, but only 87.5% conservation between rodents and human. The same TMpred program predicted the ECL2 region that is single underlined amino acids. Unlike ECL1, the ECL2 varies in length and the conservation is mainly at the ends.

Transient expression for FACS binding analysis. One day prior to transfection, 293EBNA cells were plated at 3.5×10⁶ cells/dish in 10 mL of growth medium onto 100-mm tissue culture dishes. For one 100-mm dish, 10 μg of DNA was diluted in 460 μL of Opti-MEM, mixed gently, and incubated at room temperature for 5 min. Then, 40 μL of FuGene HD transfection reagent was added to the mixture, mixed gently, and incubated at room temperature for 20 minutes. The transfection mixture was added drop-wise onto the cells and the dish was gently swirled to ensure uniform distribution of the complex.

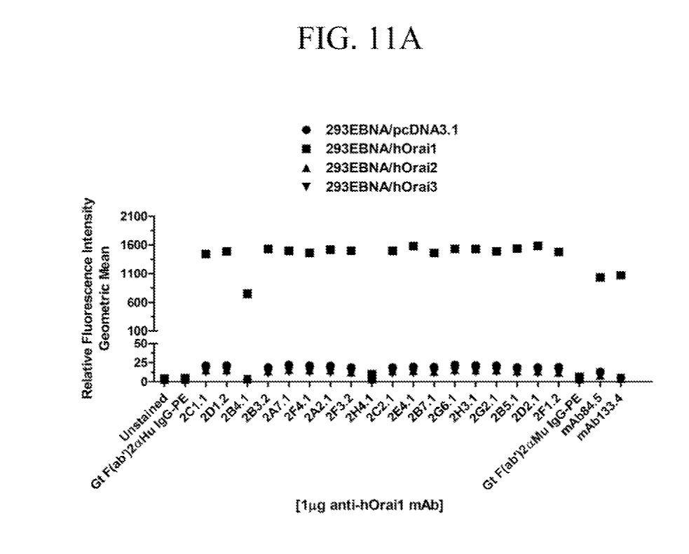

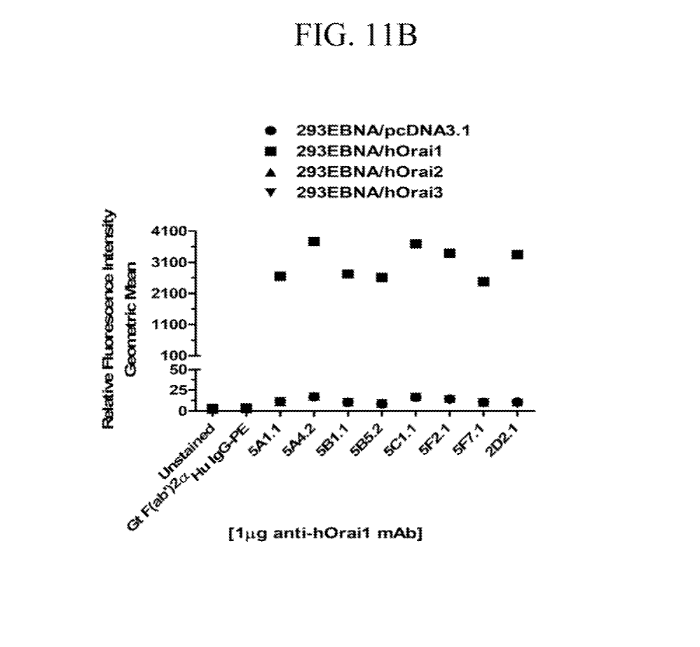

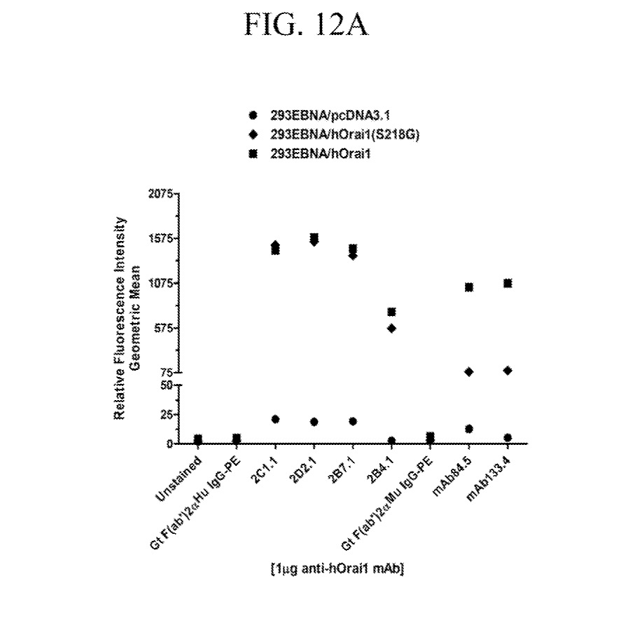

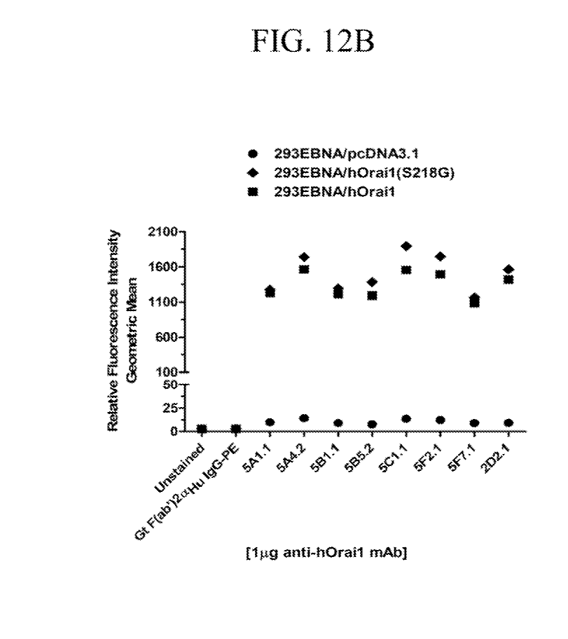

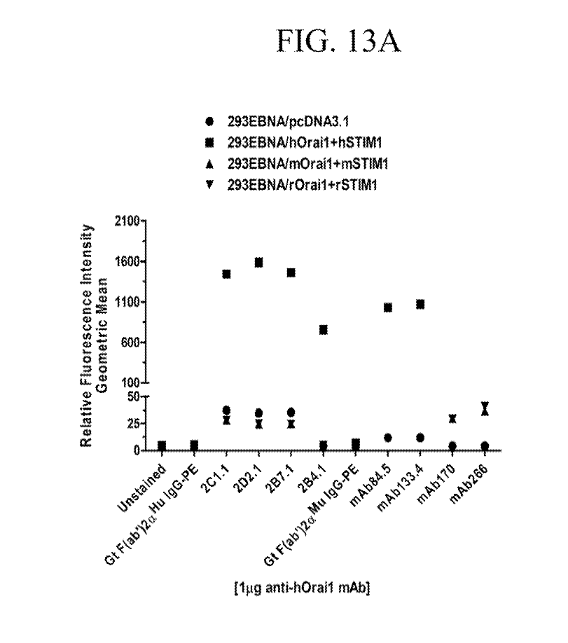

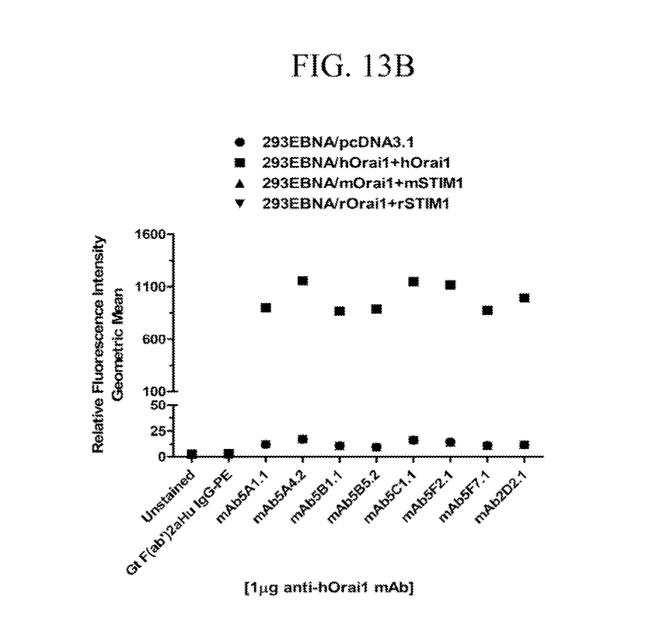

FACS binding analysis. Transfected 293EBNA cells transiently expressed hOrai1, human Orai2, or human Orai3 (results of binding comparison in FIG. 11A-B), or hOrai1(S218G) (results of binding comparison in FIG. 12A-B); or hOrai1 and hSTIM1; mOrai1 and mouse STIM1; or rat Orai1 and rat STIM1 (results of binding comparison in FIG. 13). The transfected cells were harvested at 48 hours post-transfection. Cells transfected with pcDNA3.1 were used as negative controls. Cells were washed once with ice-cold 1×PBS, resuspended in ice-cold FACS buffer (1×D-PBS+2% goat serum), and 2×10⁵ cells in 1001 were stained per antibody combination. All antibody incubation steps were performed on ice for 1 hour. Cells were first incubated with 1 μg of unlabeled mouse antibody (mAb 84.5 and mAb 133.4) or human anti-hOrai1 monoclonal antibodies, followed by a wash with 200 μL of FACS buffer. Next, the unlabelled antibody was detected using goat F(ab′)₂ anti-mouse or anti-human IgG-phycoerythrin (IgG-PE), followed by a wash with 200 μL of ice-cold FACS buffer before flow cytometry analysis. Unstained cells and cells stained with detecting antibodies were used as negative controls. The values of relative level of fluorescence were calculated using FCS Express (De Novo Software) and mean values were calculated using log-transformed data (geometric mean). Purified mAbs were assessed by FACS assay for their ability to specifically bind human Orai1, Orai2 and Orai3 proteins expressed on HEK-293 cells. FIG. 11A-B shows that there was intense staining to human Orai1 by 27 out of the 28 mAbs tested. Except for mAb 2H4.1, which significantly failed to bind any of the three Orai proteins, the Geo Mean values of all of the mAbs were comparable to each other in binding hOrai1, although the values for mAb 2B4.1, mAb 84.5 and mAb 133.4 were lower than the values for the other 24 binding mAbs. However, these purified antibodies did not recognize human Orai2 or hOrai3 expressed on the surface of HEK-293 cells even though the staining was slightly higher than the Unstained control and directly labeled secondary antibody fragment negative staining controls. This slightly higher staining relative to control was also observed with vector only transfect HEK-293 controls (293EBNA/pcDNA3.1) for most mAbs except mAb 2B4.1 and mAb 2H4.1, probably indicating that most of the mAbs were recognizing endogenously expressed human Orai1 that is known to be present in HEK-293 cells. (e.g., Sternfeld et al., Activation of muscarinic receptors reduces store-operated Ca²⁺ entry in HEK-293 cells, Cellular Signalling 19:1457-64 (2007); Fasolato et al., Store depletion triggers the calcium release-activated calcium current (ICRAC) in macrovascular endothelial cells: a comparison with Jurkat and embryonic kidney cell lines, Pfluegers Arch.-Eur. J. Physiol. 436(1):69-74 (1998)).

Human Orai1 has a single nucleotide polymorphism (SNP) encoding a serine-to-glycine substitution at position 218 of hOrai1 (SEQ ID NOS:64-65) located in the ECL2 domain (see, NCBI SNP database rs3741596). Recombinant mAbs were assessed for their ability to distinguish between the two different SNP variants of the human Orai1 protein. FIG. 12A-B shows that there was no discernible difference in binding by Campaign 1 or Campaign 2 fully human mAbs to the human Orai1 SNP variants. However, mouse mAb 84.5 and mouse mAb 133.4 bound more strongly to the wild-type human Orai1 with serine residue at position 218 than to the SNP variant with a glycine residue at position 218.

The recombinant mAbs were assessed for their ability to bind Orai1 proteins from human, mouse and rat. FIG. 13A-B shows that human mAbs from Campaign 1 and Campaign 2, and murine mAbs 84.5 and 133.4 specifically bound to human Orai1 but did not recognize mouse or rat Orai1. Here again, the low level binding above Unstained control and directly labeled secondary antibody fragment negative staining controls that was observed with all the mAbs except mAb 2B4.1 is thought to be due to endogenous expression of human Orai1 in HEK-293 cells. (E.g., Sternfeld et al., Activation of muscarinic receptors reduces store-operated Ca²⁺ entry in HEK-293 cells, Cellular Signalling 19:1457-64 (2007); Fasolato et al., Store depletion triggers the calcium release-activated calcium current (ICRAC) in macrovascular endothelial cells: a comparison with Jurkat and embryonic kidney cell lines, Pfluegers Arch.-Eur. J. Physiol. 436(1):69-74 (1998)).

### Example 8

### Further Characterization of mAb Binding Specificity to Human Orai1

Generating Chimeric Human and Mouse Orai1.

Since the monoclonal antibodies generated to human did not bind to rodent Orai1 (FIG. 13A-B), an alignment of the human and mouse Orai1 protein was generated (FIG. 14). The alignment shows 87.5% identity in amino acid sequence between human Orai1 and mouse Orai1 in the ECL1 region (double underlined). However, there is only 62.2% identity between human and mouse Orai1 protein sequences in the ECL2 region (single underlined). This was an indication that the mAbs bind to human Orai1 in the ECL2 region since there is only one amino acid difference in the 8-amino acid putative ECL1 region. Accordingly, we focused first on extracellular loop 2 as the subregion of human Orai1 of most interest in determining the site(s) on Orai1 where the monoclonal antibodies of the present invention bind.

Chimeric proteins were generated of human Orai1 bearing the mouse Orai1 ECL2 domain sequence KFLPLKRQAGQPSPTKPPAESVIVANHSDSSGITPGE (SEQ ID NO:85; i.e., amino acid residues 200 to 236 of SEQ ID NO:72) at amino acid residues 198 to 233 of SEQ ID NO:2. in place of the corresponding human Orai1 ECL2 sequence (SEQ ID NO:4), except that the glutamine at position 233 of SEQ ID NO:2 (human Orai1) was left in place instead of being substituted by glutamate as in the mouse ECL2 sequence, because it is a conservative substitution and immediately adjacent to a transmembrane domain, thus was thought probably not to play a role in binding by mAbs. These chimeric proteins are referred to herein as hOrai1-mOrai1 ECL2 chimera. To generate these chimera, primers with the sequence as depicted below (SEQ ID NOS:86-89) were used in a two part (Part A and B) PCR strategy using the previously cloned full length human Orai1 (SEQ ID NO: 1) and mouse Orai1 (SEQ ID NO:71) as templates. While the forward primer for Part A contains the SalI restriction enzyme site, the reverse primer for Part A contains Sph1. For Part B, four overlapping forward primers with the outermost primer also containing the Sph1 restriction enzyme site and one reverse primer containing the Not1 restriction site were used in four successive rounds of PCR amplification strategy to generate the DNA fragment in a standard PCR reaction.

Forward primer for Part A was:

(SEQ ID NO: 86)

5′-GGTCGACATGCATCCGGAGCCCGCCCC-3′;
and
Reverse primer for Part A was:

(SEQ ID NO: 87)

5′-GGCGGTGCATGCGCTCATGTGGTGACTCCTTGACCGAGTTGAG-3′.
Four forward primers for Part B were:

(SEQ ID NO: 88)

5′-CGCATGCACCGCCACATCGAGCTGGCCTGGGCCTTCTCCACCGTCAT
CGGCACGCTGCTCTTCCTAGCTGAGG-3′;

(SEQ ID NO: 236)

5′-CTGCTCTTCCTAGCTGAGGTGGTGCTGCTCTGCTGGGTCAAGTTCTT
GCCCCTCAAGAGGCAAGCGGGACAG-3′;

(SEQ ID NO: 237)

5′-CTCAAGAGGCAAGCGGGACAGCCAAGCCCCACCAAGCCTCCCGCTGA
ATCAGTCATCGTCGCCAACC-3′

(SEQ ID NO: 238)

5′-GAATCAGTCATCGTCGCCAACCACAGCGACAGCAGCGGCATCACCCC
GGGCCAGGCAGCTGCCATCGC-3′;
and
Reverse primer for Part B was:

(SEQ ID NO: 89)

5′-CGCGGCCGCCTAGGCATAGTGGCTGCCGGGCGTCAGGGGGTGGTCCC
CTCTGTGGTCCAGCTGGTC-3′.

The 520-bp PCR product from Part A that was digested with Salland Sph1 restriction enzymes, constitutes the 5′ fragment of the hOrai1-mOrai1 ECL2 construct. The successive rounds of PCR amplification strategy used the inner most forward primer, the reverse primer and hOrai1 as template to generate a DNA fragment. This DNA fragment was then used with the next outer primer and the reverse primer to amplify an even larger DNA fragment. This procedure was repeated two more times using the progressively more outer primers with the reverse primer to PCR-amplify ever larger DNA fragments. The four successive rounds of PCR amplification resulted in a 410-bp final product from Part B that was digested with Sph1 and Not1 and constitutes the 3′ fragment of the hOrai1-mOrai1 ECL2 constructs. The pcDNA3.1/Hygromycin vector was digested with Xho1 and Not1 restriction enzymes. The digested vector and the two restriction enzyme digested PCR products were ligated to create pcDNA3.1/Hygromycin-hOrai1-mOrai1 ECL2 vector. The DNA from hOrai1-mOrai1 ECL2 in pcDNA3.1/Hygromycin vector was sequenced and confirmed to be 100% identical to the sequence below (SEQ ID NO:90; designated hOrai1-mOrai1 ECL2 chimeric cDNA; encoding hOrai1-mOrai1 ECL2 chimeric protein of SEQ ID NO:91; ECL2 domain sequences are underlined):

hOrai1-mOrai1 ECL2 chimeric cDNA sequence:

SEQ ID NO: 90

ATGCATCCGGAGCCCGCCCCGCCCCCGAGCCGCAGCAGTCCCGAGCTTCC
CCCAAGCGGCGGCAGCACCACCAGCGGCAGCCGCCGGAGCCGCCGCCGC
AGCGGGGACGGGGAGCCCCCGGGGGCCCCGCCACCGCCGCCGTCCGCCG
TCACCTACCCGGACTGGATCGGCCAGAGTTACTCCGAGGTGATGAGCCTC
AACGAGCACTCCATGCAGGCGCTGTCCTGGCGCAAGCTCTACTTGAGCCG
CGCCAAGCTTAAAGCCTCCAGCCGGACCTCGGCTCTGCTCTCCGGCTTCG
CCATGGTGGCAATGGTGGAGGTGCAGCTGGACGCTGACCACGACTACCC
ACCGGGGCTGCTCATCGCCTTCAGTGCCTGCACCACAGTGCTGGTGGCTG
TGCACCTGTTTGCGCTCATGATCAGCACCTGCATCCTGCCCAACATCGAG
GCGGTGAGCAACGTGCACAATCTCAACTCGGTCAAGGAGTCCCCCCATG
AGCGCATGCACCGCCACATCGAGCTGGCCTGGGCCTTCTCCACCGTCATC
GGCACGCTGCTCTTCCTAGCTGAGGTGGTGCTGCTCTGCTGGGTCAAGTT
CTTGCCCCTCAAGAGGCAAGCGGGACAGCCAAGCCCCACCAAGCCTCCC
GCTGAATCAGTCATCGTCGCCAACCACAGCGACAGCAGCGGCATCACCC
CGGGCCAGGCAGCTGCCATCGCTTCGACCACCATCATGGTGCCCTTCGGC
CTGATCTTTATCGTCTTCGCCGTCCACTTCTACCGCTCACTGGTTAGCCA
TAAGACCGACCGACAGTTCCAGGAGCTCAACGAGCTGGCGGAGTTTGCCC
GCTTACAGGACCAGCTGGACCACAGAGGGGACCACCCCCTGACGCCCGG
CAGCCACTATGCCTAG//;

hOrai1-mOrai1 ECL2 chimeric protein sequence:

SEQ ID NO: 91

MHPEPAPPPSRSSPELPPSGGSTTSGSRRSRRRSGDGEPPGAPPPPPSAV
TYPDWIGQSYSEVMSLNEHSMQALSWRKLYLSRAKLKASSRTSALLSGF
AMVAMVEVQLDADHDYPPGLLIAFSACTTVLVAVHLFALMISTCILPNI
EAVSNVHNLNSVKESPHERMHRHIELAWAFSTVIGTLLFLAEVVLLCWV
KFLPLKRQAGQPSPTKPPAESVIVANHSDSSGITPGQAAAIASTTIMVPF
GLIFIVFAVHFYRSLVSHKTDRQFQELNELAEFARLQDQLDHRGDHPLT
PGSHYA//.

Conversely, chimeric proteins were also generated of mouse Orai1 bearing the human Orai1 ECL2 domain sequence (SEQ ID NO:4) at amino acid residues 200 to 236 of SEQ ID NO:72 in place of the corresponding mouse Orai1 ECL2 sequence (SEQ ID NO:85), except that the glutamate at position 236 of SEQ ID NO:72 (mouse Orai1) was left in place instead of being substituted by glutamine as in the human ECL2 sequence, because it is a conservative substitution and immediately adjacent to a transmembrane domain, thus was thought probably not to play a role in binding by mAbs. The chimeric protein is referred to herein as mOrai1-hOrai1 ECL2 chimera. To generate this chimera, one forward primer (SEQ ID NO:92) containing a BamHI restriction enzyme site and seven reverse primers with the outer most primer containing Not1 restriction enzyme site (SEQ ID NOS:93-95, and 239-242 depicted below) were used in seven successive rounds of PCR amplification strategy using the previously cloned full length mOrai1 as a template in a standard PCR reaction.

Forward primer was:

(SEQ ID NO: 92)

5′-CGGATCCTGAACCACCATGCATCCGGAGCCTGCCCCGCCCCCGAGT
CACAGCAATC-3′;
and
Reverse primers were:
The seven reverse primers were:

(SEQ ID NO: 93)

5′-GGGCTTGCTGGTGGGCCTTGGCTGGCCTGGCTGCTTCTTGAGAGGTA
AGAACTTGACCCAGCAG-3′;

(SEQ ID NO: 94)

5′-GGTGATGCCGCTGGTGCTGACGTTGGCTGCTGCGCCACTGGCGGGGG
GCTTGCTGGTGGGCCTTG-3′;

(SEQ ID NO: 95)

5′-GGAACCATGATGGCGGTGGAGGCAATGGCTGCCGCCTCACCCGGGG
TGATGCCGCTGGTGCTGAC-3′;

(SEQ ID NO: 239)

5′-GAGCGGTAGAAGTGAACAGCAAAGACGATAAAAACCAGGCCACAG
GGAACCATGATGGCGGTGGAG-3′;

(SEQ ID NO: 240)

5′-CTCATTGAGCTCCTGGAACTGCCGGTCCGTCTTATGGCTGACCAGGG
AGCGGTAGAAGTGAACAGC-3′;

(SEQ ID NO: 241)

5′-CTCTGTGGTCCAGCTGGTCCTGCAAGCGGGCAAACTCGGCCAGCTCA
TTGAGCTCCTGGAACTGC-3′;
and

(SEQ ID NO: 242)

5′-CGCGGCCGCTTAGGCATAGTGGGTGCCCGGTGTTAGAGAATGGTCCC
CTCTGTGGTCCAGCTGGTCCTGC-3′.

The strategy used the forward primer with the inner most primer first to generate a DNA fragment. This fragment was then used as a template in a PCR reaction with the forward primer and the next outer primer to yield an even larger DNA fragment. The procedure was repeated for five more rounds with each step using the forward primer and progressively more outer primers to PCR-generate ever larger DNA fragments. After seven rounds of PCR amplification, the resulting PCR product was resolved as the 930-bp band on a one percent agarose gel. The 930-bp PCR product was purified using a PCR Purification Kit (Qiagen), then was digested with BamHI and Not1 (Roche) restriction enzymes, and was purified by an agarose gel Gel Extraction Kit (Qiagen). The pcDNA3.1/Neomycin vector was digested with BamHI and Not1 restriction enzymes, and the large fragment was purified by Gel Extraction Kit. The gel purified hOrai1 fragment was ligated to the purified large fragment and transformed into One Shot® Top 10 (Invitrogen) to create a pcDNA3.1/Neomycin-hOrai1 vector. The digested vector and the two restriction enzyme digested PCR products were ligated to create pcDNA3.1/Hygromycin-mOrai1-hOrai1 ECL2 vector. The DNA from mOrai1-hOrai1 ECL2 in pcDNA3.1Hygromycin vector was sequenced to confirm and was 100% identical to the sequence below (SEQ ID NO:96; designated mOrai1-hOrai1 ECL2 chimeric cDNA; encoding mOrai1-hOrai1 ECL2 chimeric protein SEQ ID NO:97; ECL2 domain sequences are underlined):

mOrai1-hOrai1 ECL2 chimeric cDNA sequence:

SEQ ID NO: 96

ATGCATCCGGAGCCTGCCCCGCCCCCGAGTCACAGCAATCCGGAGCTTCC
CGTGAGCGGCGGCAGCAGCACTAGCGGCAGCCGCCGGAGCCGCCGCCGC
AGCGGGGACGGGGAGCCCTCGGGGGCCCCACCGCTGCCGCCGCCGCCAC
CCGCCGTCAGCTACCCGGACTGGATCGGCCAGAGTTACTCCGAGGTGAT
GAGCCTCAACGAGCACTCGATGCAGGCGCTGTCCTGGCGCAAGCTCTACT
TAAGCCGCGCCAAGCTCAAAGCTTCCAGCCGGACCTCGGCTCTGCTCTCC
GGCTTCGCCATGGTAGCGATGGTGGAAGTCCAGCTGGACACAGACCATG
ACTACCCACCAGGGTTGCTCATCGTCTTTAGTGCCTGCACCACAGTGCTA
GTGGCCGTGCACCTGTTTGCCCTCATGATCAGCACCTGCATCCTGCCCAA
CATCGAGGCTGTGAGCAACGTCCACAACCTCAACTCGGTCAAAGAGTCA
CCCCACGAGCGCATGCATCGCCACATCGAGCTGGCCTGGGCCTTCTCCAC
GGTCATCGGGACGCTGCTTTTCCTAGCAGAGGTCGTGCTGCTCTGCTGGG
TCAAGTTCTTACCTCTCAAGAAGCAGCCAGGCCAGCCAAGGCCCACCAG
CAAGCCCCCCGCCAGTGGCGCAGCAGCCAACGTCAGCACCAGCGGCATC
ACCCCGGGTGAGGCGGCAGCCATTGCCTCCACCGCCATCATGGTTCCCTG
TGGCCTGGTTTTCATCGTCTTTGCTGTTCACTTCTACCGCTCCCTGGTCA
GCCATAAGACGGACCGGCAGTTCCAGGAGCTCAATGAGCTGGCCGAGTTT
GCCCGCTTGCAGGACCAGCTGGACCACAGAGGGGACCATTCTCTAACAC
CGGGCACCCACTATGCCTAA//;

mOrai1-hOrai1 ECL2 chimeric protein sequence:

SEQ ID NO: 97

MHPEPAPPPSHSNPELPVSGGSSTSGSRRSRRRSGDGEPSGAPPLPPPPP
AVSYPDWIGQSYSEVMSLNEHSMQALSWRKLYLSRAKLKASSRTSALLSG
FAMVAMVEVQLDTDHDYPPGLLIVFSACTTVLVAVHLFALMISTCILPNI
EAVSNVHNLNSVKESPHERMHRHIELAWAFSTVIGTLLFLAEVVLLCWV
KFLPLKKQPGQPRPTSKPPASGAAANVSTSGITPGEAAAIASTAIMVPC
GLVFIVFAVHFYRSLVSHKTDRQFQELNELAEFARLQDQLDHRGDHSLT
PGTHYA//.

Transient expression for FACS binding analysis. One day prior to transfection, 293EBNA cells were plated at 3.5×10⁶ cells/dish in 10 mL of growth medium onto 100-mm tissue culture dishes. For one 100-mm dish, 10 μg of DNA was diluted in 460 μL of Opti-MEM, mixed gently, and incubated at room temperature for 5 min. Then, 40 μL of FuGene HD transfection reagent was added to the mixture, mixed gently, and incubated at room temperature for 20 minutes. The transfection mixture was added drop-wise onto the cells and the dish was gently swirled to ensure uniform distribution of the complex.

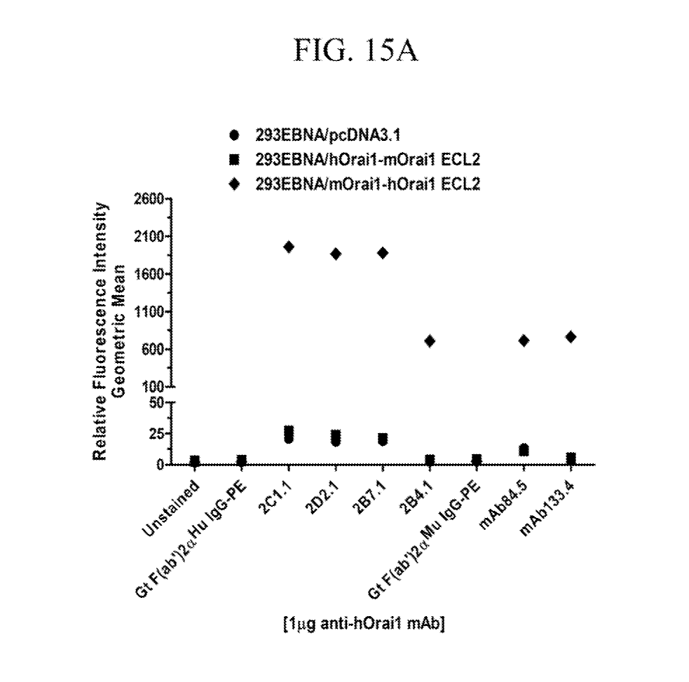

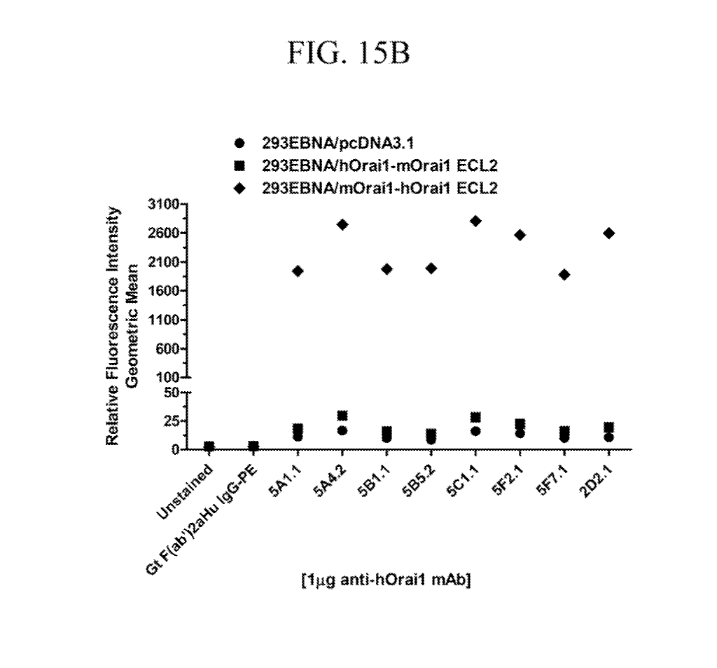

FACS binding analysis. Transfected 293EBNA cells transiently expressed hOrai1-mOrai1 ECL2 or mOrai1-hOrai1 ECL2 chimera. The transfected cells were harvested at 48 hours post-transfection. Cells transfected with pcDNA3.1 were used as negative controls. Cells were washed once with ice-cold 1×PBS, resuspended in ice-cold FACS buffer (1×D-PBS+2% goat serum), and 2×10⁵ cells in 100 μL were stained per antibody combination. All antibody incubation steps were performed on ice for 1 hour. Cells were first incubated with 1 μg of unlabeled mouse antibody (mAb84.5 and mAb 133.4) or human anti-hOrai1 monoclonal antibodies, followed by a wash with 200 μL of FACS buffer. Next, the unlabelled antibody was detected using goat F(ab′)₂ anti-mouse or anti-human IgG-phycoerythrin (IgG-PE), followed by a wash with 200 μL of ice-cold FACS buffer before flow cytometry analysis. Unstained cells and cells stained with detecting antibodies were used as negative controls. The values of relative level of fluorescence were calculated using FCS Express (De Novo Software) and mean values were calculated using log-transformed data (geometric mean). The results of the FACS analysis are shown in FIG. 15A-B, which supports the conclusion that the hOrai1 ECL2 domain is the region of hOrai1 where antibodies of the present invention bind.

Briefly, the recombinant mAbs where assessed for their ability to specifically bind the hOrai1-mOrai1 ECL2 chimera and mOrai1-hOrai1 ECL2 chimera. While the hOrai1-mOrai1 ECL2 chimera is a mutant where the mouse Orai1 ECL2 region replaced the human Orai1 ECL2 region in the human Orai1 background, the mOrai1-hOrai1 ECL2 chimera is the opposite in that the mouse Orai1 ECL2 was replaced with the human ECL2 region in the mouse Orai1 background. FIG. 15A shows that the mAb 2C1.1, mAb 2D2.1, mAb 2B7.1, mAb2B4.1, mAb 84.5 and mAb 133.4 bound strongly to mOrai1-hOrai1 ECL2 chimera but not the hOrai1-mOrai1 ECL2 chimera. Although there was slight binding to the hOrai1-mOrai1 ECL2 chimera by mAb 2C1.1, mAb 2D2.1, mAb 2B7.1 and mAb 84.5 that were above the Unstained control and directly labeled secondary antibody fragment negative staining controls, they are thought to be due to endogenous expression of human Orai1 in HEK-293 cells. (E.g., Sternfeld et al., Activation of muscarinic receptors reduces store-operated Ca²⁺entry in HEK-293 cells, Cellular Signalling 19:1457-64 (2007); Fasolato et al., Store depletion triggers the calcium release-activated calcium current (ICRAC) in macrovascular endothelial cells: a comparison with Jurkat and embryonic kidney cell lines, Pfluegers Arch.-Eur. J. Physiol. 436(1):69-74 (1998)). In FIG. 15B, the lack of significant binding by all the Campaign 2 mAbs to hOrai1-mOrai1 ECL2 chimera, a mutant where the whole protein is human except the ECL2 region is mouse, is additional strong evidence that the mAbs bind to human Orai1 exclusively in the ECL2 region.

Conversely, FIG. 15A-B shows that all the mAbs bind strongly to mOrai1-hOrai1 ECL2 chimera, a mutant where the ECL2 region is human and the rest of the protein's amino acid sequence is mouse Orai1. FIG. 13 indicated that the mAbs do not specifically bind to the mouse Orai1 protein. The gain of binding by all of the mAbs to the mOrai1-hOrai1 ECL2 shown in FIG. 15A-B provides conclusive evidence that the mAbs bind to human Orai1 exclusively in the ECL2 region since the human ECL2 region in the context of the mouse Orai1 protein has to fold similarly to the wild-type human Orai1 protein to be recognized by the mAbs. Therefore, taken together the gain of binding observed in the mOrai1-hOrai1 ECL2 and the loss of binding in the hOrai1-mOrai1 ECL2, these results show that the human ECL2 is sufficient for binding by the anti-hOrai1 mAbs tested.

Mutation of hOrai1-mOrai1 ECL2 chimera. To determine the region within the human extracellular loop 2 domain where the anti-hOrai1 monoclonal antibodies bind, we employed site directed mutagenesis with QuikChange Multi Site-Directed Mutagenesis Kit (Agilent Technologies, Stratagene Products Division, La Jolla, Calif.) per manufacturer's instruction to generate a series of mutants using the hOrai1-mOrai1 ECL2 and mOrai1-hOrai1 ECL2 chimera constructs as templates. The first series of mutants started with hOrai1-mOrai1 ECL2 chimera not bound by the monoclonal anti-hOrai1 antibodies and advanced to hOrai1-mOrai1 ECL2 chimera with mutations that were bound by the anti-hOrai1 antibodies by FACS binding analysis. Starting with the hOrai1-mOrai1 ECL2 chimera, we designed a series of mutants where the mouse amino acids were swap for their corresponding human counterparts based on the non-conserved region between human and mouse Orai1 proteins (FIG. 14).

Using pcDNA3.1/Hygromycin-hOrai1-mOrai1 ECL2 vector and primers with the sequences SEQ ID NOS:100-101 below, we generated hOrai1-mOrai1 ECL2 where amino acid residues 215 to 219 of SEQ ID NO:72 (mOrai1), with the amino acid sequence KPPAE (SEQ ID NO:98), were mutated to the corresponding amino acid sequence of residues 213 to 217 of SEQ ID NO:2 (hOrai1), having the sequence SKPPA (SEQ ID NO:99), referred to as hOrai1-mOrai1 ECL2 (SKPPA) protein (SEQ ID NO:103), encoded by hOrai1-mOrai1 ECL2 (SKPPA) cDNA (SEQ ID NO:102).

The forward primer sequence was:

(SEQ ID NO: 100)

5′-GGGACAGCCAAGCCCCACCAGCAAGCCCCCTGCATCAGTCATCGTC
GCCAACC-3′//;
and
the reverse primer sequence was:

(SEQ ID NO: 101)

5′-GGTTGGCGACGATGACTGATGCAGGGGGCTTGCTGGTGGGGCTTGG
CTGTCCC-3′.

The sequence of hOrai1-mOrai1 ECL2 (SKPPA) cDNA is the following (ECL2 domain is underlined, SKAPPA (SEQ ID NO:99) coding sequence is double underlined):

SEQ ID NO: 102

ATGCATCCGGAGCCCGCCCCGCCCCCGAGCCGCAGCAGTCCCGAGCTTCC
CCCAAGCGGCGGCAGCACCACCAGCGGCAGCCGCCGGAGCCGCCGCCGC
AGCGGGGACGGGGAGCCCCCGGGGGCCCCGCCACCGCCGCCGTCCGCCG
TCACCTACCCGGACTGGATCGGCCAGAGTTACTCCGAGGTGATGAGCCTC
AACGAGCACTCCATGCAGGCGCTGTCCTGGCGCAAGCTCTACTTGAGCCG
CGCCAAGCTTAAAGCCTCCAGCCGGACCTCGGCTCTGCTCTCCGGCTTCG
CCATGGTGGCAATGGTGGAGGTGCAGCTGGACGCTGACCACGACTACCC
ACCGGGGCTGCTCATCGCCTTCAGTGCCTGCACCACAGTGCTGGTGGCTG
TGCACCTGTTTGCGCTCATGATCAGCACCTGCATCCTGCCCAACATCGAG
GCGGTGAGCAACGTGCACAATCTCAACTCGGTCAAGGAGTCCCCCCATG
AGCGCATGCACCGCCACATCGAGCTGGCCTGGGCCTTCTCCACCGTCATC
GGCACGCTGCTCTTCCTAGCTGAGGTGGTGCTGCTCTGCTGGGTCAAGTT
CTTGCCCCTCAAGAGGCAAGCGGGACAGCCAAGCCCCACCAGCAAGCCC
CCTGCATCAGTCATCGTCGCCAACCACAGCGACAGCAGCGGCATCACCC
CGGGCCAGGCAGCTGCCATCGCTTCGACCACCATCATGGTGCCCTTCGGC
CTGATCTTTATCGTCTTCGCCGTCCACTTCTACCGCTCACTGGTTAGCCA
TAAGACCGACCGACAGTTCCAGGAGCTCAACGAGCTGGCGGAGTTTGCCC
GCTTACAGGACCAGCTGGACCACAGAGGGGACCACCCCCTGACGCCCGG
CAGCCACTATGCCTAG//.

The sequence of hOrai1-mOrai1 ECL2 (SKPPA) protein is the following (ECL2 domain is underlined, SKAPPA (SEQ ID NO:99) sequence is double underlined):

SEQ ID NO: 103

MHPEPAPPPSRSSPELPPSGGSTTSGSRRSRRRSGDGEPPGAPPPPPSAV
TYPDWIGQSYSEVMSLNEHSMQALSWRKLYLSRAKLKASSRTSALLSGF
AMVAMVEVQLDADHDYPPGLLIAFSACTTVLVAVHLFALMISTCILPNIE
AVSNVHNLNSVKESPHERMHRHIELAWAFSTVIGTLLFLAEVVLLCWV
KFLPLKRQAGQPSPTSKPPASVIVANHSDSSGITPGQAAAIASTTIMVP
FGLIFIVFAVHFYRSLVSHKTDRQFQELNELAEFARLQDQLDHRGDHPL
TPGSHYA//.

Using pcDNA3.1/Hygromycin-hOrai1-mOrai1 ECL2 vector and primers with the sequences of SEQ ID NOS:104-105 below, we generated hOrai1-mOrai1 ECL2 where amino acid residues 221 to 223 of SEQ ID NO:72 (mOrai1), with the amino acid sequence VIV, were mutated to the corresponding amino acid sequence of residues 219 to 221 of SEQ ID NO:2 (hOrai1), having the sequence GAA, referred to as hOrai1-mOrai1 ECL2 (GAA) protein (SEQ ID NO:107), encoded by hOrai1-mOrai1 ECL2 (GAA) cDNA (SEQ ID NO:106).

The forward primer sequence was:

(SEQ ID NO: 104)

5′-CCTCCCGCTGAATCAGGCGCCGCCGCCAACCACAGCGAC-3′;
and

the reverse primer sequence was:

(SEQ ID NO: 105)

5′-GTCGCTGTGGTTGGCGGCGGCGCCTGATTCAGCGGGAGG-3′

The sequence of hOrai1-mOrai1 ECL2 (GAA) cDNA is the following (ECL2 domain is underlined, GAA coding sequence is double underlined):

SEQ ID NO: 106

ATGCATCCGGAGCCCGCCCCGCCCCCGAGCCGCAGCAGTCCCGAGCTTCC
CCCAAGCGGCGGCAGCACCACCAGCGGCAGCCGCCGGAGCCGCCGCCGC
AGCGGGGACGGGGAGCCCCCGGGGGCCCCGCCACCGCCGCCGTCCGCCG
TCACCTACCCGGACTGGATCGGCCAGAGTTACTCCGAGGTGATGAGCCTC
AACGAGCACTCCATGCAGGCGCTGTCCTGGCGCAAGCTCTACTTGAGCCG
CGCCAAGCTTAAAGCCTCCAGCCGGACCTCGGCTCTGCTCTCCGGCTTCG
CCATGGTGGCAATGGTGGAGGTGCAGCTGGACGCTGACCACGACTACCC
ACCGGGGCTGCTCATCGCCTTCAGTGCCTGCACCACAGTGCTGGTGGCTG
TGCACCTGTTTGCGCTCATGATCAGCACCTGCATCCTGCCCAACATCGAG
GCGGTGAGCAACGTGCACAATCTCAACTCGGTCAAGGAGTCCCCCCATG
AGCGCATGCACCGCCACATCGAGCTGGCCTGGGCCTTCTCCACCGTCATC
GGCACGCTGCTCTTCCTAGCTGAGGTGGTGCTGCTCTGCTGGGTCAAGTT
CTTGCCCCTCAAGAGGCAAGCGGGACAGCCAAGCCCCACCAAGCCTCCC
GCTGAATCAGGCGCCGCCGCCAACCACAGCGACAGCAGCGGCATCACCC
CGGGCCAGGCAGCTGCCATCGCTTCGACCACCATCATGGTGCCCTTCGGC
CTGATCTTTATCGTCTTCGCCGTCCACTTCTACCGCTCACTGGTTAGCCA
TAAGACCGACCGACAGTTCCAGGAGCTCAACGAGCTGGCGGAGTTTGCCC
GCTTACAGGACCAGCTGGACCACAGAGGGGACCACCCCCTGACGCCCGG
CAGCCACTATGCCTAG//.

The sequence of hOrai1-mOrai1 ECL2 (GAA) protein is the following (ECL2 domain is underlined, GAA sequence is double underlined):

SEQ ID NO: 107

MHPEPAPPPSRSSPELPPSGGSTTSGSRRSRRRSGDGEPPGAPPPPPSAV
TYPDWIGQSYSEVMSLNEHSMQALSWRKLYLSRAKLKASSRTSALLSGF
AMVAMVEVQLDADHDYPPGLLIAFSACTTVLVAVHLFALMISTCILPNI
EAVSNVHNLNSVKESPHERMHRHIELAWAFSTVIGTLLFLAEVVLLCWV
KFLPLKRQAGQPSPTKPPAESGAAANHSDSSGITPGQAAAIASTTIMV
PFGLIFIVFAVHFYRSLVSHKTDRQFQELNELAEFARLQDQLDHRGDH
PLTPGSHYA//.

Using pcDNA3.1/Hygromycin-hOrai1-mOrai1 ECL2 vector and primers with the sequences SEQ ID NOS:110-111 below, we generated hOrai1-mOrai1 ECL2 where amino acid residues 218 to 223 of SEQ ID NO:72 (mOrai1), with the amino acid sequence AESVIV (SEQ ID NO:108), were mutated to the corresponding amino acid sequence of residues 216 to 221 of SEQ ID NO:2 (hOrai1), having the sequence PASGAA (SEQ ID NO:109), referred to as hOrai1-mOrai1 ECL2 (PASGAA) protein (SEQ ID NO: 113), encoded by hOrai1-mOrai1

ECL2 (PASGAA) cDNA (SEQ ID NO: 112).

The forward primer sequence was:

(SEQ ID NO: 110)

5′-CCCACCAAGCCTCCCCCTGCATCAGGCGCCGCCGCCAACCACAGCG
A-3′//;
and
the reverse primer sequence was:

(SEQ ID NO: 111)

5′-TCGCTGTGGTTGGCGGCGGCGCCTGATGCAGGGGGAGGCTTGGTGG
G-3′.

The sequence of hOrai1-mOrai1 ECL2 (PASGAA) cDNA is the following (ECL2 domain is underlined, PASGAA (SEQ ID NO:109) coding sequence is double underlined):

SEQ ID NO: 112

ATGCATCCGGAGCCCGCCCCGCCCCCGAGCCGCAGCAGTCCCGAGCTTCC
CCCAAGCGGCGGCAGCACCACCAGCGGCAGCCGCCGGAGCCGCCGCCGC
AGCGGGGACGGGGAGCCCCCGGGGGCCCCGCCACCGCCGCCGTCCGCCG
TCACCTACCCGGACTGGATCGGCCAGAGTTACTCCGAGGTGATGAGCCTC
AACGAGCACTCCATGCAGGCGCTGTCCTGGCGCAAGCTCTACTTGAGCCG
CGCCAAGCTTAAAGCCTCCAGCCGGACCTCGGCTCTGCTCTCCGGCTTCG
CCATGGTGGCAATGGTGGAGGTGCAGCTGGACGCTGACCACGACTACCC
ACCGGGGCTGCTCATCGCCTTCAGTGCCTGCACCACAGTGCTGGTGGCTG
TGCACCTGTTTGCGCTCATGATCAGCACCTGCATCCTGCCCAACATCGAG
GCGGTGAGCAACGTGCACAATCTCAACTCGGTCAAGGAGTCCCCCCATG
AGCGCATGCACCGCCACATCGAGCTGGCCTGGGCCTTCTCCACCGTCATC
GGCACGCTGCTCTTCCTAGCTGAGGTGGTGCTGCTCTGCTGGGTCAAGTT
CTTGCCCCTCAAGAGGCAAGCGGGACAGCCAAGCCCCACCAAGCCTCCC
CCTGCATCAGGCGCCGCCGCCAACCACAGCGACAGCAGCGGCATCACCC
CGGGCCAGGCAGCTGCCATCGCTTCGACCACCATCATGGTGCCCTTCGGC
CTGATCTTTATCGTCTTCGCCGTCCACTTCTACCGCTCACTGGTTAGCCA
TAAGACCGACCGACAGTTCCAGGAGCTCAACGAGCTGGCGGAGTTTGCCC
GCTTACAGGACCAGCTGGACCACAGAGGGGACCACCCCCTGACGCCCGG
CAGCCACTATGCCTAG//.

The sequence of hOrai1-mOrai1 ECL2 (PASGAA) protein is the following (ECL2 domain is underlined, PASGAA (SEQ ID NO:109) sequence is double underlined):

SEQ ID NO: 113

MHPEPAPPPSRSSPELPPSGGSTTSGSRRSRRRSGDGEPPGAPPPPPSAV
TYPDWIGQSYSEVMSLNEHSMQALSWRKLYLSRAKLKASSRTSALLSGF
AMVAMVEVQLDADHDYPPGLLIAFSACTTVLVAVHLFALMISTCILPNI
EAVSNVHNLNSVKESPHERMHRHIELAWAFSTVIGTLLFLAEVVLLC
WVKFLPLKRQAGQPSPTKPPPASGAAANHSDSSGITPGQAAAIAST
TIMVPFGLIFIVFAVHFYRSLVSHKTDRQFQELNELAEFARLQDQLDH
RGDHPLTPGSHYA//.

Using pcDNA3.1/Hygromycin-hOrai1-mOrai1 ECL2 vector and primers with the sequences of SEQ ID NOS:114-115 below, we generated hOrai1-mOrai1 ECL2 where amino acid residues 226 to 228 of SEQ ID NO:72 (mOrai1), with the amino acid sequence HSD, were mutated to the corresponding amino acid sequence of residues 224 to 226 of SEQ ID NO:2 (hOrai1), having the sequence VST, referred to as hOrai1-mOrai1 ECL2 (VST) protein (SEQ ID NO:117), encoded by hOrai1-mOrai1 ECL2 (VST) cDNA (SEQ ID NO:116).

The forward primer sequence was:

(SEQ ID NO: 114)

5′-GCGCCGCCGCCAACGTCAGCACCAGCAGCGGCATCA-3′;
and
the reverse primer sequence was:

(SEQ ID NO: 115)

5′-TGATGCCGCTGCTGGTGCTGACGTTGGCGGCGGCGC-3′

The sequence of hOrai1-mOrai1 ECL2 (VST) cDNA is the following (ECL2 domain is underlined, VST coding sequence is double underlined):

SEQ ID NO: 116

ATGCATCCGGAGCCCGCCCCGCCCCCGAGCCGCAGCAGTCCCGAGCTTCC
CCCAAGCGGCGGCAGCACCACCAGCGGCAGCCGCCGGAGCCGCCGCCGC
AGCGGGGACGGGGAGCCCCCGGGGGCCCCGCCACCGCCGCCGTCCGCCG
TCACCTACCCGGACTGGATCGGCCAGAGTTACTCCGAGGTGATGAGCCTC
AACGAGCACTCCATGCAGGCGCTGTCCTGGCGCAAGCTCTACTTGAGCCG
CGCCAAGCTTAAAGCCTCCAGCCGGACCTCGGCTCTGCTCTCCGGCTTCG
CCATGGTGGCAATGGTGGAGGTGCAGCTGGACGCTGACCACGACTACCC
ACCGGGGCTGCTCATCGCCTTCAGTGCCTGCACCACAGTGCTGGTGGCTG
TGCACCTGTTTGCGCTCATGATCAGCACCTGCATCCTGCCCAACATCGAG
GCGGTGAGCAACGTGCACAATCTCAACTCGGTCAAGGAGTCCCCCCATG
AGCGCATGCACCGCCACATCGAGCTGGCCTGGGCCTTCTCCACCGTCATC
GGCACGCTGCTCTTCCTAGCTGAGGTGGTGCTGCTCTGCTGGGTCAAGTT
CTTGCCCCTCAAGAGGCAAGCGGGACAGCCAAGCCCCACCAAGCCTCCC
GCTGAATCAGTCATCGTCGCCAACGTCAGCACCAGCAGCGGCATCACCC
CGGGCCAGGCAGCTGCCATCGCTTCGACCACCATCATGGTGCCCTTCGGC
CTGATCTTTATCGTCTTCGCCGTCCACTTCTACCGCTCACTGGTTAGCCA
TAAGACCGACCGACAGTTCCAGGAGCTCAACGAGCTGGCGGAGTTTGCCC
GCTTACAGGACCAGCTGGACCACAGAGGGGACCACCCCCTGACGCCCGG
CAGCCACTATGCCTAG//.

The sequence of hOrai1-mOrai1 ECL2 (VST) protein is the following (ECL2 domain is underlined, VST sequence is double underlined):

SEQ ID NO: 117

MHPEPAPPPSRSSPELPPSGGSTTSGSRRSRRRSGDGEPPGAPPPPPSAV
TYPDWIGQSYSEVMSLNEHSMQALSWRKLYLSRAKLKASSRTSALLSGFA
MVAMVEVQLDADHDYPPGLLIAFSACTTVLVAVHLFALMISTCILPNIEA
VSNVHNLNSVKESPHERMHRHIELAWAFSTVIGTLLFLAEVVLLCWVKFL
PLKRQAGQPSPTKPPAESVIVANVSTSSGITPGQAAAIASTTIMVPFGLI
FIVFAVHFYRSLVSHKTDRQFQELNELAEFARLQDQLDHRGDHPLTPGS
HYA//.

Using pcDNA3.1/Hygromycin-hOrai1-mOrai1 ECL2 vector and primers with the sequences SEQ ID NOS:120-121 below, we generated hOrai1-mOrai1 ECL2 where amino acid residues 223 to 228 of SEQ ID NO:72 (mOrai1), with the amino acid sequence VANHSD (SEQ ID NO:118), were mutated to the corresponding amino acid sequence of residues 221 to 226 of SEQ ID NO:2 (hOrai1), having the sequence AANVST (SEQ ID NO:119), referred to as hOrai1-mOrai1 ECL2 (AANVST) protein (SEQ ID NO:123), encoded by hOrai1-mOrai1 ECL2 (AANVST) cDNA (SEQ ID NO:122).

The forward primer sequence was:

(SEQ ID NO: 120)

5′-GCGCCGCCGCCAACGTCAGCACCAGCAGCGGCATCA-3′//;
and
the reverse primer sequence was:

(SEQ ID NO: 121)

5′-TGATGCCGCTGCTGGTGCTGACGTTGGCGGCGGCGC-3′.

The sequence of hOrai1-mOrai1 ECL2 (AANVST) cDNA is the following (ECL2 domain is underlined, AANVST (SEQ ID NO:119) coding sequence is double underlined):

SEQ ID NO: 122

ATGCATCCGGAGCCCGCCCCGCCCCCGAGCCGCAGCAGTCCCGAGCTTCC
CCCAAGCGGCGGCAGCACCACCAGCGGCAGCCGCCGGAGCCGCCGCCGC
AGCGGGGACGGGGAGCCCCCGGGGGCCCCGCCACCGCCGCCGTCCGCCG
TCACCTACCCGGACTGGATCGGCCAGAGTTACTCCGAGGTGATGAGCCTC
AACGAGCACTCCATGCAGGCGCTGTCCTGGCGCAAGCTCTACTTGAGCCG
CGCCAAGCTTAAAGCCTCCAGCCGGACCTCGGCTCTGCTCTCCGGCTTCG
CCATGGTGGCAATGGTGGAGGTGCAGCTGGACGCTGACCACGACTACCC
ACCGGGGCTGCTCATCGCCTTCAGTGCCTGCACCACAGTGCTGGTGGCTG
TGCACCTGTTTGCGCTCATGATCAGCACCTGCATCCTGCCCAACATCGAG
GCGGTGAGCAACGTGCACAATCTCAACTCGGTCAAGGAGTCCCCCCATG
AGCGCATGCACCGCCACATCGAGCTGGCCTGGGCCTTCTCCACCGTCATC
GGCACGCTGCTCTTCCTAGCTGAGGTGGTGCTGCTCTGCTGGGTCAAGTT
CTTGCCCCTCAAGAGGCAAGCGGGACAGCCAAGCCCCACCAAGCCTCCC
GCTGAATCAGTCATCGCCGCCAACGTCAGCACCAGCAGCGGCATCACCC
CGGGCCAGGCAGCTGCCATCGCTTCGACCACCATCATGGTGCCCTTCGGC
CTGATCTTTATCGTCTTCGCCGTCCACTTCTACCGCTCACTGGTTAGCCA
TAAGACCGACCGACAGTTCCAGGAGCTCAACGAGCTGGCGGAGTTTGCCC
GCTTACAGGACCAGCTGGACCACAGAGGGGACCACCCCCTGACGCCCGG
CAGCCACTATGCCTAG//.

The sequence of hOrai1-mOrai1 ECL2 (AANVST) protein is the following (ECL2 domain is underlined, AANVST (SEQ ID NO:119) sequence is double underlined):

SEQ ID NO: 123

MHPEPAPPPSRSSPELPPSGGSTTSGSRRSRRRSGDGEPPGAPPPPPSAV
TYPDWIGQSYSEVMSLNEHSMQALSWRKLYLSRAKLKASSRTSALLSGFA
MVAMVEVQLDADHDYPPGLLIAFSACTTVLVAVHLFALMISTCILPNIEA
VSNVHNLNSVKESPHERMHRHIELAWAFSTVIGTLLFLAEVVLLCWVKFL
PLKRQAGQPSPTKPPAESVIAANVSTSSGITPGQAAAIASTTIMVPFGLI
FIVFAVHFYRSLVSHKTDRQFQELNELAEFARLQDQLDHRGDHPLTP
GSHYA//.

Using pcDNA3.1/Hygromycin-hOrai1-mOrai1 ECL2 (SKPPA) vector and primers with the sequences SEQ ID NOS:126-127 below, we generated hOrai1-mOrai1 ECL2 where amino acid residues 212 to 219 of SEQ ID NO:72 (mOrai1), with the amino acid sequence SPTKPPAE (SEQ ID NO: 124), were mutated to the corresponding amino acid sequence of residues 210 to 217 of SEQ ID NO:2 (hOrai1), having the sequence RPTSKPPA (SEQ ID NO:125), referred to as hOrai1-mOrai1 ECL2 (RPTSKPPA) protein (SEQ ID NO: 129), encoded by hOrai1-mOrai1 ECL2 (RPTSKPPA) cDNA (SEQ ID NO:128).

The forward primer sequence was:

(SEQ ID NO: 126)

5′-GGGACAGCCAAGGCCCACCAGCAAG-3′//;
and
the reverse primer sequence was:

(SEQ ID NO: 127)

5′-CTTGCTGGTGGGCCTTGGCTGTCCC-3′.

The sequence of hOrai1-mOrai1 ECL2 (RPTSKPPA) cDNA is the following (ECL2 domain is underlined, RPTSKPPA (SEQ ID NO: 125) coding sequence is double underlined):

SEQ ID NO: 128

ATGCATCCGGAGCCCGCCCCGCCCCCGAGCCGCAGCAGTCCCGAGCTTCC
CCCAAGCGGCGGCAGCACCACCAGCGGCAGCCGCCGGAGCCGCCGCCGC
AGCGGGGACGGGGAGCCCCCGGGGGCCCCGCCACCGCCGCCGTCCGCCG
TCACCTACCCGGACTGGATCGGCCAGAGTTACTCCGAGGTGATGAGCCTC
AACGAGCACTCCATGCAGGCGCTGTCCTGGCGCAAGCTCTACTTGAGCCG
CGCCAAGCTTAAAGCCTCCAGCCGGACCTCGGCTCTGCTCTCCGGCTTCG
CCATGGTGGCAATGGTGGAGGTGCAGCTGGACGCTGACCACGACTACCC
ACCGGGGCTGCTCATCGCCTTCAGTGCCTGCACCACAGTGCTGGTGGCTG
TGCACCTGTTTGCGCTCATGATCAGCACCTGCATCCTGCCCAACATCGAG
GCGGTGAGCAACGTGCACAATCTCAACTCGGTCAAGGAGTCCCCCCATG
AGCGCATGCACCGCCACATCGAGCTGGCCTGGGCCTTCTCCACCGTCATC
GGCACGCTGCTCTTCCTAGCTGAGGTGGTGCTGCTCTGCTGGGTCAAGTT
CTTGCCCCTCAAGAGGCAAGCGGGACAGCCAAGGCCCACCAGCAAGCCC
CCTGCATCAGTCATCGTCGCCAACCACAGCGACAGCAGCGGCATCACCC
CGGGCCAGGCAGCTGCCATCGCTTCGACCACCATCATGGTGCCCTTCGGC
CTGATCTTTATCGTCTTCGCCGTCCACTTCTACCGCTCACTGGTTAGCCA
TAAGACCGACCGACAGTTCCAGGAGCTCAACGAGCTGGCGGAGTTTGCCC
GCTTACAGGACCAGCTGGACCACAGAGGGGACCACCCCCTGACGCCCGG
CAGCCACTATGCCTAG//.

The sequence of hOrai1-mOrai1 ECL2 (RPTSKPPA) protein is the following (ECL2 domain is underlined, RPTSKPPA (SEQ ID NO: 125) sequence is double underlined):

SEQ ID NO: 129

MHPEPAPPPSRSSPELPPSGGSTTSGSRRSRRRSGDGEPPGAPPPPPSAV
TYPDWIGQSYSEVMSLNEHSMQALSWRKLYLSRAKLKASSRTSALLSGFA
MVAMVEVQLDADHDYPPGLLIAFSACTTVLVAVHLFALMISTCILPNIEA
VSNVHNLNSVKESPHERMHRHIELAWAFSTVIGTLLFLAEVVLLCWVKFL
PLKRQAGQPRPTSKPPASVIVANHSDSSGITPGQAAAIASTTIMVPFGLI
FIVFAVHFYRSLVSHKTDRQFQELNELAEFARLQDQLDHRGDHPLTP
GSHYA//.

Using pcDNA3.1/Hygromycin-hOrai1-mOrai1 ECL2 (RPTSKPPA) vector and primers with the sequences SEQ ID NOS:187-188 below, we generated hOrai1-mOrai1 ECL2 where amino acid residues 212 to 223 of SEQ ID NO:72 (mOrai1), with the amino acid sequence SPTKPPAESVIV (SEQ ID NO:189), were mutated to the corresponding amino acid sequence of residues 210 to 221 of SEQ ID NO:2 (hOrai1), having the sequence RPTSKPPASGAA (SEQ ID NO:190), referred to as hOrai1-mOrai1 ECL2 (RPTSKPPASGAA) protein (SEQ ID NO:192), encoded by hOrai1-mOrai1 ECL2 (RPTSKPPASGAA) cDNA (SEQ ID NO:191).

The forward primer sequence was:

(SEQ ID NO: 187)

5′-AAGCCCCCTGCATCAGGCGCCGCCGCCAACCACAGCGAC-3′//;
and
the reverse primer sequence was:

(SEQ ID NO: 188)

5′-GTCGCTGTGGTTGGCGGCGGCGCCTGATGCAGGGGGCTT-3′.

The sequence of hOrai1-mOrai1 ECL2 (RPTSKPPASGAA) cDNA is the following (ECL2 domain is underlined, RPTSKPPASGAA (SEQ ID NO:190) coding sequence is double underlined):

SEQ ID NO: 191

ATGCATCCGGAGCCCGCCCCGCCCCCGAGCCGCAGCAGTCCCGAGCTTCC
CCCAAGCGGCGGCAGCACCACCAGCGGCAGCCGCCGGAGCCGCCGCCGC
AGCGGGGACGGGGAGCCCCCGGGGGCCCCGCCACCGCCGCCGTCCGCCG
TCACCTACCCGGACTGGATCGGCCAGAGTTACTCCGAGGTGATGAGCCTC
AACGAGCACTCCATGCAGGCGCTGTCCTGGCGCAAGCTCTACTTGAGCCG
CGCCAAGCTTAAAGCCTCCAGCCGGACCTCGGCTCTGCTCTCCGGCTTCG
CCATGGTGGCAATGGTGGAGGTGCAGCTGGACGCTGACCACGACTACCC
ACCGGGGCTGCTCATCGCCTTCAGTGCCTGCACCACAGTGCTGGTGGCTG
TGCACCTGTTTGCGCTCATGATCAGCACCTGCATCCTGCCCAACATCGAG
GCGGTGAGCAACGTGCACAATCTCAACTCGGTCAAGGAGTCCCCCCATG
AGCGCATGCACCGCCACATCGAGCTGGCCTGGGCCTTCTCCACCGTCATC
GGCACGCTGCTCTTCCTAGCTGAGGTGGTGCTGCTCTGCTGGGTCAAGTT
CTTGCCCCTCAAGAGGCAAGCGGGACAGCCAAGGCCCACCAGCAAGCCC
CCTGCATCAGGCGCCGCCGCCAACCACAGCGACAGCAGCGGCATCACCC
CGGGCCAGGCAGCTGCCATCGCTTCGACCACCATCATGGTGCCCTTCGGC
CTGATCTTTATCGTCTTCGCCGTCCACTTCTACCGCTCACTGGTTAGCCA
TAAGACCGACCGACAGTTCCAGGAGCTCAACGAGCTGGCGGAGTTTGCCC
GCTTACAGGACCAGCTGGACCACAGAGGGGACCACCCCCTGACGCCCGG
CAGCCACTATGCCTAG//.

The sequence of hOrai1-mOrai1 ECL2 (RPTSKPPASGAA) protein is the following (ECL2 domain is underlined, RPTSKPPASGAA (SEQ ID NO:190) sequence is double underlined):

SEQ ID NO: 192

MHPEPAPPPSRSSPELPPSGGSTTSGSRRSRRRSGDGEPPGAPPPPPSAV
TYPDWIGQSYSEVMSLNEHSMQALSWRKLYLSRAKLKASSRTSALLSGFA
MVAMVEVQLDADHDYPPGLLIAFSACTTVLVAVHLFALMISTCILPNIEA
VSNVHNLNSVKESPHERMHRHIELAWAFSTVIGTLLFLAEVVLLCWVKF
LPLKRQAGQPRPTSKPPASGAAANHSDSSGITPGQAAAIASTTIMVPFG
LIFIVFAVHFYRSLVSHKTDRQFQELNELAEFARLQDQLDHRGDHPLTPG
SHYA//.

Using pcDNA3.1/Hygromycin-hOrai1-mOrai1 ECL2 (PASGAA) vector and primers with the sequences SEQ ID NOS:193-194 below, we generated hOrai1-mOrai1 ECL2 where amino acid residues 218 to 228 of SEQ ID NO:72 (mOrai1), with the amino acid sequence AESVIVANHSD (SEQ ID NO:195), were mutated to the corresponding amino acid sequence of residues 216 to 226 of SEQ ID NO:2 (hOrai1), having the sequence PASGAAANVST (SEQ ID NO:196), referred to as hOrai1-mOrai1 ECL2 (PASGAAANVST) protein (SEQ ID NO: 198), encoded by hOrai1-mOrai1 ECL2 (PASGAAANVST) cDNA (SEQ ID NO:197).

The forward primer sequence was:

(SEQ ID NO: 193)

5′-GCGCCGCCGCCAACGTCAGCACCAGCAGCGGCATCA-3′//;
and
the reverse primer sequence was:

(SEQ ID NO: 194)

5′-TGATGCCGCTGCTGGTGCTGACGTTGGCGGCGGCGC-3′.

The sequence of hOrai1-mOrai1 ECL2 (PASGAAANVST) cDNA is the following (ECL2 domain is underlined, PASGAAANVST (SEQ ID NO:196) coding sequence is double underlined):

SEQ ID NO: 197

ATGCATCCGGAGCCCGCCCCGCCCCCGAGCCGCAGCAGTCCCGAGCTTCC
CCCAAGCGGCGGCAGCACCACCAGCGGCAGCCGCCGGAGCCGCCGCCGC
AGCGGGGACGGGGAGCCCCCGGGGGCCCCGCCACCGCCGCCGTCCGCCG
TCACCTACCCGGACTGGATCGGCCAGAGTTACTCCGAGGTGATGAGCCTC
AACGAGCACTCCATGCAGGCGCTGTCCTGGCGCAAGCTCTACTTGAGCCG
CGCCAAGCTTAAAGCCTCCAGCCGGACCTCGGCTCTGCTCTCCGGCTTCG
CCATGGTGGCAATGGTGGAGGTGCAGCTGGACGCTGACCACGACTACCC
ACCGGGGCTGCTCATCGCCTTCAGTGCCTGCACCACAGTGCTGGTGGCTG
TGCACCTGTTTGCGCTCATGATCAGCACCTGCATCCTGCCCAACATCGAG
GCGGTGAGCAACGTGCACAATCTCAACTCGGTCAAGGAGTCCCCCCATG
AGCGCATGCACCGCCACATCGAGCTGGCCTGGGCCTTCTCCACCGTCATC
GGCACGCTGCTCTTCCTAGCTGAGGTGGTGCTGCTCTGCTGGGTCAAGTT
CTTGCCCCTCAAGAGGCAAGCGGGACAGCCAAGCCCCACCAAGCCTCCC
CCTGCATCAGGCGCCGCCGCCAACGTCAGCACCAGCAGCGGCATCACCC
CGGGCCAGGCAGCTGCCATCGCTTCGACCACCATCATGGTGCCCTTCGGC
CTGATCTTTATCGTCTTCGCCGTCCACTTCTACCGCTCACTGGTTAGCCA
TAAGACCGACCGACAGTTCCAGGAGCTCAACGAGCTGGCGGAGTTTGCCC
GCTTACAGGACCAGCTGGACCACAGAGGGGACCACCCCCTGACGCCCGG
CAGCCACTATGCCTAG//

The sequence of hOrai1-mOrai1 ECL2 (PASGAAANVST) protein is the following (ECL2 domain is underlined, PASGAAANVST (SEQ ID NO:196) sequence is double underlined):

SEQ ID NO:. 198

MHPEPAPPPSRSSPELPPSGGSTTSGSRRSRRRSGDGEPPGAPPPPPSAV
TYPDWIGQSYSEVMSLNEHSMQALSWRKLYLSRAKLKASSRTSALLSGFA
MVAMVEVQLDADHDYPPGLLIAFSACTTVLVAVHLFALMISTCILPNIEA
VSNVHNLNSVKESPHERMHRHIELAWAFSTVIGTLLFLAEVVLLCWVKFL
PLKRQAGQPSPTKPPPASGAAANVSTSSGITPGQAAAIASTTIMVPFGLI
FIVFAVHFYRSLVSHKTDRQFQELNELAEFARLQDQLDHRGDHPLTPGSH
YA//

Using pcDNA3.1/Hygromycin-hOrai1-mOrai1 ECL2 (RPTSKPPA) vector and primers with the sequences SEQ ID NOS:199-200 below, we generated hOrai1-mOrai1 ECL2 where amino acid residues 206 to 219 of SEQ ID NO:72 (mOrai1), with the amino acid sequence RQAGQPSPTKPPAE (SEQ ID NO:201), were mutated to the corresponding amino acid sequence of residues 204 to 217 of SEQ ID NO:2 (hOrai1), having the sequence KQPGQPRPTSKPPA (SEQ ID NO:202), referred to as hOrai1-mOrai1 ECL2 (KQPGQPRPTSKPPA) protein (SEQ ID NO:204), encoded by hOrai1-mOrai1 ECL2 (KQPGQPRPTSKPPA) cDNA (SEQ

The forward primer sequence was:

(SEQ ID NO: 199)

5′-AGTTCTTGCCCCTCAAGAAGCAACCGGGACAGCC-3′//;
and
the reverse primer sequence was:

(SEQ ID NO: 200)

5′-GGCTGTCCCGGTTGCTTCTTGAGGGGCAAGAACT-3′.

The sequence of hOrai1-mOrai1 ECL2 (KQPGQPRPTSKPPA) cDNA is the following (ECL2 domain is underlined, KQPGQPRPTSKPPA (SEQ ID NO:202) coding sequence is double underlined):

SEQ ID NO: 203

ATGCATCCGGAGCCCGCCCCGCCCCCGAGCCGCAGCAGTCCCGAGCTTCC
CCCAAGCGGCGGCAGCACCACCAGCGGCAGCCGCCGGAGCCGCCGCCGC
AGCGGGGACGGGGAGCCCCCGGGGGCCCCGCCACCGCCGCCGTCCGCCG
TCACCTACCCGGACTGGATCGGCCAGAGTTACTCCGAGGTGATGAGCCTC
AACGAGCACTCCATGCAGGCGCTGTCCTGGCGCAAGCTCTACTTGAGCCG
CGCCAAGCTTAAAGCCTCCAGCCGGACCTCGGCTCTGCTCTCCGGCTTCG
CCATGGTGGCAATGGTGGAGGTGCAGCTGGACGCTGACCACGACTACCC
ACCGGGGCTGCTCATCGCCTTCAGTGCCTGCACCACAGTGCTGGTGGCTG
TGCACCTGTTTGCGCTCATGATCAGCACCTGCATCCTGCCCAACATCGAG
GCGGTGAGCAACGTGCACAATCTCAACTCGGTCAAGGAGTCCCCCCATG
AGCGCATGCACCGCCACATCGAGCTGGCCTGGGCCTTCTCCACCGTCATC
GGCACGCTGCTCTTCCTAGCTGAGGTGGTGCTGCTCTGCTGGGTCAAGTT
CTTGCCCCTCAAGAAGCAACCGGGACAGCCAAGGCCCACCAGCAAGCCC
CCTGCATCAGTCATCGTCGCCAACCACAGCGACAGCAGCGGCATCACCC
CGGGCCAGGCAGCTGCCATCGCTTCGACCACCATCATGGTGCCCTTCGGC
CTGATCTTTATCGTCTTCGCCGTCCACTTCTACCGCTCACTGGTTAGCCA
TAAGACCGACCGACAGTTCCAGGAGCTCAACGAGCTGGCGGAGTTTGCCC
GCTTACAGGACCAGCTGGACCACAGAGGGGACCACCCCCTGACGCCCGG
CAGCCACTATGCCTAG//.

The sequence of hOrai1-mOrai1 ECL2 (KQPGQPRPTSKPPA) protein is the following (ECL2 domain is underlined, KQPGQPRPTSKPPA (SEQ ID NO:202) sequence is double underlined):

SEQ ID NO: 204

MHPEPAPPPSRSSPELPPSGGSTTSGSRRSRRRSGDGEPPGAPPPPPSAV
TYPDWIGQSYSEVMSLNEHSMQALSWRKLYLSRAKLKASSRTSALLSGFA
MVAMVEVQLDADHDYPPGLLIAFSACTTVLVAVHLFALMISTCILPNIEA
VSNVHNLNSVKESPHERMHRHIELAWAFSTVIGTLLFLAEVVLLCWVKFL
PLKKQPGQPRPTSKPPASVIVANHSDSSGITPGQAAAIASTTIMVPFGLI
FIVFAVHFYRSLVSHKTDRQFQELNELAEFARLQDQLDHRGDHPLTPGSH
YA//.

Using pcDNA3.1/Hygromycin-hOrai1-mOrai1 ECL2 (KQPGQPRPTSKPPA) vector and primers with the sequences SEQ ID NOS:205-206 below, we generated hOrai1-mOrai1 ECL2 where amino acid residues 206 to 223 of SEQ ID NO:72 (mOrai1), with the amino acid sequence RQAGQPSPTKPPAESVIV (SEQ ID NO:207), were mutated to the corresponding amino acid sequence of residues 204 to 221 of SEQ ID NO:2 (hOrai1), having the sequence KQPGQPRPTSKPPASGAA (SEQ ID NO:208), referred to as hOrai1-mOrai1 ECL2 (KQPGQPRPTSKPPASGAA) protein (SEQ ID NO:210), encoded by hOrai1-mOrai1 ECL2 (KQPGQPRPTSKPPASGAA) cDNA (SEQ ID NO:209).

The forward primer sequence was:

(SEQ ID NO: 205)

5′-AAGCCCCCTGCATCAGGCGCCGCCGCCAACCACAGCGAC-3′//;
and
the reverse primer sequence was:

(SEQ ID NO: 206)

5′-GTCGCTGTGGTTGGCGGCGGCGCCTGATGCAGGGGGCTT-3′.

The sequence of hOrai1-mOrai1 ECL2 (KQPGQPRPTSKPPASGAA) cDNA is the following (ECL2 domain is underlined, KQPGQPRPTSKPPASGAA (SEQ ID NO:208) coding sequence is double underlined):

SEQ ID NO: 209

ATGCATCCGGAGCCCGCCCCGCCCCCGAGCCGCAGCAGTCCCGAGCTTCC
CCCAAGCGGCGGCAGCACCACCAGCGGCAGCCGCCGGAGCCGCCGCCGC
AGCGGGGACGGGGAGCCCCCGGGGGCCCCGCCACCGCCGCCGTCCGCCG
TCACCTACCCGGACTGGATCGGCCAGAGTTACTCCGAGGTGATGAGCCTC
AACGAGCACTCCATGCAGGCGCTGTCCTGGCGCAAGCTCTACTTGAGCCG
CGCCAAGCTTAAAGCCTCCAGCCGGACCTCGGCTCTGCTCTCCGGCTTCG
CCATGGTGGCAATGGTGGAGGTGCAGCTGGACGCTGACCACGACTACCC
ACCGGGGCTGCTCATCGCCTTCAGTGCCTGCACCACAGTGCTGGTGGCTG
TGCACCTGTTTGCGCTCATGATCAGCACCTGCATCCTGCCCAACATCGAG
GCGGTGAGCAACGTGCACAATCTCAACTCGGTCAAGGAGTCCCCCCATG
AGCGCATGCACCGCCACATCGAGCTGGCCTGGGCCTTCTCCACCGTCATC
GGCACGCTGCTCTTCCTAGCTGAGGTGGTGCTGCTCTGCTGGGTCAAGTT
CTTGCCCCTCAAGAAGCAACCGGGACAGCCAAGGCCCACCAGCAAGCCC
CCTGCATCAGGCGCCGCCGCCAACCACAGCGACAGCAGCGGCATCACCC
CGGGCCAGGCAGCTGCCATCGCTTCGACCACCATCATGGTGCCCTTCGGC
CTGATCTTTATCGTCTTCGCCGTCCACTTCTACCGCTCACTGGTTAGCCA
TAAGACCGACCGACAGTTCCAGGAGCTCAACGAGCTGGCGGAGTTTGCCC
GCTTACAGGACCAGCTGGACCACAGAGGGGACCACCCCCTGACGCCCGG
CAGCCACTATGCCTAG//.

The sequence of hOrai1-mOrai1 ECL2 (KQPGQPRPTSKPPASGAA) protein is the following (ECL2 domain is underlined, KQPGQPRPTSKPPASGAA (SEQ ID NO:208) sequence is double underlined):

SEQ ID NO: 210

MHPEPAPPPSRSSPELPPSGGSTTSGSRRSRRRSGDGEPPGAPPPPPSAV
TYPDWIGQSYSEVMSLNEHSMQALSWRKLYLSRAKLKASSRTSALLSGFA
MVAMVEVQLDADHDYPPGLLIAFSACTTVLVAVHLFALMISTCILPNIEA
VSNVHNLNSVKESPHERMHRHIELAWAFSTVIGTLLFLAEVVLLCWVKFL
PLKKQPGQPRPTSKPPASGAAANHSDSSGITPGQAAAIASTTIMVPFGLI
FIVFAVHFYRSLVSHKTDRQFQELNELAEFARLQDQLDHRGDHPLTPGSH
YA//.

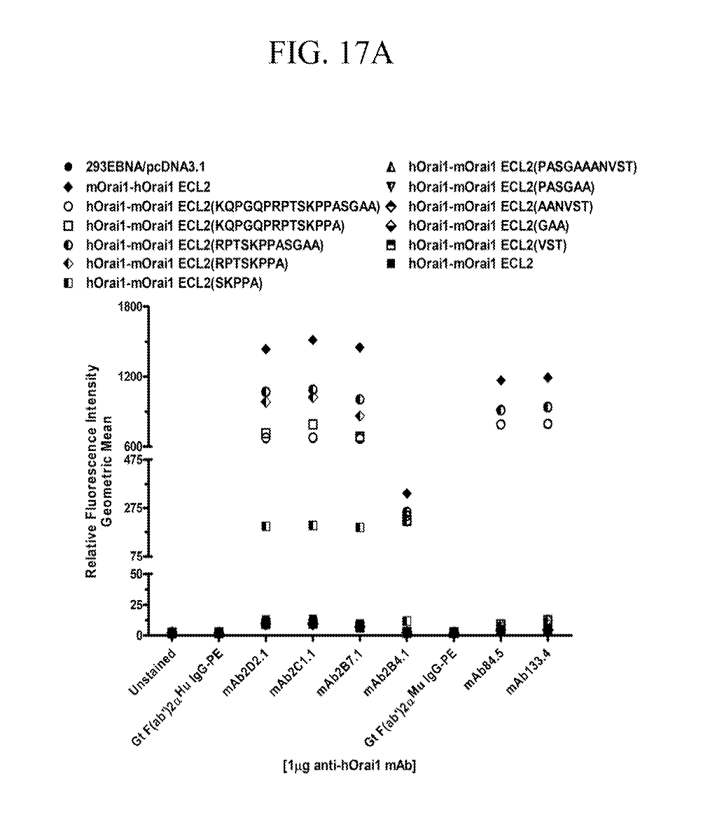

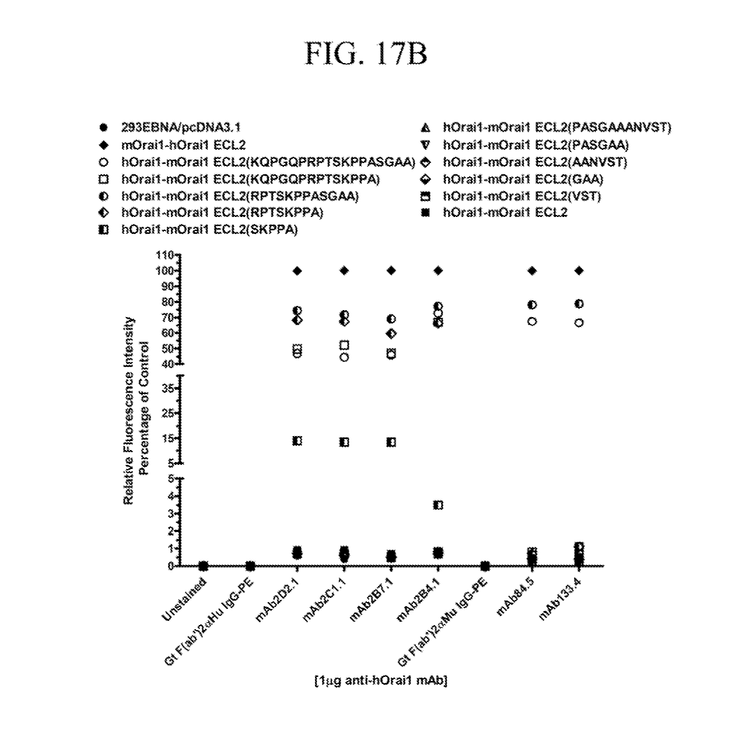

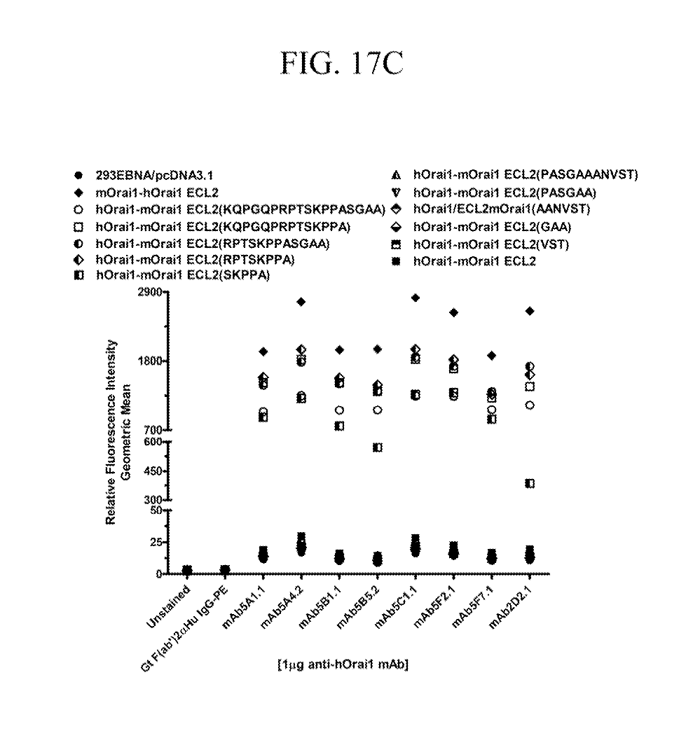

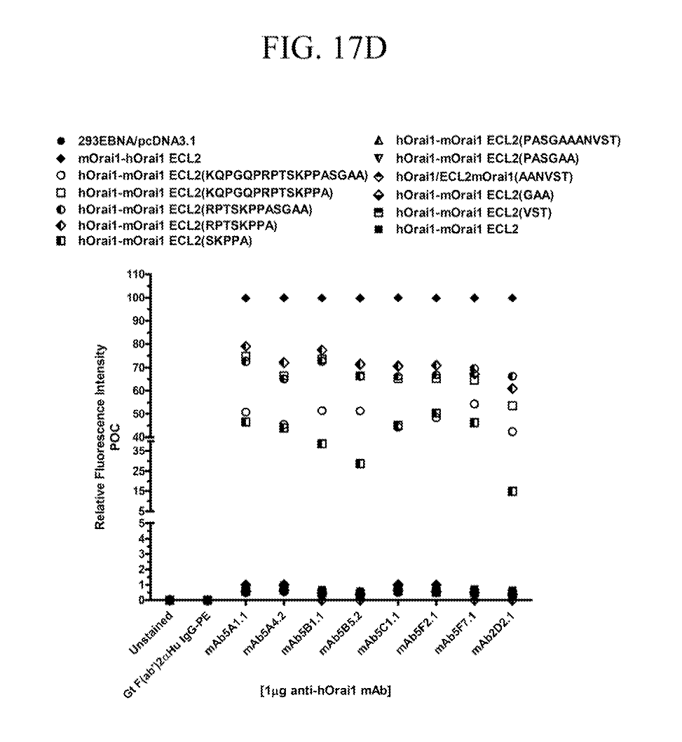

Representative results of FACS analysis of binding to the mutant hOrai1-mOrai1 ECL2 chimera described above by exemplary anti-hOrai1 antibodies of the present invention are shown in FIG. 17A-D, Table 10A and Table 10B below. Table 10A (Campaign 1) and Table 10B (Campaign 2) show the gain in binding ability of exemplary monoclonal antibodies to hOrai 1-mOrai 1 ECL2 chimera mutants as determined by FACS. FIG. 17A shows the raw Geo Mean data from the experiment in which we started with the hOrai-mOrai1 ECL2 chimera and made a series of mutants changing the mouse amino acids in the ECL2 to corresponding human amino acids where there is a difference between the two species to look for “gain of binding” mutants by the recombinant mAbs from Campaign 1 as well as from purified mAb 84.5 and mAb133.4. FIG. 17C shows similar “gain of binding” data from the seven purified mAbs from Campaign 2 along with recombinant mAb 2D2.1 from Campaign 1 for comparison. The “gain of binding” experiment was intended to tell us more definitively the subregions in the human ECL2 that are important for binding by the inventive mAbs, since the gain of binding can only be achieved by retaining the proper conformation in a chimeric background. The Geo Means of the Unstained control and the directly labeled secondary antibody control binding to cells transfected with chimera constructs and the pcDNA3.1 vector control were all low and together represent “Negative Controls”. Because of differences in the Geo Means of the mAbs binding to hOrai1-mOrai1 ECL2 (SEQ ID NO:91) in some cases, we controlled for background binding to any endogenously expressed hOrai1 by re-plotting the Geo Means as a percent of control (POC) using the background staining as zero and the binding to the mOrai1-hOrai1 ECL2 (SEQ ID NO:97; Ch. 12), having a fully human Orai1 ECL2 sequences, as the highest attainable for that mAb to calculate the percent of control (POC) for each sample. FIG. 17B and FIG. 17D are such plots of the POC and so all the values for binding to the mOrai1-hOrai1 ECL2 are at 100% but in some cases the value for binding to the hOrai1-mOrai1 ECL2 (SEQ ID NO:91) is not zero due presumably to endogenous human Orai1 expression in HEK-293 cells. (E.g., Sternfeld et al., Activation of muscarinic receptors reduces store-operated Ca²⁺ entry in HEK-293 cells, Cellular Signalling 19:1457-64 (2007); Fasolato et al., Store depletion triggers the calcium release-activated calcium current (ICRAC) in macrovascular endothelial cells: a comparison with Jurkat and embryonic kidney cell lines, Pfluegers Arch.-Eur. J. Physiol. 436(1):69-74 (1998)). Table 10A is a schematic representation of FIG. 17B and Table 10B is a schematic representation of FIG. 17D, where the binding results are recorded as (+++) denoting binding POC from 40% to 100%, (++) for POC of 5% to less than 40%, (+) for POC from 1% to less than 5% and (−) as lack of binding with POC less than 1%. The tops of Table 10A-B show an alignment between the human and mouse Orai1 protein in the ECL2 region only with the human amino acids represented in capital letters, the mouse ECL2 amino acids are all lower case letters and the underlined amino acids denotes differences between human and mouse protein sequences. The chimeras (Ch.) are numbered on the left hand side starting with the hOrai1-mOrai1 ECL2 (SEQ ID NO:91) as Ch. 1 and the mOrai1-hOrai1 ECL2 (SEQ ID NO:97) as Ch. 12. The Ch. 2 to Ch. 11 are hOrai1-mOrai1 ECL2 mutants, wherein specific mouse amino acids are replaced with their human counterparts. The human to mouse amino acid changes in the table are represented by upper case letters and the dashes denote no changes. For Table 10A under “Binding”, the recombinant human mAb2D2.1, mAb2C1.1 and mAb2B7.1 from Campaign 1 are grouped together in the left column, human mAb2B4.1 from Campaign lis by itself in the middle column and mouse mAb84.5 and 133.4 are grouped together in the right-most column. For Table 10B under “Binding”, the recombinant human mAb2D2.1 from Campaign 1 is provided for comparison in the left column, the purified mAb 5B1.1 and mAb 5B5.2 from Campaign 2 are in the middle column and the rest of the purified mAbs from Campaign 2 are in the right column. The RFI-POC was calculated from the relative fluorescence intensity geometric mean (Geo Mean) using the algorithm (Algorithm I, below) of Geo Mean of a mAb binding to cells expressing a chimera minus average Geo Mean of Negative Controls, then divided by Geo Mean of the particular mAb binding to Ch. 12 (mOrai-hOrai1 ECL2; SEQ ID NO:97), the entire quantity multiplied by 100.

Algorithm I (inserting “mAb 2D2.1” and “Ch. 2” as examples of a particular mAb and sample chimera of interest, for which others of interest can be substituted):

$$RFI \text{-} POC of 2 D 2.1 binding to Ch . 2 = \frac{\begin{matrix}Geo Mean of 2 D 2.1 binding to Ch . 2 - \\ Average Geo Mean of Negative Controls\end{matrix}}{Geo Mean of 2 D 2.1 binding to Ch . 12} \times 100$$

TABLE 10A
Binding of monoclonal antibodies from Campaign 1 to hOrai1-mOrai1
ECL2 chimera mutants as determined by FACS.

| | | Binding | | |
|---|---|---|---|---|
| Ch. | 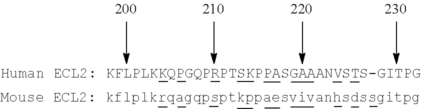 | 2D2.1 2C1.1 2B7.1 | 2B4.1 | 84.5 133.4 |
| 1 | hOrai1/mOrai1 ECL2(kflplkrqagqpsptkppaesvivanhsdssgitpg) | − | − | − |
| 2 | hOrai1/mOrai1 ECL2(------K-P---R--SK-PA-GAA------------) | +++ | ++ | +++ |
| 3 | hOrai1/mOrai1 ECL2(------K-P---R--SK-PA----------------) | +++ | ++ | − |
| 4 | hOrai1/mOrai1 ECL2(------------R--SK-PA-GAA------------) | +++ | ++ | +++ |
| 5 | hOrai1/mOrai1 ECL2(------------R--SK-PA----------------) | +++ | ++ | − |
| 6 | hOrai1/mOrai1 ECL2(---------------SK-PA----------------) | ++ | + | − |
| 7 | hOrai1/mOrai1 ECL2(------------------PA-GAA--V-T-------) | − | − | − |
| 8 | hOrai1/mOrai1 ECL2(------------------PA-GAA------------) | − | − | − |
| 9 | hOrai1/mOrai1 ECL2(----------------------AA--V-T-------) | − | − | − |
| 10 | hOral1/mOrai1 ECL2(---------------------GAA------------) | − | − | − |
| 11 | hOrai1/mOrai1 ECL2(--------------------------V-T-------) | − | − | − |
| 12 | mOrai1/hOrai1 ECL2(KFLPLKKQPGQPRPTSKPPASGAAANVSTS-GITPG) | +++ | ++ | +++ |

TABLE 10B
Binding of monoclonal antibodies from Campaign 2 to hOrai1-mOrai1
ECL2 chimera mutants as determined by FACS.

| | | Binding | | |
|---|---|---|---|---|
| Ch. | 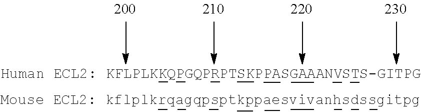 | 2D2.1 | 5B1.1 5B5.2 | 5A1.1 5A4.2 5C1.1 5F2.1 5F7.1 |
| 1 | hOrai1/mOrai1 ECL2(kflplkrqagqpsptkppaesvivanhsdssgitpg) | − | − | − |
| 2 | hOrai1/mOrai1 ECL2(------K-P---R--SK-PA-GAA------------) | +++ | +++ | +++ |
| 3 | hOrai1/mOrai1 ECL2(------K-P---R--SK-PA----------------) | +++ | +++ | +++ |
| 4 | hOrai1/mOrai1 ECL2(------------R--SK-PA-GAA------------) | +++ | +++ | +++ |
| 5 | hOrai1/mOrai1 ECL2(------------R--SK-PA----------------) | +++ | +++ | +++ |
| 6 | hOrai1/mOrai1 ECL2(---------------SK-PA----------------) | ++ | ++ | +++ |
| 7 | hOrai1/mOrai1 ECL2(------------------PA-GAA--V-T-------) | − | − | − |
| 8 | hOrai1/mOrai1 ECL2(------------------PA-GAA------------) | − | − | − |
| 9 | hOrai1/mOrai1 ECL2(----------------------AA--V-T-------) | − | − | − |
| 10 | hOrai1/mOrall ECL2(---------------------GAA------------) | − | − | − |
| 11 | hOrai1/mOrai1 ECL2(--------------------------V-T-------) | − | − | − |
| 12 | mOrai1/hOrai1 ECL2(KFLPLKKQPGQPRPTSKPPASGAAANVSTS-GITPG) | +++ | +++ | +++ |

Table 10A shows that none of the mAbs bind to Ch. 7 through 11, indicating that this region may not play a role in the binding by recombinant mAbs from Campaign 1 as well as from purified mAb 84.5 and mAb133.4. Table 10B also shows similar lack of binding to Ch. 7 through 11 by the purified mAbs from Campaign 2. However, it could not be ruled out that the region from amino acid residues 216 to 226 of SEQ ID NO:2 may play a minor role in the binding but not enough affinity is gained to be visualized in a FACS binding experiment. There is a gain of binding observed for hOrai1-mOrai1 ECL2 (SKPPA) (Ch. 6; SEQ ID NO:103) with mAb 2C1.1, mAb 2D2.1, 2B7.1, mAb and 2B4.1 from Campaign 1 and purified mAb 5B1.1 and mAb 5B5.2 from Campaign 2 that is only a fraction of the binding seen for mOral-hOrai1 ECL2 (Ch. 12; SEQ ID NO:97), indicating that this region of amino acid residues 213 to 217 of SEQ ID NO:2 (human Orai1 sequence) is important for their binding. Interestingly, the gain of binding observed in FIG. 17D and Table 10B to mOrai1-hOrai1 ECL2 (SKPPA) (SEQ ID NO:103; Ch.6) by purified mAb 5A1.1, mAb 5A4.2, mAb 5C1.1, mAb 5F2.1 and mAb 5F7.1 is almost as strong as their binding to mOral-hOrai1 ECL2 (SEQ ID NO:97; Ch. 12). This indicates a major difference between recombinant mAb 2C1.1, mAb 2D2.1, mAb 2B7.1 and mAb 2B4.1 from Campaign 1 and purified mAb 5B1.1 and mAb 5B5.2 from Campaign 2 compared to purified mAb 5A1.1, mAb 5A4.2, mAb 5C1.1, mAb 5F2.1 and mAb5F7.1 from Campaign 2 (FIGS. 17B and 17D and Table 10A-B).

If we extend the humanization of the mouse ECL2 region with amino acid residues 210 to 217 of SEQ ID NO:2 (Ch.5; hOrai1-mOrai1 ECL2 (RPTSKPPA), SEQ ID NO:129), then the binding by mAb 2C1.1, mAb 2D2.1, 2B7.1 and mAb 2B4.1 from Campaign 1 is almost as strong as the binding by these mAbs to Chl2. (mOrai1-hOrai1 ECL2, SEQ ID NO:97) (FIG. 17B and Table 10A). In addition, similar binding to mOrai1-hOrai1 ECL2 (RPTSKPPA) (Ch. 5; SEQ ID NO:129) by purified mAbs from Campaign 2 was observed (FIG. 17D and Table 10B).

However, if the humanization is extended even further in amino-terminal direction with hOrai1-mOrai1 ECL2 (KQPGQPRPTSKPPASGAA) (Ch. 2; SEQ ID NO:210) and hOrai1-mOrai1 ECL2 (KQPGQPRPTSKPPA) (Ch. 3; SEQ ID NO:204) to a region encompassing amino acids 204 to 221 of SEQ ID NO:2, then there is a slight decrease in the POC for the mAb 2C1.1, mAb 2D2.1, 2B7.1 and mAb 2B4.1 as compared to hOrai1-mOrai1 ECL2 (RPTSKPPA) (Ch.5; SEQ ID NO:129). The binding of purified mAbs from Campaign 2 to mOrai1-hOrai1 ECL2 (KQPGQPRPTSKPPASGAA) (Ch. 2; SEQ ID NO:210) and mOrai1-hOrai1 ECL2 (KQPGQPRPTSKPPA) (Ch. 3; SEQ ID NO:204) was comparable to their binding to mOrai1-hOrai1 ECL2 (RPTSKPPA) (Ch. 5; SEQ ID NO:129), except for mAb 5B1.1 and mAb 5B5.2, which bound to mOrai1-hOrai1 ECL2 (KQPGQPRPTSKPPASGAA) (Ch. 2; SEQ ID NO:210) and mOrai1-hOrai1 ECL2 (KQPGQPRPTSKPPA) (Ch. 3; SEQ ID NO:204) slightly less strongly than mOrai1-hOrai1 ECL2 (RPTSKPPA) (Ch. 5; SEQ ID NO:129) (FIG. 17D and Table 10B). However, the binding of mAb 5A1.1, mAb 5A4.2, mAb 5C1.1, mAb 5F2.1 and mAb 5F7.1 from Campaign 2 were slightly different from Campaign 1 mAbs in that the Campaign 2 mAbs bound to hOrai1-mOrai1 ECL2 (SKPPA) (Ch. 6; SEQ ID NO:103) almost as robustly as they bound to mOral-hOrai1 ECL2 (Ch. 12; SEQ ID NO:97) (Table 10B). The data indicate that the subregion from amino acids 204 to 206 of SEQ ID NO:2 was not important for binding the recombinant mAbs from Campaign 1 and the purified mAbs from Campaign 2.

In addition, if the humanization is extended in the carboxy-terminus direction with hOrai1-mOrai1 ECL2 (RPTSKPAASGAA) (Ch. 4; SEQ ID NO: 192) and hOrai1-mOrai1 ECL2 (KQPGQPRPTSKPPASGAA) (Ch. 2; SEQ ID NO:210), then there is no difference in binding by the recombinant mAbs from Campaign 1 and purified mAbs from Campaign 2 indicating that the subregion of amino acids 218 to 221 of SEQ ID NO:2 is not important for binding (FIG. 17B and FIG. 17D and Table 10A-B). Therefore, we concluded from the “gain of binding” experiment that a subset of amino acid residues 207 to 217 of SEQ ID NO:2 is critical for binding by mAb 2C1.1, mAb 2D2.1, mAb 2B7.1 and mAb 2B4.1 from Campaign 1 and the purified mAb 5A1.1, mAb 5A4.2, mAb 5B1.1, mAb 5B5.2, mAb 5C1.1, mAb 5F2.1 and mAb5F7.1 from Campaign 2.

For the mAb 84.5 and mAb 133.4, the gain of binding observed with the hOrai1-mOrai1 ECL2 (KQPGQPRPTSKPPASGAA) (Ch. 2; SEQ ID NO:210) and hOrai1-mOrai1 ECL2 (RPTSKPPASGAA) (Ch.4; SEQ ID NO: 192) but not with the hOrai1-mOrai1 ECL2 (KQPGQPRPTSKPPA) (Ch. 3; SEQ ID NO:204) and hOrai1-mOrai1 ECL2 (RPTSKPPA) (Ch. 5; SEQ ID NO: 129) indicates that region from amino acids 219 to 221 of SEQ ID NO:2 (human Oral) is important for binding and may play a more critical role when compared to the region preceding it from amino acids 204 to 218 (Table 10A). In addition, FIG. 17B shows in more detail that there is negligible difference in binding of mAb 84.5 and mAb. 133.4 to hOrai1-mOrai1 ECL2 (KQPGQPRPTSKPPASGAA) (Ch. 2; SEQ ID NO:210) and hOrai1-mOrai1 ECL2 (RPTSKPAASGAA) (Ch. 4; SEQ ID NO: 192) indicating that the region from amino acids 204 to 206 of SEQ ID NO:2 does not contribute significantly to the binding. Therefore, we can conclude from the “gain of binding” experiment that a subset of amino acid residues 207 to 223 of SEQ ID NO:2 (human Orai1) is critical for binding by mAb 84.5 and mAb 133.4 with a subset of amino acid residues 218 to 223 of SEQ ID NO:2 playing a more critical role in binding.

Mutation of mOrai1-hOrai1 ECL2 Chimera.

The next series of mutants was designed to probe where in the subregions of the human extracellular loop 2 do the monoclonal antibodies of the present invention bind by looking for loss of binding in the mutants. To accomplish this, we started with mOrai1-hOrai1 ECL2 chimera, which the monoclonal antibodies bind to and designed a series of mutants where the human amino acids in the extracellular loop 2 were swapped for their corresponding mouse counterparts based on the non-conserved regions between human and mouse Orai1 protein (FIG. 14) and look for loss of binding.

Using pcDNA3.1/Hygromycin-mOrai1-hOrai1 ECL2 vector and primers with the sequences SEQ ID NOS:130-131 below, we generated mOrai1-hOrai1 ECL2 where amino acid residues 213 to 217 of SEQ ID NO:2 (hOrai1), with the amino acid sequence SKPPA (SEQ ID NO:99), were mutated to the corresponding amino acid sequence of residues 215 to 219 of SEQ ID NO:72 (mOrai1), having the sequence KPPAE (SEQ ID NO:98), referred to as mOrai1-hOrai1 ECL2 (KPPAE) protein (SEQ ID NO:133), encoded by mOrai1-hOrai1 ECL2 (KPPAE) cDNA (SEQ ID NO:132).

The forward primer sequence was:

(SEQ ID NO: 130)

5′-CAGCCAAGGCCCACCAAGCCGCCCGCCGAGAGTGGCGCAGCAGC-
3′//;
and
the reverse primer sequence was:

(SEQ ID NO: 131)

5′-GCTGCTGCGCCACTCTCGGCGGGCGGCTTGGTGGGCCTTGGCTG-
3′.

The sequence of mOrai1-hOrai1 ECL2 (KPPAE) cDNA is the following (ECL2 domain is underlined, KPPAE (SEQ ID NO:98) coding sequence is double underlined):

SEQ ID NO: 132

ATGCATCCGGAGCCTGCCCCGCCCCCGAGTCACAGCAATCCGGAGCTTCC
CGTGAGCGGCGGCAGCAGCACTAGCGGCAGCCGCCGGAGCCGCCGCCGC
AGCGGGGACGGGGAGCCCTCGGGGGCCCCACCGCTGCCGCCGCCGCCAC
CCGCCGTCAGCTACCCGGACTGGATCGGCCAGAGTTACTCCGAGGTGAT
GAGCCTCAACGAGCACTCGATGCAGGCGCTGTCCTGGCGCAAGCTCTACT
TAAGCCGCGCCAAGCTCAAAGCTTCCAGCCGGACCTCGGCTCTGCTCTCC
GGCTTCGCCATGGTAGCGATGGTGGAAGTCCAGCTGGACACAGACCATG
ACTACCCACCAGGGTTGCTCATCGTCTTTAGTGCCTGCACCACAGTGCTA
GTGGCCGTGCACCTGTTTGCCCTCATGATCAGCACCTGCATCCTGCCCAA
CATCGAGGCTGTGAGCAACGTCCACAACCTCAACTCGGTCAAAGAGTCA
CCCCACGAGCGCATGCATCGCCACATCGAGCTGGCCTGGGCCTTCTCCAC
GGTCATCGGGACGCTGCTTTTCCTAGCAGAGGTCGTGCTGCTCTGCTGGG
TCAAGTTCTTACCTCTCAAGAAGCAGCCAGGCCAGCCAAGGCCCACCAA
GCCGCCCGCCGAGAGTGGCGCAGCAGCCAACGTCAGCACCAGCGGCATC
ACCCCGGGTGAGGCGGCAGCCATTGCCTCCACCGCCATCATGGTTCCCTG
TGGCCTGGTTTTCATCGTCTTTGCTGTTCACTTCTACCGCTCCCTGGTCA
GCCATAAGACGGACCGGCAGTTCCAGGAGCTCAATGAGCTGGCCGAGTTT
GCCCGCTTGCAGGACCAGCTGGACCACAGAGGGGACCATTCTCTAACAC
CGGGCACCCACTATGCCTAA//.

The sequence of mOrai1-hOrai1 ECL2 (KPPAE) protein is the following (ECL2 domain is underlined, KPPAE (SEQ ID NO:98) sequence is double underlined):

SEQ ID NO: 133

MHPEPAPPPSHSNPELPVSGGSSTSGSRRSRRRSGDGEPSGAPPLPPPPP
AVSYPDWIGQSYSEVMSLNEHSMQALSWRKLYLSRAKLKASSRTSALLSG
FAMVAMVEVQLDTDHDYPPGLLIVFSACTTVLVAVHLFALMISTCILPNI
EAVSNVHNLNSVKESPHERMHRHIELAWAFSTVIGTLLFLAEVVLLCWVK
FLPLKKQPGQPRPTKPPAESGAAANVSTSGITPGEAAAIASTAIMVPCGL
VFIVFAVHFYRSLVSHKTDRQFQELNELAEFARLQDQLDHRGDHSLTPGT
HYA//.

Using pcDNA3.1/Hygromycin-mOrai1-hOrai1 ECL2 vector and primers with the sequences SEQ ID NOS:134-135 below, we generated mOrai1-hOrai1 ECL2 where amino acid residues 219 to 221 of SEQ ID NO:2 (hOrai1), with the amino acid sequence GAA, were mutated to the corresponding amino acid sequence of residues 221 to 223 of SEQ ID NO:72 (mOrai1), having the sequence VIV, referred to as mOrai1-hOrai1 ECL2 (VIV) protein (SEQ ID NO: 137), encoded by mOrai1-hOrai1 ECL2 (VIV) cDNA (SEQ ID NO:136).

The forward primer sequence was:

(SEQ ID NO: 134)

5′-AAGCCCCCCGCCAGTGTCATAGTAGCCAACGTCAGCACC-3′//;
and
the reverse primer sequence was:

(SEQ ID NO: 135)

5′-GGTGCTGACGTTGGCTACTATGACACTGGCGGGGGGCTT-3′.

The sequence of mOrai1-hOrai1 ECL2 (VIV) cDNA is the following (ECL2 domain is underlined, VIV coding sequence is double underlined):

SEQ ID NO: 136

ATGCATCCGGAGCCTGCCCCGCCCCCGAGTCACAGCAATCCGGAGCTTCC
CGTGAGCGGCGGCAGCAGCACTAGCGGCAGCCGCCGGAGCCGCCGCCGC
AGCGGGGACGGGGAGCCCTCGGGGGCCCCACCGCTGCCGCCGCCGCCAC
CCGCCGTCAGCTACCCGGACTGGATCGGCCAGAGTTACTCCGAGGTGAT
GAGCCTCAACGAGCACTCGATGCAGGCGCTGTCCTGGCGCAAGCTCTACT
TAAGCCGCGCCAAGCTCAAAGCTTCCAGCCGGACCTCGGCTCTGCTCTCC
GGCTTCGCCATGGTAGCGATGGTGGAAGTCCAGCTGGACACAGACCATG
ACTACCCACCAGGGTTGCTCATCGTCTTTAGTGCCTGCACCACAGTGCTA
GTGGCCGTGCACCTGTTTGCCCTCATGATCAGCACCTGCATCCTGCCCAA
CATCGAGGCTGTGAGCAACGTCCACAACCTCAACTCGGTCAAAGAGTCA
CCCCACGAGCGCATGCATCGCCACATCGAGCTGGCCTGGGCCTTCTCCAC
GGTCATCGGGACGCTGCTTTTCCTAGCAGAGGTCGTGCTGCTCTGCTGGG
TCAAGTTCTTACCTCTCAAGAAGCAGCCAGGCCAGCCAAGGCCCACCAG
CAAGCCCCCCGCCAGTGTCATAGTAGCCAACGTCAGCACCAGCGGCATC
ACCCCGGGTGAGGCGGCAGCCATTGCCTCCACCGCCATCATGGTTCCCTG
TGGCCTGGTTTTCATCGTCTTTGCTGTTCACTTCTACCGCTCCCTGGTCA
GCCATAAGACGGACCGGCAGTTCCAGGAGCTCAATGAGCTGGCCGAGTTT
GCCCGCTTGCAGGACCAGCTGGACCACAGAGGGGACCATTCTCTAACAC
CGGGCACCCACTATGCCTAA//.

The sequence of mOrai1-hOrai1 ECL2 (VIV) protein is the following (ECL2 domain is underlined, VIV sequence is double underlined):

SEQ ID NO: 137
MHPEPAPPPSHSNPELPVSGGSSTSGSRRSRRRSGDGEPSGAPPLPPPPP
AVSYPDWIGQSYSEVMSLNEHSMQALSWRKLYLSRAKLKASSRTSALLSG
FAMVAMVEVQLDTDHDYPPGLLIVFSACTTVLVAVHLFALMISTCILPNI
EAVSNVHNLNSVKESPHERMHRHIELAWAFSTVIGTLLFLAEVVLLCWVK
FLPLKKQPGQPRPTSKPPASVIVANVSTSGITPGEAAAIASTAIMVPCGL
VFIVFAVHFYRSLVSHKTDRQFQELNELAEFARLQDQLDHRGDHSLTPGT
HYA//.

Using pcDNA3.1/Hygromycin-mOrai1-hOrai1 ECL2 vector and primers with the sequences SEQ ID NOS:138-139 below, we generated mOrai1-hOrai1 ECL2 where amino acid residues 216 to 221 of SEQ ID NO:2 (hOrai1), with the amino acid sequence PASGAA (SEQ ID NO:109), were mutated to the corresponding amino acid sequence of residues 218 to 223 of SEQ ID NO:72 (mOrai1), having the sequence AESVIV (SEQ ID NO:108), referred to as mOrai1-hOrai1 ECL2 (AESVIV) protein (SEQ ID NO:141), encoded by mOrai1-hOrai1 ECL2 (AESVIV) cDNA (SEQ ID NO:140).

The forward primer sequence was:

(SEQ ID NO: 138)

5′-GCCCACCAGCAAGCCCGCCGAGAGTGTCATAGTAGCCAACGTCAGCA
CC-3′//;
and
the reverse primer sequence was:

(SEQ ID NO: 139)

5′-GGTGCTGACGTTGGCTACTATGACACTCTCGGCGGGCTTGCTGGTGG
GC-3′.

The sequence of mOrai1-hOrai1 ECL2 (AESVIV) cDNA is the following (ECL2 domain is underlined, AESVIV (SEQ ID NO:108) coding sequence is double underlined):

SEQ ID NO: 140

ATGCATCCGGAGCCTGCCCCGCCCCCGAGTCACAGCAATCCGGAGCTTCC
CGTGAGCGGCGGCAGCAGCACTAGCGGCAGCCGCCGGAGCCGCCGCCGC
AGCGGGGACGGGGAGCCCTCGGGGGCCCCACCGCTGCCGCCGCCGCCAC
CCGCCGTCAGCTACCCGGACTGGATCGGCCAGAGTTACTCCGAGGTGAT
GAGCCTCAACGAGCACTCGATGCAGGCGCTGTCCTGGCGCAAGCTCTACT
TAAGCCGCGCCAAGCTCAAAGCTTCCAGCCGGACCTCGGCTCTGCTCTCC
GGCTTCGCCATGGTAGCGATGGTGGAAGTCCAGCTGGACACAGACCATG
ACTACCCACCAGGGTTGCTCATCGTCTTTAGTGCCTGCACCACAGTGCTA
GTGGCCGTGCACCTGTTTGCCCTCATGATCAGCACCTGCATCCTGCCCAA
CATCGAGGCTGTGAGCAACGTCCACAACCTCAACTCGGTCAAAGAGTCA
CCCCACGAGCGCATGCATCGCCACATCGAGCTGGCCTGGGCCTTCTCCAC
GGTCATCGGGACGCTGCTTTTCCTAGCAGAGGTCGTGCTGCTCTGCTGGG
TCAAGTTCTTACCTCTCAAGAAGCAGCCAGGCCAGCCAAGGCCCACCAG
CAAGCCCGCCGAGAGTGTCATAGTAGCCAACGTCAGCACCAGCGGCATC
ACCCCGGGTGAGGCGGCAGCCATTGCCTCCACCGCCATCATGGTTCCCTG
TGGCCTGGTTTTCATCGTCTTTGCTGTTCACTTCTACCGCTCCCTGGTCA
GCCATAAGACGGACCGGCAGTTCCAGGAGCTCAATGAGCTGGCCGAGTTT
GCCCGCTTGCAGGACCAGCTGGACCACAGAGGGGACCATTCTCTAACAC
CGGGCACCCACTATGCCTAA//.

The sequence of mOrai1-hOrai1 ECL2 (AESVIV) protein is the following (ECL2 domain is underlined, AESVIV (SEQ ID NO:108) sequence is double underlined):

SEQ ID NO: 141

MHPEPAPPPSHSNPELPVSGGSSTSGSRRSRRRSGDGEPSGAPPLPPPPP
AVSYPDWIGQSYSEVMSLNEHSMQALSWRKLYLSRAKLKASSRTSALLSG
FAMVAMVEVQLDTDHDYPPGLLIVFSACTTVLVAVHLFALMISTCILPNI
EAVSNVHNLNSVKESPHERMHRHIELAWAFSTVIGTLLFLAEVVLLCWVK
FLPLKKQPGQPRPTSKPAESVIVANVSTSGITPGEAAAIASTAIMVPCGL
VFIVFAVHFYRSLVSHKTDRQFQELNELAEFARLQDQLDHRGDHSLTPGT
HYA//.

Using pcDNA3.1/Hygromycin-mOrai1-hOrai1 ECL2 vector and primers with the sequences SEQ ID NOS:142-143 below, we generated mOrai1-hOrai1 ECL2 where amino acid residues 224 to 226 of SEQ ID NO:2 (hOrai1), with the amino acid sequence VST, were mutated to the corresponding amino acid sequence of residues 226 to 228 of SEQ ID NO:72 (mOrai1), having the sequence HSD, referred to as mOrai1-hOrai1 ECL2 (HSD) protein (SEQ ID NO: 145), encoded by mOrai1-hOrai1 ECL2 (HSD) cDNA (SEQ ID NO:144).

The forward primer sequence was:

(SEQ ID NO: 142)

5′-GGCGCAGCAGCCAACCACAGCGACAGCGGCATCACCCC-3′//;
and
the reverse primer sequence was:

(SEQ ID NO: 143)

5′-GGGGTGATGCCGCTGTCGCTGTGGTTGGCTGCTGCGCC-3′.

The sequence of mOrai1-hOrai1 ECL2 (HSD) cDNA is the following (ECL2 domain is underlined, HSD coding sequence is double underlined):

SEQ ID NO: 144

ATGCATCCGGAGCCTGCCCCGCCCCCGAGTCACAGCAATCCGGAGCTTCC
CGTGAGCGGCGGCAGCAGCACTAGCGGCAGCCGCCGGAGCCGCCGCCGC
AGCGGGGACGGGGAGCCCTCGGGGGCCCCACCGCTGCCGCCGCCGCCAC
CCGCCGTCAGCTACCCGGACTGGATCGGCCAGAGTTACTCCGAGGTGAT
GAGCCTCAACGAGCACTCGATGCAGGCGCTGTCCTGGCGCAAGCTCTACT
TAAGCCGCGCCAAGCTCAAAGCTTCCAGCCGGACCTCGGCTCTGCTCTCC
GGCTTCGCCATGGTAGCGATGGTGGAAGTCCAGCTGGACACAGACCATG
ACTACCCACCAGGGTTGCTCATCGTCTTTAGTGCCTGCACCACAGTGCTA
GTGGCCGTGCACCTGTTTGCCCTCATGATCAGCACCTGCATCCTGCCCAA
CATCGAGGCTGTGAGCAACGTCCACAACCTCAACTCGGTCAAAGAGTCA
CCCCACGAGCGCATGCATCGCCACATCGAGCTGGCCTGGGCCTTCTCCAC
GGTCATCGGGACGCTGCTTTTCCTAGCAGAGGTCGTGCTGCTCTGCTGGG
TCAAGTTCTTACCTCTCAAGAAGCAGCCAGGCCAGCCAAGGCCCACCAG
CAAGCCCCCCGCCAGTGGCGCAGCAGCCAACCACAGCGACAGCGGCATC
ACCCCGGGTGAGGCGGCAGCCATTGCCTCCACCGCCATCATGGTTCCCTG
TGGCCTGGTTTTCATCGTCTTTGCTGTTCACTTCTACCGCTCCCTGGTCA
GCCATAAGACGGACCGGCAGTTCCAGGAGCTCAATGAGCTGGCCGAGTTT
GCCCGCTTGCAGGACCAGCTGGACCACAGAGGGGACCATTCTCTAACAC
CGGGCACCCACTATGCCTAA//.

The sequence of mOrai1-hOrai1 ECL2 (HSD) protein is the following (ECL2 domain is underlined, HSD sequence is double underlined):

SEQ ID NO: 145

MHPEPAPPPSHSNPELPVSGGSSTSGSRRSRRRSGDGEPSGAPPLPPPPP
AVSYPDWIGQSYSEVMSLNEHSMQALSWRKLYLSRAKLKASSRTSALLSG
FAMVAMVEVQLDTDHDYPPGLLIVFSACTTVLVAVHLFALMISTCILPNI
EAVSNVHNLNSVKESPHERMHRHIELAWAFSTVIGTLLFLAEVVLLCWVK
FLPLKKQPGQPRPTSKPPASGAAANHSDSGITPGEAAAIASTAIMVPCGL
VFIVFAVHFYRSLVSHKTDRQFQELNELAEFARLQDQLDHRGDHSLTPGT
HYA//.

Using pcDNA3.1/Hygromycin-mOrai1-hOrai1 ECL2 (KPPAE) vector and primers with the sequences SEQ ID NOS:211-212 below, we generated mOrai1-hOrai1 ECL2 where amino acid residues 210 to 217 of SEQ ID NO:2 (hOrai1), with the amino acid sequence RPTSKPPA (SEQ ID NO:125), were mutated to the corresponding amino acid sequence of residues 212 to 219 of SEQ ID NO:72 (mOrai1), having the sequence SPTKPPAE (SEQ ID NO: 124), referred to as mOrai1-hOrai1 ECL2 (SPTKPPAE) protein (SEQ ID NO:214), encoded by mOrai1-hOrai1 ECL2 (SPTKPPAE) cDNA (SEQ ID NO:213).

The forward primer sequence was:

(SEQ ID NO: 211)

5′-GGCCAGCCAAGCCCCACCAAGCC-3′//;
and
the reverse primer sequence was:

(SEQ ID NO: 212)

5′-GGCTTGGTGGGGCTTGGCTGGCC-3′.

The sequence of mOrai1-hOrai1 ECL2 (SPTKPPAE) cDNA is the following (ECL2 domain is underlined, SPTKPPAE (SEQ ID NO:124) coding sequence is double underlined):

SEQ ID NO: 213

ATGCATCCGGAGCCTGCCCCGCCCCCGAGTCACAGCAATCCGGAGCTTCC
CGTGAGCGGCGGCAGCAGCACTAGCGGCAGCCGCCGGAGCCGCCGCCGC
AGCGGGGACGGGGAGCCCTCGGGGGCCCCACCGCTGCCGCCGCCGCCAC
CCGCCGTCAGCTACCCGGACTGGATCGGCCAGAGTTACTCCGAGGTGAT
GAGCCTCAACGAGCACTCGATGCAGGCGCTGTCCTGGCGCAAGCTCTACT
TAAGCCGCGCCAAGCTCAAAGCTTCCAGCCGGACCTCGGCTCTGCTCTCC
GGCTTCGCCATGGTAGCGATGGTGGAAGTCCAGCTGGACACAGACCATG
ACTACCCACCAGGGTTGCTCATCGTCTTTAGTGCCTGCACCACAGTGCTA
GTGGCCGTGCACCTGTTTGCCCTCATGATCAGCACCTGCATCCTGCCCAA
CATCGAGGCTGTGAGCAACGTCCACAACCTCAACTCGGTCAAAGAGTCA
CCCCACGAGCGCATGCATCGCCACATCGAGCTGGCCTGGGCCTTCTCCAC
GGTCATCGGGACGCTGCTTTTCCTAGCAGAGGTCGTGCTGCTCTGCTGGG
TCAAGTTCTTACCTCTCAAGAAGCAGCCAGGCCAGCCAAGCCCCACCAA
GCCGCCCGCCGAGAGTGGCGCAGCAGCCAACGTCAGCACCAGCGGCATC
ACCCCGGGTGAGGCGGCAGCCATTGCCTCCACCGCCATCATGGTTCCCTG
TGGCCTGGTTTTCATCGTCTTTGCTGTTCACTTCTACCGCTCCCTGGTCA
GCCATAAGACGGACCGGCAGTTCCAGGAGCTCAATGAGCTGGCCGAGTTT
GCCCGCTTGCAGGACCAGCTGGACCACAGAGGGGACCATTCTCTAACAC
CGGGCACCCACTATGCCTAA//.

The sequence of mOrai1-hOrai1 ECL2 (SPTKPPAE) protein is the following (ECL2 domain is underlined, SPTKPPAE (SEQ ID NO: 124) sequence is double underlined):

SEQ ID NO: 214

MHPEPAPPPSHSNPELPVSGGSSTSGSRRSRRRSGDGEPSGAPPLPPPPP
AVSYPDWIGQSYSEVMSLNEHSMQALSWRKLYLSRAKLKASSRTSALLSG
FAMVAMVEVQLDTDHDYPPGLLIVFSACTTVLVAVHLFALMISTCILPNI
EAVSNVHNLNSVKESPHERMHRHIELAWAFSTVIGTLLFLAEVVLLCWVK
FLPLKKQPGQPSPTKPPAESVIVANVSTSGITPGEAAAIASTAIMVPCGL
VFIVFAVHFYRSLVSHKTDRQFQELNELAEFARLQDQLDHRGDHSLTPGT
HYA//.

Using pcDNA3.1/Hygromycin-mOrai1-hOrai1 ECL2 (SPTKPPAE) vector and primers with the sequences SEQ ID NOS:215-216 below, we generated mOrai1-hOrai1 ECL2 where amino acid residues 210 to 221 of SEQ ID NO:2 (hOrai1), with the amino acid sequence RPTSKPPASGAA (SEQ ID NO:190), were mutated to the corresponding amino acid sequence of residues 212 to 223 of SEQ ID NO:72 (mOrai1), having the sequence SPTKPPAESVIV (SEQ ID NO: 189), referred to as mOrai1-hOrai1 ECL2 (SPTKPPAESVIV) protein (SEQ ID NO:218), encoded by mOrai1-hOrai1 ECL2 (SPTKPPAESVIV) cDNA (SEQ ID NO:217).

The forward primer sequence was:

(SEQ ID NO: 215)

5′-CCGCCCGCCGAGAGTGTCATAGTAGCCAACGTCAGCACC-3′//;
and
the reverse primer sequence was:

(SEQ ID NO: 216)

5′-GGTGCTGACGTTGGCTACTATGACACTCTCGGCGGGCGG-3′.

The sequence of mOrai1-hOrai1 ECL2 (SPTKPPAESVIV) cDNA is the following (ECL2 domain is underlined, SPTKPPAESVIV (SEQ ID NO: 189) coding sequence is double underlined):

SEQ ID NO: 217

ATGCATCCGGAGCCTGCCCCGCCCCCGAGTCACAGCAATCCGGAGCTTCC
CGTGAGCGGCGGCAGCAGCACTAGCGGCAGCCGCCGGAGCCGCCGCCGC
AGCGGGGACGGGGAGCCCTCGGGGGCCCCACCGCTGCCGCCGCCGCCAC
CCGCCGTCAGCTACCCGGACTGGATCGGCCAGAGTTACTCCGAGGTGAT
GAGCCTCAACGAGCACTCGATGCAGGCGCTGTCCTGGCGCAAGCTCTACT
TAAGCCGCGCCAAGCTCAAAGCTTCCAGCCGGACCTCGGCTCTGCTCTCC
GGCTTCGCCATGGTAGCGATGGTGGAAGTCCAGCTGGACACAGACCATG
ACTACCCACCAGGGTTGCTCATCGTCTTTAGTGCCTGCACCACAGTGCTA
GTGGCCGTGCACCTGTTTGCCCTCATGATCAGCACCTGCATCCTGCCCAA
CATCGAGGCTGTGAGCAACGTCCACAACCTCAACTCGGTCAAAGAGTCA
CCCCACGAGCGCATGCATCGCCACATCGAGCTGGCCTGGGCCTTCTCCAC
GGTCATCGGGACGCTGCTTTTCCTAGCAGAGGTCGTGCTGCTCTGCTGGG
TCAAGTTCTTACCTCTCAAGAAGCAGCCAGGCCAGCCAAGCCCCACCAA
GCCGCCCGCCGAGAGTGTCATAGTAGCCAACGTCAGCACCAGCGGCATC
ACCCCGGGTGAGGCGGCAGCCATTGCCTCCACCGCCATCATGGTTCCCTG
TGGCCTGGTTTTCATCGTCTTTGCTGTTCACTTCTACCGCTCCCTGGTCA
GCCATAAGACGGACCGGCAGTTCCAGGAGCTCAATGAGCTGGCCGAGTTT
GCCCGCTTGCAGGACCAGCTGGACCACAGAGGGGACCATTCTCTAACAC
CGGGCACCCACTATGCCTAA//.

The sequence of mOrai1-hOrai1 ECL2 (SPTKPPAESVIV) protein is the following (ECL2 domain is underlined, SPTKPPAESVIV (SEQ ID NO: 189) sequence is double underlined):

SEQ ID NO: 218

MHPEPAPPPSHSNPELPVSGGSSTSGSRRSRRRSGDGEPSGAPPLPPPPP
AVSYPDWIGQSYSEVMSLNEHSMQALSWRKLYLSRAKLKASSRTSALLSG
FAMVAMVEVQLDTDHDYPPGLLIVFSACTTVLVAVHLFALMISTCILPNI
EAVSNVHNLNSVKESPHERMHRHIELAWAFSTVIGTLLFLAEVVLLCWVK
FLPLKKQPGQPSPTKPPAESVIVANVSTSGITPGEAAAIASTAIMVPCGL
VFIVFAVHFYRSLVSHKTDRQFQELNELAEFARLQDQLDHRGDHSLTPGT
HYA//.

Using pcDNA3.1/Hygromycin-mOrai1-hOrai1 ECL2 (AESVIV) vector and primers with the sequences SEQ ID NOS:219-220 below, we generated mOrai1-hOrai1 ECL2 where amino acid residues 216 to 226 of SEQ ID NO:2 (hOrai1), with the amino acid sequence PASGAAANVST (SEQ ID NO:196), were mutated to the corresponding amino acid sequence of residues 218 to 228 of SEQ ID NO:72 (mOrai1), having the sequence AESVIVANHSD (SEQ ID NO:195), referred to as mOrai1-hOrai1 ECL2 (AESVIVANHSD) protein (SEQ ID NO:222), encoded by mOrai1-hOrai1 ECL2 (AESVIVANHSD) cDNA (SEQ ID NO:221).

The forward primer sequence was:

(SEQ ID NO: 219)

5′-GTGTCATAGTAGCCAACCACAGCGACAGCGGCATCACCCCGG-
3′//;
and
the reverse primer sequence was:

(SEQ ID NO: 220)

5′-CCGGGGTGATGCCGCTGTCGCTGTGGTTGGCTACTATGACAC-3′.

The sequence of mOrai1-hOrai1 ECL2 (AESVIVANHSD) cDNA is the following (ECL2 domain is underlined, AESVIVANHSD (SEQ ID NO:195) coding sequence is double underlined):

SEQ ID NO: 221

ATGCATCCGGAGCCTGCCCCGCCCCCGAGTCACAGCAATCCGGAGCTTCC
CGTGAGCGGCGGCAGCAGCACTAGCGGCAGCCGCCGGAGCCGCCGCCGC
AGCGGGGACGGGGAGCCCTCGGGGGCCCCACCGCTGCCGCCGCCGCCAC
CCGCCGTCAGCTACCCGGACTGGATCGGCCAGAGTTACTCCGAGGTGAT
GAGCCTCAACGAGCACTCGATGCAGGCGCTGTCCTGGCGCAAGCTCTACT
TAAGCCGCGCCAAGCTCAAAGCTTCCAGCCGGACCTCGGCTCTGCTCTCC
GGCTTCGCCATGGTAGCGATGGTGGAAGTCCAGCTGGACACAGACCATG
ACTACCCACCAGGGTTGCTCATCGTCTTTAGTGCCTGCACCACAGTGCTA
GTGGCCGTGCACCTGTTTGCCCTCATGATCAGCACCTGCATCCTGCCCAA
CATCGAGGCTGTGAGCAACGTCCACAACCTCAACTCGGTCAAAGAGTCA
CCCCACGAGCGCATGCATCGCCACATCGAGCTGGCCTGGGCCTTCTCCAC
GGTCATCGGGACGCTGCTTTTCCTAGCAGAGGTCGTGCTGCTCTGCTGGG
TCAAGTTCTTACCTCTCAAGAAGCAGCCAGGCCAGCCAAGGCCCACCAG
CAAGCCCGCCGAGAGTGTCATAGTAGCCAACCACAGCGACAGCGGCATC
ACCCCGGGTGAGGCGGCAGCCATTGCCTCCACCGCCATCATGGTTCCCTG
TGGCCTGGTTTTCATCGTCTTTGCTGTTCACTTCTACCGCTCCCTGGTCA
GCCATAAGACGGACCGGCAGTTCCAGGAGCTCAATGAGCTGGCCGAGTTT
GCCCGCTTGCAGGACCAGCTGGACCACAGAGGGGACCATTCTCTAACAC
CGGGCACCCACTATGCCTAA//.

The sequence of mOrai1-hOrai1 ECL2 (AESVIVANHSD) protein is the following (ECL2 domain is underlined, AESVIVANHSD (SEQ ID NO: 195) sequence is double underlined):

SEQ ID NO: 222

MHPEPAPPPSHSNPELPVSGGSSTSGSRRSRRRSGDGEPSGAPPLPPPPP
AVSYPDWIGQSYSEVMSLNEHSMQALSWRKLYLSRAKLKASSRTSALLSG
FAMVAMVEVQLDTDHDYPPGLLIVFSACTTVLVAVHLFALMISTCILPNI
EAVSNVHNLNSVKESPHERMHRHIELAWAFSTVIGTLLFLAEVVLLCWVK
FLPLKKQPGQPRPTSKPAESVIVANHSDSGITPGEAAAIASTAIMVPCGL
VFIVFAVHFYRSLVSHKTDRQFQELNELAEFARLQDQLDHRGDHSLTPGT
HYA//.

Using pcDNA3.1/Hygromycin-mOrai1-hOrai1 ECL2 (SPTKPPAE) vector and primers with the sequences SEQ ID NOS:223-224 below, we generated mOrai1-hOrai1 ECL2 where amino acid residues 204 to 217 of SEQ ID NO:2 (hOrai1), with the amino acid sequence KQPGQPRPTSKPPA (SEQ ID NO:202), were mutated to the corresponding amino acid sequence of residues 206 to 219 of SEQ ID NO:72 (mOrai1), having the sequence RQAGQPSPTKPPAE (SEQ ID NO:201), referred to as mOrai1-hOrai1 ECL2 (RQAGQPSPTKPPAE) protein (SEQ ID NO:226), encoded by mOrai1-hOrai1 ECL2 (RQAGQPSPTKPPAE) cDNA (SEQ ID NO:225).

The forward primer sequence was:

(SEQ ID NO: 223)

5′-CTTACCTCTCAAGAGGCAGGCAGGCCAGCCAAGC-3′//;
and
the reverse primer sequence was:

(SEQ ID NO: 224)

5′-GCTTGGCTGGCCTGCCTGCCTCTTGAGAGGTAAG-3′.

The sequence of mOrai1-hOrai1 ECL2 (RQAGQPSPTKPPAE) cDNA is the following (ECL2 domain is underlined, RQAGQPSPTKPPAE (SEQ ID NO:201) coding sequence is double underlined):

SEQ ID NO: 225

ATGCATCCGGAGCCTGCCCCGCCCCCGAGTCACAGCAATCCGGAGCTTCC
CGTGAGCGGCGGCAGCAGCACTAGCGGCAGCCGCCGGAGCCGCCGCCGC
AGCGGGGACGGGGAGCCCTCGGGGGCCCCACCGCTGCCGCCGCCGCCAC
CCGCCGTCAGCTACCCGGACTGGATCGGCCAGAGTTACTCCGAGGTGAT
GAGCCTCAACGAGCACTCGATGCAGGCGCTGTCCTGGCGCAAGCTCTACT
TAAGCCGCGCCAAGCTCAAAGCTTCCAGCCGGACCTCGGCTCTGCTCTCC
GGCTTCGCCATGGTAGCGATGGTGGAAGTCCAGCTGGACACAGACCATG
ACTACCCACCAGGGTTGCTCATCGTCTTTAGTGCCTGCACCACAGTGCTA
GTGGCCGTGCACCTGTTTGCCCTCATGATCAGCACCTGCATCCTGCCCAA
CATCGAGGCTGTGAGCAACGTCCACAACCTCAACTCGGTCAAAGAGTCA
CCCCACGAGCGCATGCATCGCCACATCGAGCTGGCCTGGGCCTTCTCCAC
GGTCATCGGGACGCTGCTTTTCCTAGCAGAGGTCGTGCTGCTCTGCTGGG
TCAAGTTCTTACCTCTCAAGAGGCAGGCAGGCCAGCCAAGCCCCACCAA
GCCGCCCGCCGAGAGTGGCGCAGCAGCCAACGTCAGCACCAGCGGCATC
ACCCCGGGTGAGGCGGCAGCCATTGCCTCCACCGCCATCATGGTTCCCTG
TGGCCTGGTTTTCATCGTCTTTGCTGTTCACTTCTACCGCTCCCTGGTCA
GCCATAAGACGGACCGGCAGTTCCAGGAGCTCAATGAGCTGGCCGAGTTT
GCCCGCTTGCAGGACCAGCTGGACCACAGAGGGGACCATTCTCTAACAC
CGGGCACCCACTATGCCTAA//.

The sequence of mOrai1-hOrai1 ECL2 (RQAGQPSPTKPPAE) protein is the following (ECL2 domain is underlined, RQAGQPSPTKPPAE (SEQ ID NO:201) sequence is double underlined):

SEQ ID NO: 226

MHPEPAPPPSHSNPELPVSGGSSTSGSRRSRRRSGDGEPSGAPPLPPPP
PAVSYPDWIGQSYSEVMSLNEHSMQALSWRKLYLSRAKLKASSRTSALLS
GFAMVAMVEVQLDTDHDYPPGLLIVFSACTTVLVAVHLFALMISTCILP
NIEAVSNVHNLNSVKESPHERMHRHIELAWAFSTVIGTLLFLAEVVLL
CWVKFLPLKRQAGQPSPTKPPAESGAAANVSTSGITPGEAAAIASTA
IMVPCGLVFIVFAVHFYRSLVSHKTDRQFQELNELAEFARLQDQLDHR
GDHSLTPGTHYA//.

Using pcDNA3.1/Hygromycin-mOrai1-hOrai1 ECL2 (SPTKPPAESVIV) vector and primers with the sequences SEQ ID NOS:227-228 below, we generated mOrai1-hOrai1 ECL2 where amino acid residues 210 to 226 of SEQ ID NO:2 (hOrai1), with the amino acid sequence RPTSKPPASGAAANVST (SEQ ID NO:229), were mutated to the corresponding amino acid sequence of residues 212 to 228 of SEQ ID NO:72 (mOrai1), having the sequence SPTKPPAESVIVANHSD (SEQ ID NO:230), referred to as mOrai1-hOrai1 ECL2 (SPTKPPAESVIVANHSD) protein (SEQ ID NO:232), encoded by mOrai1-hOrai1 ECL2(SPTKPPAESVIVANHSD) cDNA (SEQ ID NO:231).

The forward primer sequence was:

(SEQ ID NO: 227)

5′-GTGTCATAGTAGCCAACCACAGCGACAGCGGCATCACCCCGG-
3′//;
and
the reverse primer sequence was:

(SEQ ID NO: 228)

5′-CCGGGGTGATGCCGCTGTCGCTGTGGTTGGCTACTATGACAC-3′.

The sequence of mOrai1-hOrai1 ECL2 (SPTKPPAESVIVANHSD) cDNA is the following (ECL2 domain is underlined, SPTKPPAESVIVANHSD (SEQ ID NO:230) coding sequence is double underlined):

SEQ ID NO: 231

ATGCATCCGGAGCCTGCCCCGCCCCCGAGTCACAGCAATCCGGAGCTTCC
CGTGAGCGGCGGCAGCAGCACTAGCGGCAGCCGCCGGAGCCGCCGCCGC
AGCGGGGACGGGGAGCCCTCGGGGGCCCCACCGCTGCCGCCGCCGCCAC
CCGCCGTCAGCTACCCGGACTGGATCGGCCAGAGTTACTCCGAGGTGAT
GAGCCTCAACGAGCACTCGATGCAGGCGCTGTCCTGGCGCAAGCTCTACT
TAAGCCGCGCCAAGCTCAAAGCTTCCAGCCGGACCTCGGCTCTGCTCTCC
GGCTTCGCCATGGTAGCGATGGTGGAAGTCCAGCTGGACACAGACCATG
ACTACCCACCAGGGTTGCTCATCGTCTTTAGTGCCTGCACCACAGTGCTA
GTGGCCGTGCACCTGTTTGCCCTCATGATCAGCACCTGCATCCTGCCCAA
CATCGAGGCTGTGAGCAACGTCCACAACCTCAACTCGGTCAAAGAGTCA
CCCCACGAGCGCATGCATCGCCACATCGAGCTGGCCTGGGCCTTCTCCAC
GGTCATCGGGACGCTGCTTTTCCTAGCAGAGGTCGTGCTGCTCTGCTGGG
TCAAGTTCTTACCTCTCAAGAAGCAGCCAGGCCAGCCAAGCCCCACCAA
GCCGCCCGCCGAGAGTGTCATAGTAGCCAACCACAGCGACAGCGGCATC
ACCCCGGGTGAGGCGGCAGCCATTGCCTCCACCGCCATCATGGTTCCCTG
TGGCCTGGTTTTCATCGTCTTTGCTGTTCACTTCTACCGCTCCCTGGTCA
GCCATAAGACGGACCGGCAGTTCCAGGAGCTCAATGAGCTGGCCGAGTTT
GCCCGCTTGCAGGACCAGCTGGACCACAGAGGGGACCATTCTCTAACAC
CGGGCACCCACTATGCCTAA//.

The sequence of mOrai1-hOrai1 ECL2 (SPTKPPAESVIVANHSD) protein is the following (ECL2 domain is underlined, SPTKPPAESVIVANHSD (SEQ ID NO:230) sequence is double underlined):

SEQ ID NO: 232

MHPEPAPPPSHSNPELPVSGGSSTSGSRRSRRRSGDGEPSGAPPLPPPPP
AVSYPDWIGQSYSEVMSLNEHSMQALSWRKLYLSRAKLKASSRTSALLSG
FAMVAMVEVQLDTDHDYPPGLLIVFSACTTVLVAVHLFALMISTCILPNI
EAVSNVHNLNSVKESPHERMHRHIELAWAFSTVIGTLLFLAEVVLLCWV
KFLPLKKQPGQPSPTKPPAESVIVANHSDSGITPGEAAAIASTAIMVPCG
LVFIVFAVHFYRSLVSHKTDRQFQELNELAEFARLQDQLDHRGDHSLT
PGTHYA//.

FIG. 15A-B show that the human ECL2 is sufficient for binding by all the mAbs since they all bound to mOrai1-hOrai1 ECL2 chimera but not to mouse Orai1. We wanted to ascertain where the inventive mAbs specifically bind to the human Orai1 ECL2 region. To accomplish this we started with the mOrai1-hOrai1 ECL2 chimera and made various mutants replacing the human amino acids with the mouse amino acids where there is a difference between the two species (see, FIG. 14). A convenient way to visualize where the changes where made in this “loss of binding” analysis is in Table 11A-B. Since, the mOrai1-hOrai1 ECL2 chimera that is bound by all the mAbs, the mutants that are no longer bound by the mAbs indicate that the subregions where the changes were made in the mutants play an important role in the binding by the mAbs.

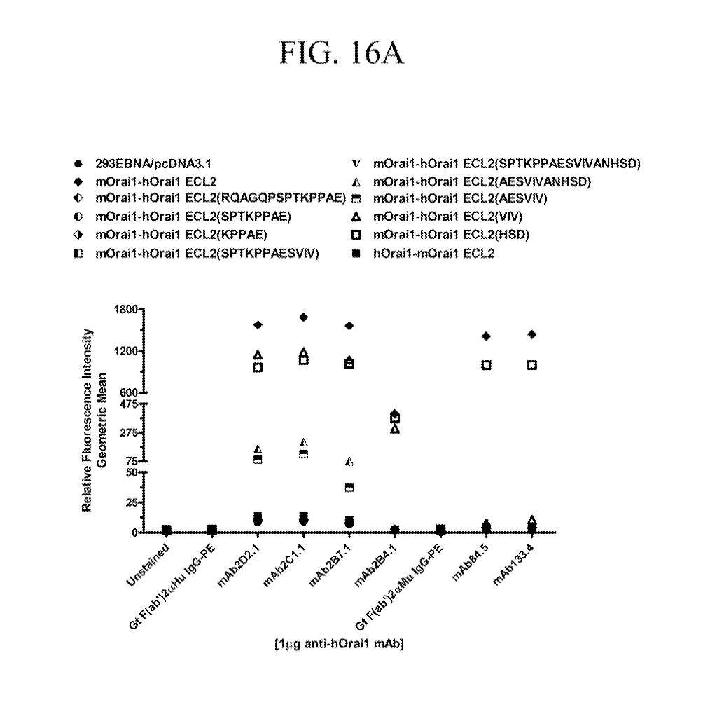

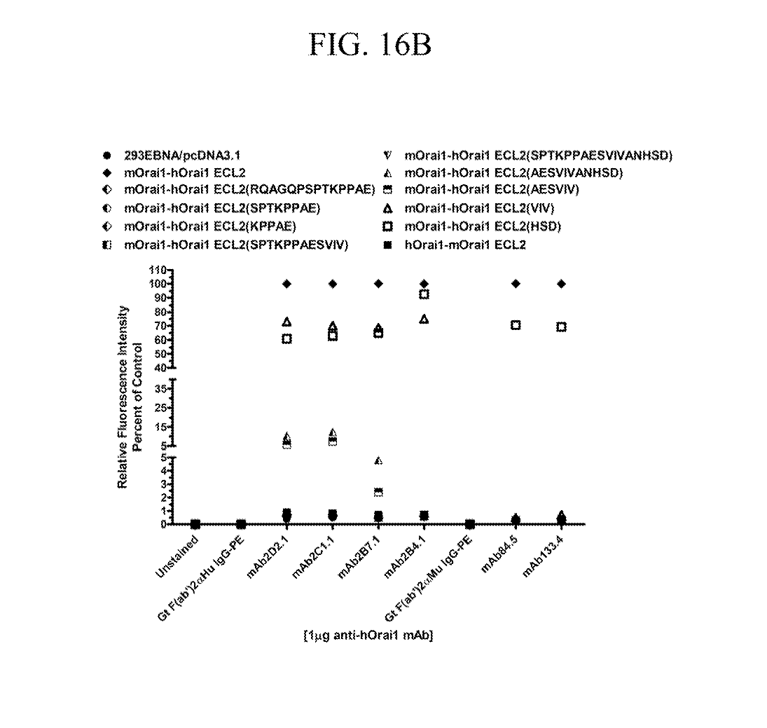

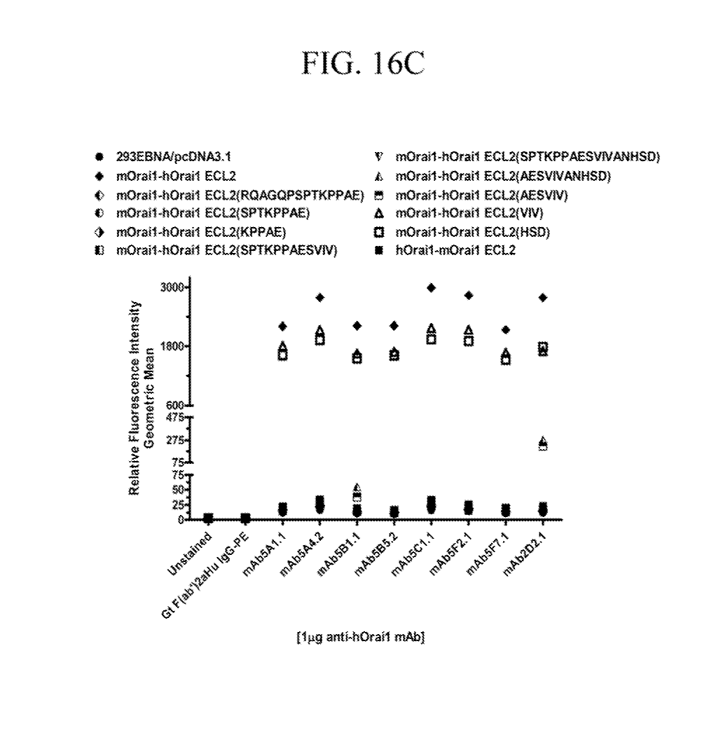

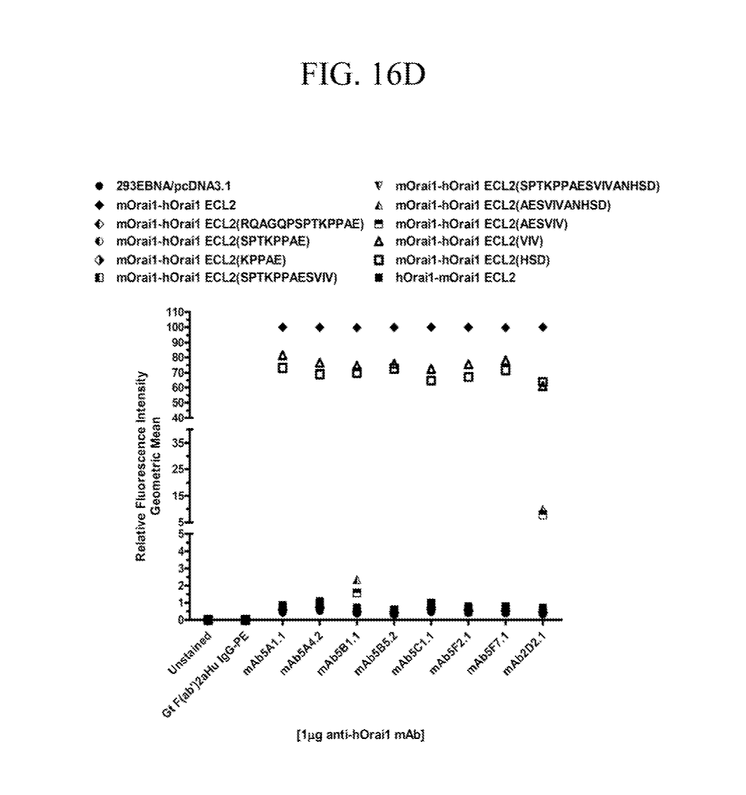

Table 11A-B shows loss of binding ability of monoclonal antibodies from Campaign 1 (Table 11A) and Campaign 2 (Table 11B) to mOrai1-hOrai1 ECL2 chimera mutants as determined by FACS. Table 11A and Table 11B feature a schematic representation of the POC data from FIG. 16B and FIG. 16D, respectively, where the binding results are recorded as (+++) denoting binding POC from 40% to 100%, (++) for POC of 5% to less than 40%, (+) for POC from 1% to less than 5% and (−) as lack of binding with POC less than 1%. The top of Table 11A-B shows an alignment between the human and mouse Orai1 protein in the ECL2 region only with the human amino acids represented in capital letters, the mouse ECL2 amino acids are all lower case letters and the underlined amino acids denotes differences between human and mouse protein sequences. The chimera constructs (Ch.) are numbered on the left hand side starting with the mOrai1-hOrai1 ECL2 as 1 and the hOrai1-mOrai1 ECL2 as Ch. 11. The chimeric Ch. 2 to 10 are mOrai1-hOrai1 ECL2 mutants with specific human amino acids that are replaced with their mouse counterparts where there is a difference in sequence between the two species in the ECL2 region. The human to mouse amino acid changes in the table are represented by lower case letters and the dashes denote no changes. For Table 11A under “Binding”, the human mAb2D2.1, mAb2C1.1 and mAb2B7.1 are grouped together in the left column, human mAb2B4.1 is by itself in the middle column and mouse mAb84.5 and 133.4 are grouped together in the right column. For Table 11B under “Binding”, the mAb 2D2.1 from Campaign 1 is provided for comparison in the left column, the middle column is occupied by purified mAb 5B1.1 and the rest of the Campaign 2 purified mAbs are grouped together in the right-most column. The Geo Means of the Unstained control and the directly labeled secondary antibody control binding to cells transfected with chimera constructs and the pcDNA3.1 vector control were all low and all together represent “Negative Controls”. The RFI-POC was calculated from the relative fluorescence intensity geometric mean (Geo Mean) using the algorithm (Algorithm II, below) of Geo Mean of a mAb binding to cells expressing a chimera minus the average Geo Mean of Negative Controls, then divided by the Geo Mean of the particular mAb binding to Ch. 1 (mOrai-hOrai1 ECL2, SEQ ID NO:97), the entire quantity multiplied by 100. (Algorithm II).

Algorithm II (inserting “mAb 2D2.1” and “Ch.2” as an example of particular mAb and sample chimera of interest, for which others of interest can be substituted):

$$RFI \text{-} POC of 2 D 2.1 binding to Ch . 2 = \frac{\begin{matrix}Geo Mean of 2 D 2.1 binding to Ch . 2 - \\ Average Geo Mean of Negative Controls\end{matrix}}{Geo Mean of 2 D 2.1 binding to Ch . 1} \times 100$$

TABLE 11A
Binding by monoclonal antibodies (Campaign 1) to mOrai1-hOrai1
ECL2 chimera mutants as determined by FACS.

| | | Binding | | |
|---|---|---|---|---|
| Ch. | 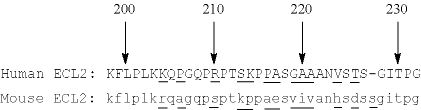 | 2D2.1 2C1.1 2B7.1 | 2B4.1 | 84.5 133.4 |
| 1 | mOrai1/hOrai1 ECL2(KFLPLKKQPGQPRPTSKPPASGAAANVSTS-GITPG) | +++ | ++ | +++ |
| 2 | mOrai1/hOrai1 ECL2(------r-a---s--kp-ae----------------) | − | − | − |
| 3 | mOrai1/hOrai1 ECL2(------------s--kp-ae----------------) | − | − | − |
| 4 | mOrai1/hOrai1 ECL2(---------------kp-ae----------------) | − | − | − |
| 5 | mOrai1/hOrai1 ECL2(------------s--kp-ae-viv------------) | − | − | − |
| 6 | mOrai1/hOrai1 ECL2(------------s--kp-ae-viv--hsd-------) | − | − | − |
| 7 | mOrai1/hOrai1 ECL2(------------------ae-viv--hsd-------) | ++/+ | − | − |
| 8 | mOrai1/hOrai1 ECL2(------------------ae-viv------------) | ++/+ | − | − |
| 9 | mOrai1/hOrai1 ECL2(---------------------viv------------) | +++ | ++ | − |
| 10 | mOrai1/hOrai1 ECL2(--------------------------hsd-------) | +++ | ++ | +++ |
| 11 | hOrai1/mOrai1 ECL2(kflplkrqagqpsptkppaesvivanhsdssgitpg) | − | − | − |

TABLE 11B
Binding by monoclonal antibodies (Campaign 2) to mOrai1-hOrai1
ECL2 chimera mutants as determined by FACS.

| | | Binding | | |
|---|---|---|---|---|
| Ch. | 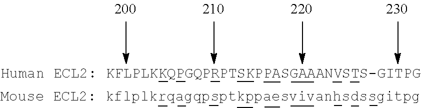 | 2D2.1 | 5B1.1 | 5A1.1 5A4.2 5B5.2 5C1.1 5F2.1 5F7.4 |
| 1 | mOrai1/hOrai1 ECL2(KFLPLKKQPGQPRPTSKPPASGAAANVSTS-GITPG) | +++ | +++ | +++ |
| 2 | mOrai1/hOrai1 ECL2(------r-a---s--kp-ae----------------) | − | − | − |
| 3 | mOrai1/hOrai1 ECL2(------------s--kp-ae----------------) | − | − | − |
| 4 | mOrai1/hOrai1 ECL2(---------------kp-ae----------------) | − | − | − |
| 5 | mOrai1/hOrai1 ECL2(------------s--kp-ae-viv------------) | − | − | − |
| 6 | mOrai1/hOrai1 ECL2(------------s--kp-ae-viv--hsd-------) | − | − | − |
| 7 | mOrai1/hOrai1 ECL2(------------------ae-viv--hsd-------) | ++/+ | + | − |
| 8 | mOrai1/hOrai1 ECL2(------------------ae-viv------------) | ++/+ | + | − |
| 9 | mOrai1/hOrai1 ECL2(---------------------viv------------) | +++ | +++ | +++ |
| 10 | mOrai1/hOrai1 ECL2(--------------------------hsd-------) | +++ | +++ | +++ |
| 11 | hOrai1/mOrai1 ECL2(kflplkrqagqpsptkppaesvivanhsdssgitpg) | − | − | − |

FIG. 16A shows that raw Geo Mean data of the “loss of binding” experiment to ascertain the subregions within ECL2 that are important in the binding by recombinant mAbs from Campaign 1 as well as from purified mAb 84.5 and mAb133.4. FIG. 16C shows similar loss of binding data from the seven purified mAbs from Campaign 2 along with recombinant mAb 2D2.1 from Campaign 1 for comparison. The Geo Means of the Unstained control and the directly labeled secondary antibody control binding to cells transfected with chimera constructs and the pcDNA3.1 vector control were all low and together represent “Negative Controls”. The Geo Means of the mAbs to the mOrai1-hOrai1 chimera represents the maximal binding ranging from the low hundreds to the low thousands depending on the mAbs. As seen in the other figures, the staining of recombinant mAb2B4.1 from Campaign 1 is significantly lower than and is around 30% compare to the other mAbs. Because of differences in the Geo Means of the mAbs binding to mOrai1-hOrai1 ECL2, we felt the need to re-plot the Geo Means as a percent of control (POC) using the background staining as zero and the mOrai1-hOrai1 ECL2 depending on the mAb as the highest attainable for the particular mAb to calculate the percent of control (POC) for each sample. FIG. 16B and FIG. 16D show plots of the POC for Campaign 1 human recombinant mAbs, purified mouse mAbs 84.5 and 133.4, and purified human mAbs from Campaign 2, respectively. In FIG. 16D, the value for the mOrai1-hOrai1 ECL2 is set at 100% but the value for the hOrai1-mOrai1 ECL2 was not zero probably due to endogenous human Orai1 expression in HEK-293 cells. (E.g., Stemfeld et al., Activation of muscarinic receptors reduces store-operated Ca²⁺entry in HEK-293 cells, Cellular Signalling 19:1457-64 (2007); Fasolato et al., Store depletion triggers the calcium release-activated calcium current (ICRAC) in macrovascular endothelial cells: a comparison with Jurkat and embryonic kidney cell lines, Pfluegers Arch.-Eur. J. Physiol. 436(1):69-74 (1998)).

It is clearly seen from Table 11A-B, that all the human recombinant mAbs from Campaign 1, and also purified mAbs 84.5 and 133.4 and purified human mAbs from Campaign 2, lose binding to chimeras Ch.2 to Ch. 6 implying that a subset of amino acid residues 204 to 217 of SEQ ID NO:2 is important for their binding. Conversely, the binding observed to the mOrai1-hOrai1 ECL2 (HSD) (Ch. 10) was comparable to the binding to mOrai1-hOrai1 ECL2 (Ch. 1) by all the mAbs, which indicates that the subregion from amino acids 224 to 226 of SEQ ID NO:2 is not involved in the binding (FIG. 16B and FIG. 16D and Table 11A-B). For mAb 84.5 and mAb 133.4, there was complete loss of binding to mOrai1-hOrai1 ECL2 (VIV) (Ch. 9) mutant that was not observed with all the recombinant mAbs from Campaign 1 and purified mAb from Campaign 2, indicating that the subregion from amino acid residues 219 to 221 of SEQ ID NO:2 plays an important role in the binding by mAb 84.5 and mAb 133.4 (FIG. 16A and FIG. 16C). While there was still some binding by the recombinant mAbs 2C1.1, 2D2.1 and 2B7.1 from Campaign 1 and purified mAb 5B1.1 from Campaign 2 to mOrai1-hOrai1 ECL2 (AESVIVANHSD) (Ch. 7) and mOrai1-hOrai1 ECL2 (AESVIV) (Ch. 8) mutants, the binding was a small fraction of what was observed with these mAbs in binding to mOrai1-hOrai1 ECL2 (Ch. 1; FIG. 16B and FIG. 16D and Table 11A-B). This indicates that the subregion from amino acid residues 216 to 221 of SEQ ID NO:2 plays some role in the binding by mAb2C1.1, mAb 2D2.1 and mAb 2B7.1 and mAb 5B1.1. The lack of binding by mAb 2B4.1 to Ch. 7 and Ch. 8 may have been due to relatively low binding affinity of mAb 2B4.1. On the other hand, the lack of binding to Ch. 7 and Ch. 8 by the purified mAbs 5A1.1, 5A4.2, 5B5.2, 5C1.1, 5F2.1 and 5F7.1 from Campaign 2 is real and reflects the subregion from 216 to 221 of SEQ ID NO:2 plays a critical role in their binding (FIG. 16D and Table 11B). The mAb 5B1.1 still binds but weakly to mOrai1-hOrai1 ECL2 (AESVIVANHSD) (Ch. 7) and to mOrai1-hOrai1 ECL2 (AESVIV) (Ch. 8), which is reminiscent of mAb 2B7.1 from Campaign 1. While amino acid residue 218 of SEQ ID NO:2 is important for the binding of recombinant mAbs from Campaign 1 and the purified mAbs from Campaign 2 (FIGS. 12A and 12B). Furthermore, a subset of positions 219 to 221 of SEQ ID NO:2 is important for the binding of mAb 84.5 and mAb 133.4, but not for the recombinant mAbs from Campaign 1 and the purified mAbs from Campaign 2 (Table 11A-B and FIG. 16A-D). Therefore from the “loss of binding” experiment, we concluded that a subset of amino acid residues 204 to 217 of SEQ ID NO:2 (the human Orai1 sequence) is important for the binding by the recombinant mAbs 2D2.1, 2C1.1, 2B7.1 and 2B4.1 from Campaign 1 and by the purified mAbs 5A1.1, 5A4.2, 5B1.1, 5B5.2, 5C1.1, 5F2.1 and 5F7.1 and a subset of amino acids 204 to 223 of SEQ ID NO:2 is critical for the binding of mAb 84.5 and mAb 133.4.

Interpreting the “loss of binding” experiments (FIG. 16A-D and Table 11A-B) together with the “gain of binding” experiments (FIG. 17A-D and Table 10A-B) minimizes any possibility that an improper presentation of the mutants on the surface of HEK-293 cells could have been involved in the results we observed. In summary, the “loss of binding” experiment for the human recombinant mAb 2C1.1, mAb 2D2.1, mAb 2B7.1 and mAb 2B4.1 from Campaign 1 and the human purified mAb 5A1.1, mAb 5A4.2, mAb 5B1.1, mAb 5B5.2, mAb 5C1.1, mAb 5F2.1 and mAb5F7.1 from Campaign 2 indicated that a subset of amino acid residues 204 to 217 of SEQ ID NO:2 (human Orai1) is important for binding. The “gain of binding” experiment was consistent with it and narrowed this to a subset of amino acid residues 207-217 of SEQ ID NO:2. While the footprint for binding by the mAbs from Campaign 1 and 2 was the same, the emphasis of the binding was slightly different in that the purified mAb 5A1.1, mAb 5A4.2, mAb 5C1.1, mAb 5F2.1 and mAb 5F7.1 bind more strongly to the subregion from 213 to 217 of SEQ ID NO:2. The binding to human Orai1 by recombinant mAb 2C1.1, mAb 2D2.1, mAb 2B7.1 and mAb 2B4.1 from Campaign 1 and the purified mAb 5B1.1 and 5B5.2 from Campaign 2 emphasized more strongly a subset of residues 207 to 213 (FIGS. 17B and 17D and Table 10A-B). The binding by mAb 2B4.1 was only around 30% of the other Campaign 1 mAbs (FIG. 16A and FIG. 17A). The “loss of binding” experiment for mAb 84.5 and mAb 133.4 demonstrated that a subset of amino acid residues 204 to 223 of SEQ ID NO:2 was important for binding. The conclusion from the “gain of binding” experiment for mAb 84.5 and mAb 133.4 supported this conclusion and further narrowed to a subset of amino acid residues 207 to 223 of SEQ ID NO:2. In addition, a subset of amino acid residues 218 to 221 of SEQ ID NO:2 is critically important for the binding by mAb 84.5 and mAb 133.4 (FIG. 12A and FIG. 17A). Interestingly, the importance of this region for mAb84.5 and mAb 133.4 is not shared for the recombinant mAbs from Campaign 1 and the purified mAbs fgrom Campaign 2 and is a distinction between the murine and the human mAbs against human Orai1 channel that we characterized.

### Example 9

### Assessment of Commercially Available Anti-Orai1 Antibodies in Binding Human Orai1

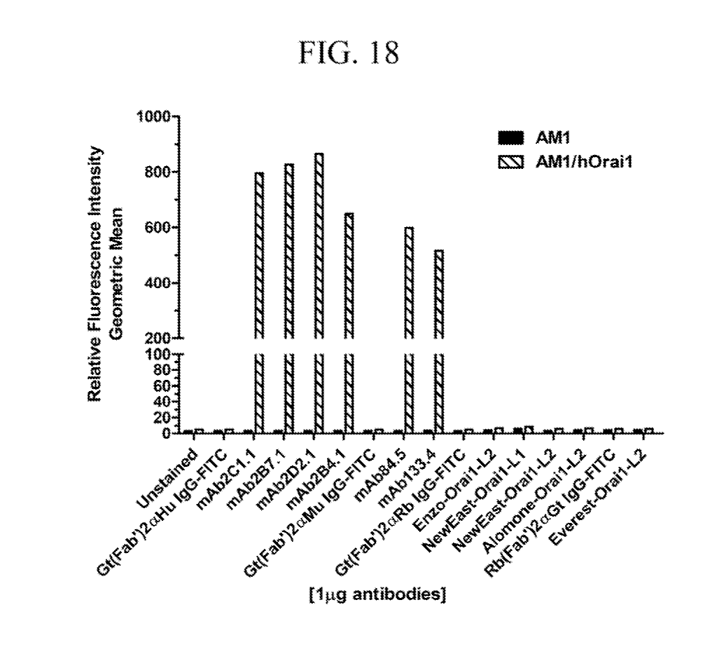

AM1-CHO parental and AM1/hOrai1/hSTIM1-YFP cells (see, Example 1) were used to assess binding of several commercially available polyclonal anti-Orai1 antibodies to human Orai1. Table 12 lists the commercially available antibodies to human Orai1 that were tested, which according to the manufacturers' product inserts, were raised against various peptides from hOrai1 ECL1 and ECL2 domains. The antibodies were polyclonal antibodies raised in rabbits or goats, and none were monoclonal antibodies. Based on the results shown in FIG. 18, the binding of the commercially available antibodies was deemed “not detectable” using AM1/CHO expressing human Orai1 in a FACS binding assay as described herein.

Cells were washed once with ice-cold 1×PBS, resuspended in ice-cold FACS buffer (1×D-PBS+2% goat serum) and 2×10⁵ cell in 1001 were stained per antibody combination. All antibody incubation steps were performed on ice for 1 hour. Cells were first incubated with 1 μg of unlabeled mouse anti-hOrai1 (mAb 84.5 or mAb 133.4) or human anti-hOrai1 (mAb 2C1.1, mAb 2B7.1, mAb 2D2.1, or mAb 2B4.1) monoclonal antibodies, or the respective commercially available goat or rabbit polyclonal antibodies shown in Table 12, followed by a wash with 200 μL of FACS buffer. Next, the unlabelled antibody was detected using goat [“Gt”]F(ab′)₂ anti-mouse [“Mu”]IgG-FITC, goat F(ab′)₂ anti-human [“Hu”]IgG-FITC, goat F(ab′)₂ anti-rabbit [“Rb”]IgG-FITC, or rabbit F(ab′)₂ anti-goat IgG-FITC, as appropriate depending on the mammalian source of the antibodies tested, followed by a wash with 200 μL of ice-cold FACS buffer before flow cytometry analysis. Unstained cells and cells stained with detecting antibodies were used as negative controls. The values of relative level of fluorescence were calculated using FCS Express (De Novo Software) and mean values were calculated using log-transformed data (geometric mean). A binding comparison from the results is shown in FIG. 18, which illustrates that there was no detectable binding to human Orai1 expressed on the surface of AMl/hOrai1/hSTIM1-YFP cells by any of the commercially available antibodies, compared to negative controls, while mAbs of the present invention bound strongly to human Orai1 expressed on the surface of AM1/CHO cells.

TABLE 12
Binding of commercially available polyclonal antibodies to human Orai1
expressed on AM1/hOrai1/hSTIM1-YFP cells (see Example 1). The product number
of each antibody is listed in the leftmost column in parentheses. Source animals and
antigen used are as described in the vendor's product insert for each.

| Name | Source | Antigen | Vendor | Binding |
|---|---|---|---|---|
| Anti-human Orai1 | Rabbit polyclonal | a.a. 203-214 of | Alomone Labs Ltd. | ND |
| (extracellular) | Ab | human Orai1 | Jerusalem, Israel | |
| (ACC-060) | | | | |
| Anti-Orai1 | Rabbit polyclonal | a.a. 203-214 of | Enzo Life Sciences | ND |
| (extracellular) Antibody | Ab | human Orai1 | International, Inc. | |
| (SA-647) | | | Plymouth Meeting, PA | |
| Goat anti-ORAIl/ | Goat polyclonal Ab | a.a. 203-215 of | Everest Biotech Ltd. | ND |
| CRAM1 antibody | | human Orai1 | Oxfordshire, UK | |
| (EB09022) | | | | |
| Orai1 Rabbit Polyclonal | Rabbit polyclonal | A peptide from | NewEast Biosciences | ND |
| Antibody (Orai1-L2) | Ab | the extracellular | Malvern, PA | |
| (21002) | | loop 2 region of | | |
| | | human Orai1 | | |
| Orai1 Rabbit Polyclonal | Rabbit polyclonal | A peptide from | NewEast Biosciences | ND |
| Antibody (Orai1-L1) | Ab | the extracellular | Malvern, PA | |
| (21001) | | loop 1 region of | | |
| | | human Orai1 | | |

ND: Not Detectable

### Example 10

### Further Characterization of Commercially Available Anti-Orai1 Polyclonal Antibodies in Binding mOrai1-hOrai1 ECL2 and hOrai1-mOrai1 ECL2 Chimeric Mutants

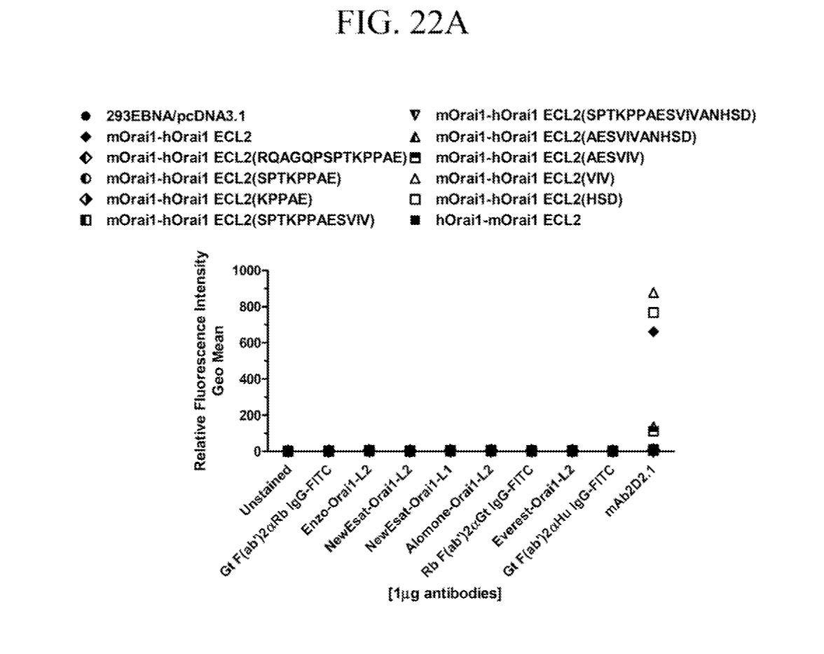

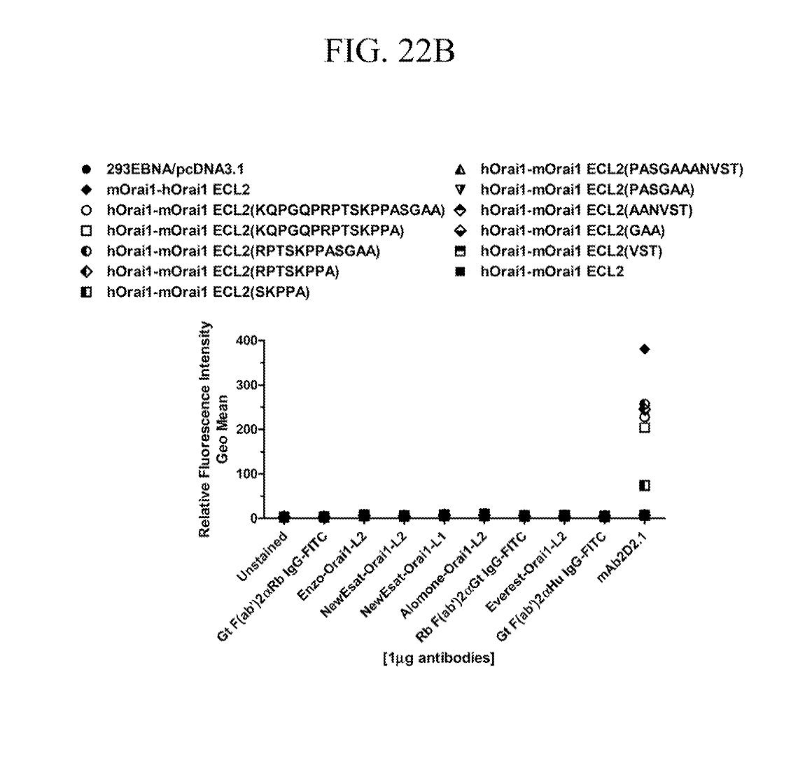

The “loss of binding” and the “gain of binding” experiments described herein above indicated that a subset of amino acid residues 204 to 217 of SEQ ID NO:2 is critical for the binding by the human recombinant and purified mAbs generated from Campaign 1 and Campaign 2, respectively (FIG. 16A-D, Table 11A-B, FIG. 17A-D, Table 10A-B) and a subset of amino acids 204 to 223 of SEQ ID NO:2 is important for the binding of mAb 84.5 and mAb 133.4 (FIG. 16A-D and Table 10A and 11A). Therefore we examined the commercially available anti-hOrai1 antibodies (see, Table 12 in Example 9) for their ability to bind to the hOrai1-mOrai1 ECL2, hOrai1-mOrai1 ECL2 chimeric mutants (Ch. 2 to Ch. 11) (Table 10A-B), mOrai1-hOrai1 ECL2 and mOrai1-hOrai1 ECL2 chimeric mutants (Ch. 2 to Ch. 11) (Table 11A-B) (see Example 8) as described in the “loss of binding” and the “gain of binding” experiments by embodiments of the inventive mAbs. Table 12 (in Example 9 herein) lists the commercially available antibodies to human Orai1 that were tested, which according to the manufacturers' product inserts, were raised against various peptides from hOrai1 ECL1 and ECL2 domains. The antibodies were polyclonal antibodies raised in rabbits or goats, and none were monoclonal antibodies. Based on the results shown in FIG. 22A-B, the binding of the commercially available antibodies was deemed “not detectable” using 293EBNA expressing hOrai1-mOrai1 ECL2, hOrai1-mOrai1ECL2 chimeric mutants (Ch. 2 to Ch. 11) and mOrai1-hOrai1 ECL2 or mOrai1-hOrai1 ECL2 chimeric mutants (Ch. 2 to Ch. 11) in a FACS binding assay as described herein, while mAb2D2.1 of the present invention bound strongly to mOrai1-hOrai1 ECL2 (SEQ ID NO:97), hOrai1-mOrai1ECL2 (KQPGQPRPTSKPPASGAA) (Ch. 2; SEQ ID NO:210), mOrai1-hOrai1 ECL2 (KQPGQPRPTSKPPA) (Ch. 3; SEQ ID NO:204), hOrai1-mOrai1 ECL2 (RPTSKPAASGAA) (Ch. 4; SEQ ID NO:192), mOrai1-hOrai1 ECL2 (RPTSKPPA) (Ch. 5; SEQ ID NO:129), mOrai1-hOrai1 ECL2 (VIV) (Ch. 9; SEQ ID NO:137) or mOrai1-hOrai1 ECL2 (HSD) (Ch. 10; SEQ ID NO: 145) and a small fraction of binding by mAb2D2.1 to hOrai1-mOrai1 ECL2 (SKPPA) (Ch. 6; SEQ ID NO:103), mOrai1-hOrai1 ECL2 (AESVIVANHSD) (Ch. 7; SEQ ID NO: 222) or mOrai1-hOrai1 ECL2 (AESVIV) (Ch. 8; SEQ ID NO: 141) which are consistent with what were observed in FIGS. 16A-D and FIGS. 17A-D. Methods are further described below.

Transient Expression for FACS Binding Analysis.

One day prior to transfection, 293EBNA cells were plated at 3.5×10⁶ cells/dish in 10 mL of growth medium onto 100-mm tissue culture dishes. For one 100-mm dish, 10 μg of DNA was diluted in 460 μL of Opti-MEM, mixed gently, and incubated at room temperature for 5 min. Then, 40 μL of FuGene HD transfection reagent was added to the mixture, mixed gently, and incubated at room temperature for 20 minutes. The transfection mixture was added drop-wise onto the cells and the dish was gently swirled to ensure uniform distribution of the complex.

FACS Binding Analysis.

Transfected 293EBNA cells transiently expressed hOrai1-mOrai1 ECL2, hOrai1-mOrai1 ECL2 chimeric mutants, mOrai1-hOrai1 ECL2 or mOrai1-hOrai1 ECL2 chimeric mutants. The transfected cells were harvested at 48 hours post-transfection. Cells transfected with pcDNA3.1 were used as negative controls. Cells were washed once with ice-cold 1×PBS, resuspended in ice-cold FACS buffer (1×D-PBS+2% goat serum), and 2×10⁵ cells in 100 μL were stained per antibody combination. All antibody incubation steps were performed on ice for 1 hour. Cells were first incubated with 1 μg of the commercially available antibodies to human Orai1 followed by a wash with 200 μL of FACS buffer. Next, the unlabelled antibody was detected using goat F(ab′)₂ anti-rabbit [“Rb”]IgG-FITC, or rabbit F(ab′)₂ anti-goat IgG-FITC, as appropriate depending on the mammalian source of the antibodies tested, followed by a wash with 200 μL of ice-cold FACS buffer before flow cytometry analysis. Cells stained with unlabeled human anti-hOrai1 monoclonal antibodies (mAb 2D2.1) and detected with goat F(ab′)₂ anti-human [“Hu”]IgG-FITC was used as a positive control. Unstained cells and cells stained with detecting antibodies were used as negative controls. The values of relative level of fluorescence were calculated using FCS Express (De Novo Software) and mean values were calculated using log-transformed data (geometric mean). A binding comparison from the results is shown in FIG. 22A-B, which illustrates that there was no detectable binding to human Orai1 expressed on the surface of 293EBNA cells expressing hOrai1-mOrai1 ECL2, hOrai1-mOrai1ECL2 chimeric mutants (Ch. 2 to Ch. 11), mOrai1-hOrai1 ECL2 and mOrai1-hOrai1 ECL2 chimeric mutants (Ch. 2 to Ch. 11) by any of the commercially available antibodies, compared to negative controls.

### Example 11

### Evaluation of Commercially Available Anti-Orai1 Polyclonal Antibodies in Detecting hOrai1Proteins by Western Analysis

We further assessed commercially available anti-hOrai1 antibodies (see, Table 12 in Example 9) for their ability to detect hOrai1 proteins by Western analysis under native, reducing and non-reducing conditions with HEK-293, HEK-293/hOrai1/hSTIM1 BB6.3, Jurkat, AM1/CHO and AM1/hOrai1 cell lysates.

Preparation of Whole Cell Lysates.

HEK-293, HEK-293/hOrai1/hSTIM1 BB6.3, Jurkat, AM1/CHO and AM1/hOrai1 cells were harvested and rinsed twice with ice-cold 1×PBS, then solubilized in cell lysis buffer (1% Triton X-100, 0.1 M NaCl, 0.05 M Tris.HCl (pH8.0), 1 mM Na₃VO₄) containing Protease Inhibitor Cocktail (Roche). The particulate material was centrifuged at 14,000 rpm for 15 min at 4° C. and supernatants were stored at −80° C.

Western Analysis of Commercially Available Polyclonal Antibodies in Detecting hOrai1 Proteins

For detecting native confirmation of hOrai1, approximately 5 μg of cell lysates from each of HEK-293, HEK-293/hOrai1/hSTIM1 BB6.3, Jurkat, AM1/CHO and AM1/hOrai1 in Novex® Native Tris-Glycine Sample buffer (Invitrogen) were separated by electrophoresis through 4-20% Tris-Glycine polyacrylamide gels (Invitrogen) in Novex® Tris-Glycine Native Running Buffer (Invitrogen) and transferred to nitrocellulose filters (Invitrogen). For detecting denatured hOrai1 proteins, approximately 5 μg of cell lysates from each of HEK-293, HEK-293/hOrai1/hSTIM1 BB6.3, Jurkat, AM1/CHO and AM1/hOrai1 in Novex® Tris-Glycine SDS Sample buffer (Invitrogen) under non-reducing or reducing (NuPAGE® Sample Reducing Agent, Invitrogen) conditions were separated by electrophoresis through 4-20% Tris-Glycine polyacrylamide gels (Invitrogen) in Novex® Tris-Glycine Running Buffer (Invitrogen) and transferred to nitrocellulose filters (Invitrogen). The immunoblots were incubated in a 1:500 dilution of rabbit anti-hOrai1 polyclonal antibody from Alomone Labs or Enzo Life Sciences or in a 1:500 dilution of goat anti-hOrai1 antibody from Everest Biotech Ltd or in a 1:2000 dilution of rabbit anti-hOrai1 polyclonal antibodies from NewEast Biosciences, followed by an incubation in a 1:20,000 dilution of horseradish peroxidase-conjugated goat anti-rabbit IgG or rabbit anti-goat IgG (Thermo Scientific) or in a 1:20,000 dilution of horseradish peroxidase-conjugated rabbit anti-goat IgG or rabbit anti-goat IgG (Thermo Scientific). The proteins were visualized using an enhanced luminescence system (SuperSignal West Pico Chemiluminescent Substrate from Thermo Scientific).

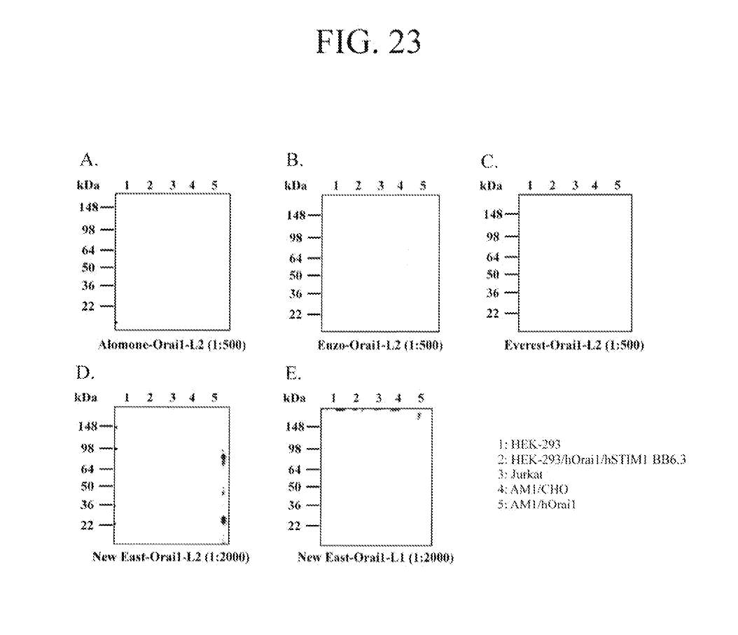

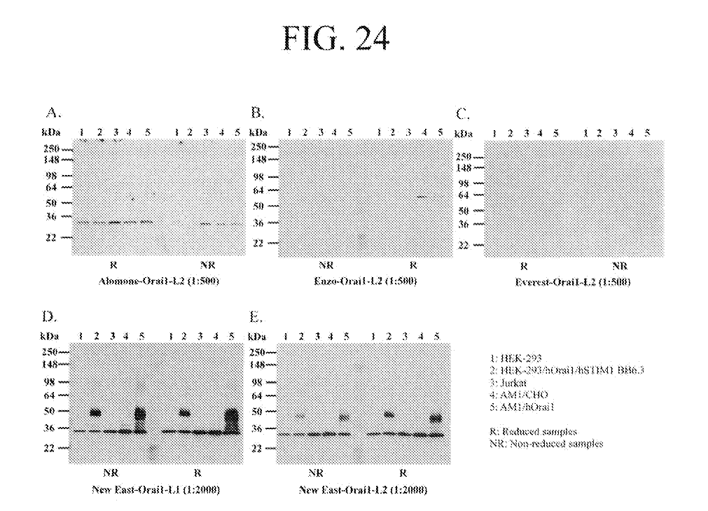

FIG. 23A-E shows that all commercially available antibodies were unable to detect hOrai1 proteins in native conformation. If the immunoblot analysis was performed under reducing and non-reducing conditions, one immunoreactive species of approximately 48 kDa, corresponding to the expected size for the glycosylated form of hOrai 1, was detected by New East Orai1-L1 and New East Orai1-L2 antibodies (FIG. 24D-E) in HEK-293/hOrai1/hSTIM1 BB6.3 and AM1/hOrai1 cell lysates, but not by Alomone-Orai1-L2, Enzo-Orai1-L2 and Everest-Orai1-L2 antibodies (FIG. 24A-C). AM1/CHO cells do not express endogenous hOrai1, a band at approximately 33 kDa was detected by all commercially available antibodies (FIG. 24A-E), indicating that the immunoreactive species observed at 33 kDa across HEK-293, HEK-293/hOrai1/hSTIM1 BB6.3, Jurkat, AM1/CHO and AM1/hOrai1 cell lysates was a non-specific band (FIG. 24A-E).

### Example 12

### Evaluation of Recombinant Human Anti-hOrai1 Monoclonal Antibodies in Detecting hOrai1Proteins by Western Analysis

We assessed recombinant human anti-hOrai1 mAbs generated from Campaign 1 and Campaign 2 for their ability to detect hOrai1 proteins by Western analysis under native, reducing and non-reducing conditions with HEK-293, HEK-293/hOrai1/hSTIM1 BB6.3, Jurkat, AM1/CHO and AM1/hOrai1 cell lysates, to compare with the commercially available antibodies listed in Table 12 in Example 9 (compare, Example 11 herein).

Preparation of Whole Cell Lysates.

HEK-293, HEK-293/hOrai1/hSTIM1 BB6.3, Jurkat, AM1/CHO and AM1/hOrai1 cells were harvested and rinsed twice with ice-cold 1×PBS, then solubilized in cell lysis buffer (1% Triton X-100, 0.1 M NaCl, 0.05 M Tris.HCl (pH8.0), 1 mM Na₃VO₄) containing Protease Inhibitor Cocktail (Roche). The particulate material was centrifuged at 14,000 rpm for 15 min at 4° C. and supernatants were stored at −80° C.

Western Analysis of Recombinant Anti-hOrai1 mAbs in Detecting hOrai1 Proteins

For detecting native confirmation of hOrai1, approximately 5 μg of cell lysates from each of HEK-293, HEK-293/hOrai1/hSTIM1 BB6.3, Jurkat, AM1/CHO and AM1/hOrai1 in Novex® Native Tris-Glycine Sample buffer (Invitrogen) were separated by electrophoresis through 4-20% Tris-Glycine polyacrylamide gels (Invitrogen) in Novex® Tris-Glycine Native Running Buffer (Invitrogen) and transferred to nitrocellulose filters (Invitrogen). For detecting denatured hOrai1 proteins, approximately 5 μg of cell lysates from each of HEK-293, HEK-293/hOrai1/hSTIM1 BB6.3, Jurkat, AM1/CHO and AM1/hOrai1 in Novex®Tris-Glycine SDS Sample buffer (Invitrogen) under non-reducing or reducing (NuPAGE® Sample Reducing Agent, Invitrogen) conditions were separated by electrophoresis through 4-20% Tris-Glycine polyacrylamide gels (Invitrogen) in Novex®Tris-Glycine Running Buffer (Invitrogen) and transferred to nitrocellulose filters (Invitrogen). The immunoblots were incubated in a 1:2000 dilution mAb 2B7.1, mAb 2C1.1, mAb 2D2.1 or mAb 5F7.1, followed by an incubation in a 1:20,000 dilution of horseradish peroxidase-conjugated goat anti-human IgG (Thermo Scientific). The proteins were visualized using an enhanced luminescence system (SuperSignal West Pico Chemiluminescent Substrate from Thermo Scientific).

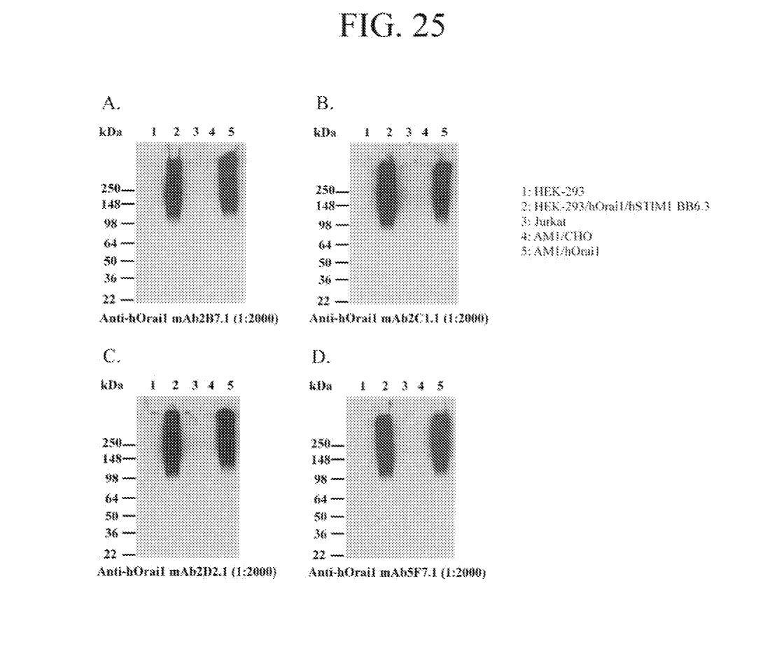

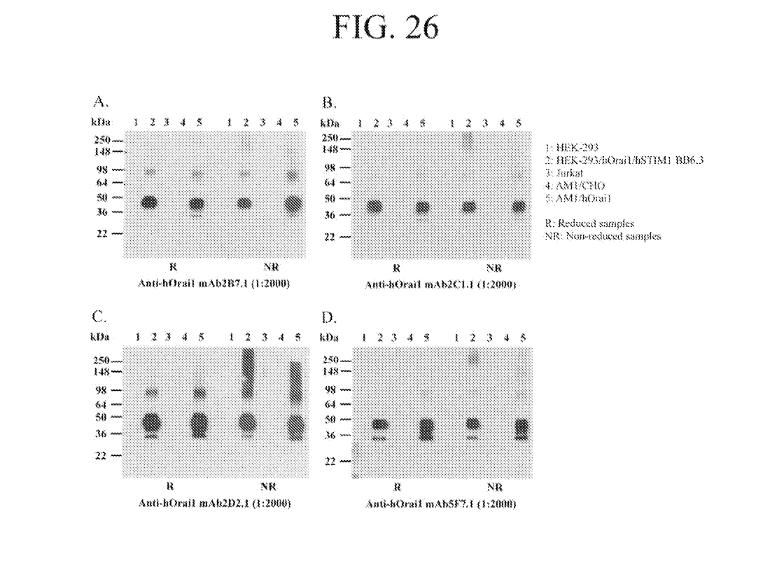

Representative results are shown in FIG. 25A-D showing an immunoreactive species of approximately 192 kDa, corresponding to oligomers of hOrai1, was detected by mAb 2B7.1, mAb 2C1.1, mAb 2D2.1 and mAb 5F7.1 in HEK-293/hOrai1/hSTIM1 BB6.3 and AM1/hOrai1 cell lysates, which illustrates the ability of human anti-hOrai1 antibodies of the present invention to detect hOrai1 proteins in native confirmation which are consistent with what were observed in FACS binding results as described herein. Subsequently, the recombinant anti-hOrai1 antibodies were evaluated for their ability to identify hOrai1 under reducing and non-reducing conditions. Representative results are shown in FIG. 26A-D showing an immunoreactive species of approximately 48 kDa, corresponding to the expected size for the glycosylated form of hOrai1, was detected by mAb 2B7.1, mAb 2C1.1, mAb 2D2.1 and mAb 5F7.1 in HEK-293/hOrai1/hSTIM1 BB6.3 and AM1/hOrai1 cell lysates. The recombinant anti-hOrai1 antibodies also reveal two bands at approximately 33 kDa and 96 kDa, corresponding to the unglycosylated hOrai1 and oligomer hOrai1 proteins, respectively (FIG. 26A-D).

### Example 13

### Inhibition of Cytokine Release by Inventive Antibodies

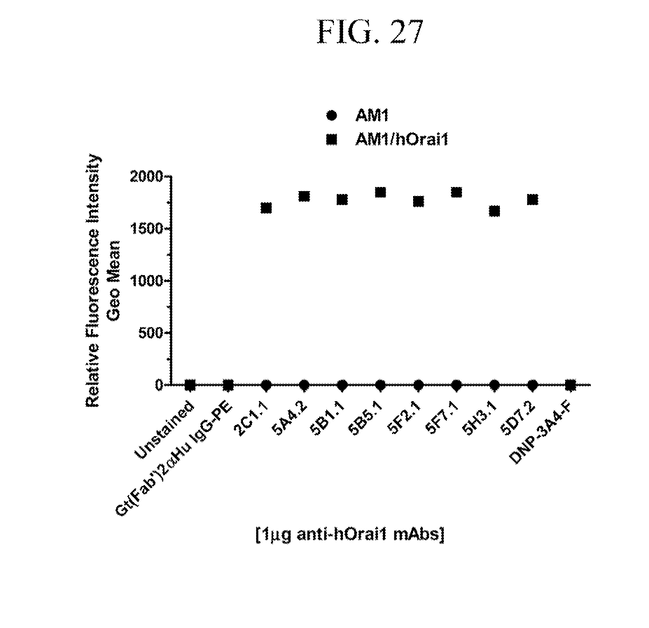

Out of the 14 mAbs identified from Campaign 2 for potent inhibition of cytokine release from human whole blood assay, analysis of their corresponding heavy and light chain antibody sequences indicated that there were seven unique monoclonal antibodies, referred to as mAb 5F7.1, mAb 5H3.1, mAb 5F2.1, mAb 5B1.1, mAb 5B5.1, mAb 5A4.2 and mAb 5D7.2. Binding to human Orai1 by recombinant antibodies was assessed by the FACS method described herein. Recombinant monoclonal antibodies were transiently expressed in HEK 293-6E cells, purified and characterized by the methods described in Example 4 herein above. The recombinant mAbs were first assessed to confirm their specific binding to human Orai1 expressed on the surface of AM1/CHO cells. FIG. 27 shows mAb 5F7.1, mAb 5H3.1, mAb 5F2.1, mAb 5B1.1, mAb 5B5.1, mAb 5A4.2 and mAb 5D7.2 binding to parental CHO was negligible, with a low relative fluorescence intensity geometric mean (geo mean) value that was comparable to the unstained control, directly labeled secondary reagent-only staining control and isotype control mAb, DNP-3A4-F-G2 (Human anti-DNP antibody described in Walker et al., WO 2010/108153 A2, at Example 12 therein). The geo mean values for the AM1/hOrai1 were also low for the unstained control, secondary reagent-only and isotype control. However, there was significant specific binding as indicated by the huge increase in values of the geo mean for mAb 5F7.1, mAb 5H3.1, mAb 5F2.1, mAb 5B1.1, mAb 5B5.1, mAb 5A4.2 and mAb 5D7.2, which was comparable to binding of recombinant mAb 2C1.1 from Campaign 1.

Recombinant mAbs were assessed for their ability to block cytokine release from human whole blood assay at various concentrations. These mAbs dose-dependently inhibited both IL-2 and IFN-γ release in the human whole blood assay system.

Table 13 (below) shows the half-maximal inhibitory concentrations (IC50) of the recombinant mAbs 5F7.1, 5H3.1, 5F2.1, 5B1.1, 5B5.1, 5A4.2 and 5D7.2 in blocking IL-2 and IFN-gamma secretion from thapsigargin-treated human whole blood. Comparing the IC50s of purified mAbs from Table 8B (in Example 4 herein above) with the IC50s of recombinant mAbs in Table 13, it was observed that the potency was comparable in inhibiting cytokine release from human whole blood assay. On the other hand, as expected, no inhibition was observed with isotype control mAb, DNP-3A4-F-G2 (Human anti-DNP antibody described in Walker et al., WO 2010/108153 A2, at Example 12 therein).

TABLE 13
IC50s of the recombinant mAbs 5F7.1, 5H3.1, 5F2.1, 5B1.1, 5B5.1,
5A4.2 and 5D7.2 in inhibiting Interleukin-2 (IL-2) and interferon-
gamma (IFN-γ) release in the whole blood assay system.

| | IL-2 | | IFN-γ | |
|---|---|---|---|---|
| | Donor A, | Donor B, | Donor A, | Donor B, IC₅₀, |
| Clone # | IC₅₀, nM | IC₅₀,nM | IC₅₀, nM | nM |
| 5F7.1 | 1.32 | 2.52 | 0.28 | 10.67 |
| 5H3.1 | 0.87 | 1.59 | 0.10 | 2.55 |
| 5F2.1 | 2.42 | 2.43 | 11.07 | 9.84 |
| 5B1.1 | 2.08 | 9.52 | 0.52 | 34.78 |
| 5B5.1 | 1.34 | 1.14 | 0.80 | 42.89 |
| 5A4.2 | 1.42 | 0.87 | 0.59 | 3.59 |
| 5D7.2 | 3.17 | 0.80 | 2.67 | 1.17 |
| DNP-3A4-F-G2 | — | — | — | — |

### Example 14

### Assessment of the Binding Specificity of Human Anti-hOrai1 mAbs

Molecular Cloning of cynoOrai

The cynomolgus Orai1 (cynoOrai1; SEQ ID NO:306), encoded by the following cDNA sequence:

SEQ ID NO. 305

| | |
|---|---|
| 1 | ATGCATCCGG AGCCCGCCCC GCCCCCGAGC CGCAGCAGCC |
| | CCGAGCTTCC |
| 51 | CCCGAGCGGC GGCAGCACCA CCAGCGGTAG CCGCCGGAGC |
| | CGCCGCCGCA |
| 101 | GCGGGGACGG GGAGCCTCCG GGAGCCCCGC CGCCGCCGCC |
| | GCCGCCGCCG |
| 151 | CCGCCGCCCG CCGTCACCTA CCCGGACTGG ATCGGCCAGA |
| | GTTACTCCGA |
| 201 | GGTGATGAGT CTCAACGAGC ACTCCATGCA GGCGCTGTCC |
| | TGGCGCAAGC |
| 251 | TCTATTTGAG CCGCGCCAAG CTCAAAGCCT CCAGCCGGAC |
| | CTCGGCTCTG |
| 301 | CTCTCCGGCT TCGCCATGGT GGCAATGGTG GAGGTGCAGC |
| | TGGACGCTGA |
| 351 | CCACGACTAC CCGCCAGGGC TGCTCATCGC CTTCAGTGCC |
| | TGCACCACGG |
| 401 | TGCTGGTGGC TGTGCACCTG TTTGCACTCA TGATCAGCAC |
| | CTGCATCCTG |
| 451 | CCCAACATCG AGGCGGTGAG CAACGTGCAC AACCTCAACT |
| | CGGTCAAGGA |
| 501 | GTCCCCCCAC GAGCGCATGC ACCGCCACAT CGAGCTGGCC |
| | TGGGCCTTCT |
| 551 | CCACCGTCAT CGGCACGCTG CTTTTCCTGG CCGAGGTCGT |
| | GCTGCTCTGC |
| 601 | TGGGTCAAGT TCTTGCCCCT CAAGAAGCAG CCAGGCCAGC |
| | CGAGGCCCAC |
| 651 | CAGCAAGCCC CCCGCCAGTG GTGCAGCCGC CAACGTCAGC |
| | ACCAGCGGCA |
| 701 | TCACCCCGGG CCAGGCAGCC GCCATCGCCT CGACCACCAT |
| | CATGGTGCCC |
| 751 | TTCGGCCTGA TCTTTATTGT CTTCGCCGTC CACTTCTACC |
| | GCTCACTGGT |
| 801 | CAGCCATAAG ACGGACCGAC AGTTCCAGGA GCTCAACGAG |
| | CTGGCGGAGT |
| 851 | TTGCTCGCTT ACAGGACCAG CTGGACCACA GAGGGGACCA |
| | CCCCCTGACG |
| 901 | CCCGGCAGCC ACTATGCCTA G//. |

was cloned into the pcDNA3.1/Neomycin (Invitrogen, Carlsbad, Calif.) for expression in mammalian cells.

The cynomoglous ORAI1 (cynoOrai1) was constructed using standard PCR technology. Briefly, primers with the sequence as depicted below (SEQ ID NOS: 307-310) were used in a two parts (Part A and B) PCR strategy using cynomologus monkey skeletal muscle cDNA from Biochain Inc. as a template.

Forward primer for Part A was:

(SEQ ID NO: 307)

5′-GATGCATCCGGAGCCCGC-3′;
and
Reverse primer for Part A was:

(SEQ ID NO: 308)

5′-GCTCGTTGAGCTCCTGGAAC-3′
Forward primer for Part B was

(SEQ ID NO: 309)

5′-CCTCAACGAGCACTCCATGCAGG-3′;
and
Reverse primer for Part A was:

(SEQ ID NO: 310)

5′-CTCTTAGAGGACAGTTTCAAAGTG-3′

The resulting 841-bp PCR product from part A and 890-bp PCR product from part B were then purified and subcloned into pCR2.1TOPO (Invitrogen).

Subsequently, primers with the sequence as depicted below (SEQ ID NOS:308, 309, 311, 312) were used in a two-part (Part C and D) PCR strategy using the part A and part B products in pCR2.1TOPO vector as a template.

Forward primer for Part C was:

(SEQ ID NO.: 311)

5′-GAAGCTTTGAACCACCATGCATCCGGAGCCCGCCCCGCCCCCGAGCC
GCAG-3′;
and
Reverse primer for Part C was:

(SEQ ID NO: 308)

5′-GCTCGTTGAGCTCCTGGAAC-3′.
Forward primer for Part D was:

(SEQ ID NO: 309)

5′-CCTCAACGAGCACTCCATGCAGG-3′
Reverse primer for Part C was:

(SEQ ID NO: 312)

5′GCGGCCGCCTAGGCATAGTGGCTGCC 3′.

The resulting 857-bp PCR product from part C and 771-bp product from part D were then purified and subcloned into pCR2.1TOPO (Invitrogen). The 857-bp PCR product from part C in pCR2.1TOPO was digested with HindIII and NcoI restriction enzymes, constitutes the 5′ fragment of cynoOrai1 construct and the 771-bp PCR product from part D in pCR2.1TOPO was digested with NcoI and Not1 restriction enzymes, constitutes the 3′ fragment of cynoOrai1 construct. The pcDNA3.1/Neomycin expression vector was digested with HindIII and Not1 restriction enzymes. The digested PCR product and vector were ligated to create a pcDNA3.1/Neomycin-cynoOrai1. The insert was sequenced and determined to be 100% identical to the cynoOrai1 cDNA coding sequence (SEQ ID NO:305, encoding the cynoOrai1 protein sequence SEQ ID NO:306):

(SEQ ID NO: 306)

| | |
|---|---|
| 1 | MHPEPAPPPS RSSPELPPSG GSTTSGSRRS RRRSGDGEPP |
| | GAPPPPPPPP |
| 51 | PPPAVTYPDW IGQSYSEVMS LNEHSMQALS WRKLYLSRAK |
| | LKASSRTSAL |
| 101 | LSGFAMVAMV EVQLDADHDY PPGLLIAFSA CTTVLVAVHL |
| | FALMISTCIL |
| 151 | PNIEAVSNVH NLNSVKESPH ERMHRHIELA WAFSTVIGTL |
| | LFLAEVVLLC |
| 201 | WVKFLPLKKQ PGQPRPTSKP PASGAAANVS TSGITPGQAA |
| | AIASTTIMVP |
| 251 | FGLIFIVFAV HFYRSLVSHK TDRQFQELNE LAEFARLQDQ |
| | LDHRGDHPLT |
| 301 | PGSHYA//. |

Generating Human Orai1 Single Nucleotide Polymorphism Variant N223S.

To generate human Orai1 (N223S) variant protein (SEQ ID NO:317), two oligonucleotide primers (SEQ ID NO:314 and SEQ ID NO:315), depicted below, were used in a site directed mutagenesis PCR reaction using the QuikChange Multi Site-Directed Mutagenesis Kit (Agilent Technologies, Stratagene Products Division, La Jolla, Calif.), with all PCR amplification conditions as recommended by the manufacturer:

Forward primer:

(SEQ ID NO: 314)

5′-GGCGCAGCAGCCAGCGTCAGCACCA-3′
and
Reverse primer:

(SEQ ID NO: 315)

5′-TGGTGCTGACGCTGGCTGCTGCGCC-3′.

The template that was used for the site-directed mutagenesis was the full length human Orai1 wild-type construct (SEQ ID NO:1), which was previously cloned into pcDNA3.1/Hygromycin and the resulting construct is referred to as hOrai1(N223S) cDNA (SEQ ID NO:316; encoding hOrai1(N223S) protein (SEQ ID NO:317)). The insert was sequenced and determined to be 100% identical to the hOrai1(N223S) cDNA coding sequence (SEQ ID NO:316):

SEQ ID NO: 316

ATGCATCCGGAGCCCGCCCCGCCCCCGAGCCGCAGCAGTCCCGAGCTTCC
CCCAAGCGGCGGCAGCACCACCAGCGGCAGCCGCCGGAGCCGCCGCCGC
AGCGGGGACGGGGAGCCCCCGGGGGCCCCGCCACCGCCGCCGTCCGCCG
TCACCTACCCGGACTGGATCGGCCAGAGTTACTCCGAGGTGATGAGCCTC
AACGAGCACTCCATGCAGGCGCTGTCCTGGCGCAAGCTCTACTTGAGCCG
CGCCAAGCTTAAAGCCTCCAGCCGGACCTCGGCTCTGCTCTCCGGCTTCG
CCATGGTGGCAATGGTGGAGGTGCAGCTGGACGCTGACCACGACTACCC
ACCGGGGCTGCTCATCGCCTTCAGTGCCTGCACCACAGTGCTGGTGGCTG
TGCACCTGTTTGCGCTCATGATCAGCACCTGCATCCTGCCCAACATCGAG
GCGGTGAGCAACGTGCACAATCTCAACTCGGTCAAGGAGTCCCCCCATG
AGCGCATGCACCGCCACATCGAGCTGGCCTGGGCCTTCTCCACCGTCATC
GGCACGCTGCTCTTCCTAGCTGAGGTGGTGCTGCTCTGCTGGGTCAAGTT
CTTGCCCCTCAAGAAGCAGCCAGGCCAGCCAAGGCCCACCAGCAAGCCC
CCCGCCAGTGGCGCAGCAGCCAGCGTCAGCACCAGCGGCATCACCCCGG
GCCAGGCAGCTGCCATCGCCTCGACCACCATCATGGTGCCCTTCGGCCTG
ATCTTTATCGTCTTCGCCGTCCACTTCTACCGCTCACTGGTTAGCCATAA
GACCGACCGACAGTTCCAGGAGCTCAACGAGCTGGCGGAGTTTGCCCGCT
TACAGGACCAGCTGGACCACAGAGGGGACCACCCCCTGACGCCCGGCAG
CCACTATGCCTAG//.

Transient Expression for FACS Binding Analysis.

One day prior to transfection, 293EBNA cells were plated at 3.5×10⁶ cells/dish in 10 mL of growth medium onto 100-mm tissue culture dishes. For one 100-mm dish, 10 μg of DNA was diluted in 460 μL of Opti-MEM, mixed gently, and incubated at room temperature for 5 min. Then, 40 μL of FuGene HD transfection reagent was added to the mixture, mixed gently, and incubated at room temperature for 20 minutes. The transfection mixture was added drop-wise onto the cells and the dish was gently swirled to ensure uniform distribution of the complex.

FACS Binding Analysis.

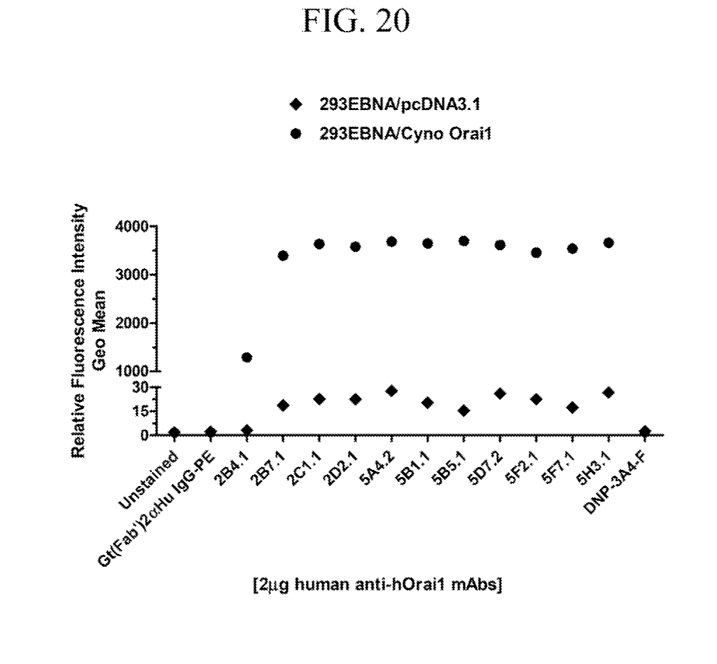

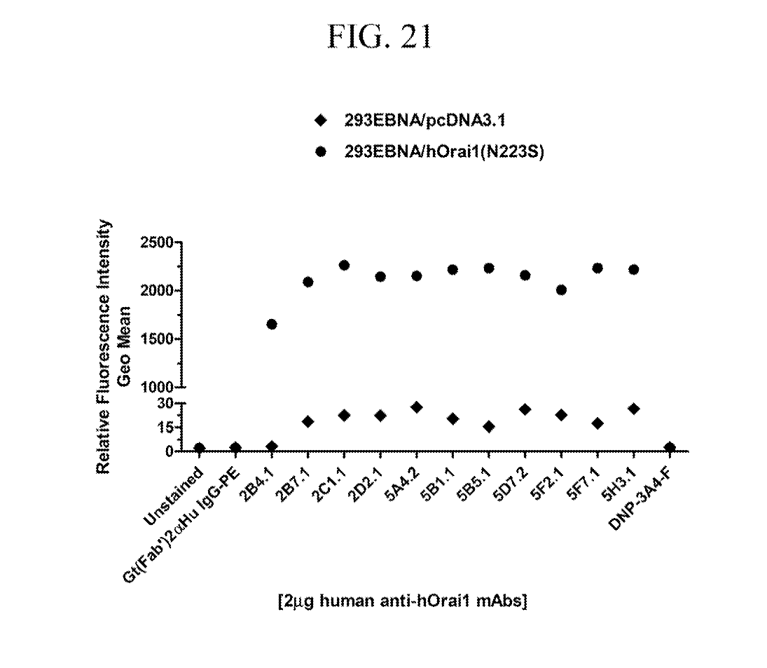

Transfected 293EBNA cells transiently expressed Cyno Orai1 (results of binding in FIG. 20) or hOrai1(N223S) (results of binding in FIG. 21). The transfected cells were harvested at 48 hours post-transfection. Cells transfected with pcDNA3.1 were used as negative controls. Cells were washed once with ice-cold 1×PBS, resuspended in ice-cold FACS buffer (1×D-PBS+2% goat serum), and 2×10⁵ cells in 100 μl were stained per antibody combination. All antibody incubation steps were performed on ice for 1 hour. Cells were first incubated with 2 μg of unlabeled human anti-hOrai1 monoclonal antibodies, followed by a wash with 200 μL of FACS buffer. Next, the unlabelled antibody was detected using goat F(ab′)₂ anti-human IgG-phycoerythrin (IgG-PE), followed by a wash with 200 μL of ice-cold FACS buffer before flow cytometry analysis. Unstained cells, cells stained with detecting antibodies and cells stained with isotype control antibody were used as negative controls. The values of relative level of fluorescence were calculated using FCS Express (De Novo Software) and mean values were calculated using log-transformed data (geometric mean).

The recombinant mAbs were assessed for their ability to bind cyno Orai1 protein. FIG. 20 shows that there was intense staining of human Orai1 by embodiments of inventive anti-Orai1 mAbs, which was not seen with control vector-transfected parental cells. Slightly higher staining was observed with vector-only-transfected HEK-293 controls (293EBNA/pcDNA3.1) for most mAbs (except mAb 2B4.1) compared to the isotype control antibody (anti-DNP-3A4-F), unstained control and directly labeled secondary antibody fragment negative staining controls. This probably indicates that most of the mAbs were recognizing endogenously expressed human Orai1 that is known to be present in HEK-293 cells. (e.g., Sternfeld et al., Activation of muscarinic receptors reduces store-operated Ca²⁺entry in HEK-293 cells, Cellular Signalling 19:1457-64 (2007); Fasolato et al., Store depletion triggers the calcium release-activated calcium current (ICRAC) in macrovascular endothelial cells: a comparison with Jurkat and embryonic kidney cell lines, Pfluegers Arch.-Eur. J. Physiol. 436(1):69-74 (1998)).

Human Orai1 has a single nucleotide polymorphism (SNP) encoding an asparagine-to-serine substitution at position 223 of hOrai1 (SEQ ID NOS:316-317) located in the ECL2 domain (see, NCBI SNP database rs75603737). Recombinant mAbs were assessed for their ability to bind the (N223S) variant of the human Orai1 protein. FIG. 21 shows that embodiments of inventive anti-Orai1 mAbs bound to the human Orai1 SNP variants and did not recognize control vector-transfected parental cells. Again, low staining was observed with vector-only-transfected HEK-293 controls (293EBNA/pcDNA3.1) for most mAbs except mAb 2B4.1 than the isotype control antibody, unstained control and directly labeled secondary antibody fragment negative staining controls, which probably indicated that most of the mAbs were recognizing endogenously expressed human Orai1 that is known to be present in HEK-293 cells. (e.g., Sternfeld et al., Activation of muscarinic receptors reduces store-operated Ca²⁺entry in HEK-293 cells, Cellular Signalling 19:1457-64 (2007); Fasolato et al., Store depletion triggers the calcium release-activated calcium current (ICRAC) in macrovascular endothelial cells: a comparison with Jurkat and embryonic kidney cell lines, Pfluegers Arch.-Eur. J. Physiol. 436(1):69-74 (1998)).

### Example 15

### Assessment of Human anti-hOrai1 mAbs Binding Affinity to hOrai1

The recombinant anti-hOrai1 mAbs were evaluated for their affinity to the native human Orai1 expressed on a stable cell line AM1-CHO/hOrai1/hSTIM1-YFP.

Equilibrium Set Up for FACS K_{d} Measurement

Two equilibrium sets were set up where the antibodies were titrated and incubated with two different constant cell concentrations, one at 10K cells per well and the other at 400K cells per well. The antibodies were titrated in cell media with 0.05% Sodium Azide across 96 well v-bottom plate from 200 nM, 1:4 for 10 wells in four duplicate rows in the final volume of 60 μL per well. The cells were dissociated with cell dissociation solution, washed twice with ice-cold 1×PBS and counted using a Hemocytometer. To the top two rows of the v-bottom plates with the titrated antibody solutions 10K of cells per well were added in 60 μL of the media with 0.05% Sodium Azide. To the next two rows of the v-bottom plates with the titrated antibody solutions 400K of cells per well were added in 60 μL of medium with 0.05% Sodium Azide. The plates were sealed with parafilm and incubated at 37° C. overnight shaking.

FACS Analysis

The equilibrium plates were centrifuged at 500 g for 3 minutes and cell pellets were washed twice with 200 μL of ice cold FACS buffer (1×D-PBS+2% FBS). The cells were then incubated with a secondary antibody, goat anti-human Fc Cy5 at 5 μg/mL and 100 μL per well for 1 h on ice in the dark. Following the incubation, the cells were washed twice with 200 μL of ice-cold FACS buffer before flow cytometry analysis. The GeoMean values gated on live cell population measure the bound [Ab]. The inverse of the GeoMean values that reflects theoretical free [Ag] in the equilibrium solution was then calculated. The inverse values of the GeoMean read were used in KinExA® Pro software for analysis and K_{d} calculation for each antibody. The free [Ag] measure was plotted against the starting concentration of titrating component and from these plots at two different cell concentrations the K_{d} was obtained from curve fitting using n-curve analysis in KinExA Pro software. The 95% confidence interval is given as K_{d} low and K_{d} high. Shown in Table 14 (below) are the equilibrium dissociation constant (K_{d}) values of invented mAbs determined by FACS K_{d} measurement. These antibodies displayed K_{d} in the low nanomolar to picomolar concentration range binding to hOrai1. As expected, lowest binding affinity was observed for mAb 2B4.1 which is not a blocking mAb.

TABLE 14
FACS K_{d} measurement of recombinant anti-hOrai1 mAbs for hOrai1
expressed on AM1-CHO/hOrai1/hSTIM1-YFP cells. The K_{d} low and K_{d}
high values show the K_{d} bounds for each measurement. The ratio 10K
below 1.0 indicates that at 10K cells per well the experiment was
run under K_{d}-controlled conditions.

| | N curve analysis | | | | |
|---|---|---|---|---|---|
| Clone | | | | | Ratio |
| Name | K_{d} | % error | K_{d} Low | K_{d} High | 10K |
| 5H3.1 | 649 pM | 5.3 | 309 pM | 1.3 nM | 0.3 |
| 5F2.1 | 767 pM | 5.4 | 384 pM | 1.5 nM | 0.3 |
| 5B1.1 | 443 pM | 5.3 | 222 pM | 794 pM | 0.3 |
| 5B5.1 | 735 pM | 4.7 | 395 pM | 1.3 nM | 0.2 |
| 5A4.2 | 1.2 nM | 5.0 | 641 pM | 2.2 nM | 0.2 |
| 5D7.2 | 483 pM | 5.5 | 221 pM | 965 pM | 0.4 |
| 2B7.1 | 133 pM | 3.5 | 74 pM | 219 pM | 1.5 |
| 5F7.1 | 185 pM | 6.5 | 65 pM | 471 pM | 1.1 |
| 2C1.1 | 520 pM | 6.0 | 224 pM | 1.1 nM | 0.1 |
| 2B4.1 | 1.8 nM | 6.4 | 841 pM | 3.8 nM | 0.5 |
| 2D2.1 | 9.8 pM | 9.1 | <35 fM | 160 pM | 5.3 |

Equilibrium set up for KinExA affinity measurement. Based on the results of FACS K_{d} measurement (see above), four representative recombinant anti-hOrai1 mAbs were selected for further Kinetic affinity assessment by Kinetic Exclusion Assay (KinExA) in which the K_{d} was determined from the concentration of free antibody that remains in solution after equilibrium has been established between the antibody and the cell-surface-expressed antigen. The more-resource intensive, KinExA assay provides a more sensitive determination of binding affinity than the FACS-based assay system described above. The Kinetic Exclusion Assay method of Rathanaswami et al. (2008) was followed. (Rathanaswami et al., High affinity binding measurements of antibodies to cell-surface-expressed antigens, Analytical Biochemistry 373:52-60 (2008), which is incorporated herein by reference in its entirety). Briefly, two different equilibrium sets were set up where the cells were titrated and incubated with two different constant antibody concentrations, one at 20 μM and the other at 500 μM. The cells were dissociated with cell dissociation solution, washed twice with ice-cold 1×PBS and counted using a Hemocytometer. Cell titrations and antibody solutions were set up in media with 0.05% Sodium Azide. Cells were titrated from two or four million per milliliter concentration, 1:3, for 10 points in 15-mL Falcon tubes. For the low [Ab] equilibrium set 7 mL of 40 μM Ab was mixed with 7 mL of each cell titration solution diluting the final cell and Ab concentration in half. For the high [Ab] equilibrium set 750 μL of 1 nM Ab was mixed with 750 μL of each cell titration solution diluting the final cell and Ab concentration in half. For each equilibrium set a blank cell media only sample and no cell added sample would be included for reference points. The equilibrium sets were incubated for 48 hours at 37° C., with shaking.

Equilibrium Sample Preparation for KinExA Analysis.

After 48 hours incubation of the equilibrium sets at 37° C. shaking, the supernatants were separated from the cell pellets via centrifugation at 500×g for 5 minutes. Prior to the centrifugation, to each tube about 1 million of untransfected CHO-AM1D filler cells were added to ensure more efficient pelleting of the cells. The low [Ab] equilibrium set of samples was then de-gassed using a vacuum chamber for 1 hour at room temperature. The supernatants of both high [Ab] and low [Ab] equilibrium sets were then run through a KinExA 3200 machine.

KinExA Analysis.

Each equilibrium sample set was read in triplicate on the KinExA machine. For low [Ab] equilibrium samples 4 mL was run of each sample in triplicate. For high [Ab] equilibrium samples 300 μL was run of each sample in triplicate. PMMA (Polymethyl Methacrylate Particles) beads were coated with Gt anti Human Fc Ab and subsequently blocked with a blocking solution (1×PBS pH7.4+10 mg/mL BSA+0.05% Sodium Azide). For each equilibrium sample the free [Ab] would be detected by first running the coated beads through the flow cell, then after quick wash with the running buffer (1×PBS pH7.4+1% BSA+0.05% Sodium Azide) the equilibrium samples were passed through the beads followed by a quick wash with the running buffer. Then the secondary detection Ab, Gt anti Hu (H+ L) Cy5, was run through the flow cell at 1 μg/mL and 1000 μL per run. The KinExA voltage output signal was then used in KinExA software to calculate the K_{d} values for each antibody. From the plots at two different initial total [Ab] concentrations the K_{d} was obtained from curve fitting using n-curve analysis in KinExA Pro software (Sapidyne Instruments Inc., Boise Id.). The 95% confidence interval is given as K_{d} low and K_{d} high.

Table 15 (below) shows the equilibrium dissociation constant (K_{d}) values of four representative recombinant anti-hOrai1 mAbs determined by KinExA K_{d} measurement. These antibodies displayed K_{d} in the low picomolar concentration range binding to hOrai1.

Assessment of Rank of Binding Affinity for Several Embodiments of Human anti-hOrai1 mAbs

Equilibrium Set Up for Binding Measurement.

UltraLink Biosupport (Pierce cat#53110) was pre-coated with goat-anti-huFc (Jackson Immuno Research cat#109-005-098) then blocked with BSA. 30 μM of anti-Orai1 antobodies was incubated with 3.0×10′, 1.0×10′, and 3.0×10⁴ cell/ml of AM1-CHO/hOrai1/hSTIM1-YFP cells in 1% FBS, 0.05% sodium azide, and DMEM. Samples containing Ab and whole cells were rocked for 4 hours at room temperature. The whole cells and antibody-cell complexes were separated from unbound free Ab using Beckman GS-6R centrifuge at approximately 220×g for 5 min. The supernatant was filtered through 0.22 μm filter before passing the goat-anti-huFc coated beads. The amount of the bead-bound Ab were quantified by fluorescent (Cy5) labeled goat anti-hulgG (H+ L) antibody (Jackson Immuno Research cat#109-175-088). The binding signal is proportional to the concentration of free Ab in solution at each cell density. The relative binding signal 100% represents 30 μM antibody alone. The decreased signal indicates the antibody binding with AM1-CHO/hOrai 1/hSTIM1-YFP cells.

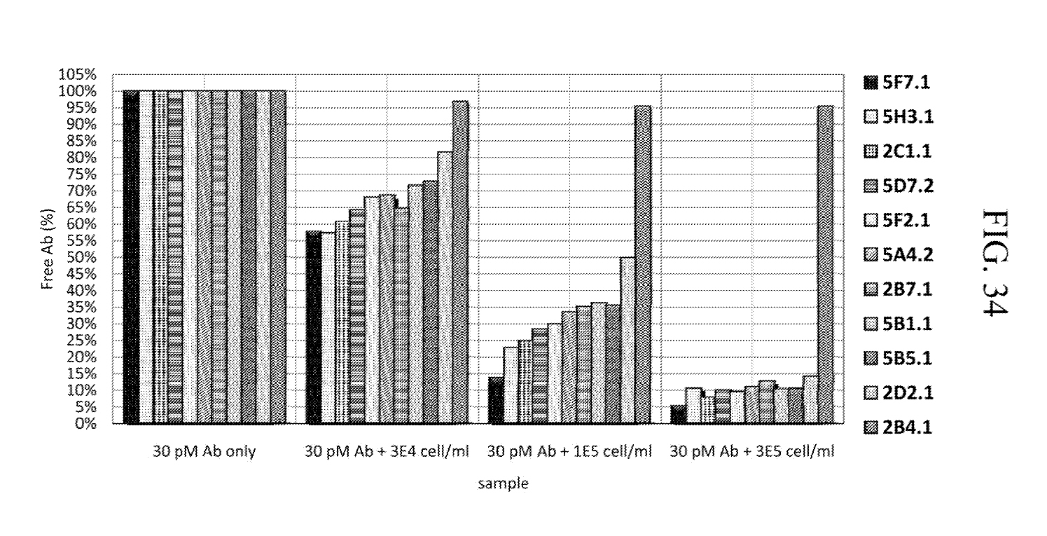

Briefly, the recombinant anti-hOrai1 mAbs were evaluated for their binding to the native human Orai1 (SEQ ID NO:2) expressed on a stable mammalian cell line AM1-CHO/hOrai1/hSTIM1-YFP by KinExA to determine the rank of anti-hOrai1 mAbs' affinity. FIG. 34 shows the percent of free mAb was 100% for no cell added samples and the percent of free mAb decreases with increasing density of added AM1-CHO/hOrai1/hSTIM1-YFP cells, indicating that anti-hOrai1 mAbs bound to hOrai1 on AM1-CHO/hOrai1/hSTIM1-YFP cells. In each set, the percent of free mAb was lower for high affinity binders and was higher for low affinity binder. Based on this analysis, the rank of binding affinity for anti-hOrai1 mAbs tested was 5F7.1>5H3.1=2C1.1=5D7.2=5F2.1=5A4.2=2B7.1=5B1.1=5B5.1>2D2.1>>2B4.1.

TABLE 15
KinExA K_{d} measurement of recombinant anti-hOrai1 mAbs for hOrai1
expressed on AM1-CHO/hOrai1/hSTIM1-YFP cells. The K_{d} low and
K_{d} high values show the K_{d} bounds for each measurement.
The ratio 20 pM [Ab]/K_{d} below 1.0 indicates that at 20 pM [Ab],
the experiment was run under K_{d}-controlled conditions.

| | N curve analysis | | | | |
|---|---|---|---|---|---|
| | | | | | Ratio Low |
| Clone | | | | | [Ab] |
| Name | K_{d} | % error | K_{d} Low | K_{d} High | (20 pM)/K_{d} |
| 5F7.1 | 19 pM | 3.1 | 10 pM | 34 pM | 1 |
| 2B7.1 | 100 pM | 1.5 | 74 pM | 136 pM | 0.2 |
| 2D2.1 | 99 pM | 3.1 | 56 pM | 187 pM | 0.2 |
| 2C1.1 | 40 pM | 2.8 | 24 pM | 65 pM | 0.5 |

### Example 16

### Assessment of Pharmacokinetic Profile of Anti-hOrai1 mAb 2C1.1 in Human Xeno GVHD Mice

Human Xeno GVHD Mice

Non-obese diabetic (NOD) severe combined immunodeficient (scid) interleukin (IL)-2 receptor gamma knockout (IL2Rgamma^{-/-}) mice (NOD/SCID/IL2Rgamma^{-/-}), commonly known as NSG mice, are severely immunocompromised, featuring absence of mature T cells and B cells, and lack of natural killer (NK) cells. NSG mice are also deficient for several high-affinity receptors for cytokines (including IL2, IL4, IL7, IL9, IL15, and IL21) that block the development of NK cells and further impair innate immunity. The compound immunodeficiencies in NSG mice permit the engraftment of a wide range of primary human cells, and enable sophisticated modeling of many areas of human biology and disease. Human xeno-graft-versus-host disease (GVHD) mouse model is established by transferring human peripheral blood mononuclear cells (PBMC) into NSG mice. These mice develop a disease that mimics human GVHD in many ways. One hundred percent of these mice develop xenogeneic GVHD following injection of as few as 5×10⁶ human PBMC, regardless of the PBMC donor used (King et al, Human peripheral blood leucocyte non-obese diabetic-severe combined immunodeficiency interleukin-2 receptor gamma chain gene mouse model of xengeneic graft-versus-host-like disease and the role of the host major histocompatibility complex, Clinical and Experimental Immunology, 157:104-118 (2009)).

Female NSG mice (#005557, 6-8 weeks old) were purchased from Jackson Laboratory (Bar Harber, Me.). All animal procedures were conducted in accordance with the protocols approved by the local animal care committee. To avoid graft rejection, recipient mice in all experiments underwent whole body sub-lethally irradiation with 200 Rads Cs-137. Frozen human PBMCs were purchased from Hemacare Inc. (Leukopac donor (#0750011), Van Nuys, Calif.). The cells were washed twice with cold PBS and 20 million of human PBMCs in 2001 of PBS were transferred into each recipient mouse via tail intravenous (i.v.) injection.

Collection of Serum Samples from Human Xeno GVHD Mice

Approximately 250 μl of blood from each NSG mice transferred with human PBMC dosed with anti-hOrai1 mAb 2C1.1 was collected in Microtainer® serum separator tubes at 0, 0.083, 0.5, 2, 4, 8, 24, 48, 96, 168, and 336 hours post-dose. Each sample was maintained at room temperature following collection, and following a 30-40-minute clotting period, samples were centrifuged at 2-8° C. at 11,500 rpm for about 10 minutes using a calibrated Eppendorf 5417R Centrifuge System (Brinkmann Instruments, Inc., Westbury, N.Y.). The collected serum was then transferred into a pre-labeled, cryogenic storage tube and stored at −60° C. to −80° C. for future analysis.

Generation of 6×His-Human Orai1 in pTT5 Mammalian Expression Vector.

The 6×-His-human Orai1 (6×H-hOrai1; SEQ ID NO:318), encoded by the following cDNA sequence:

SEQ ID NO: 318

ATGAAACATCATCACCATCACCATCACATGCATCCGGAGCCCGCCCCGCC
CCCGAGCCGCAGCAGTCCCGAGCTTCCCCCAAGCGGCGGCAGCACCACCA
GCGGCAGCCGCCGGAGCCGCCGCCGCAGCGGGGACGGGGAGCCCCCGGGG
GCCCCGCCACCGCCGCCGTCCGCCGTCACCTACCCGGACTGGATCGGCCA
GAGTTACTCCGAGGTGATGAGCCTCAACGAGCACTCCATGCAGGCGCTGT
CCTGGCGCAAGCTCTACTTGAGCCGCGCCAAGCTTAAAGCCTCCAGCCGG
ACCTCGGCTCTGCTCTCCGGCTTCGCCATGGTGGCAATGGTGGAGGTGCA
GCTGGACGCTGACCACGACTACCCACCGGGGCTGCTCATCGCCTTCAGTG
CCTGCACCACAGTGCTGGTGGCTGTGCACCTGTTTGCGCTCATGATCAGC
ACCTGCATCCTGCCCAACATCGAGGCGGTGAGCAACGTGCACAATCTCAA
CTCGGTCAAGGAGTCCCCCCATGAGCGCATGCACCGCCACATCGAGCTGG
CCTGGGCCTTCTCCACCGTCATCGGCACGCTGCTCTTCCTAGCTGAGGTG
GTGCTGCTCTGCTGGGTCAAGTTCTTGCCCCTCAAGAAGCAGCCAGGCCA
GCCAAGGCCCACCAGCAAGCCCCCCGCCAGTGGCGCAGCAGCCAACGTCA
GCACCAGCGGCATCACCCCGGGCCAGGCAGCTGCCATCGCCTCGACCACC
ATCATGGTGCCCTTCGGCCTGATCTTTATCGTCTTCGCCGTCCACTTCTA
CCGCTCACTGGTTAGCCATAAGACCGACCGACAGTTCCAGGAGCTCAACG
AGCTGGCGGAGTTTGCCCGCTTACAGGACCAGCTGGACCACAGAGGGGAC
CACCCCCTGACGCCCGGCAGCCACTATGCCTAGTAACTCGAGGATCCGCG
GAAAGAAGAAGAAGAAGAAGAA//.

was cloned into a CMV-based mammalian expression vector pTT5 (National Research Council, Canada).

Briefly, two oligonucleotide primers with the sequences depicted below (SEQ ID NO:304 and SEQ ID NO:320) were used in a Polymerase Chain Reaction (PCR) method using hOrai1 as a template.

Forward primer:

(SEQ ID NO: 304)

5′-GGTCGACTGAACCACCATGCATCATCATCACCACCACCATCCGGAGC
CCGCCCCGCCCCCGAG-3′
and
Reverse primer:

(SEQ ID NO: 320)

5′-CTGACGCCCGGCAGCCACTATGCCTAGGCGGCCGC-3′.

The resulting 948-bp PCR product was purified and digested with SalI and Not1 restriction enzymes. The TT5 vector was also digested with SalI and NotI restriction enzymes. The digested PCR product and vector were ligated to create a pTT5-6×H-hOrai1 vector. The insert was sequenced and determined to be 100% identical to the 6×His-human Orai1 cDNA coding sequence (SEQ ID NO:318; encoding the 6×His-human Orai11 protein sequence SEQ ID NO:319).

SEQ ID NO: 319

MKHHHHHHHMHPEPAPPPSRSSPELPPSGGSTTSGSRRSRRRSGDGEPPG
APPPPPSAVTYPDWIGQSYSEVMSLNEHSMQALSWRKLYLSRAKLKASSR
TSALLSGFAMVAMVEVQLDADHDYPPGLLIAFSACTTVLVAVHLFALMIS
TCILPNIEAVSNVHNLNSVKESPHERMHRHIELAWAFSTVIGTLLFLAEV
VLLCWVKFLPLKKQPGQPRPTSKPPASGAAANVSTSGITPGQAAAIASTT
IMVPFGLIFIVFAVHFYRSLVSHKTDRQFQELNELAEFARLQDQLDHRGD
HPLTPGSHYA//.

The 6×His is shown above at amino acid residues 3-8 of SEQ ID NO:319, which is single underlined and has the sequence of HHHHHH (SEQ ID NO:321).

Transient Expression for Generating 293-6E Expressing 6×H-hOrai1

One day prior to transfection, 293-6E cells were innoculated at 1×10⁶ cells/ml in 1,000 mL of growth medium. 0.5 μg of DNA per mL of culture was added to F17 medium, then 1.5 μg PEImax reagent per mL of culture was added to the mixture, mixed gently, and incubated at room temperature for 15 min. The transfection mixture was added to the cells and cultures were maintained on an orbital shaker at 110 rpm in an incubator set at 37° C. and 5% CO2. Tryptone N1 was added 24 hours post-transfection and cells were harvested 48 hours post-transfection.

Preparation of Human Orai1 Cell Membrane

293-6E cells transiently transfected with pTT5-6×H-hOrai1 were harvested 48-hr post-transfection and rinsed twice with ice-cold 1×PBS, before being resuspended in hypotonic lysis buffer (25 mM HEPES pH7.4, 3 mM MgCl₂) supplemented with Protease Inhibitor Cocktail (Roche) and homogenized in a Glas-Col homogenizer. The suspension was centrifuged at 20,000 rpm in JA20 rotor for 12 min at 4° C. The pellet was resuspended in hypotonic lysis buffer (25 mM HEPES pH7.4, 3 mM MgCl₂) supplemented with Protease Inhibitor Cocktail, re-homogenized, sheared with 25G needle and re-centrifuged. The pellet was resuspended in final pellet buffer (25 mM HEPES pH7.4, 3 mM MgCl₂, 10% (w/v) sucrose) supplemented with Protease Inhibitor Cocktail, and passed through a 25G needle 2-3 times, then was stored at −80° C. until use.

Enzyme-Linked Immunosorbent Assay (ELISA) for Detecting Anti-hOrai1 mAb in Serum Samples.

To measure the serum sample concentrations from the PK study samples, the following method was used: ½ area white plate (Corning 3693) was coated with 10 μg/ml of 6×H-hOrai1 cell membrane in PBS and then incubated overnight at 4° C. The plate was then washed and blocked with I-Block™ (Applied Biosystems) overnight at 4° C. If samples needed to be diluted, then they were diluted in mouse serum. The standards and samples were then diluted 1:50 in I -Block™+5% BSA (i.e. 10 μl of standards and samples into 490 μl of diluting buffer). The plate was washed and 50 μl samples of pretreated standards and samples were transferred into a hOrai1 cell membrane coated plate and incubated for 1.5 h at room temperature. The plate was washed, then 50 μl of 200 ng/ml of anti-hu Lambda light chain antibody, clone L-2H1.1-HRP conjugate in I-Block™+5% BSA was added and incubated for 1.5 h. The plate was washed, then 50 μl of Pico substrate (Thermo Fisher) were added, after which the plate was immediately analyzed with a luminometer.

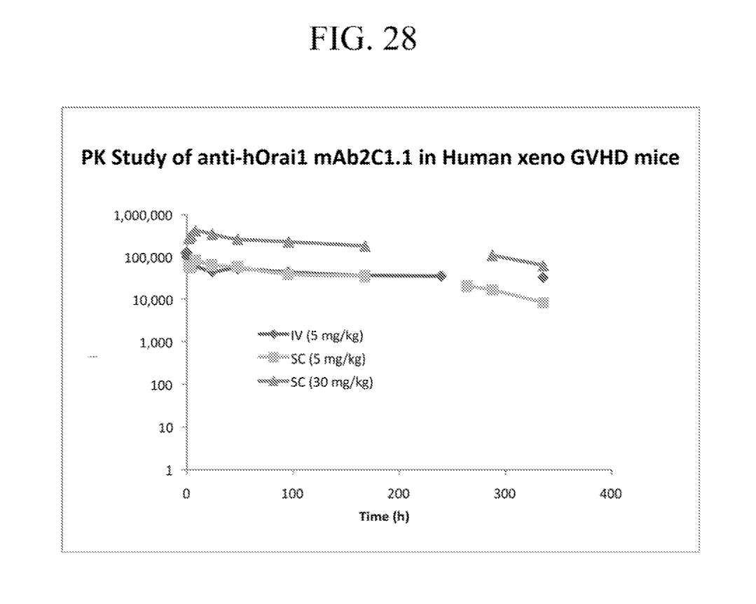

Briefly, the pharmacokinetic (PK) profile of the anti-hOrai1 mAb 2C1.1 was determined in human xeno GVHD mice by injecting 5 mg/kg intravenously, 5 mg/kg and 30 mg/kg subcutaneously. The PK samples were assayed for anti-hOrai1 mAb2C1.1 level using developed ELISA method. Time concentration data were analyzed using non-compartmental methods with WinNonLin® (Enterprise version 5.1.1, 2006, Pharsight® Corp. Mountain View, Calif.). The resulting pharmacokinetic profile shows exposure over two weeks with half life of about 7 to 25 days (FIG. 28). However the variability of the data from the 2^{nd} week was very high due to animals' high death rate. The PK parameters of anti-hOrai1 mAb 2C1.1 in NSG mice transferred with human PBMC are summarized in Table 16 (below).

TABLE 16
Pharmacokinetic parameters of anti-hOrai1 mAb 2C1.1 in human xeno
GVHD mice via intravenous (IV) or subcutaneous (SC) injection.

| Route | | IV | SC | SC |
|---|---|---|---|---|
| Dose | (mg/kg) | 5 | 5 | 30 |
| T_{1/2} | (h) | 601 | 154 | 181 |
| T_{1/2(eff)} | (h) | 592 | 150 | 179 |
| Tmax | (h) | 0.08 | 8 | 8 |
| Cmax | (ng/mL) | 125,446 | 83,619 | 406,562 |
| MRT | (h) | 854 | 217 | 259 |
| CL | (mL/h/kg) | 0.121 | 0.336 | 0.059 |
| AUC₀₋ₜ | (ngh/mL) | 13,702,108 | 11,672,562 | 61,631,242 |
| AUC_{1-inf} | (ngh/mL) | 41,341,606 | 14,873,468 | 85,214,654 |
| Vss | (mL/Kg) | 103 | | |
| F | | NA | 0.9 | |

### Example 17

### Effect of Anti-hOrai1 mAb in Human Xeno GVHD Model

Induction of GVHD and in vivo treatment. Non-obese diabetic (NOD) severe combined immunodeficient (scid) interleukin (IL)-2 receptor gamma knockout (IL2Rgamma^{-/-}) mice (NOD/SCID/IL2Rgamma^{-/-}), commonly known as NSG mice, are severely immunocompromised, featuring absence of mature T cells and B cells, and lack of natural killer (NK) cells. NSG mice are also deficient for several high-affinity receptors for cytokines (including IL2, IL4, IL7, IL9, IL15, and IL21) that block the development of NK cells and further impair innate immunity. The compound immunodeficiencies in NSG mice permit the engraftment of a wide range of primary human cells, and enable sophisticated modeling of many areas of human biology and disease. Human xeno-graft-versus-host disease (GVHD) mouse model is established by transferring human peripheral blood mononuclear cells (PBMC) into NSG mice. These mice develop a disease that mimics human GVHD in many ways. One hundred percent of these mice develop xenogeneic GVHD following injection of as few as 5×10⁶ human PBMC, regardless of the PBMC donor used (King et al, Human peripheral blood leucocyte non-obese diabetic-severe combined immunodeficiency interleukin-2 receptor gamma chain gene mouse model of xengeneic graft-versus-host-like disease and the role of the host major histocompatibility complex, Clinical and Experimental Immunology, 157:104-118 (2009)).

Thirty-four female NSG mice (#005557, 6-8 weeks old) were purchased from Jackson Laboratory (Bar Harber, Me.). All animal procedures were conducted in accordance with the protocols approved by the local animal care committee. To avoid graft rejection, recipient mice in all experiments underwent whole body sub-lethally irradiation with 200 Rads Cs-137. After irradiation, mice were randomly divided into 5 groups of 4-8 mice. Group 1 (n=4) didn't receive any treatment, nor human PBMC transfer. Frozen human PBMCs were purchased from Hemacare Inc. (Leukopac donor (#0750011), Van Nuys, Calif.). The cells were washed twice with cold PBS and 20 million of human PBMCs in 2001 of PBS and were transferred into each recipient mouse via tail intravenous injection. Recipients receiving human PBMC 4 hours after irradiation were divided into 4 groups of 8 mice and treated with isotype control huIgG2, 90 mg/kg (mpk) anti-KLH huIgG2 (described in Walker et al., WO 2010/108153 A2) which served as a negative control, or 5 mpk Orencia® (abatacept; Bristol-Myers Squibb) which served as a positive control or anti-hOrai1 mAb2C1.1 at 30 and 90 mpk. All the mice from group 2 through 5 were transferred with human peripheral blood mononuclear cells (PBMC) 4 hours after the irradiation. In the treatment groups, mice received mAbs via intraperitoneal injection (i.p.) on day 0 after irradiation prior to human PBMC transfer and on day 5. Body weight of each mouse was measured on day 0, 3, 5 and daily from day 7 to day 10. Body weight at day 0 was used as baseline which was expressed as 100 percent and the mean body weight change was expressed as percentage of day 0 weight. Experiment ended on day 10. Mice were euthanized with inhalation of CO₂. Blood was collected through cardiac puncture using a 1-ml syringe with a 25G⅞ inch needle. Blood was then put into a serum collection tube with separator. After clotting at room temperature for 30 to 60 minutes, samples were spun down at 10000 rpm for 5 at 4 C. Serums were transferred to a different tube and frozen down at −80C for further cytokine and drug level measurement. Spleens were harvested for FACS analysis (clinical immunology).

Levels of cytokine in the serum were determined using a 7-spot (IL-2, IL-4, IL-5, IL-10, IL-17, IFNgammar and TNFa) electrochemilluminescent immunoassay from MesoScale Discovery according to the manufacture's instruction.

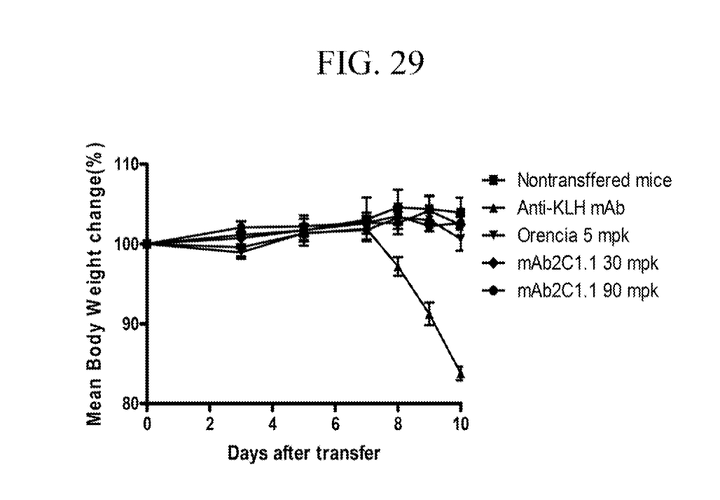

FIG. 29 shows mice treated with isotype control mAb, anti-KLH mAb, began to lose body weight at day-7. The decline in body weight occurred rapidly, reaching their maxima at day-10. At this point mice had typically lost about 20% of their body weight. Irradiated controls that were not transferred with human PBMC did not show weight loss for the duration of the study. Orencia is a clinically used biologic drug. Animals received Orencia at 5 mpk prevent weight loss and the mean body weight was comparable to that of nontransferred mice. Mice given anti-hOrai1 mAb 2C1.1 at 30 and 90 mpk did not exhibit weight loss as compared with isotype control mAb and the mean body weight was similar to that of nontransferred mice.

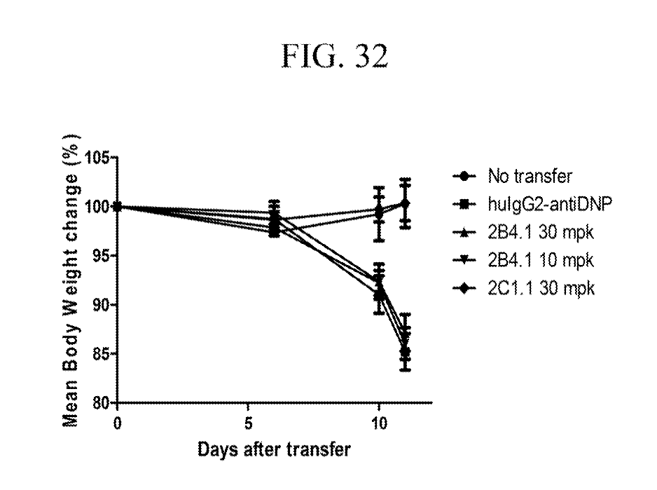

The mAb 2B4.1 is a weak binder with binding affinity at approximately 1.8 nM by FACS Kd affinity measurement (see, Table 14 in Example 15 herein). In the in vitro functional assessments, mAb 2B4.1 does not inhibited interleuklin-2 and interferon-gamma secretion in thapsigargin-treated human whole blood (FIG. 5A-D), but is a weak inhibitor in NFAT-mediated luciferase assay (FIG. 6C) and produced substantially less block of CRAC current compared to mAb 2C1.1 (data not shown). We examined the effect of mAb 2B4.1 in human xeno GVHD model. Results are shown in FIG. 32, which shows that mice treated with isotype control mAb, anti-DNP mAb, began to lose weight at day-6 and lost about 15% of the weight by day-11. Irradiated controls that were not transferred with human PBMC did not show weight loss for the duration of the study. Mice that received anti-hOrai1 mAb 2C1.1 at 30 mpk did not display weight loss similar to the nontransferred group. In contrast, mice given anti-hOrai1 mAb 2B4.1 at 10 and 30 mpk experienced weight loss similar to the isotype controls. These mice began to lose weight at day-6 and lost about 15% of the weight by day-11 comparable to the isotype control mice.

### Example 18

### Assessment of Human T Cell Engraftment and Inflammatory Cytokine Production in Anti-hOrai1 mAb-Treated Human xeno GVHD mice

FACS analysis for phenotyping of human and mouse PBMC. Experiment ended at day-10. Mice were euthanized with inhalation of CO₂. Spleens were harvested and the red blood cells were lysed using red blood cell (RBC) lysis buffer. The tubes were centrifuged at 300 relative centrifugal force (RCF) in a horizontal rotor (swing-out head) centrifuge at ambient temperature (18 to 25° C.). Splenocytes were diluted in RPMI+10% FBS and 3×10⁵ cells in 100 μL were stained per antibody combination. Cells were first incubated with the antibodies specific for human and mouse T cell specific surface markers, FITC Mouse Anti-Human CD4, APC Mouse Anti-Human CD8, PerCP Mouse Anti-Human CD45 or PE Rat Anti-Mouse CD45, for 30 minutes followed by a wash with 200 μL of PBS/0.5% BSA. Cells were resuspended in a final volume of 150 μL of PBS/0.5% BSA and were fixed with 50 μLs of 5% Paraformaldehyde/0.5% BSA and kept in the dark before flow cytometry analysis. The data were analyzed using BD Software.

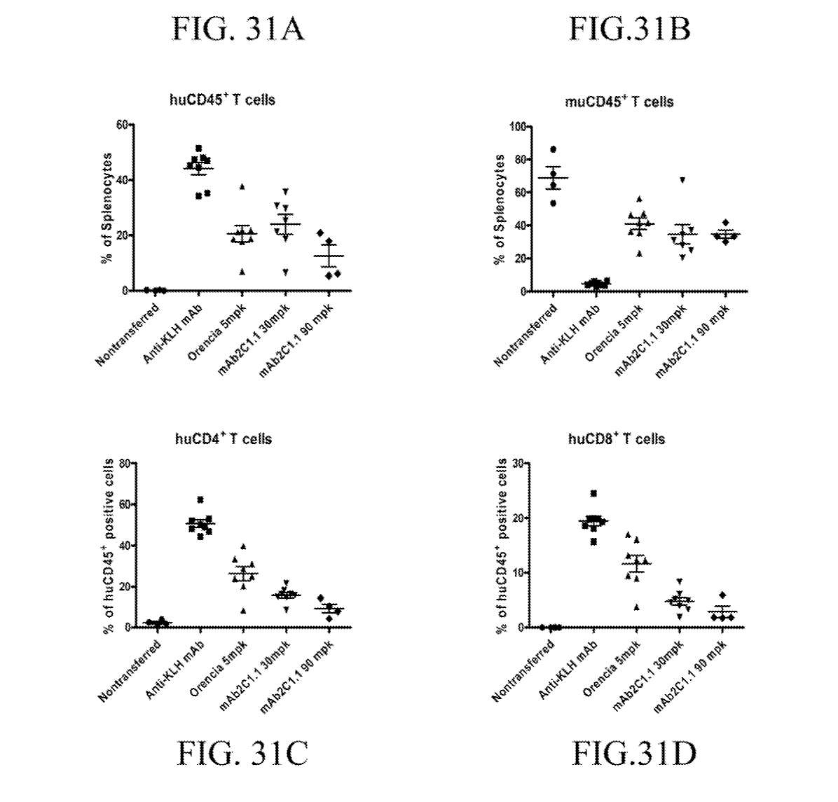

While mouse CD45⁺ T cells were detected in the spleens of irradiated controls that were not transferred with human PBMC (FIG. 31B), the human CD45⁺ T cells and the cells expressing CD4 and CD8 were undetectable (FIG. 31A and FIG. 31C-D, respectively). FIG. 31A-D shows that T cell engraftment was observed in the recipients treated with isotype control (anti-KLH mAb, described in Walker et al., WO 2010/108153 A2) in which the engraftment of human CD45⁺ T cells were detected in the spleens at approximately 45% (FIG. 31A), whereas mouse CD45⁺ T cells were at about 4% (FIG. 31B). Approximately 50% of human CD45⁺ T cells in the spleens expressed CD4 (FIG. 31C) and 20% expressed CD8 (FIG. 31D). Animals that received Orencia® (abatacept; Bristol-Myers Squibb) at 5 mpk reduced the percentage of human CD45⁺to approximately 22% and attenuated the percentage of human CD45⁺ T cells that expressed CD4 and CD8 to about 25% and 12%, respectively, compared with isotype control recipients (FIG. 31A). The percentage of host mouse CD45⁺ T cells in the spleens were about 10-fold higher in recipients treated with Orencia® (abatacept) than in recipients treated with isotype control mAb (FIG. 31B). Mice given anti-hOrai1 mAb 2C1.1 at 30 and 90 mpk dose-dependently decreased the percentage of human CD45⁺ T cells and the cells expressing CD4 and CD8 in the spleens in comparison with isotype control recipients (FIG. 31A, FIG. 31C, and FIG. 31D, respectively). In recipients treated, respectively, with 30 and 90 mpk of anti-hOrai1 mAb2C1.1, the percentage of human CD45⁺ T cells in the spleens declined to approximately 25% and 13% (FIG. 31A), in which the CD4+ T cells decreased to about 16% and 10% (FIG. 31C) and the CD8⁺ T cells decreased to about 5% and 3% (FIG. 31D). These findings indicate that anti-hOrai1 mAb 2C1.1 effectively prevent T cell engraftment in NSG mice that were transferred with human PBMC. The percentage of host mouse CD45⁺ T cells in recipients treated with 30 and 90 mpk of anti-hOrai1 mAb 2C1.1 were significantly higher than in recipients treated with isotype control mAb, at approximately 37% and 35%, respectively (FIG. 31B).

Assessment of Inflammatory Cytokine Production.

Experiment ended at day-10. Mice were euthanized with inhalation of CO₂. Blood was collected through cardiac puncture using 1 ml syringe with a 25G⅞ inch needle. Blood was then put into a serum collection tube with separator. After clotting at room temperature for 30 to 60 minutes, samples were spun down at 10,000 rpm for 5 minutes at 4° C. Serums were transferred to a different tube and frozen down at −80° C. for cytokine measurement.

Levels of cytokine in the serum were determined using a 7-spot (IL-2, IL-4, IL-5, IL-10, IL-17, IFN-γ and TNF-α) electrochemilluminescent immunoassay from Meso Scale Discovery (Gaithersburg, Md.) according to the manufacture's instruction.

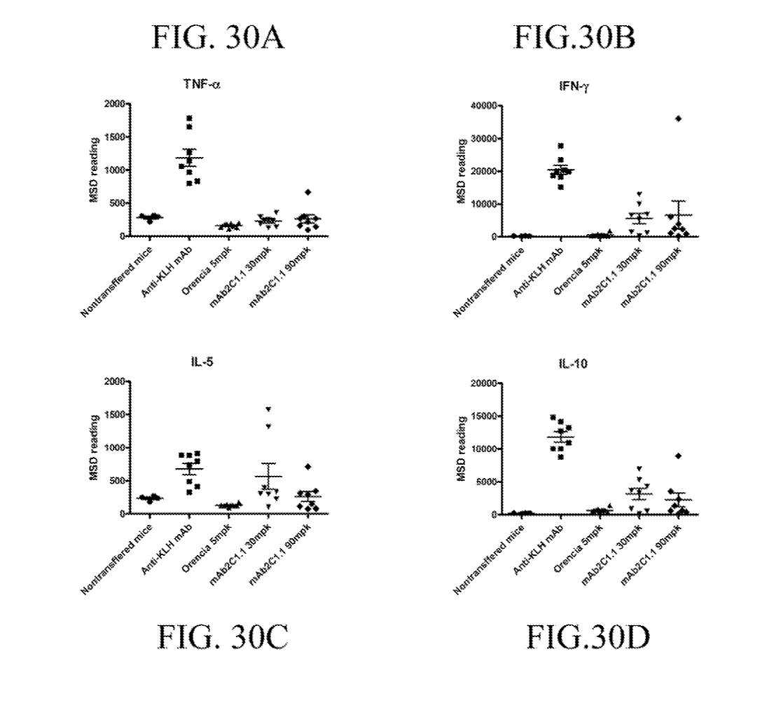

FIG. 30A-D shows that levels of TNF-α, IFN-γ, IL-5 and IL-10, respectively, were elevated in the sera of the mice treated with isotype control antibody (anti-KLH mAb), compared to disease-free mice, i.e., nontransferred mice. Animals given 5 mpk Orencia® (abatacept) significantly blocked the production of inflammatory cytokines, TNF-α, IFN-γ, IL-5 and IL-10. Mice treated with anti-hOrai1 mAb 2C1.1 at 30 and 90 mpk significantly attenuated the inflammatory cytokine production, TNF-α, IFN-γ, IL-5 and IL-10. The levels of IL-2, IL-4 and IL-17 were undetectable in all groups (data not shown).

### Example 19

### Assessment of Anti-hOrai1 mAb in Binding Endogenous Human Orai1 on Jurkat Cells

FACS Binding Analysis.

Jurkat cells were washed once and resuspended in ice-cold FACS buffer (1×D-PBS+2% goat serum), and 3×10⁵ cells in 1001 were stained per antibody combination. All antibody incubation steps were performed on ice for 1 hour. Cells were first incubated with 1 μg of unlabeled human anti-hOrai1 monoclonal antibodies (mAb 2C1.1, mAb 2D2.1, mAb 5F7.1 and mAb 2B4.1) or isotype control mAb (DNP-3A4-F-G2), followed by a wash with 200 μL of FACS buffer. Next, the unlabelled antibody was detected using goat F(ab′)₂ anti-human IgG-phycoerythrin (IgG-PE), followed by a wash with 200 μL of ice-cold FACS buffer before flow cytometry analysis. Unstained cells and cells stained with detecting antibodies were used as negative controls. The values of relative level of fluorescence were calculated using FCS Express (De Novo Software) and mean values were calculated using log-transformed data (geometric mean).

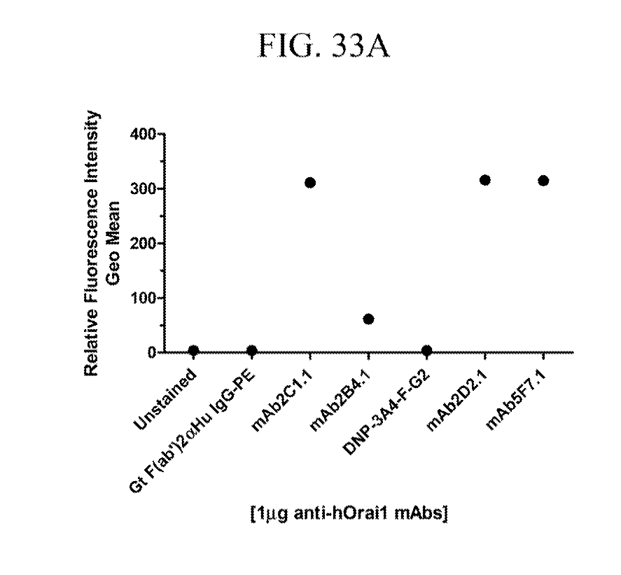

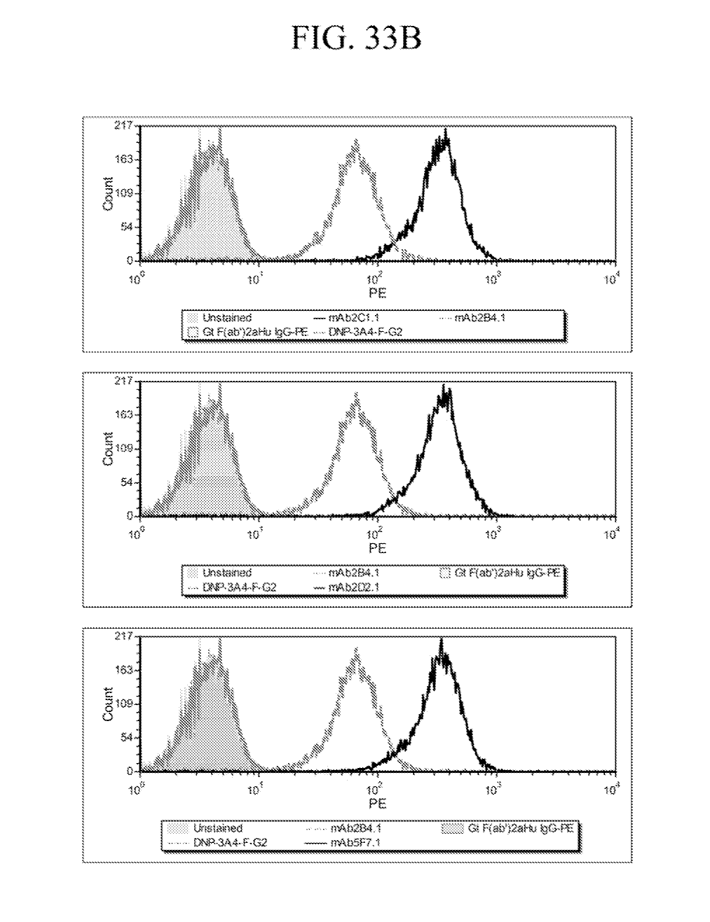

Human Orai1 is known to be present in Jurkat cells (Fasolato et al., Store depletion triggers the calcium release-activated calcium current (ICRAC) in macrovascular endothelial cells: a comparison with Jurkat and embryonic kidney cell lines, Pfluegers Arch.-Eur. J. Physiol. 436(1):69-74 (1998)). We assess anti-hOrai1 mAbs for their ability to detect endogenous human Orai1 on Jurkat cells. FIG. 33A shows that four anti-hOrai1 mAbs (mAb 2C1.1, mAb 2D.1, mAb 5F7.1 and mAb 2B4.1) tested recognized endogenously expressed human Orai1 on the surface of Jurkat cells. The Geo Mean values of three high affinity binders, mAb 2C1.1, mAb 2D.1 and mAb 5F7.1, were comparable to each other in binding hOrai1, while the values for the weak binder, mAb 2B4.1, was significantly lower than the values for the three high affinity binders. The binding of unstained control, directly labeled secondary antibody fragment negative staining control and isotype control mAb was deemed “not detectable”. FIG. 33B shows the FACS profile of each high affinity binder compared with mAb 2B4.1, unstained control, directly labeled secondary antibody fragment negative staining control and isotype control mAb, displaying about one log rightward shift for each high affinity binder compared to mAb 2B4.1.

### Example 20

### Electrophysiological Study of ICRAC Blockage by Inventive Antigen Binding Protein

The HEK-293/hOral/hSTIM1 BB6.3 cell line, stably expressing human STIM1 and human Orai1, was used for IC50 determination of CRAC current block by anti-hOrai1 mAb 2C1.1. The cells were grown in a medium consisted of DMEM, 10% FBS, 1 mM Na pyruvate, 1×MEM NEAA, 5 μg/ml Blasticidin, 100 μg/ml Zeocin, 200 μg/ml Hygromycin.

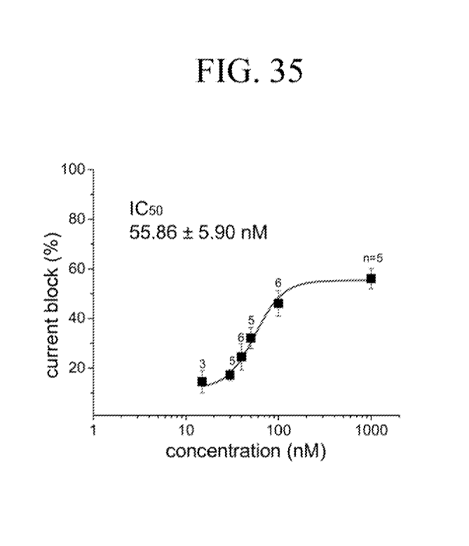

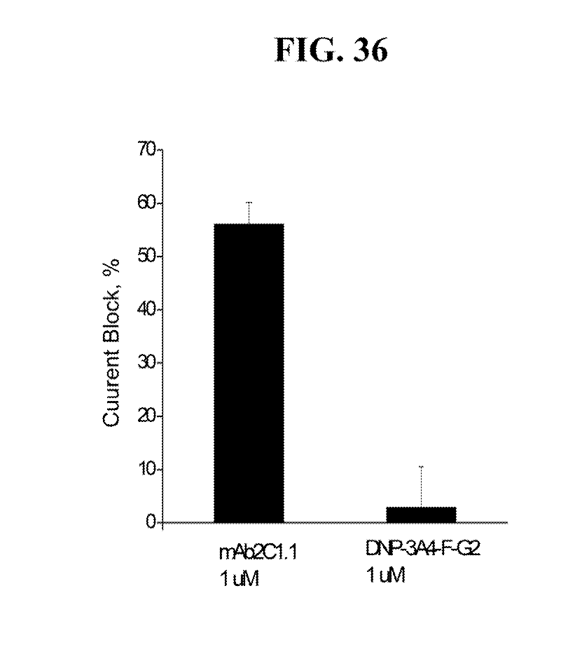

The currents were recorded in the whole-cell configuration using PatchXpress® 7000A automated parallel patch clamp system (Molecular Devices Inc.). The extracellular solution consisted of 110 mM NaCl, 10 mM CaCl₂, 3 mM KCl, 2 mM MgCl₂, 10 mM CsC1, 10 mM D-glucose, 10 mM HEPES (pH 7.4). The intracellular solution consisted of 95 mM cesium glutamate, 8 mM NaCl, 8 mM MgCl₂, 2 mM sodium pyruvate, 10 mM BAPTA, 10 mM HEPES (pH 7.2). All recordings were carried out at room temperature (21-23° C.). The holding potential was +30 mV. The voltage protocol was as follows: 10-msec step to −100 mV followed by 100-msec ramp from −100 to +100 mV; the protocol was applied every 5 sec. The recorded currents were leak subtracted using data collected in the extracellular solution containing 10 μM GgCl₃. The inhibitory effect of mAb 2C1.1 antibody on ICRAC current was measured at 6 concentrations of mAb 2C1.1 (n=3-6) (FIG. 35). The extracellular solution containing mAb 2C1.1 antibody was changed 3 times with a 1-minute interval to account for any potential nonspecific binding. The percentage of ICRAC current block was plotted against the antibody concentration (FIG. 35); the IC50 was calculated to be equal to 55.86+5.90 nM. In contrast, human anti-DNP antibody (anti-DNP-3A4-F-G2, described in Walker et al., WO 2010/108153 A2), applied at 1 μM concentration, did not show a significant affect on the ICRAC current (FIG. 36).

### Example 21

### Functional Assessment of Anti-Human Orai1 monoclonal Antibodies in Inhibiting Cytokine Release from Thapsigargin-Treated Cynomolgus Whole Blood

Ex vivo assay to examine impact of CRAC inhibitor antibody on secretion of IL-4, IL-5 and IL-17. Cynomolgus whole blood was obtained from three healthy, male cynomolgus monkeys in a heparin vacutainer. DMEM complete media was Iscoves DMEM (with L-glutamine and 25 mM Hepes buffer) containing 0.1% human albumin (Gemini Bioproducts, #800-120), 55 μM 2-mercaptoethanol (Gibco), and 1×Pen-Strep-Gln (PSG, Gibco, Cat#10378-016). Thapsigargin was obtained from Alomone Labs (Israel). A 10 mM stock solution of thapsigargin in 100% DMSO was diluted with DMEM complete media to a 40 μM, 4× solution to provide the 4× thapsigargin stimulus for calcium mobilization. The 2C1.1 monoclonal antibody was diluted in DMEM complete media to a starting 4× concentration of 2000 nM, and serially diluted through 1:4 dilutions for a total of 10 concentrations (4× concentrations were 2000 nM, 375 nM, 93.75 nM, 23.44 nM, 5.86 nM, 1.46 nM, 0.37 nM, 0.09 nM, 0.02 nM, and 0.01 nM). The assay was set up in a 96-well Falcon 3075 flat-bottom microtiter plate. 501 of DMEM complete media was added to columns 11 and 12 as controls, while columns 1-10 received 501 of the 4×2C1.1 titration. To initiate the experiment, 1001 per well of whole blood from each monkey was added to three rows of the microtiter plate. The plate was then incubated at 37° C., 5% CO₂ for one hour. After one hour, the plate was removed and 501 of the 4× thapsigargin stimulus (40 μM) was added to wells in columns 1-11. Column 12 received 50 ul of DMEM complete media. The plates were placed back at 37° C., 5% CO₂ for 48 hours. To determine the amount of IL-4, IL-5 and IL-17 secreted in whole blood, 100 μL of the supernatant (conditioned media) from each well of the 96-well plate was transferred to a storage plate. For MSD electrochemiluminesence analysis of cytokine production, 25 μl of the supernatants (conditioned media) were added to MSD Multi-Spot Custom Coated plates (www.meso-scale.com). The working electrodes on these plates were coated with seven Capture Antibodies (hIL-2, hIL-4, hIL-5, hIL-10, hINFα, hIFNg and hIL-17) in advance. After blocking plates with MSD Human Serum Cytokine Assay Diluent, and then washing three times with PBS containing 0.05% of Tween-20, 25 μl of conditioned media is added to the MSD plate. The plates were covered and placed on a shaking platform for 10 minutes. Next, 25 μl of a cocktail of Detection Antibodies in MSD Antibody Diluent were added to each well. The cocktail contained seven Detection Antibodies (hIL-2, hIL-4, hIL-5, hIL-10, hINFα, hIFNg and hIL-17) at 1 μg/ml each. The plates were covered and placed on a shaking platform overnight (in the dark). The next morning the plates were washed three times with PBS buffer. 150 μl per well of 2×MSD Read Buffer T was added to the plate before reading on the MSD Sector Imager. Since the wells in column 11 of each plate received only the thapsigargin stimulus and no inhibitor, the average MSD response here was used to calculate the “Test” value in a row for each animal. The calculated “Negative Control” value for each animal, was calculated from the average MSD response in column 12 of each row, which received neither thapsigargin nor inhibitor. The “Positive Control” value for the animal was derived from the average MSD response from the wells in columns 11 which contained thapsigargin stimulus, but no inhibitor. Percent of Control (POC) is a measure of the relative inhibition of samples with 2C1.1. Therefore, 100 POC represents maximal response to thapsigargin alone. In contrast, 0 POC represents the amount of cytokine produced in the absence of stimuli. To calculate percent of control (POC), the following formula is used: [(“Test”)−(“Negative Control”)]/[(“Positive Control”)−(“Negative Control”)]×100. Although we describe here measurement of cytokine production using a high throughput MSD electrochemilumenescence assay, one of skill in the art can readily envision lower throughput ELISA assays are equally applicable for measuring cytokine production.

IC50 values for inhibition of IL-4, IL-5, and IL-17 release in the whole blood functional assay for each of the three cynomolgus monkey donors are shown in Table 17 below.

TABLE 17
IC50s (nM) of mAb 2C1.1 in inhibiting Interleukin-4 (IL-4), Interleukin-5
(IL-5), and Interleukin-17 (IL-17) release in the cynomolgus whole
blood assay system.

| | IL-4 | IL-5 | IL-17 |
|---|---|---|---|
| Cyno # 1 | 0.921 | 1.564 | 0.9386 |
| Cyno # 2 | 6.337 | 15.16 | 0.9502 |
| Cyno # 3 | 0.4292 | 0.8153 | 0.3199 |

### ABBREVIATIONS

Abbreviations used throughout this specification are as defined below, unless otherwise defined in specific circumstances.

Ac acetyl (used to refer to acetylated residues)
AcBpa acetylated p-benzoyl-L-phenylalanine
ACN acetonitrile
AcOH acetic acid
ADCC antibody-dependent cellular cytotoxicity
Aib aminoisobutyric acid
bA beta-alanine
Bpa p-benzoyl-L-phenylalanine
BrAc bromoacetyl (BrCH₂C(O)
BSA Bovine serum albumin

### Bzl Benzyl

Cap Caproic acid
CBC complete blood count
COPD Chronic obstructive pulmonary disease
CTL Cytotoxic T lymphocytes

### DCC Dicylcohexylcarbodiimide

Dde 1-(4,4-dimethyl-2,6-dioxo-cyclohexylidene)ethyl
DNP 2,4-dinitrophenol
DOPC 1,2-Dioleoyl-sn-Glycero-3-phosphocholine
DOPE 1,2-Dioleoyl-sn-Glycero-3-phosphoethanolamine
DPPC 1,2-Dipalmitoyl-sn-Glycero-3-phosphocholine
DSPC 1,2-Distearoyl-sn-Glycero-3-phosphocholine

### DTT Dithiothreitol

EAE experimental autoimmune encephalomyelitis
ECL enhanced chemiluminescence
ESI-MS Electron spray ionization mass spectrometry
FACS fluorescence-activated cell sorting
Fmoc fluorenylmethoxycarbonyl
GHT glycine, hypoxanthine, thymidine

### HOBt 1-Hydroxybenzotriazole

HPLC high performance liquid chromatography
HSL homoserine lactone
IB inclusion bodies
KCa calcium-activated potassium channel (including IKCa, BKCa, SKCa)

### KLH Keyhole Limpet Hemocyanin

Kv voltage-gated potassium channel
Lau Lauric acid
LPS lipopolysaccharide
LYMPH lymphocytes
MALDI-MS Matrix-assisted laser desorption ionization mass spectrometry
Me methyl
MeO methoxy
MeOH methanol
MHC major histocompatibility complex
MMP matrix metalloproteinase

### MW Molecular Weight

### MWCO Molecular Weight Cut Off

1-Nap 1-napthylalanine
NEUT neutrophils
Nle norleucine
NMP N-methyl-2-pyrrolidinone
OAc acetate
PAGE polyacrylamide gel electrophoresis
PBMC peripheral blood mononuclear cell
PBS Phosphate-buffered saline
Pbf 2,2,4,6,7-pendamethyldihydrobenzofuran-5-sulfonyl
PCR polymerase chain reaction
PD pharmacodynamic
Pec pipecolic acid
PEG Poly(ethylene glycol)
pGlu pyroglutamic acid
Pic picolinic acid
PK pharmacokinetic
pY phosphotyrosine
RBS ribosome binding site
RT room temperature (about 25° C.)
Sar sarcosine
SDS sodium dodecyl sulfate
STK serine-threonine kinases
t-Boc tert-Butoxycarbonyl
tBu tert-Butyl
TCR T cell receptor
TFA trifluoroacetic acid
THF thymic humoral factor
Trt trityl

1-56. (canceled)

57. An isolated antigen binding protein that specifically binds to SEQ ID NO:4 in the extracellular loop (ECL) 2 of native human Orai1.

58. The isolated antigen binding protein of Claim 57 that:
(a) specifically binds to a native human Orai1 polypeptide,
(b) specifically binds to a polypeptide having an amino acid sequence consisting of
(i) SEQ ID NO:210;
(ii) SEQ ID NO:204;
(iii) SEQ ID NO:192;
(iv) SEQ ID NO:129; or
(v) SEQ ID NO:103; and
(c) does not specifically bind to a polypeptide having an amino acid sequence consisting of
(vi) SEQ ID NO:91;
(vii) SEQ ID NO:198;
(viii) SEQ ID NO:113;
(ix) SEQ ID NO:123;
(x) SEQ ID NO:107; or
(xi) SEQ ID NO:117.

59. The isolated antigen binding protein of Claim 57 or Claim 58, wherein the isolated antigen binding protein comprises an antibody or antibody fragment.

60. The isolated antigen binding protein of Claim 59, comprising an immunoglobulin heavy chain variable region and an immunoglobulin light chain variable region, wherein:
(a) the heavy chain variable region comprises an amino acid sequence at least 95% identical to SEQ ID NO:40, SEQ ID NO:39, SEQ ID NO:41, SEQ ID NO:42, SEQ ID NO:250, SEQ ID NO:251; SEQ ID NO:252, SEQ ID NO:253, SEQ ID NO:254, or SEQ ID NO:255; or
(b) the light chain variable region comprises an amino acid sequence at least 95% identical to SEQ ID NO:36, SEQ ID NO:37, SEQ ID NO:38, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, or SEQ ID NO:265; or
(c) the heavy chain variable region of (a) and the light chain variable region of (b).

61. The isolated antigen binding protein of Claim 59, comprising an immunoglobulin heavy chain variable region and an immunoglobulin light chain variable region, the heavy chain variable region comprising three complementarity determining regions designated CDRH1, CDRH2 and CDRH3, and the light chain variable region comprising three CDRs designated CDRL1, CDRL2 and CDRL3, wherein:
(a) CDRH1 comprises the amino acid sequence of SEQ ID NO:43, SEQ ID NO:44, SEQ ID NO:45, SEQ ID NO:266, or SEQ ID NO:267;
(b) CDRH2 comprises the amino acid sequence of SEQ ID NO:47, SEQ ID NO:46, SEQ ID NO:48, SEQ ID NO:49, SEQ ID NO:268, SEQ ID NO:269, SEQ ID NO:270, SEQ ID NO:271, or SEQ ID NO:272;
(c) CDRH3 comprises the amino acid sequence of SEQ ID NO:50, SEQ ID NO:51, SEQ ID NO:52, SEQ ID NO:273, SEQ ID NO:274, SEQ ID NO:275, SEQ ID NO:276, or SEQ ID NO:277;
(d) CDRL1 comprises the amino acid sequence of SEQ ID NO:53, SEQ ID NO:54, SEQ ID NO:55, SEQ ID NO:278, SEQ ID NO:279, SEQ ID NO:280, SEQ ID NO:281, SEQ ID NO:282, or SEQ ID NO:283;
(e) CDRL2 comprises the amino acid sequence of SEQ ID NO:56, SEQ ID NO:57, SEQ ID NO:284, SEQ ID NO:285, SEQ ID NO:286, SEQ ID NO:287, SEQ ID NO:288, or SEQ ID NO:289; and
(f) CDRL3 comprises the amino acid sequence of SEQ ID NO:58, SEQ ID NO:59, SEQ ID NO:290, SEQ ID NO:291, SEQ ID NO:292, SEQ ID NO:293, SEQ ID NO:294, SEQ ID NO:295, SEQ ID NO:296, or SEQ ID NO:297.

62. The isolated antigen binding protein of Claim 59, comprising:
(a) an immunoglobulin heavy chain comprising the amino acid sequence of SEQ ID NO:34, SEQ ID NO: 29, SEQ ID NO:33, or SEQ ID NO:35; or
(b) an immunoglobulin light chain comprising the amino acid sequence of SEQ ID NO:31, SEQ ID NO: 30, or SEQ ID NO:32; or
(c) the immunoglobulin heavy chain of (a) and the immunoglobulin light chain of (b).

63. The isolated antigen binding protein of Claim 57 or Claim 58,
wherein the isolated antigen binding protein inhibits human calcium response-activated calcium (CRAC) channel activity.

64. The isolated antigen binding protein of Claim 57 or Claim 58, wherein the isolated antigen binding protein inhibits release of IL-2, IFN-gamma, or both, in thapsigargin-treated human whole blood.

65. The isolated antigen binding protein of Claim 57 or Claim 58, wherein the protein inhibits NFAT-mediated expression.

66. The isolated antigen binding protein of Claim 57 or Claim 58, comprising an IgG1, IgG2, IgG3 or IgG4.

67. The isolated antigen binding protein of Claim 59, comprising a monoclonal antibody.

68. The isolated antigen binding protein of Claim 67, comprising a chimeric or humanized antibody.

69. The isolated antigen binding protein of Claim 67, comprising a human antibody.

70. The isolated antigen binding protein of Claim 59, comprising:
(a) an immunoglobulin heavy chain comprising the amino acid sequence of SEQ ID NO:326, SEQ ID NO: 325, SEQ ID NO:327, SEQ ID NO:328, SEQ ID NO:343, SEQ ID NO:344, SEQ ID NO:345, SEQ ID NO:346, SEQ ID NO:347, or SEQ ID NO:348, or comprising the foregoing sequence from which one, two, three, four or five amino acid residues are lacking from the N-terminal or C-terminal, or both; or
(b) an immunoglobulin light chain comprising the amino acid sequence of SEQ ID NO: 322, SEQ ID NO:323, SEQ ID NO:324, SEQ ID NO:333, SEQ ID NO:334, SEQ ID NO:335, SEQ ID NO:336, SEQ ID NO:337, SEQ ID NO:338, SEQ ID NO:339, SEQ ID NO:340, SEQ ID NO:341, or SEQ ID NO:342, or comprising the foregoing sequence from which one, two, three, four or five amino acid residues are lacking from the N-terminal or C-terminal, or both; or
(c) the immunoglobulin heavy chain of (a) and the immunoglobulin light chain of (b).

71. A pharmaceutical composition comprising an antigen binding protein of Claim 57 or Claim 58; and a pharmaceutically acceptable diluent, excipient or carrier.

72. An isolated nucleic acid that encodes the antigen binding protein of Claims 57, 58, or 59.

73. The isolated nucleic acid of Claim 72, wherein the isolated nucleic acid encodes an antigen binding protein comprising an immunoglobulin heavy chain variable region comprising an amino acid sequence at least 95% identical to SEQ ID NO:40, SEQ ID NO:39, SEQ ID NO:41, SEQ ID NO:42, SEQ ID NO:250, SEQ ID NO:251; SEQ ID NO:252, SEQ ID NO:253, SEQ ID NO:254, or SEQ ID NO:255.

74. The isolated nucleic acid of Claim 72, wherein the isolated nucleic acid encodes an immunoglobulin heavy chain variable region and N-terminal signal sequence, the nucleic acid having SEQ ID NO:23, SEQ ID NO:25, or SEQ ID NO:27.

75. The isolated nucleic acid of Claim 72, wherein the isolated nucleic acid encodes an antigen binding protein comprising an immunoglobulin light chain variable region comprising an amino acid sequence at least 95% identical to SEQ ID NO:36, SEQ ID NO:37, SEQ ID NO:38, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, or SEQ ID NO:265.

76. The isolated nucleic acid of Claim 72, wherein the isolated nucleic acid encodes an immunoglobulin light chain variable region and N-terminal signal sequence, the nucleic acid having SEQ ID NO:15, SEQ ID NO:17, or SEQ ID NO:19.

77. The isolated nucleic acid of Claim 72, that encodes an immunoglobulin heavy chain variable region, wherein the isolated nucleic acid comprises coding sequences for three complementarity determining regions, designated CDRH1, CDRH2 and CDRH3, and wherein:
(a) CDRH1 comprises the amino acid sequence of SEQ ID NO:43, SEQ ID NO:44, SEQ ID NO:45, SEQ ID NO:266, or SEQ ID NO:267;
(b) CDRH2 comprises the amino acid sequence of SEQ ID NO:47, SEQ ID NO:46, SEQ ID NO:48, SEQ ID NO:49, SEQ ID NO:268, SEQ ID NO:269,
SEQ ID NO:270, SEQ ID NO:271, or SEQ ID NO:272; and
(c) CDRH3 comprises the amino acid sequence of SEQ ID NO:50, SEQ ID NO:51, SEQ ID NO:52, SEQ ID NO:273, SEQ ID NO:274, SEQ ID NO:275, SEQ ID NO:276, or SEQ ID NO:277.

78. The isolated nucleic acid of Claim 72, that encodes an immunoglobulin light chain variable region, wherein the isolated nucleic acid comprises coding sequences for three complementarity determining regions, designated CDRL1, CDRL2 and CDRL3, and wherein:
(a) CDRL1 comprises the amino acid sequence of SEQ ID NO:53, SEQ ID NO:54, SEQ ID NO:55, SEQ ID NO:278, SEQ ID NO:279, SEQ ID NO:280, SEQ ID NO:281, SEQ ID NO:282, or SEQ ID NO:283;
(b) CDRL2 comprises the amino acid sequence of SEQ ID NO:56, SEQ ID NO:57, SEQ ID NO:284, SEQ ID NO:285, SEQ ID NO:286, SEQ ID NO:287, SEQ ID NO:288, or SEQ ID NO:289; and
(c) CDRL3 comprises the amino acid sequence of SEQ ID NO:58, SEQ ID NO:59, SEQ ID NO:290, SEQ ID NO:291, SEQ ID NO:292, SEQ ID NO:293, SEQ ID NO:294, SEQ ID NO:295, SEQ ID NO:296, or SEQ ID NO:297.

79. The isolated nucleic acid of Claim 72, wherein the isolated nucleic acid encodes an antigen binding protein comprising an immunoglobulin heavy chain comprising the amino acid sequence of SEQ ID NO: 29, SEQ ID NO:33, SEQ ID NO:34, or SEQ ID NO:35.

80. The isolated nucleic acid of Claim 72, wherein the isolated nucleic acid encodes an antigen binding protein comprising an immunoglobulin light chain comprising the amino acid sequence of SEQ ID NO: 30, SEQ ID NO:31, or SEQ ID NO:32.

81. A vector comprising the isolated nucleic acid of Claim 72.

82. The vector of Claim 81, comprising an expression vector.

83. An isolated host cell comprising the expression vector of claim 82.

84. A method comprising:
(a) culturing the host cell of claim 83 in a culture medium under conditions permitting expression of the antigen binding protein encoded by the expression vector; and
(b) recovering the antigen binding protein from the culture medium.

85. A hybridoma, wherein the hybridoma produces the antigen binding protein of Claim 67.

86. A method, comprising:
(a) culturing the hybridoma of claim 85 in a culture medium under conditions permitting expression of the antigen binding protein by the hybridoma; and
(b) recovering the antigen binding protein from the culture medium.

87. A method of treating an immune disorder in a patient, comprising administering an effective amount of the antigen binding protein of any of Claims 57, 58, or 59 to the patient, wherein the immune disorder is selected from T cell-mediated autoimmunity, transplant rejection, graft versus host disease (GVHD), rheumatoid arthritis, multiple sclerosis, type-1 diabetes, systemic lupus erythematosus, psoriasis, inflammatory bowel disease (IBD), asthma, allergic rhinitis, eosinophilic disease, autoimmune central nervous system (CNS) inflammation, and inflammation-induced liver injury.

88. A method of treating a disorder related to venous or arterial thrombus formation in a patient, comprising administering an effective amount of the antigen binding protein of any of Claims 57, 58, or 59 to the patient, wherein the disorder is selected from arterial thrombosis, myocardial infarction, stroke, ischemic reperfusion injury, ischemic brain infarction, inflammation, complement activation, fibrinolysis, angiogenesis related to FXII-induced kinin formation, hereditary angioedema, bacterial infection of the lung, trypanosome infection, hypotensitive shock, pancreatitis, chagas disease, thrombocytopenia and articular gout.

89. A method of treating breast cancer in a patient, comprising administering an effective amount of the antigen binding protein of any of Claims 57, 58, or 59 to the patient.

90. The isolated antigen binding protein of Claim 57 or Claim 58, wherein the antigen binding protein specifically binds to SEQ ID NO:2 expressed by a mammalian cell, with a K_{d} of 200 μM or less, as determined by a Kinetic Exclusion Assay.

91. The isolated antigen binding protein of Claim 57 or Claim 58, wherein the antigen binding protein specifically binds to SEQ ID NO:2 expressed by a mammalian cell, with a K_{d} of 105 μM or less, as determined by a Kinetic Exclusion Assay.

92. The isolated antigen binding protein of Claim 57 or Claim 58, wherein the antigen binding protein specifically binds to SEQ ID NO:2 expressed by a mammalian cell, with a KI of 50 μM or less, as determined by a Kinetic Exclusion Assay.


SEQUENCE LISTING (348 sequences)

SEQ ID NO: 1 - DNA, 906 bp, Homo sapiens
  misc_feature: ORAI1
atgcatccggagcccgccccgcccccgagccgcagcagtcccgagcttcccccaagcggcggcagcaccaccagcggcagccgccggagccgccgccgcagcggggacggggagcccccgggggccccgccaccgccgccgtccgccgtcacctacccggactggatcggccagagttactccgaggtgatgagcctcaacgagcactccatgcaggcgctgtcctggcgcaagctctacttgagccgcgccaagcttaaagcctccagccggacctcggctctgctctccggcttcgccatggtggcaatggtggaggtgcagctggacgctgaccacgactacccaccggggctgctcatcgccttcagtgcctgcaccacagtgctggtggctgtgcacctgtttgcgctcatgatcagcacctgcatcctgcccaacatcgaggcggtgagcaacgtgcacaatctcaactcggtcaaggagtccccccatgagcgcatgcaccgccacatcgagctggcctgggccttctccaccgtcatcggcacgctgctcttcctagctgaggtggtgctgctctgctgggtcaagttcttgcccctcaagaagcagccaggccagccaaggcccaccagcaagccccccgccagtggcgcagcagccaacgtcagcaccagcggcatcaccccgggccaggcagctgccatcgcctcgaccaccatcatggtgcccttcggcctgatctttatcgtcttcgccgtccacttctaccgctcactggttagccataagactgaccgacagttccaggagctcaacgagctggcggagtttgcccgcttacaggaccagctggaccacagaggggaccaccccctgacgcccggcagccactatgcctag

SEQ ID NO: 2 - protein, 301 aa, Homo sapiens
MHPEPAPPPSRSSPELPPSGGSTTSGSRRSRRRSGDGEPPGAPPPPPSAVTYPDWIGQSYSEVMSLNEHSMQALSWRKLYLSRAKLKASSRTSALLSGFAMVAMVEVQLDADHDYPPGLLIAFSACTTVLVAVHLFALMISTCILPNIEAVSNVHNLNSVKESPHERMHRHIELAWAFSTVIGTLLFLAEVVLLCWVKFLPLKKQPGQPRPTSKPPASGAAANVSTSGITPGQAAAIASTTIMVPFGLIFIVFAVHFYRSLVSHKTDRQFQELNELAEFARLQDQLDHRGDHPLTPGSHYA

SEQ ID NO: 3 - protein, 8 aa, Homo sapiens
  MISC_FEATURE: Putative ORAI1 ECL1 domain sequence
DADHDYPP

SEQ ID NO: 4 - protein, 36 aa, Homo sapiens
  MISC_FEATURE: Putative ORAI1 ECL2 domain sequence
KFLPLKKQPGQPRPTSKPPASGAAANVSTSGITPGQ

SEQ ID NO: 5 - DNA, 2058 bp, Homo sapiens
  misc_feature: STIM1
atggatgtatgcgtccgtcttgccctgtggctcctctggggactcctcctgcaccagggccagagcctcagccatagtcacagtgagaaggcgacaggaaccagctcgggggccaactctgaggagtccactgcagcagagttttgccgaattgacaagcccctgtgtcacagtgaggatgagaaactcagcttcgaggcagtccgtaacatccacaaactgatggacgatgatgccaatggtgatgtggatgtggaagaaagtgatgagttcctgagggaagacctcaattaccatgacccaacagtgaaacacagcaccttccatggtgaggataagctcatcagcgtggaggacctgtggaaggcatggaagtcatcagaagtatacaattggaccgtggatgaggtggtacagtggctgatcacatatgtggagctgcctcagtatgaggagaccttccggaagctgcagctcagtggccatgccatgccaaggctggctgtcaccaacaccaccatgacagggactgtgctgaagatgacagaccggagtcatcggcagaagctgcagctgaaggctctggatacagtgctctttgggcctcctctcttgactcgccataatcacctcaaggacttcatgctggtggtgtctatcgttattggtgtgggcggctgctggtttgcctatatccagaaccgttactccaaggagcacatgaagaagatgatgaaggacttggaggggttacaccgagctgagcagagtctgcatgaccttcaggaaaggctgcacaaggcccaggaggagcaccgcacagtggaggtggagaaggtccatctggaaaagaagctgcgcgatgagatcaaccttgctaagcaggaagcccagcggctgaaggagctgcgggagggtactgagaatgagcggagccgccaaaaatatgctgaggaggagttggagcaggttcgggaggccttgaggaaagcagagaaggagctagaatctcacagctcatggtatgctccagaggcccttcagaagtggctgcagctgacacatgaggtggaggtgcaatattacaacatcaagaagcaaaatgctgagaagcagctgctggtggccaaggagggggctgagaagataaaaaagaagagaaacacactctttggcaccttccacgtggcccacagctcttccctggatgatgtagatcataaaattctaacagctaagcaagcactgagcgaggtgacagcagcattgcgggagcgcctgcaccgctggcaacagatcgagatcctctgtggcttccagattgtcaacaaccctggcatccactcactggtggctgccctcaacatagaccccagctggatgggcagtacacgccccaaccctgctcacttcatcatgactgacgacgtggatgacatggatgaggagattgtgtctcccttgtccatgcagtcccctagcctgcagagcagtgttcggcagcgcctgacggagccacagcatggcctgggatctcagagggatttgacccattccgattcggagtcctccctccacatgagtgaccgccagcgtgtggcccccaaacctcctcagatgagccgtgctgcagacgaggctctcaatgccatgacttccaatggcagccaccggctgatcgagggggtccacccagggtctctggtggagaaactgcctgacagccctgccctggccaagaaggcattactggcgctgaaccatgggctggacaaggcccacagcctgatggagctgagcccctcagccccacctggtggctctccacatttggattcttcccgttctcacagccccagctccccagacccagacacaccatctccagttggggacagccgagccctgcaagccagccgaaacacacgcattccccacctggctggcaagaaggctgtggctgaggaggataatggctctattggcgaggaaacagactccagcccaggccggaagaagtttcccctcaaaatctttaagaagcctcttaagaagtag

SEQ ID NO: 6 - protein, 685 aa, Homo sapiens
MDVCVRLALWLLWGLLLHQGQSLSHSHSEKATGTSSGANSEESTAAEFCRIDKPLCHSEDEKLSFEAVRNIHKLMDDDANGDVDVEESDEFLREDLNYHDPTVKHSTFHGEDKLISVEDLWKAWKSSEVYNWTVDEVVQWLITYVELPQYEETFRKLQLSGHAMPRLAVTNTTMTGTVLKMTDRSHRQKLQLKALDTVLFGPPLLTRHNHLKDFMLVVSIVIGVGGCWFAYIQNRYSKEHMKKMMKDLEGLHRAEQSLHDLQERLHKAQEEHRTVEVEKVHLEKKLRDEINLAKQEAQRLKELREGTENERSRQKYAEEELEQVREALRKAEKELESHSSWYAPEALQKWLQLTHEVEVQYYNIKKQNAEKQLLVAKEGAEKIKKKRNTLFGTFHVAHSSSLDDVDHKILTAKQALSEVTAALRERLHRWQQIEILCGFQIVNNPGIHSLVAALNIDPSWMGSTRPNPAHFIMTDDVDDMDEEIVSPLSMQSPSLQSSVRQRLTEPQHGLGSQRDLTHSDSESSLHMSDRQRVAPKPPQMSRAADEALNAMTSNGSHRLIEGVHPGSLVEKLPDSPALAKKALLALNHGLDKAHSLMELSPSAPPGGSPHLDSSRSHSPSSPDPDTPSPVGDSRALQASRNTRIPHLAGKKAVAEEDNGSIGEETDSSPGRKKFPLKIFKKPLKK

SEQ ID NO: 7 - DNA, 720 bp, Artificial Sequence
  Yellow fluorescent protein coding sequence
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcgtgcagtgcttcgcccgctaccccgaccacatgcgccagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtag

SEQ ID NO: 8 - protein, 239 aa, Artificial Sequence
  Synthetic Construct
MVSKGEELFTGVVPILVELDGDVNGHKFSVSGEGEGDATYGKLTLKFICTTGKLPVPWPTLVTTFGYGVQCFARYPDHMRQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 9 - DNA, 2775 bp, Artificial Sequence
  hSTIM1 yellow fluorescent protein coding sequence
atggatgtatgcgtccgtcttgccctgtggctcctctggggactcctcctgcaccagggccagagcctcagccatagtcacagtgagaaggcgacaggaaccagctcgggagccaacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcgtgcagtgcttcgcccgctaccccgaccacatgcgccagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtctgaggagtcgactgcagcagagttttgccgaattgacaagcccctgtgtcacagtgaggatgagaaactcagcttcgaggcagtccgtaacatccacaaactgatggacgatgatgccaatggtgatgtggatgtggaagaaagtgatgagttcctgagggaagacctcaattaccatgacccaacagtgaaacacagcaccttccatggtgaggataagctcatcagcgtggaggacctgtggaaggcatggaagtcatcagaagtatacaattggaccgtggatgaggtggtacagtggctgatcacatatgtggagctgcctcagtatgaggagaccttccggaagctgcagctcagtggccatgccatgccaaggctggctgtcaccaacaccaccatgacagggactgtgctgaagatgacagaccggagtcatcggcagaagctgcagctgaaggctctggatacagtgctctttgggcctcctctcttgactcgccataatcacctcaaggacttcatgctggtggtgtctatcgttattggtgtgggcggctgctggtttgcctatatccagaaccgttactccaaggagcacatgaagaagatgatgaaggacttggaggggttacaccgagctgagcagagtctgcatgaccttcaggaaaggctgcacaaggcccaggaggagcaccgcacagtggaggtggagaaggtccatctggaaaagaagctgcgcgatgagatcaaccttgctaagcaggaagcccagcggctgaaggagctgcgggagggtactgagaatgagcggagccgccaaaaatatgctgaggaggagttggagcaggttcgggaggccttgaggaaagcagagaaggagctagaatctcacagctcatggtatgctccagaggcccttcagaagtggctgcagctgacacatgaggtggaggtgcaatattacaacatcaagaagcaaaatgctgagaagcagctgctggtggccaaggagggggctgagaagataaaaaagaagagaaacacactctttggcaccttccacgtggcccacagctcttccctggatgatgtagatcataaaattctaacagctaagcaagcactgagcgaggtgacagcagcattgcgggagcgcctgcaccgctggcaacagatcgagatcctctgtggcttccagattgtcaacaaccctggcatccactcactggtggctgccctcaacatagaccccagctggatgggcagtacacgccccaaccctgctcacttcatcatgactgacgacgtggatgacatggatgaggagattgtgtctcccttgtccatgcagtcccctagcctgcagagcagtgttcggcagcgcctgacggagccacagcatggcctgggatctcagagggatttgacccattccgattcggagtcctccctccacatgagtgaccgccagcgtgtggcccccaaacctcctcagatgagccgtgctgcagacgaggctctcaatgccatgacttccaatggcagccaccggctgatcgagggggtccacccagggtctctggtggagaaactgcctgacagccctgccctggccaagaaggcattactggcgctgaaccatgggctggacaaggcccacagcctgatggagctgagcccctcagccccacctggtggctctccacatttggattcttcccgttctcacagccccagctccccagacccagacacaccatctccagttggggacagccgagccctgcaagccagccgaaacacacgcattccccacctggctggcaagaaggctgtggctgaggaggataatggctctattggcgaggaaacagactccagcccaggccggaagaagtttcccctcaaaatctttaagaagcctcttaagaagtag

SEQ ID NO: 10 - protein, 924 aa, Artificial Sequence [sequence omitted]

SEQ ID NO: 11 - DNA, 39 bp, Artificial Sequence
  Forward primer sequence
cggatcctgaaccaccatgcatccggagcccgccccgcc

SEQ ID NO: 12 - DNA, 31 bp, Artificial Sequence
  Reverse primer sequence
gcggccgcctaggcatagtggctgccgggcg

SEQ ID NO: 13 - DNA, 318 bp, Homo sapiens
  misc_feature: CL1 CONSTANT REGION coding sequence
ggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 14 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 15 - DNA, 399 bp, Artificial Sequence
  2D2.1 VL coding sequence
atggacatgagggtgcccgctcagctcctggggctcctgctgctgtggctgagaggtgcgcgctgtcagtctgtgctgacgcagccgccctcagtgtctggggccccagggcagagggtcaccatctcctgcactgggagcagctccaatatcggggcaggttataatgtacactggtaccagcaatttccaagaacagaccccaaactcctcatctatgtttacaacattcggccctcaggggtccctgaccgattctctggctccaggtctggcacctctgcctccctggccatcactgggctccagactgaagatgaggctgattattactgccagtcctatgacagcagcctgagtggtgtggttttcggcggagggaccaagctgaccgtccta

SEQ ID NO: 16 - protein, 133 aa, Artificial Sequence
  Synthetic Construct
MDMRVPAQLLGLLLLWLRGARCQSVLTQPPSVSGAPGQRVTISCTGSSSNIGAGYNVHWYQQFPRTDPKLLIYVYNIRPSGVPDRFSGSRSGTSASLAITGLQTEDEADYYCQSYDSSLSGVVFGGGTKLTVL

SEQ ID NO: 17 - DNA, 399 bp, Artificial Sequence
  2B7.1 VL coding sequence
atggacatgagggtgcccgctcagctcctggggctcctgctgctgtggctgagaggtgcgcgctgtcagtctgtgctgacgcagccgccctcagtgtctggggccccagggcagagggtcaccatctcctgcactgggagcagctccaatatcgggacaggttataatgtacactggtaccagcaatttccaagaacagaccccaaactcctcatctatgtttacaacattcggccctcaggggtccctgaccgattctctggctccaggtctggcacctctgcctccctggccatcactggactccagactgaagatgaggctgattattactgccagtcctatgacagcagcctgagtggtgtggttttcggcggagggaccaagctgaccgtccta

SEQ ID NO: 18 - protein, 133 aa, Artificial Sequence
  Synthetic Construct
MDMRVPAQLLGLLLLWLRGARCQSVLTQPPSVSGAPGQRVTISCTGSSSNIGTGYNVHWYQQFPRTDPKLLIYVYNIRPSGVPDRFSGSRSGTSASLAITGLQTEDEADYYCQSYDSSLSGVVFGGGTKLTVL

SEQ ID NO: 19 - DNA, 399 bp, Artificial Sequence
  2B4.1 VL coding sequence
atggacatgagggtgcccgctcagctcctggggctcctgctgctgtggctgagaggtgcgcgctgtcagtctgtgctgacgcagccgccctcagtgtctggggccccagggcagagggtcaccatctcctgcactgggagcaattccaacatcgggacaggttatgatgttcactggtaccagaaacttccaggaacagcccccagactcctcatctatagtcacttcaatcggccctcaggggtccctgaccgattctctggctccacgtctggcacctcagcctccctggccatcactgggctccaggctgaggatgaggctgattattactgccagtcctatgacagcagcctgagtggttcggtgttcggcggagggaccaagctgaccgtccta

SEQ ID NO: 20 - protein, 133 aa, Artificial Sequence
  Synthetic Construct
MDMRVPAQLLGLLLLWLRGARCQSVLTQPPSVSGAPGQRVTISCTGSNSNIGTGYDVHWYQKLPGTAPRLLIYSHFNRPSGVPDRFSGSTSGTSASLAITGLQAEDEADYYCQSYDSSLSGSVFGGGTKLTVL

SEQ ID NO: 21 - DNA, 978 bp, Homo sapiens
  misc_feature: IgG2 constant region coding sequence
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 22 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 23 - DNA, 426 bp, Artificial Sequence
  2D2.1 VH coding sequence
atggacatgagggtgcccgctcagctcctggggctcctgctgctgtggctgagaggtgcgcgctgtcaggtgcagctacagcagtggggcgcaggactgttgaagccttcggagaccctgtccctcacctgcgctgtctatggtgggtccttcagtggttactactggagctggatccgccagccccccggaaaggggctggagtggattggggaaatcgatcatagtggaaggataaattacaacccggccctcaagagtcgactcaccatatcagttgacacgtccaagaaccagttctccctgaagctgagctctgtgaccgccgcggacacggctgtgtattactgtgcgagagcaggttcggggggttatgaggactggttcgacccctggggccagggaaccctggtcaccgtctctagt

SEQ ID NO: 24 - protein, 142 aa, Artificial Sequence
  Synthetic Construct
MDMRVPAQLLGLLLLWLRGARCQVQLQQWGAGLLKPSETLSLTCAVYGGSFSGYYWSWIRQPPGKGLEWIGEIDHSGRINYNPALKSRLTISVDTSKNQFSLKLSSVTAADTAVYYCARAGSGGYEDWFDPWGQGTLVTVSS

SEQ ID NO: 25 - DNA, 426 bp, Artificial Sequence
  2C1.1 VH coding sequence
atggacatgagggtgcccgctcagctcctggggctcctgctgctgtggctgagaggtgcgcgctgtcaggtgcagctacagcagtggggcgcaggactgttgaagccttcggagactctgtccctcacctgcgctgtctatggtgggtccttcagtggttactactggagctggatccgccagccccccggaaaggggctggagtggattggggaaatcgatcatagtggaagcaccaactacaacccggccctcaagagtcgactcaccatatcagttgacacgtccaagaaccagttctccctgaagctgagctctgtgaccgccgcggacacggctgtgtattactgtgcgagagcaggttcggggggttatgaggactggttcgacccctggggccagggaaccctggtcaccgtctctagt

SEQ ID NO: 26 - protein, 142 aa, Artificial Sequence
  Synthetic Construct
MDMRVPAQLLGLLLLWLRGARCQVQLQQWGAGLLKPSETLSLTCAVYGGSFSGYYWSWIRQPPGKGLEWIGEIDHSGSTNYNPALKSRLTISVDTSKNQFSLKLSSVTAADTAVYYCARAGSGGYEDWFDPWGQGTLVTVSS

SEQ ID NO: 27 - DNA, 426 bp, Artificial Sequence
  2G1.1 VH coding sequence
atggacatgagggtgcccgctcagctcctggggctcctgctgctgtggctgagaggtgcgcgctgtgaggtgcagctggtggagtctgggggaggcttggtccagcctggggggtccctgagactctcctgtgcagcctctggattcacctttagtagctattggatgagctgggtccgccaggctccagggaaggggctggagtgggtggccaacataaagcacgatggaagtgagaaatactatgtggactctgtgaagggccgattcaccatctccagagacaatgccaagaactcactgcatctgcaaatgatcagcctgagagccgaggacacggctgtgtattactgtgcgaaaagatatagcggtggctggaccttctttgactactggggccagggaaccctggtcaccgtctctagt

SEQ ID NO: 28 - protein, 142 aa, Artificial Sequence
  Synthetic Construct
MDMRVPAQLLGLLLLWLRGARCEVQLVESGGGLVQPGGSLRLSCAASGFTFSSYWMSWVRQAPGKGLEWVANIKHDGSEKYYVDSVKGRFTISRDNAKNSLHLQMISLRAEDTAVYYCAKRYSGGWTFFDYWGQGTLVTVSS

SEQ ID NO: 29 - protein, 466 aa, Artificial Sequence
  H4 (2B4.1 HC)
MDMRVPAQLLGLLLLWLRGARCQVQLVQSGAEVKKPGASVKVSCKASGYTFTDYYMHWVRQAPGQGLEWMGWINPNSGGTNYVQKFQDRVTMTRDTSITTAYMELTRLRSDDTAVYYCAREEGDYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 30 - protein, 239 aa, Artificial Sequence
  L3 (2B4.1 LC)
MDMRVPAQLLGLLLLWLRGARCQSVLTQPPSVSGAPGQRVTISCTGSNSNIGTGYDVHWYQKLPGTAPRLLIYSHFNRPSGVPDRFSGSTSGTSASLAITGLQAEDEADYYCQSYDSSLSGSVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 31 - protein, 239 aa, Artificial Sequence
  L1 (HC OF 2D2.1, 2C1.1, AND 2G1.1)
MDMRVPAQLLGLLLLWLRGARCQSVLTQPPSVSGAPGQRVTISCTGSSSNIGAGYNVHWYQQFPRTDPKLLIYVYNIRPSGVPDRFSGSRSGTSASLAITGLQTEDEADYYCQSYDSSLSGVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 32 - protein, 239 aa, Artificial Sequence
  L2
MDMRVPAQLLGLLLLWLRGARCQSVLTQPPSVSGAPGQRVTISCTGSSSNIGTGYNVHWYQQFPRTDPKLLIYVYNIRPSGVPDRFSGSRSGTSASLAITGLQTEDEADYYCQSYDSSLSGVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 33 - protein, 468 aa, Artificial Sequence
  H1
MDMRVPAQLLGLLLLWLRGARCQVQLQQWGAGLLKPSETLSLTCAVYGGSFSGYYWSWIRQPPGKGLEWIGEIDHSGRINYNPALKSRLTISVDTSKNQFSLKLSSVTAADTAVYYCARAGSGGYEDWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 34 - protein, 468 aa, Artificial Sequence
  H2
MDMRVPAQLLGLLLLWLRGARCQVQLQQWGAGLLKPSETLSLTCAVYGGSFSGYYWSWIRQPPGKGLEWIGEIDHSGSTNYNPALKSRLTISVDTSKNQFSLKLSSVTAADTAVYYCARAGSGGYEDWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 35 - protein, 468 aa, Artificial Sequence
  H3
MDMRVPAQLLGLLLLWLRGARCEVQLVESGGGLVQPGGSLRLSCAASGFTFSSYWMSWVRQAPGKGLEWVANIKHDGSEKYYVDSVKGRFTISRDNAKNSLHLQMISLRAEDTAVYYCAKRYSGGWTFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 36 - protein, 111 aa, Artificial Sequence
  VL1
QSVLTQPPSVSGAPGQRVTISCTGSSSNIGAGYNVHWYQQFPRTDPKLLIYVYNIRPSGVPDRFSGSRSGTSASLAITGLQTEDEADYYCQSYDSSLSGVVFGGGTKLTVL

SEQ ID NO: 37 - protein, 111 aa, Artificial Sequence
  VL2
QSVLTQPPSVSGAPGQRVTISCTGSSSNIGTGYNVHWYQQFPRTDPKLLIYVYNIRPSGVPDRFSGSRSGTSASLAITGLQTEDEADYYCQSYDSSLSGVVFGGGTKLTVL

SEQ ID NO: 38 - protein, 111 aa, Artificial Sequence
  VL3
QSVLTQPPSVSGAPGQRVTISCTGSNSNIGTGYDVHWYQKLPGTAPRLLIYSHFNRPSGVPDRFSGSTSGTSASLAITGLQAEDEADYYCQSYDSSLSGSVFGGGTKLTVL

SEQ ID NO: 39 - protein, 120 aa, Artificial Sequence
  VH1
QVQLQQWGAGLLKPSETLSLTCAVYGGSFSGYYWSWIRQPPGKGLEWIGEIDHSGRINYNPALKSRLTISVDTSKNQFSLKLSSVTAADTAVYYCARAGSGGYEDWFDPWGQGTLVTVSS

SEQ ID NO: 40 - protein, 120 aa, Artificial Sequence
  VH2
QVQLQQWGAGLLKPSETLSLTCAVYGGSFSGYYWSWIRQPPGKGLEWIGEIDHSGSTNYNPALKSRLTISVDTSKNQFSLKLSSVTAADTAVYYCARAGSGGYEDWFDPWGQGTLVTVSS

SEQ ID NO: 41 - protein, 120 aa, Artificial Sequence
  VH3
EVQLVESGGGLVQPGGSLRLSCAASGFTFSSYWMSWVRQAPGKGLEWVANIKHDGSEKYYVDSVKGRFTISRDNAKNSLHLQMISLRAEDTAVYYCAKRYSGGWTFFDYWGQGTLVTVSS

SEQ ID NO: 42 - protein, 118 aa, Artificial Sequence
  VH4
QVQLVQSGAEVKKPGASVKVSCKASGYTFTDYYMHWVRQAPGQGLEWMGWINPNSGGTNYVQKFQDRVTMTRDTSITTAYMELTRLRSDDTAVYYCAREEGDYGMDVWGQGTTVTVSS

SEQ ID NO: 43 - protein, 5 aa, Artificial Sequence
  CDRH1-1
GYYWS

SEQ ID NO: 44 - protein, 5 aa, Artificial Sequence
  CDRH1-2
SYWMS

SEQ ID NO: 45 - protein, 5 aa, Artificial Sequence
  CDRH1-3
DYYMH

SEQ ID NO: 46 - protein, 16 aa, Artificial Sequence
  CDRH2-1
EIDHSGRINYNPALKS

SEQ ID NO: 47 - protein, 16 aa, Artificial Sequence
  CDRH2-2
EIDHSGSTNYNPALKS

SEQ ID NO: 48 - protein, 17 aa, Artificial Sequence
  CDRH2-3
NIKHDGSEKYYVDSVKG

SEQ ID NO: 49 - protein, 17 aa, Artificial Sequence
  CDRH2-4
WINPNSGGTNYVQKFQD

SEQ ID NO: 50 - protein, 12 aa, Artificial Sequence
  CDRH3-1
AGSGGYEDWFDP

SEQ ID NO: 51 - protein, 11 aa, Artificial Sequence
  CDRH3-2
RYSGGWTFFDY

SEQ ID NO: 52 - protein, 9 aa, Artificial Sequence
  CDRH3-3
EEGDYGMDV

SEQ ID NO: 53 - protein, 14 aa, Artificial Sequence
  CDRL1-1
TGSSSNIGAGYNVH

SEQ ID NO: 54 - protein, 14 aa, Artificial Sequence
  CDRL1-2
TGSSSNIGTGYNVH

SEQ ID NO: 55 - protein, 14 aa, Artificial Sequence
  CDRL1-3
TGSNSNIGTGYDVH

SEQ ID NO: 56 - protein, 7 aa, Artificial Sequence
  CDRL2-1
VYNIRPS

SEQ ID NO: 57 - protein, 7 aa, Artificial Sequence
  CDRL2-2
SHFNRPS

SEQ ID NO: 58 - protein, 11 aa, Artificial Sequence
  CDRL3-1
QSYDSSLSGVV

SEQ ID NO: 59 - protein, 11 aa, Artificial Sequence
  CDRL3-2
QSYDSSLSGSV

SEQ ID NO: 60 - DNA, 765 bp, Homo sapiens
  misc_feature: ORAI2 (NCBI Reference sequence NM_032831)
atgagtgctgagcttaacgtgcctatcgacccctctgctcctgcctgccctgagcccggccataagggcatggattaccgggactgggtccgccgcagctacctggaactggtcacctctaaccaccactcggtacaggccctgtcgtggcggaagctctacctgagcagggccaagctgaaggcctccagcaggacctccgccctcctctccggctttgccatggtggccatggtggaggtgcagctggagacgcagtaccagtacccgcggccgctgctgattgccttcagcgcctgcaccacggtgctggtggccgtgcacctgttcgccctcctcatcagcacctgcatcctgcccaatgtggaggccgtgagcaacatccacaacctgaactccatcagcgagtccccgcatgagcgcatgcacccctacatcgagctggcctggggcttctccaccgtgcttggcatcctactcttcctggccgaggtggtgctgctctgctggatcaagttcctccccgtggatgcccggcgccagcctggccccccacctggccctgggagtcacacgggctggcaggccgccctggtgtccaccatcatcatggtgcccgtgggcctcatcttcgtggtcttcaccatccacttctaccgctccctggtgcgccacaaaacggagcgccacaaccgcgagatcgaggagctccacaagctcaaggtccagctggacgggcatgagcgcagcctgcaggtcttgtga

SEQ ID NO: 61 - protein, 254 aa, Homo sapiens
MSAELNVPIDPSAPACPEPGHKGMDYRDWVRRSYLELVTSNHHSVQALSWRKLYLSRAKLKASSRTSALLSGFAMVAMVEVQLETQYQYPRPLLIAFSACTTVLVAVHLFALLISTCILPNVEAVSNIHNLNSISESPHERMHPYIELAWGFSTVLGILLFLAEVVLLCWIKFLPVDARRQPGPPPGPGSHTGWQAALVSTIIMVPVGLIFVVFTIHFYRSLVRHKTERHNREIEELHKLKVQLDGHERSLQVL

SEQ ID NO: 62 - DNA, 888 bp, Homo sapiens
  misc_feature: ORAI3 (NCBI Reference sequence NM_152288)
atgaagggcggcgagggggacgcgggcgagcaggccccgctgaaccctgagggcgagagccctgcaggctcggccacgtaccgggagttcgtgcaccgcggctacctggacctcatgggggccagtcagcactcgctgcgggcgctcagctggcgccgcctctacctcagccgggccaagctcaaagcttccagccgcacgtctgccttgctctcgggcttcgccatggtggccatggtggaggtgcagctggagagtgaccacgagtacccaccaggcctgctggtggccttcagtgcctgcaccaccgtgctggtggctgtgcacctctttgcactcatggtctccacgtgtctgctgccccacattgaagctgtgagcaacatccacaacctcaactctgtccaccagtcgccacaccagagactgcaccgctacgtggagctggcctggggcttctccactgccctgggcacctttctcttccttgctgaagttgtcctggttggttgggtcaagtttgtgcccattggggctcccttggacacaccgacccccatggtgcccacatcccgggtgcccgggactctggcaccagtggctacctcccttagtccagcttccaatctcccacggtcctctgcgtctgcagcaccgtcccaggctgagccagcctgcccaccccggcaagcctgtggtggtggtggggcccatgggccaggctggcaagcagccatggcctccacagccatcatggtacccgtggggctcgtgtttgtggcctttgccctgcatttctaccgctccttggtggcacacaagacagaccgctacaagcaggaactagaggaactgaatcgcctgcagggggagctgcaggctgtgtga

SEQ ID NO: 63 - protein, 295 aa, Homo sapiens [sequence omitted]

SEQ ID NO: 64 - DNA, 906 bp, Homo sapiens
  misc_feature: ORAI1 (S218G) coding sequence
atgcatccggagcccgccccgcccccgagccgcagcagtcccgagcttcccccaagcggcggcagcaccaccagcggcagccgccggagccgccgccgcagcggggacggggagcccccgggggccccgccaccgccgccgtccgccgtcacctacccggactggatcggccagagttactccgaggtgatgagcctcaacgagcactccatgcaggcgctgtcctggcgcaagctctacttgagccgcgccaagcttaaagcctccagccggacctcggctctgctctccggcttcgccatggtggcaatggtggaggtgcagctggacgctgaccacgactacccaccggggctgctcatcgccttcagtgcctgcaccacagtgctggtggctgtgcacctgtttgcgctcatgatcagcacctgcatcctgcccaacatcgaggcggtgagcaacgtgcacaatctcaactcggtcaaggagtccccccatgagcgcatgcaccgccacatcgagctggcctgggccttctccaccgtcatcggcacgctgctcttcctagctgaggtggtgctgctctgctgggtcaagttcttgcccctcaagaagcagccaggccagccaaggcccaccagcaagccccccgccggtggcgcagcagccaacgtcagcaccagcggcatcaccccgggccaggcagctgccatcgcctcgaccaccatcatggtgcccttcggcctgatctttatcgtcttcgccgtccacttctaccgctcactggttagccataagactgaccgacagttccaggagctcaacgagctggcggagtttgcccgcttacaggaccagctggaccacagaggggaccaccccctgacgcccggcagccactatgcctag

SEQ ID NO: 65 - protein, 301 aa, Homo sapiens
MHPEPAPPPSRSSPELPPSGGSTTSGSRRSRRRSGDGEPPGAPPPPPSAVTYPDWIGQSYSEVMSLNEHSMQALSWRKLYLSRAKLKASSRTSALLSGFAMVAMVEVQLDADHDYPPGLLIAFSACTTVLVAVHLFALMISTCILPNIEAVSNVHNLNSVKESPHERMHRHIELAWAFSTVIGTLLFLAEVVLLCWVKFLPLKKQPGQPRPTSKPPAGGAAANVSTSGITPGQAAAIASTTIMVPFGLIFIVFAVHFYRSLVSHKTDRQFQELNELAEFARLQDQLDHRGDHPLTPGSHYA

SEQ ID NO: 66 - DNA, 43 bp, Artificial Sequence
  Forward primer sequence
ccaccagcaagccccccgccggtggcgcagcagccaacgtcag

SEQ ID NO: 67 - DNA, 42 bp, Artificial Sequence
  Reverse primer sequence
ctgacgttggctgctgcgccaccggcggggggcttgctggtg

SEQ ID NO: 68 - DNA, 38 bp, Artificial Sequence
  Forward primer sequence
ggatcctgaaccaccatgcatccggagcctgccccgcc

SEQ ID NO: 69 - DNA, 31 bp, Artificial Sequence
  Reverse primer sequence
gcggccgcttaggcatagtgggtgcccggtg

SEQ ID NO: 70 - protein, 36 aa, Homo sapiens
  MISC_FEATURE: Putative ORAI1 (S218G) ECL2 domain sequence
KFLPLKKQPGQPRPTSKPPAGGAAANVSTSGITPGQ

SEQ ID NO: 71 - DNA, 915 bp, Mus musculus
  misc_feature: ORAI1-NCBI reference sequence NM_1754 23
atgcatccggagcctgccccgcccccgagtcacagcaatccggagcttcccgtgagcggcggcagcagcactagcggcagccgccggagccgccgccgcagcggggacggggagccctcgggggccccaccgctgccgccgccgccacccgccgtcagctacccggactggatcggccagagttactccgaggtgatgagcctcaacgagcactcgatgcaggcgctgtcctggcgcaagctctacttaagccgcgccaagctcaaagcttccagccggacctcggctctgctctccggcttcgccatggtagcgatggtggaagtccagctggacacagaccatgactacccaccagggttgctcatcgtctttagtgcctgcaccacagtgctagtggccgtgcacctgtttgccctcatgatcagcacctgcatcctgcccaacatcgaggctgtgagcaacgtccacaacctcaactcggtcaaagagtcaccccacgagcgcatgcatcgccacatcgagctggcctgggccttctccacggtcatcgggacgctgcttttcctagcagaggtcgtgctgctctgctgggtcaagttcttacctctcaagaggcaagcgggacagccaagccccaccaagcctcccgctgaatcagtcatcgtcgccaaccacagcgacagcagcggcatcaccccgggtgaggcggcagccattgcctccaccgccatcatggttccctgtggcctggtttttatcgtctttgctgttcacttctaccgctccctggtcagccataagacggaccggcagttccaggagctcaatgagctggccgagtttgcccgcttgcaggaccagctggaccacagaggggaccattctctaacaccgggcacccactatgcctag

SEQ ID NO: 72 - protein, 304 aa, Mus musculus
MHPEPAPPPSHSNPELPVSGGSSTSGSRRSRRRSGDGEPSGAPPLPPPPPAVSYPDWIGQSYSEVMSLNEHSMQALSWRKLYLSRAKLKASSRTSALLSGFAMVAMVEVQLDTDHDYPPGLLIVFSACTTVLVAVHLFALMISTCILPNIEAVSNVHNLNSVKESPHERMHRHIELAWAFSTVIGTLLFLAEVVLLCWVKFLPLKRQAGQPSPTKPPAESVIVANHSDSSGITPGEAAAIASTAIMVPCGLVFIVFAVHFYRSLVSHKTDRQFQELNELAEFARLQDQLDHRGDHSLTPGTHYA

SEQ ID NO: 73 - DNA, 2058 bp, Mus musculus
  misc_feature: STIM1-NCBI reference sequence NM_009 287
atggatgtgtgcgcccgtcttgccctgtggcttctttgggggctccttctgcatcagggccagagtctcagccatagtcacagtgaaaagaatacaggagctagctccggggcgacttctgaagagtctaccgaagcagagttttgccgaattgacaagcccctgtgccacagtgaggatgagaagctcagctttgaggccgtccgaaacatccataagctgatggatgacgatgccaatggtgatgtggatgtggaagaaagtgatgagttcctaagggaagacctcaattaccatgacccaacagtgaaacatagcaccttccatggtgaggataagcttatcagcgtggaggacctgtggaaggcgtggaaatcatcagaagtgtacaactggactgtggatgaggtgatacagtggctcattacgtatgtggagctgccacagtatgaggagaccttccggaagttgcagcttactggccacgccatgccaaggctagcagtaaccaacaccaccatgacagggactgtactgaagatgacagatcggagccacaggcagaagctgcagctgaaggccctggacacagtgctgtttgggcctcctctcttgactcggcataatcacctgaaggacttcatgctggtggtgtctatcgttattggtgtgggtggctgctggtttgcctatatccagaaccgttactctaaggagcacatgaagaaaatgatgaaggatctggaagggttacaccgggctgagcagagtctgcatgaccttcaggaaaggctgcacaaggcccaggaggagcaccgaactgtggaagtagagaaagtccacctggagaagaagctgcgagatgagatcaaccttgccaagcaggaagctcagcggctgaaggagctgagggagggtactgagaatgagaggagccgtcaaaaatatgctgaggaagagctggagcaggttcgggaggccttgaggaaagcagagaaggagctggaatcacacagctcatggtatgctcctgaggccctgcagaagtggctgcagctgacccatgaggtggaggtgcagtactacaacatcaagaagcaaaatgcagagaggcagctgctggtggccaaggagggggctgagaaaataaaaaagaagagaaacacgctttttggtaccttccatgtggcccacagctcttccctggatgatgtggatcataaaatcctaactgctaagcaagctctgagtgaggtgacagcggcactgagggagcgcctgcaccggtggcagcagatcgagatcctctgcggtttccagattgtcaataaccccggcatccactccttggtggctgctctcaacatcgaccccagctggatgggcagcacccgccctaaccccgcccacttcatcatgactgacgatgtggatgacatggatgaggagattgtgtcgcccttgtccatgcagtcccccagcctgcagagcagtgtccggcagcgcctgacggagccacagcttggcctgggatctcagagggatttgacccattccgattcggagtcctccctccacatgagtgaccgccagcgtgtggcccccaagcctcctcagatgggccgtgctgcagatgaagctctcaatgccatgccttccaatggcagccatcggctgattgagggggtccatccaggatctctggtggagaaactgcctgacagccctgctctggccaagaagacatttatggcgttgaaccatggcctagacaaggcccacagcctgatggagctgaacccctcagtcccacctggtggctccccacttttggattcttcccattctcttagccccagttccccagacccagacacaccatctccagttggggacaaccgagctctgcagggtagccgaaacacacgaattccccacttggctggcaagaaggcaatggctgaggaggataatggttccattggtgaggagacagactccagtccaggcaggaagaagtttcctctcaaaatttttaagaagcctcttaagaagtag

SEQ ID NO: 74 - protein, 685 aa, Mus musculus
MDVCARLALWLLWGLLLHQGQSLSHSHSEKNTGASSGATSEESTEAEFCRIDKPLCHSEDEKLSFEAVRNIHKLMDDDANGDVDVEESDEFLREDLNYHDPTVKHSTFHGEDKLISVEDLWKAWKSSEVYNWTVDEVIQWLITYVELPQYEETFRKLQLTGHAMPRLAVTNTTMTGTVLKMTDRSHRQKLQLKALDTVLFGPPLLTRHNHLKDFMLVVSIVIGVGGCWFAYIQNRYSKEHMKKMMKDLEGLHRAEQSLHDLQERLHKAQEEHRTVEVEKVHLEKKLRDEINLAKQEAQRLKELREGTENERSRQKYAEEELEQVREALRKAEKELESHSSWYAPEALQKWLQLTHEVEVQYYNIKKQNAERQLLVAKEGAEKIKKKRNTLFGTFHVAHSSSLDDVDHKILTAKQALSEVTAALRERLHRWQQIEILCGFQIVNNPGIHSLVAALNIDPSWMGSTRPNPAHFIMTDDVDDMDEEIVSPLSMQSPSLQSSVRQRLTEPQLGLGSQRDLTHSDSESSLHMSDRQRVAPKPPQMGRAADEALNAMPSNGSHRLIEGVHPGSLVEKLPDSPALAKKTFMALNHGLDKAHSLMELNPSVPPGGSPLLDSSHSLSPSSPDPDTPSPVGDNRALQGSRNTRIPHLAGKKAMAEEDNGSIGEETDSSPGRKKFPLKIFKKPLKK

SEQ ID NO: 75 - DNA, 915 bp, Rattus norvegicus
  misc_feature: ORAI1-NCBI reference sequence NM_001 013982
atgcatccggagcctgccccgcccccgaacaacagcaatccggagcttcccttgagcggcggcagcagcactagcggcagccgccggagccgccgccgcagcggggacggggagcccacgggggccccaccgctgccgccgccgcccgccgtcagctacccggactggatcggccagagttactccgaggtgatgagcctcaacgagcactcgatgcaggcgctgtcctggcgcaagctctacttgagccgcgccaagctcaaagcctccagccgaacctccgctctgctctccggcttcgccatggtagcgatggtggaagtccagctggacacggatcatgactacccgccagggttactcatcgtcttcagcgcctgcaccacagtgctggtggccgtgcacctgttcgccctcatgatcagtacctgcatcctgcccaacatcgaggctgtgagcaacgtccacaacctcaactccgtcaaggagtcgccgcacgagcgcatgcaccgccacatcgagctggcttgggccttctccaccgtcatcgggacgctgcttttcctggccgaagtcgtgctgctctgctgggtgaagttcttaccgctcaagaggcaggcgggacagccaagccccaccaagccacccaccgaaccggccgtcgtcgtcgccaacagcagcaacaacggtggcatcaccccgggtgaggcggctgccatcgcctccactgccatcatggtcccctgtggcctggtgtttatcgtctttgccgttcacttctaccgctcactggtcagccataagacggaccgacagttccaggagctcaacgagctggccgagtttgcccgcttgcaggaccagctggaccacagaggggaccattctctaacaccgggcacccactatgcctaa

SEQ ID NO: 76 - protein, 304 aa, Rattus norvegicus
MHPEPAPPPNNSNPELPLSGGSSTSGSRRSRRRSGDGEPTGAPPLPPPPAVSYPDWIGQSYSEVMSLNEHSMQALSWRKLYLSRAKLKASSRTSALLSGFAMVAMVEVQLDTDHDYPPGLLIVFSACTTVLVAVHLFALMISTCILPNIEAVSNVHNLNSVKESPHERMHRHIELAWAFSTVIGTLLFLAEVVLLCWVKFLPLKRQAGQPSPTKPPTEPAVVVANSSNNGGITPGEAAAIASTAIMVPCGLVFIVFAVHFYRSLVSHKTDRQFQELNELAEFARLQDQLDHRGDHSLTPGTHYA

SEQ ID NO: 77 - DNA, 2058 bp, Rattus norvegicus
  misc_feature: STIM1-NCBI reference sequence XM_341 896
atggatgtgtgcgctcgtcttgccctgtggcttctttgggggctccttctgcatcagggccagagtctcagccatagtcacagtgagaagaatacaggagctagctcgggggcgacttctgaagagtctaccgaagcagagttttgccggattgacaagcccctgtgccacagtgaagatgagaagctcagctttgaggccgtccgcaacatccacaagctgatggatgatgatgccaatggtgatgtggatgtggaagaaagtgatgagttcctaagggaagacctcaattaccatgaccctacagtgaaacacagcaccttccatggtgaggacaagcttatcagcgtggaggacctatggaaggcatggaaggcatcagaagtgtataactggaccgtggatgaggtgatacagtggctgattacgtatgtggagctgccacagtatgaggagactttccggaagttgcagcttactggccatgccatgccaaggctagctgtaacaaacaccaccatgacagggactgtcctgaagatgacagaccggagccacaggcagaagctgcagctgaaggctctggacacagtgctgttcgggcctcctctcttgactcgccacaatcacctgaaggacttcatgctggtggtatctatcgtgattggtgtgggtggctgctggtttgcctatatccagaaccgttactctaaggagcacatgaagaaaatgatgaaggatctggaaggattgcaccgagctgagcagagtctgcatgaccttcaggaaaggctgcacaaggcccaggaggagcaccgaacagtggaagtagagaaggtccacctggagaagaagctgcgtgacgagatcaatcttgccaagcaggaagctcagcggctgaaggagctgagggagggtaccgagaatgagaggagccgccaaaaatatgctgaggaagagctggagcaggttcgggaggctttgaggaaagcagagaaggagctggaatcacacagctcctggtatgctcctgaggccctgcagaagtggctgcagctgacacatgaggtggaggtgcagtactacaacatcaagaagcaaaatgcagagaggcagctgctggtggccaaggagggggctgagaaaataaaaaagaagagaaacacgctttttggtaccttccatgtggcccacagctcgtccctggatgacgtggatcataaaatcttaactgctaagcaggcactgagtgaggtgacagcagccctgagggagcgcctgcaccggtggcagcagatcgagatcctctgtggtttccagattgtcaataaccctggcatccactccttggtggccgcgctcaacatagatcccagctggatgggcagtacccgccctaaccctgcccacttcatcatgactgacgatgtggatgacatggatgaggagatcgtgtcgcccttgtccatgcagtcccccagcctgcagagcagtgtccggcagcgcctgacggagccacagcatggcctgggatctcagagggatttgacccattccgattcggagtcctccctccacacgagtgaccgccagcgtgtggcccccaagcctcctcagatgggccgtgctgcagatgaggctctcaatgccacgtcttccaatggtagccatcggctgattgagggggtccatccaggatctctggtggagaaactgcctgacagccctgctctggccaagaagacaatcctggcgctgaaccatggcctagataaggcccacagcctgatggagctgaacccctcagtcccacctggtggctccccacttttggattcttcccattctcatagccccagctccccagacccagacacaccatctccagttggggacagccgagccctgcagggtagccgaaacacacgaattccccacttggctggcaagaaggctatggctgaggaggataatggctctattggtgaggagacagactctagtccaggcaggaagaagtttcccctcaaaatttttaagaagcctcttaagaagtag

SEQ ID NO: 78 - protein, 685 aa, Rattus norvegicus
MDVCARLALWLLWGLLLHQGQSLSHSHSEKNTGASSGATSEESTEAEFCRIDKPLCHSEDEKLSFEAVRNIHKLMDDDANGDVDVEESDEFLREDLNYHDPTVKHSTFHGEDKLISVEDLWKAWKASEVYNWTVDEVIQWLITYVELPQYEETFRKLQLTGHAMPRLAVTNTTMTGTVLKMTDRSHRQKLQLKALDTVLFGPPLLTRHNHLKDFMLVVSIVIGVGGCWFAYIQNRYSKEHMKKMMKDLEGLHRAEQSLHDLQERLHKAQEEHRTVEVEKVHLEKKLRDEINLAKQEAQRLKELREGTENERSRQKYAEEELEQVREALRKAEKELESHSSWYAPEALQKWLQLTHEVEVQYYNIKKQNAERQLLVAKEGAEKIKKKRNTLFGTFHVAHSSSLDDVDHKILTAKQALSEVTAALRERLHRWQQIEILCGFQIVNNPGIHSLVAALNIDPSWMGSTRPNPAHFIMTDDVDDMDEEIVSPLSMQSPSLQSSVRQRLTEPQHGLGSQRDLTHSDSESSLHTSDRQRVAPKPPQMGRAADEALNATSSNGSHRLIEGVHPGSLVEKLPDSPALAKKTILALNHGLDKAHSLMELNPSVPPGGSPLLDSSHSHSPSSPDPDTPSPVGDSRALQGSRNTRIPHLAGKKAMAEEDNGSIGEETDSSPGRKKFPLKIFKKPLKK

SEQ ID NO: 79 - DNA, 2057 bp, Pan troglodytes
  misc_feature: ORAI1 coding sequence-NCBI Accession XM_528 664
ctcgtcctgtgcggccggccgggtgaggccgggcccgcgtagggggcagtcggcggctgcctccggcggaggtgcctcgcggcgcccgggccggcccgcgcctcggcggcgtgctccatgcatccggagcccgccccgcccccgagcggcagcagccccgagcttcccccaagcggcggtagcaccaccagcggcagccgccggagccgccgccgcagcggggacggggagcccccgggggccccgccacctccgccgtccgccgtcacctacccggactggatcggccagagttactccgaggtgatgagcctcaacgagcactccatgcaggcgctgtcctggcgcaagctctacttgagccgcgccaagcttaaagcctccagccggacctcggctctgctctccggcttcgccatggtggcaatggtggaggtgcagctggacgctgaccacgactacccaccggggctgctcatcgccttcagtgcctgcaccacagtgctggtggctgtgcacctgtttgcgctcatgatcagcacctgcatcctgcccaacatcgaggcagtgagcaacgtgcacaatctcaactcggtcaaggagtccccccacgagcgcatgcaccgccacatcgagctggcctgggccttctccactgtcatcggcacgctgctcttcctggctgaggtggtgctgctctgctgggtcaagttcttacccctcaagaagcagccaggccagccaaggcccaccagcaagccccccgccggtggtgcagcagccaacgtcagcaccagcggcatcaccccgggccaggcagctgccatcgcctcgaccaccatcatggtgcccttcggcctgatctttatcgtctttgccgtccacttctaccgctcactggtcagccataagaccgaccgacagttccaggagctcaacgagctggcagagtttgcccgcttacaggaccagctggaccacagaggggaccaccccctgacgcccggcagccactatgcctaggcccatgtggtctgggcccttccagtgctttggccttacgcccttccccatgaccttgtcctgccgcagcctcacggacagcctgtgcagggggctgggcttcagcaaggggcagagcgtggagggaagaggctttttataagagaaatttctgcactttgaaactgtcctctaagagaataagcatttcctgttcttccagctccaggtccacctcctgttgggaggcagtggggggtcaaagtggggccacacactcgctgtgtcccctctcctcccctgtgccagtgccacctgggtgccttctcttgtcctgtccgtctcaacctccctcccgtccagcattgagtgtgtacatgtgtgtgtgacacataaatatactcataaggacacctccttcccgtgtcttgtatttgttgggcctgggctactgctcaccctggttaggtgagcctctaggaaaacttaaaacaaattttaagccaggtatggtggcacatacctgtggtctcagctattcaggaggccaaggcaggaggatctcttgagcccaggagtttgagaccccatctcaaacaaaaaatacaaaaattagccagccacggcgcctgcacttccagctcctttgagagactgaggcaggaagattgcctaagcccaggaggccaagtctgcagtgagctatagtaacaccactgcactccaacctgggcaacagagggagactctgtctctaaaaaaatagaaaaatttgccccgcatggtggctcacgcctgtaatcctagccctttggaaggctaaggcgggcagatcacttgaggtcgggagttcgagaccagcctgaccaacatggagaaaccccgtctgtactaaaaatacaaaattagctgggtttggtggcgcatgcttgtaatcccagctactcgggaggctgaggcaggagaatcgcttgaacccaggaggcggaggttgcagtgagctgagatcgcgccattgcactccagcctgggcaacaacagtgaaactccatctc

SEQ ID NO: 80 - protein, 301 aa, Pan troglodytes
MHPEPAPPPSGSSPELPPSGGSTTSGSRRSRRRSGDGEPPGAPPPPPSAVTYPDWIGQSYSEVMSLNEHSMQALSWRKLYLSRAKLKASSRTSALLSGFAMVAMVEVQLDADHDYPPGLLIAFSACTTVLVAVHLFALMISTCILPNIEAVSNVHNLNSVKESPHERMHRHIELAWAFSTVIGTLLFLAEVVLLCWVKFLPLKKQPGQPRPTSKPPAGGAAANVSTSGITPGQAAAIASTTIMVPFGLIFIVFAVHFYRSLVSHKTDRQFQELNELAEFARLQDQLDHRGDHPLTPGSHYA

SEQ ID NO: 81 - DNA, 771 bp, Macaca fascicularis
  misc_feature: ORAI1 coding sequence (partial)
cgccgtcacctacccggactggatcggccagagttactccgaggtgatgagtctcaacgagcactccatgcaggcgctgtcctggcgcaagctctatttgagccgcgccaagctcaaagcctccagccggacctcggctctgctctccggcttcgccatggtggcaatggtggaggtgcagctggacgctgaccacgactacccgccagggctgctcatcgccttcagtgcctgcaccacggtgctggtggctgtgcacctgtttgcactcatgatcagcacctgcatcctgcccaacatcgaggcggtgagcaacgtgcacaacctcaactcggtcaaggagtccccccacgagcgcatgcaccgccacatcgagctggcctgggccttctccaccgtcatcggcacgctgcttttcctggccgaggtcgtgctgctctgctgggtcaagttcttgcccctcaagaagcagccaggccagccgaggcccaccagcaagccccccgccagtggtgcagccgccaacgtcagcaccagcggcatcaccccgggccaggcagccgccatcgcctcgaccaccatcatggtgcccttcggcctgatctttattgtcttcgccgtccacttctaccgctcactggtcagccataagacggaccgacagttccaggagctcaacgagctggcggagtttgctcgcttacaggaccagctggaccacagaggggaccaccccctgacgcccggcagccactatgcctaggcggccgc

SEQ ID NO: 82 - protein, 238 aa, Macaca fascicularis
MSLNEHSMQALSWRKLYLSRAKLKASSRTSALLSGFAMVAMVEVQLDADHDYPPGLLIAFSACTTVLVAVHLFALMISTCILPNIEAVSNVHNLNSVKESPHERMHRHIELAWAFSTVIGTLLFLAEVVLLCWVKFLPLKKQPGQPRPTSKPPASGAAANVSTSGITPGQAAAIASTTIMVPFGLIFIVFAVHFYRSLVSHKTDRQFQELNELAEFARLQDQLDHRGDHPLTPGSHYA

SEQ ID NO: 83 - DNA, 1086 bp, Canis familiaris
  misc_feature: ORAI1 coding sequence-NCBI Accession XM_543 386
atgctaatcaccccgaggctgcggccaatcgcggcgcgcgccgagggcccgggccatggatctcggacgctggcttgcgcgcaagctcgcgtccgggcggccccaggcttcttgcaagcggcgcggcgggcctgcgaggagccccagccggcccgcgcctttgcaccgtgctccatgcatccggagcccgccccgcccccgaacagcaacagccccgagcttcccctcggcggaggcagcaccaccagcggcagccgccggagccgccgccgcagcggggacggggagcccccggggtcgccgcccccgccagccgccgtcacctacccggactggatcggccagacttactccgaggtgatgagcctcaacgagcactcgatgcaggcgctgtcctggcgcaagctctacttgagccgcgccaagctcaaagcctccagccggacctctgctctgctctccggcttcgccatggtggcgatggtggaggtacagctggatgccgaccacgactacccgccagggctgctgatcgccttcagcgcctgcactacagtgttagtggccgtacacctgtttgcgctcatgatcagcacctgcatcctgcccaacatcgaggcggtgagcaacgtgcataacctcaactcggtcaaggagtcgccccatgaacgcatgcaccgccacatcgagctagcctgggccttctccactgtcattggcacactcctctttctggctgaggtagtgctgctctgctgggtcaagtttttgcccctcaagaagcagccagggcagccccggcccaccagcaagcccccagctggcagcgctgttgccagcgtcaacggcagcagcactggcggcatcacccccggccaggctgctgccattgcctccaccaccatcatggtccccttcggcctggtctttatcgtctttgccgtccacttctaccgctcactggtcagccataaaacggaccggcagttccaggagctcaacgagctggccgagtttgctcgcctgcaggaccagctggaccacagaggggatcaccccctgacgcccagcagtcactatgcataa

SEQ ID NO: 84 - protein, 361 aa, Canis familiaris
MLITPRLRPIAARAEGPGHGSRTLACAQARVRAAPGFLQAARRACEEPQPARAFAPCSMHPEPAPPPNSNSPELPLGGGSTTSGSRRSRRRSGDGEPPGSPPPPAAVTYPDWIGQTYSEVMSLNEHSMQALSWRKLYLSRAKLKASSRTSALLSGFAMVAMVEVQLDADHDYPPGLLIAFSACTTVLVAVHLFALMISTCILPNIEAVSNVHNLNSVKESPHERMHRHIELAWAFSTVIGTLLFLAEVVLLCWVKFLPLKKQPGQPRPTSKPPAGSAVASVNGSSTGGITPGQAAAIASTTIMVPFGLVFIVFAVHFYRSLVSHKTDRQFQELNELAEFARLQDQLDHRGDHPLTPSSHYA

SEQ ID NO: 85 - protein, 37 aa, Mus musculus
  MISC_FEATURE: Putative ORAI1 ECL2 domain sequence
KFLPLKRQAGQPSPTKPPAESVIVANHSDSSGITPGE

SEQ ID NO: 86 - DNA, 27 bp, Artificial Sequence
  Forward primer sequence
ggtcgacatgcatccggagcccgcccc

SEQ ID NO: 87 - DNA, 43 bp, Artificial Sequence
  Reverse primer sequence
ggcggtgcatgcgctcatgtggtgactccttgaccgagttgag

SEQ ID NO: 88 - DNA, 73 bp, Artificial Sequence
  Forward primer sequence
cgcatgcaccgccacatcgagctggcctgggccttctccaccgtcatcggcacgctgctcttcctagctgagg

SEQ ID NO: 89 - DNA, 66 bp, Artificial Sequence
  Reverse primer sequence
cgcggccgcctaggcatagtggctgccgggcgtcagggggtggtcccctctgtggtccagctggtc

SEQ ID NO: 90 - DNA, 909 bp, Artificial Sequence
  hORAI1/mORAI1 ECL2 chimeric sequence
atgcatccggagcccgccccgcccccgagccgcagcagtcccgagcttcccccaagcggcggcagcaccaccagcggcagccgccggagccgccgccgcagcggggacggggagcccccgggggccccgccaccgccgccgtccgccgtcacctacccggactggatcggccagagttactccgaggtgatgagcctcaacgagcactccatgcaggcgctgtcctggcgcaagctctacttgagccgcgccaagcttaaagcctccagccggacctcggctctgctctccggcttcgccatggtggcaatggtggaggtgcagctggacgctgaccacgactacccaccggggctgctcatcgccttcagtgcctgcaccacagtgctggtggctgtgcacctgtttgcgctcatgatcagcacctgcatcctgcccaacatcgaggcggtgagcaacgtgcacaatctcaactcggtcaaggagtccccccatgagcgcatgcaccgccacatcgagctggcctgggccttctccaccgtcatcggcacgctgctcttcctagctgaggtggtgctgctctgctgggtcaagttcttgcccctcaagaggcaagcgggacagccaagccccaccaagcctcccgctgaatcagtcatcgtcgccaaccacagcgacagcagcggcatcaccccgggccaggcagctgccatcgcttcgaccaccatcatggtgcccttcggcctgatctttatcgtcttcgccgtccacttctaccgctcactggttagccataagaccgaccgacagttccaggagctcaacgagctggcggagtttgcccgcttacaggaccagctggaccacagaggggaccaccccctgacgcccggcagccactatgcctag

SEQ ID NO: 91 - protein, 302 aa, Artificial Sequence
  Synthetic Construct
MHPEPAPPPSRSSPELPPSGGSTTSGSRRSRRRSGDGEPPGAPPPPPSAVTYPDWIGQSYSEVMSLNEHSMQALSWRKLYLSRAKLKASSRTSALLSGFAMVAMVEVQLDADHDYPPGLLIAFSACTTVLVAVHLFALMISTCILPNIEAVSNVHNLNSVKESPHERMHRHIELAWAFSTVIGTLLFLAEVVLLCWVKFLPLKRQAGQPSPTKPPAESVIVANHSDSSGITPGQAAAIASTTIMVPFGLIFIVFAVHFYRSLVSHKTDRQFQELNELAEFARLQDQLDHRGDHPLTPGSHYA

SEQ ID NO: 92 - DNA, 38 bp, Artificial Sequence; identical to SEQ ID NO: 68
  Forward primer sequence

SEQ ID NO: 93 - DNA, 64 bp, Artificial Sequence
  Reverse primer sequence
gggcttgctggtgggccttggctggcctggctgcttcttgagaggtaagaacttgacccagcag

SEQ ID NO: 94 - DNA, 65 bp, Artificial Sequence
  Reverse primer sequence
ggtgatgccgctggtgctgacgttggctgctgcgccactggcggggggcttgctggtgggccttg

SEQ ID NO: 95 - DNA, 65 bp, Artificial Sequence
  Reverse primer sequence
ggaaccatgatggcggtggaggcaatggctgccgcctcacccggggtgatgccgctggtgctgac

SEQ ID NO: 96 - DNA, 912 bp, Artificial Sequence
  mORAI1/hORAI1 ECL2 chimeric sequence
atgcatccggagcctgccccgcccccgagtcacagcaatccggagcttcccgtgagcggcggcagcagcactagcggcagccgccggagccgccgccgcagcggggacggggagccctcgggggccccaccgctgccgccgccgccacccgccgtcagctacccggactggatcggccagagttactccgaggtgatgagcctcaacgagcactcgatgcaggcgctgtcctggcgcaagctctacttaagccgcgccaagctcaaagcttccagccggacctcggctctgctctccggcttcgccatggtagcgatggtggaagtccagctggacacagaccatgactacccaccagggttgctcatcgtctttagtgcctgcaccacagtgctagtggccgtgcacctgtttgccctcatgatcagcacctgcatcctgcccaacatcgaggctgtgagcaacgtccacaacctcaactcggtcaaagagtcaccccacgagcgcatgcatcgccacatcgagctggcctgggccttctccacggtcatcgggacgctgcttttcctagcagaggtcgtgctgctctgctgggtcaagttcttacctctcaagaagcagccaggccagccaaggcccaccagcaagccccccgccagtggcgcagcagccaacgtcagcaccagcggcatcaccccgggtgaggcggcagccattgcctccaccgccatcatggttccctgtggcctggttttcatcgtctttgctgttcacttctaccgctccctggtcagccataagacggaccggcagttccaggagctcaatgagctggccgagtttgcccgcttgcaggaccagctggaccacagaggggaccattctctaacaccgggcacccactatgcctaa

SEQ ID NO: 97 - protein, 303 aa, Artificial Sequence
  Synthetic Construct
MHPEPAPPPSHSNPELPVSGGSSTSGSRRSRRRSGDGEPSGAPPLPPPPPAVSYPDWIGQSYSEVMSLNEHSMQALSWRKLYLSRAKLKASSRTSALLSGFAMVAMVEVQLDTDHDYPPGLLIVFSACTTVLVAVHLFALMISTCILPNIEAVSNVHNLNSVKESPHERMHRHIELAWAFSTVIGTLLFLAEVVLLCWVKFLPLKKQPGQPRPTSKPPASGAAANVSTSGITPGEAAAIASTAIMVPCGLVFIVFAVHFYRSLVSHKTDRQFQELNELAEFARLQDQLDHRGDHSLTPGTHYA

SEQ ID NO: 98 - protein, 5 aa, Mus musculus
  MISC_FEATURE: 215-219 of SEQ ID NO72
KPPAE

SEQ ID NO: 99 - protein, 5 aa, Homo sapiens
  MISC_FEATURE: 213-217 of SEQ ID NO2
SKPPA

SEQ ID NO: 100 - DNA, 53 bp, Artificial Sequence
  Forward sequence primer
gggacagccaagccccaccagcaagccccctgcatcagtcatcgtcgccaacc

SEQ ID NO: 101 - DNA, 53 bp, Artificial Sequence
  Reverse sequence primer
ggttggcgacgatgactgatgcagggggcttgctggtggggcttggctgtccc

SEQ ID NO: 102 - DNA, 909 bp, Artificial Sequence
  hORAI1/mORAI1 ECL2 (SKPPA) sequence
atgcatccggagcccgccccgcccccgagccgcagcagtcccgagcttcccccaagcggcggcagcaccaccagcggcagccgccggagccgccgccgcagcggggacggggagcccccgggggccccgccaccgccgccgtccgccgtcacctacccggactggatcggccagagttactccgaggtgatgagcctcaacgagcactccatgcaggcgctgtcctggcgcaagctctacttgagccgcgccaagcttaaagcctccagccggacctcggctctgctctccggcttcgccatggtggcaatggtggaggtgcagctggacgctgaccacgactacccaccggggctgctcatcgccttcagtgcctgcaccacagtgctggtggctgtgcacctgtttgcgctcatgatcagcacctgcatcctgcccaacatcgaggcggtgagcaacgtgcacaatctcaactcggtcaaggagtccccccatgagcgcatgcaccgccacatcgagctggcctgggccttctccaccgtcatcggcacgctgctcttcctagctgaggtggtgctgctctgctgggtcaagttcttgcccctcaagaggcaagcgggacagccaagccccaccagcaagccccctgcatcagtcatcgtcgccaaccacagcgacagcagcggcatcaccccgggccaggcagctgccatcgcttcgaccaccatcatggtgcccttcggcctgatctttatcgtcttcgccgtccacttctaccgctcactggttagccataagaccgaccgacagttccaggagctcaacgagctggcggagtttgcccgcttacaggaccagctggaccacagaggggaccaccccctgacgcccggcagccactatgcctag

SEQ ID NO: 103 - protein, 302 aa, Artificial Sequence
  Synthetic Construct
MHPEPAPPPSRSSPELPPSGGSTTSGSRRSRRRSGDGEPPGAPPPPPSAVTYPDWIGQSYSEVMSLNEHSMQALSWRKLYLSRAKLKASSRTSALLSGFAMVAMVEVQLDADHDYPPGLLIAFSACTTVLVAVHLFALMISTCILPNIEAVSNVHNLNSVKESPHERMHRHIELAWAFSTVIGTLLFLAEVVLLCWVKFLPLKRQAGQPSPTSKPPASVIVANHSDSSGITPGQAAAIASTTIMVPFGLIFIVFAVHFYRSLVSHKTDRQFQELNELAEFARLQDQLDHRGDHPLTPGSHYA

SEQ ID NO: 104 - DNA, 39 bp, Artificial Sequence
  Forward sequence primer
cctcccgctgaatcaggcgccgccgccaaccacagcgac

SEQ ID NO: 105 - DNA, 39 bp, Artificial Sequence
  Reverse sequence primer
gtcgctgtggttggcggcggcgcctgattcagcgggagg

SEQ ID NO: 106 - DNA, 909 bp, Artificial Sequence
  hORAI1/mORAI1 ECL2 (GAA) sequence
atgcatccggagcccgccccgcccccgagccgcagcagtcccgagcttcccccaagcggcggcagcaccaccagcggcagccgccggagccgccgccgcagcggggacggggagcccccgggggccccgccaccgccgccgtccgccgtcacctacccggactggatcggccagagttactccgaggtgatgagcctcaacgagcactccatgcaggcgctgtcctggcgcaagctctacttgagccgcgccaagcttaaagcctccagccggacctcggctctgctctccggcttcgccatggtggcaatggtggaggtgcagctggacgctgaccacgactacccaccggggctgctcatcgccttcagtgcctgcaccacagtgctggtggctgtgcacctgtttgcgctcatgatcagcacctgcatcctgcccaacatcgaggcggtgagcaacgtgcacaatctcaactcggtcaaggagtccccccatgagcgcatgcaccgccacatcgagctggcctgggccttctccaccgtcatcggcacgctgctcttcctagctgaggtggtgctgctctgctgggtcaagttcttgcccctcaagaggcaagcgggacagccaagccccaccaagcctcccgctgaatcaggcgccgccgccaaccacagcgacagcagcggcatcaccccgggccaggcagctgccatcgcttcgaccaccatcatggtgcccttcggcctgatctttatcgtcttcgccgtccacttctaccgctcactggttagccataagaccgaccgacagttccaggagctcaacgagctggcggagtttgcccgcttacaggaccagctggaccacagaggggaccaccccctgacgcccggcagccactatgcctag

SEQ ID NO: 107 - protein, 302 aa, Artificial Sequence
  Synthetic Construct
MHPEPAPPPSRSSPELPPSGGSTTSGSRRSRRRSGDGEPPGAPPPPPSAVTYPDWIGQSYSEVMSLNEHSMQALSWRKLYLSRAKLKASSRTSALLSGFAMVAMVEVQLDADHDYPPGLLIAFSACTTVLVAVHLFALMISTCILPNIEAVSNVHNLNSVKESPHERMHRHIELAWAFSTVIGTLLFLAEVVLLCWVKFLPLKRQAGQPSPTKPPAESGAAANHSDSSGITPGQAAAIASTTIMVPFGLIFIVFAVHFYRSLVSHKTDRQFQELNELAEFARLQDQLDHRGDHPLTPGSHYA

SEQ ID NO: 108 - protein, 6 aa, Mus musculus
  MISC_FEATURE: 218 to 223 of SEQ ID NO72 (mORAI1)
AESVIV

SEQ ID NO: 109 - protein, 6 aa, Homo sapiens
  MISC_FEATURE: 216 to 221 of SEQ ID NO2 (hORAI1)
PASGAA

SEQ ID NO: 110 - DNA, 47 bp, Artificial Sequence
  Forward primer sequence
cccaccaagcctccccctgcatcaggcgccgccgccaaccacagcga

SEQ ID NO: 111 - DNA, 47 bp, Artificial Sequence
  Reverse primer sequence
tcgctgtggttggcggcggcgcctgatgcagggggaggcttggtggg

SEQ ID NO: 112 - DNA, 909 bp, Artificial Sequence
  hORAI1/mORAI1 ECL2 (PASGAA) sequence
atgcatccggagcccgccccgcccccgagccgcagcagtcccgagcttcccccaagcggcggcagcaccaccagcggcagccgccggagccgccgccgcagcggggacggggagcccccgggggccccgccaccgccgccgtccgccgtcacctacccggactggatcggccagagttactccgaggtgatgagcctcaacgagcactccatgcaggcgctgtcctggcgcaagctctacttgagccgcgccaagcttaaagcctccagccggacctcggctctgctctccggcttcgccatggtggcaatggtggaggtgcagctggacgctgaccacgactacccaccggggctgctcatcgccttcagtgcctgcaccacagtgctggtggctgtgcacctgtttgcgctcatgatcagcacctgcatcctgcccaacatcgaggcggtgagcaacgtgcacaatctcaactcggtcaaggagtccccccatgagcgcatgcaccgccacatcgagctggcctgggccttctccaccgtcatcggcacgctgctcttcctagctgaggtggtgctgctctgctgggtcaagttcttgcccctcaagaggcaagcgggacagccaagccccaccaagcctccccctgcatcaggcgccgccgccaaccacagcgacagcagcggcatcaccccgggccaggcagctgccatcgcttcgaccaccatcatggtgcccttcggcctgatctttatcgtcttcgccgtccacttctaccgctcactggttagccataagaccgaccgacagttccaggagctcaacgagctggcggagtttgcccgcttacaggaccagctggaccacagaggggaccaccccctgacgcccggcagccactatgcctag

SEQ ID NO: 113 - protein, 302 aa, Artificial Sequence
  Synthetic Construct
MHPEPAPPPSRSSPELPPSGGSTTSGSRRSRRRSGDGEPPGAPPPPPSAVTYPDWIGQSYSEVMSLNEHSMQALSWRKLYLSRAKLKASSRTSALLSGFAMVAMVEVQLDADHDYPPGLLIAFSACTTVLVAVHLFALMISTCILPNIEAVSNVHNLNSVKESPHERMHRHIELAWAFSTVIGTLLFLAEVVLLCWVKFLPLKRQAGQPSPTKPPPASGAAANHSDSSGITPGQAAAIASTTIMVPFGLIFIVFAVHFYRSLVSHKTDRQFQELNELAEFARLQDQLDHRGDHPLTPGSHYA

SEQ ID NO: 114 - DNA, 36 bp, Artificial Sequence
  Forward primer sequence
gcgccgccgccaacgtcagcaccagcagcggcatca

SEQ ID NO: 115 - DNA, 36 bp, Artificial Sequence
  Reverse primer sequence
tgatgccgctgctggtgctgacgttggcggcggcgc

SEQ ID NO: 116 - DNA, 909 bp, Artificial Sequence
  hORAI1/mORAI1 ECL2  (VST) sequence
atgcatccggagcccgccccgcccccgagccgcagcagtcccgagcttcccccaagcggcggcagcaccaccagcggcagccgccggagccgccgccgcagcggggacggggagcccccgggggccccgccaccgccgccgtccgccgtcacctacccggactggatcggccagagttactccgaggtgatgagcctcaacgagcactccatgcaggcgctgtcctggcgcaagctctacttgagccgcgccaagcttaaagcctccagccggacctcggctctgctctccggcttcgccatggtggcaatggtggaggtgcagctggacgctgaccacgactacccaccggggctgctcatcgccttcagtgcctgcaccacagtgctggtggctgtgcacctgtttgcgctcatgatcagcacctgcatcctgcccaacatcgaggcggtgagcaacgtgcacaatctcaactcggtcaaggagtccccccatgagcgcatgcaccgccacatcgagctggcctgggccttctccaccgtcatcggcacgctgctcttcctagctgaggtggtgctgctctgctgggtcaagttcttgcccctcaagaggcaagcgggacagccaagccccaccaagcctcccgctgaatcagtcatcgtcgccaacgtcagcaccagcagcggcatcaccccgggccaggcagctgccatcgcttcgaccaccatcatggtgcccttcggcctgatctttatcgtcttcgccgtccacttctaccgctcactggttagccataagaccgaccgacagttccaggagctcaacgagctggcggagtttgcccgcttacaggaccagctggaccacagaggggaccaccccctgacgcccggcagccactatgcctag

SEQ ID NO: 117 - protein, 302 aa, Artificial Sequence
  Synthetic Construct
MHPEPAPPPSRSSPELPPSGGSTTSGSRRSRRRSGDGEPPGAPPPPPSAVTYPDWIGQSYSEVMSLNEHSMQALSWRKLYLSRAKLKASSRTSALLSGFAMVAMVEVQLDADHDYPPGLLIAFSACTTVLVAVHLFALMISTCILPNIEAVSNVHNLNSVKESPHERMHRHIELAWAFSTVIGTLLFLAEVVLLCWVKFLPLKRQAGQPSPTKPPAESVIVANVSTSSGITPGQAAAIASTTIMVPFGLIFIVFAVHFYRSLVSHKTDRQFQELNELAEFARLQDQLDHRGDHPLTPGSHYA

SEQ ID NO: 118 - protein, 6 aa, Mus musculus
  MISC_FEATURE: 223-228 of SEQ ID NO72 (mORAI1)
VANHSD

SEQ ID NO: 119 - protein, 6 aa, Homo sapiens
  MISC_FEATURE: 221 to 226 of SEQ ID NO2 (hORAI1)
AANVST

SEQ ID NO: 120 - DNA, 36 bp, Artificial Sequence; identical to SEQ ID NO: 114
  Forward primer sequence

SEQ ID NO: 121 - DNA, 36 bp, Artificial Sequence; identical to SEQ ID NO: 115
  Reverse primer sequence

SEQ ID NO: 122 - DNA, 909 bp, Artificial Sequence
  hORAI1/mORAI1 ECL2 (AANVST) sequence
atgcatccggagcccgccccgcccccgagccgcagcagtcccgagcttcccccaagcggcggcagcaccaccagcggcagccgccggagccgccgccgcagcggggacggggagcccccgggggccccgccaccgccgccgtccgccgtcacctacccggactggatcggccagagttactccgaggtgatgagcctcaacgagcactccatgcaggcgctgtcctggcgcaagctctacttgagccgcgccaagcttaaagcctccagccggacctcggctctgctctccggcttcgccatggtggcaatggtggaggtgcagctggacgctgaccacgactacccaccggggctgctcatcgccttcagtgcctgcaccacagtgctggtggctgtgcacctgtttgcgctcatgatcagcacctgcatcctgcccaacatcgaggcggtgagcaacgtgcacaatctcaactcggtcaaggagtccccccatgagcgcatgcaccgccacatcgagctggcctgggccttctccaccgtcatcggcacgctgctcttcctagctgaggtggtgctgctctgctgggtcaagttcttgcccctcaagaggcaagcgggacagccaagccccaccaagcctcccgctgaatcagtcatcgccgccaacgtcagcaccagcagcggcatcaccccgggccaggcagctgccatcgcttcgaccaccatcatggtgcccttcggcctgatctttatcgtcttcgccgtccacttctaccgctcactggttagccataagaccgaccgacagttccaggagctcaacgagctggcggagtttgcccgcttacaggaccagctggaccacagaggggaccaccccctgacgcccggcagccactatgcctag

SEQ ID NO: 123 - protein, 302 aa, Artificial Sequence
  Synthetic Construct
MHPEPAPPPSRSSPELPPSGGSTTSGSRRSRRRSGDGEPPGAPPPPPSAVTYPDWIGQSYSEVMSLNEHSMQALSWRKLYLSRAKLKASSRTSALLSGFAMVAMVEVQLDADHDYPPGLLIAFSACTTVLVAVHLFALMISTCILPNIEAVSNVHNLNSVKESPHERMHRHIELAWAFSTVIGTLLFLAEVVLLCWVKFLPLKRQAGQPSPTKPPAESVIAANVSTSSGITPGQAAAIASTTIMVPFGLIFIVFAVHFYRSLVSHKTDRQFQELNELAEFARLQDQLDHRGDHPLTPGSHYA

SEQ ID NO: 124 - protein, 8 aa, Mus musculus
  MISC_FEATURE: 212 to 219 of SEQ ID NO72 (mORAI1)
SPTKPPAE

SEQ ID NO: 125 - protein, 8 aa, Homo sapiens
  MISC_FEATURE: 210 to 217 of SEQ ID NO2 (hORAI1)
RPTSKPPA

SEQ ID NO: 126 - DNA, 25 bp, Artificial Sequence
  Forward primer sequence
gggacagccaaggcccaccagcaag

SEQ ID NO: 127 - DNA, 25 bp, Artificial Sequence
  Reverse primer sequence
cttgctggtgggccttggctgtccc

SEQ ID NO: 128 - DNA, 909 bp, Artificial Sequence
  hORAI1/mORAI1 ECL2 (RPTSKPPA) sequence
atgcatccggagcccgccccgcccccgagccgcagcagtcccgagcttcccccaagcggcggcagcaccaccagcggcagccgccggagccgccgccgcagcggggacggggagcccccgggggccccgccaccgccgccgtccgccgtcacctacccggactggatcggccagagttactccgaggtgatgagcctcaacgagcactccatgcaggcgctgtcctggcgcaagctctacttgagccgcgccaagcttaaagcctccagccggacctcggctctgctctccggcttcgccatggtggcaatggtggaggtgcagctggacgctgaccacgactacccaccggggctgctcatcgccttcagtgcctgcaccacagtgctggtggctgtgcacctgtttgcgctcatgatcagcacctgcatcctgcccaacatcgaggcggtgagcaacgtgcacaatctcaactcggtcaaggagtccccccatgagcgcatgcaccgccacatcgagctggcctgggccttctccaccgtcatcggcacgctgctcttcctagctgaggtggtgctgctctgctgggtcaagttcttgcccctcaagaggcaagcgggacagccaaggcccaccagcaagccccctgcatcagtcatcgtcgccaaccacagcgacagcagcggcatcaccccgggccaggcagctgccatcgcttcgaccaccatcatggtgcccttcggcctgatctttatcgtcttcgccgtccacttctaccgctcactggttagccataagaccgaccgacagttccaggagctcaacgagctggcggagtttgcccgcttacaggaccagctggaccacagaggggaccaccccctgacgcccggcagccactatgcctag

SEQ ID NO: 129 - protein, 302 aa, Artificial Sequence
  Synthetic Construct
MHPEPAPPPSRSSPELPPSGGSTTSGSRRSRRRSGDGEPPGAPPPPPSAVTYPDWIGQSYSEVMSLNEHSMQALSWRKLYLSRAKLKASSRTSALLSGFAMVAMVEVQLDADHDYPPGLLIAFSACTTVLVAVHLFALMISTCILPNIEAVSNVHNLNSVKESPHERMHRHIELAWAFSTVIGTLLFLAEVVLLCWVKFLPLKRQAGQPRPTSKPPASVIVANHSDSSGITPGQAAAIASTTIMVPFGLIFIVFAVHFYRSLVSHKTDRQFQELNELAEFARLQDQLDHRGDHPLTPGSHYA

SEQ ID NO: 130 - DNA, 44 bp, Artificial Sequence
  Forward primer sequence
cagccaaggcccaccaagccgcccgccgagagtggcgcagcagc

SEQ ID NO: 131 - DNA, 44 bp, Artificial Sequence
  Reverse primer sequence
gctgctgcgccactctcggcgggcggcttggtgggccttggctg

SEQ ID NO: 132 - DNA, 912 bp, Artificial Sequence
  mORAI1/hORAI1 ECL2 (KPPAE) seqeunce
atgcatccggagcctgccccgcccccgagtcacagcaatccggagcttcccgtgagcggcggcagcagcactagcggcagccgccggagccgccgccgcagcggggacggggagccctcgggggccccaccgctgccgccgccgccacccgccgtcagctacccggactggatcggccagagttactccgaggtgatgagcctcaacgagcactcgatgcaggcgctgtcctggcgcaagctctacttaagccgcgccaagctcaaagcttccagccggacctcggctctgctctccggcttcgccatggtagcgatggtggaagtccagctggacacagaccatgactacccaccagggttgctcatcgtctttagtgcctgcaccacagtgctagtggccgtgcacctgtttgccctcatgatcagcacctgcatcctgcccaacatcgaggctgtgagcaacgtccacaacctcaactcggtcaaagagtcaccccacgagcgcatgcatcgccacatcgagctggcctgggccttctccacggtcatcgggacgctgcttttcctagcagaggtcgtgctgctctgctgggtcaagttcttacctctcaagaagcagccaggccagccaaggcccaccaagccgcccgccgagagtggcgcagcagccaacgtcagcaccagcggcatcaccccgggtgaggcggcagccattgcctccaccgccatcatggttccctgtggcctggttttcatcgtctttgctgttcacttctaccgctccctggtcagccataagacggaccggcagttccaggagctcaatgagctggccgagtttgcccgcttgcaggaccagctggaccacagaggggaccattctctaacaccgggcacccactatgcctaa

SEQ ID NO: 133 - protein, 303 aa, Artificial Sequence
  Synthetic Construct
MHPEPAPPPSHSNPELPVSGGSSTSGSRRSRRRSGDGEPSGAPPLPPPPPAVSYPDWIGQSYSEVMSLNEHSMQALSWRKLYLSRAKLKASSRTSALLSGFAMVAMVEVQLDTDHDYPPGLLIVFSACTTVLVAVHLFALMISTCILPNIEAVSNVHNLNSVKESPHERMHRHIELAWAFSTVIGTLLFLAEVVLLCWVKFLPLKKQPGQPRPTKPPAESGAAANVSTSGITPGEAAAIASTAIMVPCGLVFIVFAVHFYRSLVSHKTDRQFQELNELAEFARLQDQLDHRGDHSLTPGTHYA

SEQ ID NO: 134 - DNA, 39 bp, Artificial Sequence
  Forward primer sequence
aagccccccgccagtgtcatagtagccaacgtcagcacc

SEQ ID NO: 135 - DNA, 39 bp, Artificial Sequence
  Reverse primer sequence
ggtgctgacgttggctactatgacactggcggggggctt

SEQ ID NO: 136 - DNA, 912 bp, Artificial Sequence
  mORAI1/hORAI1 ECL2 (VIV) sequence
atgcatccggagcctgccccgcccccgagtcacagcaatccggagcttcccgtgagcggcggcagcagcactagcggcagccgccggagccgccgccgcagcggggacggggagccctcgggggccccaccgctgccgccgccgccacccgccgtcagctacccggactggatcggccagagttactccgaggtgatgagcctcaacgagcactcgatgcaggcgctgtcctggcgcaagctctacttaagccgcgccaagctcaaagcttccagccggacctcggctctgctctccggcttcgccatggtagcgatggtggaagtccagctggacacagaccatgactacccaccagggttgctcatcgtctttagtgcctgcaccacagtgctagtggccgtgcacctgtttgccctcatgatcagcacctgcatcctgcccaacatcgaggctgtgagcaacgtccacaacctcaactcggtcaaagagtcaccccacgagcgcatgcatcgccacatcgagctggcctgggccttctccacggtcatcgggacgctgcttttcctagcagaggtcgtgctgctctgctgggtcaagttcttacctctcaagaagcagccaggccagccaaggcccaccagcaagccccccgccagtgtcatagtagccaacgtcagcaccagcggcatcaccccgggtgaggcggcagccattgcctccaccgccatcatggttccctgtggcctggttttcatcgtctttgctgttcacttctaccgctccctggtcagccataagacggaccggcagttccaggagctcaatgagctggccgagtttgcccgcttgcaggaccagctggaccacagaggggaccattctctaacaccgggcacccactatgcctaa

SEQ ID NO: 137 - protein, 303 aa, Artificial Sequence
  Synthetic Construct
MHPEPAPPPSHSNPELPVSGGSSTSGSRRSRRRSGDGEPSGAPPLPPPPPAVSYPDWIGQSYSEVMSLNEHSMQALSWRKLYLSRAKLKASSRTSALLSGFAMVAMVEVQLDTDHDYPPGLLIVFSACTTVLVAVHLFALMISTCILPNIEAVSNVHNLNSVKESPHERMHRHIELAWAFSTVIGTLLFLAEVVLLCWVKFLPLKKQPGQPRPTSKPPASVIVANVSTSGITPGEAAAIASTAIMVPCGLVFIVFAVHFYRSLVSHKTDRQFQELNELAEFARLQDQLDHRGDHSLTPGTHYA

SEQ ID NO: 138 - DNA, 49 bp, Artificial Sequence
  Forward primer sequence
gcccaccagcaagcccgccgagagtgtcatagtagccaacgtcagcacc

SEQ ID NO: 139 - DNA, 49 bp, Artificial Sequence
  Reverse primer sequence
ggtgctgacgttggctactatgacactctcggcgggcttgctggtgggc

SEQ ID NO: 140 - DNA, 912 bp, Artificial Sequence
  mORAI1/hORAI1 ECL2 (AESVIV) sequence
atgcatccggagcctgccccgcccccgagtcacagcaatccggagcttcccgtgagcggcggcagcagcactagcggcagccgccggagccgccgccgcagcggggacggggagccctcgggggccccaccgctgccgccgccgccacccgccgtcagctacccggactggatcggccagagttactccgaggtgatgagcctcaacgagcactcgatgcaggcgctgtcctggcgcaagctctacttaagccgcgccaagctcaaagcttccagccggacctcggctctgctctccggcttcgccatggtagcgatggtggaagtccagctggacacagaccatgactacccaccagggttgctcatcgtctttagtgcctgcaccacagtgctagtggccgtgcacctgtttgccctcatgatcagcacctgcatcctgcccaacatcgaggctgtgagcaacgtccacaacctcaactcggtcaaagagtcaccccacgagcgcatgcatcgccacatcgagctggcctgggccttctccacggtcatcgggacgctgcttttcctagcagaggtcgtgctgctctgctgggtcaagttcttacctctcaagaagcagccaggccagccaaggcccaccagcaagcccgccgagagtgtcatagtagccaacgtcagcaccagcggcatcaccccgggtgaggcggcagccattgcctccaccgccatcatggttccctgtggcctggttttcatcgtctttgctgttcacttctaccgctccctggtcagccataagacggaccggcagttccaggagctcaatgagctggccgagtttgcccgcttgcaggaccagctggaccacagaggggaccattctctaacaccgggcacccactatgcctaa

SEQ ID NO: 141 - protein, 303 aa, Artificial Sequence
  Synthetic Construct
MHPEPAPPPSHSNPELPVSGGSSTSGSRRSRRRSGDGEPSGAPPLPPPPPAVSYPDWIGQSYSEVMSLNEHSMQALSWRKLYLSRAKLKASSRTSALLSGFAMVAMVEVQLDTDHDYPPGLLIVFSACTTVLVAVHLFALMISTCILPNIEAVSNVHNLNSVKESPHERMHRHIELAWAFSTVIGTLLFLAEVVLLCWVKFLPLKKQPGQPRPTSKPAESVIVANVSTSGITPGEAAAIASTAIMVPCGLVFIVFAVHFYRSLVSHKTDRQFQELNELAEFARLQDQLDHRGDHSLTPGTHYA

SEQ ID NO: 142 - DNA, 38 bp, Artificial Sequence
  Forward primer sequence
ggcgcagcagccaaccacagcgacagcggcatcacccc

SEQ ID NO: 143 - DNA, 38 bp, Artificial Sequence
  Reverse primer sequence
ggggtgatgccgctgtcgctgtggttggctgctgcgcc

SEQ ID NO: 144 - DNA, 912 bp, Artificial Sequence
  mORAI1/hORAI1 ECL2 (HSD) sequence
atgcatccggagcctgccccgcccccgagtcacagcaatccggagcttcccgtgagcggcggcagcagcactagcggcagccgccggagccgccgccgcagcggggacggggagccctcgggggccccaccgctgccgccgccgccacccgccgtcagctacccggactggatcggccagagttactccgaggtgatgagcctcaacgagcactcgatgcaggcgctgtcctggcgcaagctctacttaagccgcgccaagctcaaagcttccagccggacctcggctctgctctccggcttcgccatggtagcgatggtggaagtccagctggacacagaccatgactacccaccagggttgctcatcgtctttagtgcctgcaccacagtgctagtggccgtgcacctgtttgccctcatgatcagcacctgcatcctgcccaacatcgaggctgtgagcaacgtccacaacctcaactcggtcaaagagtcaccccacgagcgcatgcatcgccacatcgagctggcctgggccttctccacggtcatcgggacgctgcttttcctagcagaggtcgtgctgctctgctgggtcaagttcttacctctcaagaagcagccaggccagccaaggcccaccagcaagccccccgccagtggcgcagcagccaaccacagcgacagcggcatcaccccgggtgaggcggcagccattgcctccaccgccatcatggttccctgtggcctggttttcatcgtctttgctgttcacttctaccgctccctggtcagccataagacggaccggcagttccaggagctcaatgagctggccgagtttgcccgcttgcaggaccagctggaccacagaggggaccattctctaacaccgggcacccactatgcctaa

SEQ ID NO: 145 - protein, 303 aa, Artificial Sequence
  Synthetic Construct
MHPEPAPPPSHSNPELPVSGGSSTSGSRRSRRRSGDGEPSGAPPLPPPPPAVSYPDWIGQSYSEVMSLNEHSMQALSWRKLYLSRAKLKASSRTSALLSGFAMVAMVEVQLDTDHDYPPGLLIVFSACTTVLVAVHLFALMISTCILPNIEAVSNVHNLNSVKESPHERMHRHIELAWAFSTVIGTLLFLAEVVLLCWVKFLPLKKQPGQPRPTSKPPASGAAANHSDSGITPGEAAAIASTAIMVPCGLVFIVFAVHFYRSLVSHKTDRQFQELNELAEFARLQDQLDHRGDHSLTPGTHYA

SEQ ID NO: 146 - protein, 25 aa, Artificial Sequence
  Linker sequence “L25”
GGGGSGGGGSGGGGSGGGGSGGGGS

SEQ ID NO: 147 - protein, 20 aa, Artificial Sequence
  Linker sequence “L20”
GGGGSGGGGSGGGGSGGGGS

SEQ ID NO: 148 - protein, 6 aa, Artificial Sequence
  Linker sequence
XXNXXG

SEQ ID NO: 149 - protein, 4 aa, Artificial Sequence
  Linker sequence
GGGG

SEQ ID NO: 150 - protein, 5 aa, Artificial Sequence
  Linker sequence
GGGGG

SEQ ID NO: 151 - protein, 7 aa, Artificial Sequence
  Linker sequence
GGGGGGG

SEQ ID NO: 152 - protein, 5 aa, Artificial Sequence
  Linker sequence
GGGGS

SEQ ID NO: 153 - protein, 10 aa, Artificial Sequence
  Linker sequence
GGGGSGGGGS

SEQ ID NO: 154 - protein, 6 aa, Artificial Sequence
  Linker sequence
GGGGGK

SEQ ID NO: 155 - protein, 7 aa, Artificial Sequence
  Linker sequence
GGGGGKR

SEQ ID NO: 156 - protein, 8 aa, Artificial Sequence
  Linker sequence
GGGNGSGG

SEQ ID NO: 157 - protein, 8 aa, Artificial Sequence
  Linker sequence
GGGCGGGG

SEQ ID NO: 158 - protein, 5 aa, Artificial Sequence
  Linker sequence
GPNGG

SEQ ID NO: 159 - protein, 8 aa, Artificial Sequence
  Linker sequence
GGGKGGGG

SEQ ID NO: 160 - protein, 6 aa, Artificial Sequence
  Linker sequence
GGEGGG

SEQ ID NO: 161 - protein, 8 aa, Artificial Sequence
  Linker sequence
GGEEEGGG

SEQ ID NO: 162 - protein, 5 aa, Artificial Sequence
  Linker sequence
GEEEG

SEQ ID NO: 163 - protein, 4 aa, Artificial Sequence
  Linker sequence
GEEE

SEQ ID NO: 164 - protein, 6 aa, Artificial Sequence
  Linker sequence
GGDGGG

SEQ ID NO: 165 - protein, 7 aa, Artificial Sequence
  Linker sequence
GGDDDGG

SEQ ID NO: 166 - protein, 5 aa, Artificial Sequence
  Linker sequence
GDDDG

SEQ ID NO: 167 - protein, 4 aa, Artificial Sequence
  Linker sequence
GDDD

SEQ ID NO: 168 - protein, 21 aa, Artificial Sequence
  Linker sequence
GGGGSDDSDEGSDGEDGGGGS

SEQ ID NO: 169 - protein, 5 aa, Artificial Sequence
  Linker sequence
WEWEW

SEQ ID NO: 170 - protein, 5 aa, Artificial Sequence
  Linker sequence
FEFEF

SEQ ID NO: 171 - protein, 6 aa, Artificial Sequence
  Linker sequence
EEEWWW

SEQ ID NO: 172 - protein, 6 aa, Artificial Sequence
  Linker sequence
EEEFFF

SEQ ID NO: 173 - protein, 7 aa, Artificial Sequence
  Linker sequence
WWEEEWW

SEQ ID NO: 174 - protein, 7 aa, Artificial Sequence
  Linker sequence
FFEEEFF

SEQ ID NO: 175 - protein, 6 aa, Artificial Sequence
  Linker sequence
XXYXXG

SEQ ID NO: 176 - protein, 6 aa, Artificial Sequence
  Linker sequence
XXSXXG

SEQ ID NO: 177 - protein, 6 aa, Artificial Sequence
  Linker sequence
XXTXXG

SEQ ID NO: 178 - protein, 22 aa, Artificial Sequence
  Linker sequence
GSGSATGGSGSTASSGSGSATH

SEQ ID NO: 179 - protein, 22 aa, Artificial Sequence
  Linker sequence
HGSGSATGGSGSTASSGSGSAT

SEQ ID NO: 180 - protein, 19 aa, Artificial Sequence
  Linker sequence
AEAAAKEAAAKEAAAKAGG

SEQ ID NO: 181 - DNA, 37 bp, Artificial Sequence
  Forward primer sequence
gctagctgaaccaccatggatgtatgcgtccgtcttg

SEQ ID NO: 182 - DNA, 52 bp, Artificial Sequence
  Forward primer sequence
gggactcctcctgcaccagggccagagcctcagccatagtcacagtgagaag

SEQ ID NO: 183 - DNA, 70 bp, Artificial Sequence
  Forward primer sequence
cagccatagtcacagtgagaaggcgacaggaaccagctcgggagccaacatggtgagcaagggcgaggag

SEQ ID NO: 184 - DNA, 44 bp, Artificial Sequence
  Reverse primer sequence
cggcatggacgagctgtacaagtctgaggagtcgactgcagcag

SEQ ID NO: 185 - DNA, 27 bp, Artificial Sequence
  Forward primer sequence
ggagtcgactgcagcagagttttgccg

SEQ ID NO: 186 - DNA, 33 bp, Artificial Sequence
  Reverse primer sequence
ctttaagaagcctcttaagaagtaggcggccgc

SEQ ID NO: 187 - DNA, 39 bp, Artificial Sequence
  Forward primer sequence
aagccccctgcatcaggcgccgccgccaaccacagcgac

SEQ ID NO: 188 - DNA, 39 bp, Artificial Sequence
  Reverse primer sequence
gtcgctgtggttggcggcggcgcctgatgcagggggctt

SEQ ID NO: 189 - protein, 12 aa, Mus musculus
  MISC_FEATURE: 212 to 223 of SEQ ID NO72
SPTKPPAESVIV

SEQ ID NO: 190 - protein, 12 aa, Homo sapiens
  MISC_FEATURE: 210 to 221 of SEQ ID NO2
RPTSKPPASGAA

SEQ ID NO: 191 - DNA, 909 bp, Artificial Sequence
  hORAI1/mORAI1 ECL2 (RPTSKPPASGAA) sequence
atgcatccggagcccgccccgcccccgagccgcagcagtcccgagcttcccccaagcggcggcagcaccaccagcggcagccgccggagccgccgccgcagcggggacggggagcccccgggggccccgccaccgccgccgtccgccgtcacctacccggactggatcggccagagttactccgaggtgatgagcctcaacgagcactccatgcaggcgctgtcctggcgcaagctctacttgagccgcgccaagcttaaagcctccagccggacctcggctctgctctccggcttcgccatggtggcaatggtggaggtgcagctggacgctgaccacgactacccaccggggctgctcatcgccttcagtgcctgcaccacagtgctggtggctgtgcacctgtttgcgctcatgatcagcacctgcatcctgcccaacatcgaggcggtgagcaacgtgcacaatctcaactcggtcaaggagtccccccatgagcgcatgcaccgccacatcgagctggcctgggccttctccaccgtcatcggcacgctgctcttcctagctgaggtggtgctgctctgctgggtcaagttcttgcccctcaagaggcaagcgggacagccaaggcccaccagcaagccccctgcatcaggcgccgccgccaaccacagcgacagcagcggcatcaccccgggccaggcagctgccatcgcttcgaccaccatcatggtgcccttcggcctgatctttatcgtcttcgccgtccacttctaccgctcactggttagccataagaccgaccgacagttccaggagctcaacgagctggcggagtttgcccgcttacaggaccagctggaccacagaggggaccaccccctgacgcccggcagccactatgcctag

SEQ ID NO: 192 - protein, 302 aa, Artificial Sequence
  Synthetic Construct
MHPEPAPPPSRSSPELPPSGGSTTSGSRRSRRRSGDGEPPGAPPPPPSAVTYPDWIGQSYSEVMSLNEHSMQALSWRKLYLSRAKLKASSRTSALLSGFAMVAMVEVQLDADHDYPPGLLIAFSACTTVLVAVHLFALMISTCILPNIEAVSNVHNLNSVKESPHERMHRHIELAWAFSTVIGTLLFLAEVVLLCWVKFLPLKRQAGQPRPTSKPPASGAAANHSDSSGITPGQAAAIASTTIMVPFGLIFIVFAVHFYRSLVSHKTDRQFQELNELAEFARLQDQLDHRGDHPLTPGSHYA

SEQ ID NO: 193 - DNA, 36 bp, Artificial Sequence; identical to SEQ ID NO: 114
  Forward primer sequence

SEQ ID NO: 194 - DNA, 36 bp, Artificial Sequence; identical to SEQ ID NO: 115
  Reverse primer sequence

SEQ ID NO: 195 - protein, 11 aa, Mus musculus
  MISC_FEATURE: 218-228 of SEQ ID NO72
AESVIVANHSD

SEQ ID NO: 196 - protein, 11 aa, Homo sapiens
  MISC_FEATURE: 216-226 of SEQ ID NO2
PASGAAANVST

SEQ ID NO: 197 - DNA, 909 bp, Artificial Sequence
  hORAI1/mORAI1 ECL2 (PASGAAANVST) sequence
atgcatccggagcccgccccgcccccgagccgcagcagtcccgagcttcccccaagcggcggcagcaccaccagcggcagccgccggagccgccgccgcagcggggacggggagcccccgggggccccgccaccgccgccgtccgccgtcacctacccggactggatcggccagagttactccgaggtgatgagcctcaacgagcactccatgcaggcgctgtcctggcgcaagctctacttgagccgcgccaagcttaaagcctccagccggacctcggctctgctctccggcttcgccatggtggcaatggtggaggtgcagctggacgctgaccacgactacccaccggggctgctcatcgccttcagtgcctgcaccacagtgctggtggctgtgcacctgtttgcgctcatgatcagcacctgcatcctgcccaacatcgaggcggtgagcaacgtgcacaatctcaactcggtcaaggagtccccccatgagcgcatgcaccgccacatcgagctggcctgggccttctccaccgtcatcggcacgctgctcttcctagctgaggtggtgctgctctgctgggtcaagttcttgcccctcaagaggcaagcgggacagccaagccccaccaagcctccccctgcatcaggcgccgccgccaacgtcagcaccagcagcggcatcaccccgggccaggcagctgccatcgcttcgaccaccatcatggtgcccttcggcctgatctttatcgtcttcgccgtccacttctaccgctcactggttagccataagaccgaccgacagttccaggagctcaacgagctggcggagtttgcccgcttacaggaccagctggaccacagaggggaccaccccctgacgcccggcagccactatgcctag

SEQ ID NO: 198 - protein, 302 aa, Artificial Sequence
  Synthetic Construct
MHPEPAPPPSRSSPELPPSGGSTTSGSRRSRRRSGDGEPPGAPPPPPSAVTYPDWIGQSYSEVMSLNEHSMQALSWRKLYLSRAKLKASSRTSALLSGFAMVAMVEVQLDADHDYPPGLLIAFSACTTVLVAVHLFALMISTCILPNIEAVSNVHNLNSVKESPHERMHRHIELAWAFSTVIGTLLFLAEVVLLCWVKFLPLKRQAGQPSPTKPPPASGAAANVSTSSGITPGQAAAIASTTIMVPFGLIFIVFAVHFYRSLVSHKTDRQFQELNELAEFARLQDQLDHRGDHPLTPGSHYA

SEQ ID NO: 199 - DNA, 34 bp, Artificial Sequence
  Forward primer sequence
agttcttgcccctcaagaagcaaccgggacagcc

SEQ ID NO: 200 - DNA, 34 bp, Artificial Sequence
  Reverse primer sequence
ggctgtcccggttgcttcttgaggggcaagaact

SEQ ID NO: 201 - protein, 14 aa, Mus musculus
  MISC_FEATURE: 206 to 219 of SEQ ID NO72
RQAGQPSPTKPPAE

SEQ ID NO: 202 - protein, 14 aa, Homo sapiens
  MISC_FEATURE: 204 to 217 of SEQ ID NO2
KQPGQPRPTSKPPA

SEQ ID NO: 203 - DNA, 909 bp, Artificial Sequence
  hORAI1/mORAI1 ECL2 (KQPGQPRPTSKPPA) sequence
atgcatccggagcccgccccgcccccgagccgcagcagtcccgagcttcccccaagcggcggcagcaccaccagcggcagccgccggagccgccgccgcagcggggacggggagcccccgggggccccgccaccgccgccgtccgccgtcacctacccggactggatcggccagagttactccgaggtgatgagcctcaacgagcactccatgcaggcgctgtcctggcgcaagctctacttgagccgcgccaagcttaaagcctccagccggacctcggctctgctctccggcttcgccatggtggcaatggtggaggtgcagctggacgctgaccacgactacccaccggggctgctcatcgccttcagtgcctgcaccacagtgctggtggctgtgcacctgtttgcgctcatgatcagcacctgcatcctgcccaacatcgaggcggtgagcaacgtgcacaatctcaactcggtcaaggagtccccccatgagcgcatgcaccgccacatcgagctggcctgggccttctccaccgtcatcggcacgctgctcttcctagctgaggtggtgctgctctgctgggtcaagttcttgcccctcaagaagcaaccgggacagccaaggcccaccagcaagccccctgcatcagtcatcgtcgccaaccacagcgacagcagcggcatcaccccgggccaggcagctgccatcgcttcgaccaccatcatggtgcccttcggcctgatctttatcgtcttcgccgtccacttctaccgctcactggttagccataagaccgaccgacagttccaggagctcaacgagctggcggagtttgcccgcttacaggaccagctggaccacagaggggaccaccccctgacgcccggcagccactatgcctag

SEQ ID NO: 204 - protein, 302 aa, Artificial Sequence
  Synthetic Construct
MHPEPAPPPSRSSPELPPSGGSTTSGSRRSRRRSGDGEPPGAPPPPPSAVTYPDWIGQSYSEVMSLNEHSMQALSWRKLYLSRAKLKASSRTSALLSGFAMVAMVEVQLDADHDYPPGLLIAFSACTTVLVAVHLFALMISTCILPNIEAVSNVHNLNSVKESPHERMHRHIELAWAFSTVIGTLLFLAEVVLLCWVKFLPLKKQPGQPRPTSKPPASVIVANHSDSSGITPGQAAAIASTTIMVPFGLIFIVFAVHFYRSLVSHKTDRQFQELNELAEFARLQDQLDHRGDHPLTPGSHYA

SEQ ID NO: 205 - DNA, 39 bp, Artificial Sequence; identical to SEQ ID NO: 187
  Forward primer sequence

SEQ ID NO: 206 - DNA, 39 bp, Artificial Sequence; identical to SEQ ID NO: 188
  Reverse primer sequence

SEQ ID NO: 207 - protein, 18 aa, Mus musculus
  MISC_FEATURE: 206 to 223 of SEQ ID NO72
RQAGQPSPTKPPAESVIV

SEQ ID NO: 208 - protein, 18 aa, Homo sapiens
  MISC_FEATURE: 204 to 221 of SEQ ID NO2
KQPGQPRPTSKPPASGAA

SEQ ID NO: 209 - DNA, 909 bp, Artificial Sequence
  hORAI1/mORAI1 ECL2 ([sequence omitted]) sequence
atgcatccggagcccgccccgcccccgagccgcagcagtcccgagcttcccccaagcggcggcagcaccaccagcggcagccgccggagccgccgccgcagcggggacggggagcccccgggggccccgccaccgccgccgtccgccgtcacctacccggactggatcggccagagttactccgaggtgatgagcctcaacgagcactccatgcaggcgctgtcctggcgcaagctctacttgagccgcgccaagcttaaagcctccagccggacctcggctctgctctccggcttcgccatggtggcaatggtggaggtgcagctggacgctgaccacgactacccaccggggctgctcatcgccttcagtgcctgcaccacagtgctggtggctgtgcacctgtttgcgctcatgatcagcacctgcatcctgcccaacatcgaggcggtgagcaacgtgcacaatctcaactcggtcaaggagtccccccatgagcgcatgcaccgccacatcgagctggcctgggccttctccaccgtcatcggcacgctgctcttcctagctgaggtggtgctgctctgctgggtcaagttcttgcccctcaagaagcaaccgggacagccaaggcccaccagcaagccccctgcatcaggcgccgccgccaaccacagcgacagcagcggcatcaccccgggccaggcagctgccatcgcttcgaccaccatcatggtgcccttcggcctgatctttatcgtcttcgccgtccacttctaccgctcactggttagccataagaccgaccgacagttccaggagctcaacgagctggcggagtttgcccgcttacaggaccagctggaccacagaggggaccaccccctgacgcccggcagccactatgcctag

SEQ ID NO: 210 - protein, 302 aa, Artificial Sequence
  Synthetic Construct
MHPEPAPPPSRSSPELPPSGGSTTSGSRRSRRRSGDGEPPGAPPPPPSAVTYPDWIGQSYSEVMSLNEHSMQALSWRKLYLSRAKLKASSRTSALLSGFAMVAMVEVQLDADHDYPPGLLIAFSACTTVLVAVHLFALMISTCILPNIEAVSNVHNLNSVKESPHERMHRHIELAWAFSTVIGTLLFLAEVVLLCWVKFLPLKKQPGQPRPTSKPPASGAAANHSDSSGITPGQAAAIASTTIMVPFGLIFIVFAVHFYRSLVSHKTDRQFQELNELAEFARLQDQLDHRGDHPLTPGSHYA

SEQ ID NO: 211 - DNA, 23 bp, Artificial Sequence
  Forward primer sequence
ggccagccaagccccaccaagcc

SEQ ID NO: 212 - DNA, 23 bp, Artificial Sequence
  Reverse primer sequence
ggcttggtggggcttggctggcc

SEQ ID NO: 213 - DNA, 912 bp, Artificial Sequence
  mORAI1/hORAI1 ECL2 (SPTKPPAE) sequence
atgcatccggagcctgccccgcccccgagtcacagcaatccggagcttcccgtgagcggcggcagcagcactagcggcagccgccggagccgccgccgcagcggggacggggagccctcgggggccccaccgctgccgccgccgccacccgccgtcagctacccggactggatcggccagagttactccgaggtgatgagcctcaacgagcactcgatgcaggcgctgtcctggcgcaagctctacttaagccgcgccaagctcaaagcttccagccggacctcggctctgctctccggcttcgccatggtagcgatggtggaagtccagctggacacagaccatgactacccaccagggttgctcatcgtctttagtgcctgcaccacagtgctagtggccgtgcacctgtttgccctcatgatcagcacctgcatcctgcccaacatcgaggctgtgagcaacgtccacaacctcaactcggtcaaagagtcaccccacgagcgcatgcatcgccacatcgagctggcctgggccttctccacggtcatcgggacgctgcttttcctagcagaggtcgtgctgctctgctgggtcaagttcttacctctcaagaagcagccaggccagccaagccccaccaagccgcccgccgagagtggcgcagcagccaacgtcagcaccagcggcatcaccccgggtgaggcggcagccattgcctccaccgccatcatggttccctgtggcctggttttcatcgtctttgctgttcacttctaccgctccctggtcagccataagacggaccggcagttccaggagctcaatgagctggccgagtttgcccgcttgcaggaccagctggaccacagaggggaccattctctaacaccgggcacccactatgcctaa

SEQ ID NO: 214 - protein, 303 aa, Artificial Sequence
  Synthetic Construct
MHPEPAPPPSHSNPELPVSGGSSTSGSRRSRRRSGDGEPSGAPPLPPPPPAVSYPDWIGQSYSEVMSLNEHSMQALSWRKLYLSRAKLKASSRTSALLSGFAMVAMVEVQLDTDHDYPPGLLIVFSACTTVLVAVHLFALMISTCILPNIEAVSNVHNLNSVKESPHERMHRHIELAWAFSTVIGTLLFLAEVVLLCWVKFLPLKKQPGQPSPTKPPAESGAAANVSTSGITPGEAAAIASTAIMVPCGLVFIVFAVHFYRSLVSHKTDRQFQELNELAEFARLQDQLDHRGDHSLTPGTHYA

SEQ ID NO: 215 - DNA, 39 bp, Artificial Sequence
  Forward primer sequence
ccgcccgccgagagtgtcatagtagccaacgtcagcacc

SEQ ID NO: 216 - DNA, 39 bp, Artificial Sequence
  Reverse primer sequence
ggtgctgacgttggctactatgacactctcggcgggcgg

SEQ ID NO: 217 - DNA, 912 bp, Artificial Sequence
  mORAI1/hORAI1 ECL2 (SPTKPPAESVIV) sequence
atgcatccggagcctgccccgcccccgagtcacagcaatccggagcttcccgtgagcggcggcagcagcactagcggcagccgccggagccgccgccgcagcggggacggggagccctcgggggccccaccgctgccgccgccgccacccgccgtcagctacccggactggatcggccagagttactccgaggtgatgagcctcaacgagcactcgatgcaggcgctgtcctggcgcaagctctacttaagccgcgccaagctcaaagcttccagccggacctcggctctgctctccggcttcgccatggtagcgatggtggaagtccagctggacacagaccatgactacccaccagggttgctcatcgtctttagtgcctgcaccacagtgctagtggccgtgcacctgtttgccctcatgatcagcacctgcatcctgcccaacatcgaggctgtgagcaacgtccacaacctcaactcggtcaaagagtcaccccacgagcgcatgcatcgccacatcgagctggcctgggccttctccacggtcatcgggacgctgcttttcctagcagaggtcgtgctgctctgctgggtcaagttcttacctctcaagaagcagccaggccagccaagccccaccaagccgcccgccgagagtgtcatagtagccaacgtcagcaccagcggcatcaccccgggtgaggcggcagccattgcctccaccgccatcatggttccctgtggcctggttttcatcgtctttgctgttcacttctaccgctccctggtcagccataagacggaccggcagttccaggagctcaatgagctggccgagtttgcccgcttgcaggaccagctggaccacagaggggaccattctctaacaccgggcacccactatgcctaa

SEQ ID NO: 218 - protein, 303 aa, Artificial Sequence
  Synthetic Construct
MHPEPAPPPSHSNPELPVSGGSSTSGSRRSRRRSGDGEPSGAPPLPPPPPAVSYPDWIGQSYSEVMSLNEHSMQALSWRKLYLSRAKLKASSRTSALLSGFAMVAMVEVQLDTDHDYPPGLLIVFSACTTVLVAVHLFALMISTCILPNIEAVSNVHNLNSVKESPHERMHRHIELAWAFSTVIGTLLFLAEVVLLCWVKFLPLKKQPGQPSPTKPPAESVIVANVSTSGITPGEAAAIASTAIMVPCGLVFIVFAVHFYRSLVSHKTDRQFQELNELAEFARLQDQLDHRGDHSLTPGTHYA

SEQ ID NO: 219 - DNA, 42 bp, Artificial Sequence
  Forward primer sequence
gtgtcatagtagccaaccacagcgacagcggcatcaccccgg

SEQ ID NO: 220 - DNA, 42 bp, Artificial Sequence
  Reverse primer sequence
ccggggtgatgccgctgtcgctgtggttggctactatgacac

SEQ ID NO: 221 - DNA, 912 bp, Artificial Sequence
  mORAI1/hORAI1 ECL2 (AESVIVANHSD) sequence
atgcatccggagcctgccccgcccccgagtcacagcaatccggagcttcccgtgagcggcggcagcagcactagcggcagccgccggagccgccgccgcagcggggacggggagccctcgggggccccaccgctgccgccgccgccacccgccgtcagctacccggactggatcggccagagttactccgaggtgatgagcctcaacgagcactcgatgcaggcgctgtcctggcgcaagctctacttaagccgcgccaagctcaaagcttccagccggacctcggctctgctctccggcttcgccatggtagcgatggtggaagtccagctggacacagaccatgactacccaccagggttgctcatcgtctttagtgcctgcaccacagtgctagtggccgtgcacctgtttgccctcatgatcagcacctgcatcctgcccaacatcgaggctgtgagcaacgtccacaacctcaactcggtcaaagagtcaccccacgagcgcatgcatcgccacatcgagctggcctgggccttctccacggtcatcgggacgctgcttttcctagcagaggtcgtgctgctctgctgggtcaagttcttacctctcaagaagcagccaggccagccaaggcccaccagcaagcccgccgagagtgtcatagtagccaaccacagcgacagcggcatcaccccgggtgaggcggcagccattgcctccaccgccatcatggttccctgtggcctggttttcatcgtctttgctgttcacttctaccgctccctggtcagccataagacggaccggcagttccaggagctcaatgagctggccgagtttgcccgcttgcaggaccagctggaccacagaggggaccattctctaacaccgggcacccactatgcctaa

SEQ ID NO: 222 - protein, 303 aa, Artificial Sequence
  Synthetic Construct
MHPEPAPPPSHSNPELPVSGGSSTSGSRRSRRRSGDGEPSGAPPLPPPPPAVSYPDWIGQSYSEVMSLNEHSMQALSWRKLYLSRAKLKASSRTSALLSGFAMVAMVEVQLDTDHDYPPGLLIVFSACTTVLVAVHLFALMISTCILPNIEAVSNVHNLNSVKESPHERMHRHIELAWAFSTVIGTLLFLAEVVLLCWVKFLPLKKQPGQPRPTSKPAESVIVANHSDSGITPGEAAAIASTAIMVPCGLVFIVFAVHFYRSLVSHKTDRQFQELNELAEFARLQDQLDHRGDHSLTPGTHYA

SEQ ID NO: 223 - DNA, 34 bp, Artificial Sequence
  Forward primer sequence
cttacctctcaagaggcaggcaggccagccaagc

SEQ ID NO: 224 - DNA, 34 bp, Artificial Sequence
  Reverse primer sequence
gcttggctggcctgcctgcctcttgagaggtaag

SEQ ID NO: 225 - DNA, 912 bp, Artificial Sequence
  mORAI1/hORAI1 ECL2 (RQAGQPSPTKPPAE) sequence
atgcatccggagcctgccccgcccccgagtcacagcaatccggagcttcccgtgagcggcggcagcagcactagcggcagccgccggagccgccgccgcagcggggacggggagccctcgggggccccaccgctgccgccgccgccacccgccgtcagctacccggactggatcggccagagttactccgaggtgatgagcctcaacgagcactcgatgcaggcgctgtcctggcgcaagctctacttaagccgcgccaagctcaaagcttccagccggacctcggctctgctctccggcttcgccatggtagcgatggtggaagtccagctggacacagaccatgactacccaccagggttgctcatcgtctttagtgcctgcaccacagtgctagtggccgtgcacctgtttgccctcatgatcagcacctgcatcctgcccaacatcgaggctgtgagcaacgtccacaacctcaactcggtcaaagagtcaccccacgagcgcatgcatcgccacatcgagctggcctgggccttctccacggtcatcgggacgctgcttttcctagcagaggtcgtgctgctctgctgggtcaagttcttacctctcaagaggcaggcaggccagccaagccccaccaagccgcccgccgagagtggcgcagcagccaacgtcagcaccagcggcatcaccccgggtgaggcggcagccattgcctccaccgccatcatggttccctgtggcctggttttcatcgtctttgctgttcacttctaccgctccctggtcagccataagacggaccggcagttccaggagctcaatgagctggccgagtttgcccgcttgcaggaccagctggaccacagaggggaccattctctaacaccgggcacccactatgcctaa

SEQ ID NO: 226 - protein, 303 aa, Artificial Sequence
  Synthetic Construct
MHPEPAPPPSHSNPELPVSGGSSTSGSRRSRRRSGDGEPSGAPPLPPPPPAVSYPDWIGQSYSEVMSLNEHSMQALSWRKLYLSRAKLKASSRTSALLSGFAMVAMVEVQLDTDHDYPPGLLIVFSACTTVLVAVHLFALMISTCILPNIEAVSNVHNLNSVKESPHERMHRHIELAWAFSTVIGTLLFLAEVVLLCWVKFLPLKRQAGQPSPTKPPAESGAAANVSTSGITPGEAAAIASTAIMVPCGLVFIVFAVHFYRSLVSHKTDRQFQELNELAEFARLQDQLDHRGDHSLTPGTHYA

SEQ ID NO: 227 - DNA, 42 bp, Artificial Sequence; identical to SEQ ID NO: 219
  Forward primer sequence

SEQ ID NO: 228 - DNA, 42 bp, Artificial Sequence; identical to SEQ ID NO: 220
  Reverse primer sequence

SEQ ID NO: 229 - protein, 17 aa, Homo sapiens
  MISC_FEATURE: 210 to 226 of SEQ ID NO2
RPTSKPPASGAAANVST

SEQ ID NO: 230 - protein, 17 aa, Mus musculus
  MISC_FEATURE: 212 to 228 of SEQ ID NO72
SPTKPPAESVIVANHSD

SEQ ID NO: 231 - DNA, 912 bp, Artificial Sequence
  mORAI1/hORAI1 ECL2 (SPTKPPAESVIVANHSD) sequence
atgcatccggagcctgccccgcccccgagtcacagcaatccggagcttcccgtgagcggcggcagcagcactagcggcagccgccggagccgccgccgcagcggggacggggagccctcgggggccccaccgctgccgccgccgccacccgccgtcagctacccggactggatcggccagagttactccgaggtgatgagcctcaacgagcactcgatgcaggcgctgtcctggcgcaagctctacttaagccgcgccaagctcaaagcttccagccggacctcggctctgctctccggcttcgccatggtagcgatggtggaagtccagctggacacagaccatgactacccaccagggttgctcatcgtctttagtgcctgcaccacagtgctagtggccgtgcacctgtttgccctcatgatcagcacctgcatcctgcccaacatcgaggctgtgagcaacgtccacaacctcaactcggtcaaagagtcaccccacgagcgcatgcatcgccacatcgagctggcctgggccttctccacggtcatcgggacgctgcttttcctagcagaggtcgtgctgctctgctgggtcaagttcttacctctcaagaagcagccaggccagccaagccccaccaagccgcccgccgagagtgtcatagtagccaaccacagcgacagcggcatcaccccgggtgaggcggcagccattgcctccaccgccatcatggttccctgtggcctggttttcatcgtctttgctgttcacttctaccgctccctggtcagccataagacggaccggcagttccaggagctcaatgagctggccgagtttgcccgcttgcaggaccagctggaccacagaggggaccattctctaacaccgggcacccactatgcctaa

SEQ ID NO: 232 - protein, 303 aa, Artificial Sequence
  Synthetic Construct
MHPEPAPPPSHSNPELPVSGGSSTSGSRRSRRRSGDGEPSGAPPLPPPPPAVSYPDWIGQSYSEVMSLNEHSMQALSWRKLYLSRAKLKASSRTSALLSGFAMVAMVEVQLDTDHDYPPGLLIVFSACTTVLVAVHLFALMISTCILPNIEAVSNVHNLNSVKESPHERMHRHIELAWAFSTVIGTLLFLAEVVLLCWVKFLPLKKQPGQPSPTKPPAESVIVANHSDSGITPGEAAAIASTAIMVPCGLVFIVFAVHFYRSLVSHKTDRQFQELNELAEFARLQDQLDHRGDHSLTPGTHYA

SEQ ID NO: 233 - DNA, 66 bp, Artificial Sequence
  VK-1 signal peptide coding sequence
atggacatgagggtgcccgctcagctcctggggctcctgctgctgtggctgagaggtgcgcgctgt

SEQ ID NO: 234 - protein, 22 aa, Artificial Sequence
  Synthetic Construct
MDMRVPAQLLGLLLLWLRGARC

SEQ ID NO: 235 - protein, 9 aa, Artificial Sequence
  Integrin-binding peptide
CYGGRGDTP

SEQ ID NO: 236 - DNA, 72 bp, Artificial Sequence
  Forward primer sequence
ctgctcttcctagctgaggtggtgctgctctgctgggtcaagttcttgcccctcaagaggcaagcgggacag

SEQ ID NO: 237 - DNA, 67 bp, Artificial Sequence
  Forward primer sequence
ctcaagaggcaagcgggacagccaagccccaccaagcctcccgctgaatcagtcatcgtcgccaacc

SEQ ID NO: 238 - DNA, 68 bp, Artificial Sequence
  Forward primer sequence
gaatcagtcatcgtcgccaaccacagcgacagcagcggcatcaccccgggccaggcagctgccatcgc

SEQ ID NO: 239 - DNA, 66 bp, Artificial Sequence
  Reverse primer sequence
gagcggtagaagtgaacagcaaagacgataaaaaccaggccacagggaaccatgatggcggtggag

SEQ ID NO: 240 - DNA, 66 bp, Artificial Sequence
  Reverse primer sequence
ctcattgagctcctggaactgccggtccgtcttatggctgaccagggagcggtagaagtgaacagc

SEQ ID NO: 241 - DNA, 65 bp, Artificial Sequence
  Reverse primer sequence
ctctgtggtccagctggtcctgcaagcgggcaaactcggccagctcattgagctcctggaactgc

SEQ ID NO: 242 - DNA, 70 bp, Artificial Sequence
  Reverse primer sequence
cgcggccgcttaggcatagtgggtgcccggtgttagagaatggtcccctctgtggtccagctggtcctgc

SEQ ID NO: 243 - protein, 330 aa, Homo sapiens
  MISC_FEATURE: hIgG1z sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 244 - protein, 330 aa, Homo sapiens [sequence omitted]

SEQ ID NO: 245 - protein, 330 aa, Homo sapiens
  MISC_FEATURE: hIgG1f sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 246 - protein, 330 aa, Homo sapiens
  MISC_FEATURE: hIgG1fa sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 247 - protein, 106 aa, Homo sapiens
  MISC_FEATURE: CL-2 human LC constant domain
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 248 - protein, 106 aa, Homo sapiens
  MISC_FEATURE: CL-3 human LC constant domain
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 249 - protein, 106 aa, Homo sapiens
  MISC_FEATURE: CL-7 human LC constant domain
GQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 250 - protein, 120 aa, Artificial Sequence
  VH5
QVQLVQSGAEVKKPGASVKVPCKASGYTFTDYYINWVRQAPGQGLEWMGWINPNNGGTNYAQKFQGRVTMTRDTSISTAYMELRRLRSDDTAVYYCARERGGYEDWFDPWGQGTLVTVSS

SEQ ID NO: 251 - protein, 120 aa, Artificial Sequence
  VH6
QVQLVQSGAEVKKPGASVKVSCKASGYTFTDYYINWVRQAPGQGLEWMGWINPNSGGSSYAQKFQGRVTMTRDTSISTAHMELIRLRSDDTAVYYCARERGGIEDWFDPWGQGTLVTVSS

SEQ ID NO: 252 - protein, 120 aa, Artificial Sequence
  VH7
QVQLVQSGAEVKKPGASVKVSCKASGYTFTDYYINWVRQAPGQGLEWMGWINPNSGGTDYAQKFQGRVTMTRDTSIRTAYMELNRLTSDDTAVYYCAREYGGYEDWFDPWGQGTLVTVSS

SEQ ID NO: 253 - protein, 120 aa, Artificial Sequence
  VH8
QVQLVQSGAEVKKPGASVKVSCKASGYTFTDYYMNWVRQAPGQGLEWMGWINPNSGGTHYAQKFQGRVTMTRDTSISTAYMELSRLRSDDTAVYYCAREYGGYSDWFDPWGQGTLVTVSS

SEQ ID NO: 254 - protein, 120 aa, Artificial Sequence
  VH9
QVQLVQSGAEVKKPGASVKVSCKASGYTFTDYYMNWVRQAPGQGLEWMGWINPNSGGTKYAQKFQGRVTMTRDTSIRTAYMELSRLRSDDTAVYYCAREYGGNSDWFDPWGQGTLVTVSS

SEQ ID NO: 255 - protein, 120 aa, Artificial Sequence
  VH10
QVQLVQSGAEVKKPGASVKVSCKASGYTFTDYYMNWVRQAPGQGLEWMGWINPNSGGTKYAQKFQGRVTMTRDTSIRTAYMELSRLRSDDTAVYYCSREYGGNSDWFDPWGQGTLVTVSS

SEQ ID NO: 256 - protein, 109 aa, Artificial Sequence
  VL4
QSVLTQPPSVSGAPGQRVTISCTGSSSNIGAGFDVHWYQQLPVTAPKLLIYGNRNRPSGVPARFSGSKSGTSASLAITGLQAEDEAVYYCQSYDSSLTVFGGGTKLTVL

SEQ ID NO: 257 - protein, 111 aa, Artificial Sequence
  VL5
QSVLTQPPSVSGAPGQRVTISCTGSSSNIGAGYDVHWYQQLPGTAPKLLIYGNSNRPSGVPDRFSGSKSGTSASLAITKFQAEDEAVYYCQSYGSGLSGVVFGGGTKLTVL

SEQ ID NO: 258 - protein, 109 aa, Artificial Sequence
  VL6
SCELTQSPSVSVSPGQTARITCSGDALPKKYACCYQQKSGQAPVLVVYDDHKRPSGIPERFSGSSSGTLATLIISGAQVEDEADSYCYSTDSSGNHSWVFGGGTKLTVL

SEQ ID NO: 259 - protein, 109 aa, Artificial Sequence
  VL7
QSVLTQPPSVSGAPGQRVTISCTGSNSNIGAGFDVHWYQQLPGTVPKLLIYGNNNRPSGVPDRFSGSKSGTSASLAITGLQAEDEADYYCQSYDSRLTVFGGGTKLTVL

SEQ ID NO: 260 - protein, 112 aa, Artificial Sequence
  VL8
QSVLTQPPSVSGAPGQRVTISCTGSRSNIGAGYDVHWYQQLPRTAPKLLIYDNSNRPSGVPDRFSGSKSGSSASLAITGLQAEDEADYYCQSYDNSLSDSVLIGGGTKLTVL

SEQ ID NO: 261 - protein, 111 aa, Artificial Sequence
  VL9
QSALTQPPSASGSPGQSVTSSCTGTSSDVGGYNYVSWYQQHPGKAPKLMIYEVSKRPSGVPDWFSGSKSGNTASLTVSGLQAEDEADYYYNSYSGSNNFDVFGTGTKVTVL

SEQ ID NO: 262 - protein, 112 aa, Artificial Sequence
  VL10
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDNRLSDSVVIGGGTKLAVQ

SEQ ID NO: 263 - protein, 111 aa, Artificial Sequence
  VL11
QSALTQPPSASGSPGQSVTSSCTGTSSDVGGYNYVSWYQQQPGKAPKLMIYEVSKRPSGVPDRFSGSKSGNTASLTVSGLQAEDEADYYFSSYAGSNNFDVFGTGTKVTVL

SEQ ID NO: 264 - protein, 112 aa, Artificial Sequence
  VL12
QSVLTQPPSVSGAPGQRVTISCTGSSSNIGAGYDVHWYQQSPGTAPKLLIYGNSNRPSGVPDRFSGSKSGTSASLAITGLQAEDEADYYCQSYDNRLSDSVVIGGGTKLTVQ

SEQ ID NO: 265 - protein, 112 aa, Artificial Sequence
  VL13
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDNRLSDSVVIGGGTKLTVQ

SEQ ID NO: 266 - protein, 5 aa, Artificial Sequence
  CDRH1-4
DYYIN

SEQ ID NO: 267 - protein, 5 aa, Artificial Sequence
  CDRH1-5
DYYMN

SEQ ID NO: 268 - protein, 17 aa, Artificial Sequence
  CDRH2-5
WINPNNGGTNYAQKFQG

SEQ ID NO: 269 - protein, 17 aa, Artificial Sequence
  CDRH2-6
WINPNSGGSSYAQKFQG

SEQ ID NO: 270 - protein, 17 aa, Artificial Sequence
  CDRH2-7
WINPNSGGTDYAQKFQG

SEQ ID NO: 271 - protein, 17 aa, Artificial Sequence
  CDRH2-8
WINPNSGGTHYAQKFQG

SEQ ID NO: 272 - protein, 17 aa, Artificial Sequence
  CDRH2-9
WINPNSGGTKYAQKFQG

SEQ ID NO: 273 - protein, 11 aa, Artificial Sequence
  CDRH3-4
ERGGYEDWFDP

SEQ ID NO: 274 - protein, 11 aa, Artificial Sequence
  CDRH3-5
ERGGIEDWFDP

SEQ ID NO: 275 - protein, 11 aa, Artificial Sequence
  CDRH3-6
EYGGYEDWFDP

SEQ ID NO: 276 - protein, 11 aa, Artificial Sequence
  CDRH3-7
EYGGYSDWFDP

SEQ ID NO: 277 - protein, 11 aa, Artificial Sequence
  CDRH3-8
EYGGNSDWFDP

SEQ ID NO: 278 - protein, 14 aa, Artificial Sequence
  CDRL1-4
TGSSSNIGAGFDVH

SEQ ID NO: 279 - protein, 14 aa, Artificial Sequence
  CDRL1-5
TGSSSNIGAGYDVH

SEQ ID NO: 280 - protein, 11 aa, Artificial Sequence
  CDRL1-6
SGDALPKKYAC

SEQ ID NO: 281 - protein, 14 aa, Artificial Sequence
  CDRL1-7
TGSNSNIGAGFDVH

SEQ ID NO: 282 - protein, 14 aa, Artificial Sequence
  CDRL1-8
TGSRSNIGAGYDVH

SEQ ID NO: 283 - protein, 14 aa, Artificial Sequence
  CDRL1-9
TGTSSDVGGYNYVS

SEQ ID NO: 284 - protein, 7 aa, Artificial Sequence
  CDRL2-3
GNRNRPS

SEQ ID NO: 285 - protein, 7 aa, Artificial Sequence
  CDRL2-4
GNSNRPS

SEQ ID NO: 286 - protein, 7 aa, Artificial Sequence
  CDRL2-5
DDHKRPS

SEQ ID NO: 287 - protein, 7 aa, Artificial Sequence
  CDRL2-6
GNNNRPS

SEQ ID NO: 288 - protein, 7 aa, Artificial Sequence
  CDRL2-7
DNSNRPS

SEQ ID NO: 289 - protein, 7 aa, Artificial Sequence
  CDRL2-8
EVSKRPS

SEQ ID NO: 290 - protein, 9 aa, Artificial Sequence
  CDRL3-3
QSYDSSLTV

SEQ ID NO: 291 - protein, 11 aa, Artificial Sequence
  CDRL3-4
QSYGSGLSGVV

SEQ ID NO: 292 - protein, 12 aa, Artificial Sequence
  CDRL3-5
YSTDSSGNHSWV

SEQ ID NO: 293 - protein, 9 aa, Artificial Sequence
  CDRL3-6
QSYDSRLTV

SEQ ID NO: 294 - protein, 12 aa, Artificial Sequence
  CDRL3-7
QSYDNSLSDSVL

SEQ ID NO: 295 - protein, 11 aa, Artificial Sequence
  CDRL3-8
NSYSGSNNFDV

SEQ ID NO: 296 - protein, 12 aa, Artificial Sequence
  CDRL3-9
QSYDNRLSDSVV

SEQ ID NO: 297 - protein, 11 aa, Artificial Sequence
  CDRL3-10
SSYAGSNNFDV

SEQ ID NO: 298 - protein, 5 aa, Artificial Sequence
  CDRH1
XYXXX

SEQ ID NO: 299 - protein, 17 aa, Artificial Sequence
  CDRH2
XIXXXXXXXXXXXXXXX

SEQ ID NO: 300 - protein, 13 aa, Artificial Sequence
  CDRH3
XXXGXXXXXXXXX

SEQ ID NO: 301 - protein, 14 aa, Artificial Sequence
  CDRL1
XGXXXXXXXXXXXX

SEQ ID NO: 302 - protein, 7 aa, Artificial Sequence
  CDRL2
XXXXRPS

SEQ ID NO: 303 - protein, 12 aa, Artificial Sequence
  CDRL3
XSXXXXXXXXXX

SEQ ID NO: 304 - DNA, 63 bp, Artificial Sequence
  Primer Sequence
ggtcgactgaaccaccatgcatcatcatcaccaccaccatccggagcccgccccgcccccgag

SEQ ID NO: 305 - DNA, 921 bp, Macaca fascicularis
  misc_feature: Cyno Orai1 Encoding
atgcatccggagcccgccccgcccccgagccgcagcagccccgagcttcccccgagcggcggcagcaccaccagcggtagccgccggagccgccgccgcagcggggacggggagcctccgggagccccgccgccgccgccgccgccgccgccgccgcccgccgtcacctacccggactggatcggccagagttactccgaggtgatgagtctcaacgagcactccatgcaggcgctgtcctggcgcaagctctatttgagccgcgccaagctcaaagcctccagccggacctcggctctgctctccggcttcgccatggtggcaatggtggaggtgcagctggacgctgaccacgactacccgccagggctgctcatcgccttcagtgcctgcaccacggtgctggtggctgtgcacctgtttgcactcatgatcagcacctgcatcctgcccaacatcgaggcggtgagcaacgtgcacaacctcaactcggtcaaggagtccccccacgagcgcatgcaccgccacatcgagctggcctgggccttctccaccgtcatcggcacgctgcttttcctggccgaggtcgtgctgctctgctgggtcaagttcttgcccctcaagaagcagccaggccagccgaggcccaccagcaagccccccgccagtggtgcagccgccaacgtcagcaccagcggcatcaccccgggccaggcagccgccatcgcctcgaccaccatcatggtgcccttcggcctgatctttattgtcttcgccgtccacttctaccgctcactggtcagccataagacggaccgacagttccaggagctcaacgagctggcggagtttgctcgcttacaggaccagctggaccacagaggggaccaccccctgacgcccggcagccactatgcctag

SEQ ID NO: 306 - protein, 306 aa, Macaca fascicularis
MHPEPAPPPSRSSPELPPSGGSTTSGSRRSRRRSGDGEPPGAPPPPPPPPPPPAVTYPDWIGQSYSEVMSLNEHSMQALSWRKLYLSRAKLKASSRTSALLSGFAMVAMVEVQLDADHDYPPGLLIAFSACTTVLVAVHLFALMISTCILPNIEAVSNVHNLNSVKESPHERMHRHIELAWAFSTVIGTLLFLAEVVLLCWVKFLPLKKQPGQPRPTSKPPASGAAANVSTSGITPGQAAAIASTTIMVPFGLIFIVFAVHFYRSLVSHKTDRQFQELNELAEFARLQDQLDHRGDHPLTPGSHYA

SEQ ID NO: 307 - DNA, 18 bp, Artificial Sequence
  Primer Sequence
gatgcatccggagcccgc

SEQ ID NO: 308 - DNA, 20 bp, Artificial Sequence
  Primer Sequence
gctcgttgagctcctggaac

SEQ ID NO: 309 - DNA, 46 bp, Artificial Sequence
  Primer Sequence
cctcaacgagcactccatgcaggcctcaacgagcactccatgcagg

SEQ ID NO: 310 - DNA, 24 bp, Artificial Sequence
  Primer Sequence
ctcttagaggacagtttcaaagtg

SEQ ID NO: 311 - DNA, 51 bp, Artificial Sequence
  Primer Sequence
gaagctttgaaccaccatgcatccggagcccgccccgcccccgagccgcag

SEQ ID NO: 312 - DNA, 26 bp, Artificial Sequence
  Primer Sequence
gcggccgcctaggcatagtggctgcc

SEQ ID NO: 313 - protein, 36 aa, Homo sapiens
  MISC_FEATURE: Human Orai1 variant (N223S) (Residues 198-233 of SEQ ID NO317)
KFLPLKKQPGQPRPTSKPPASGAAASVSTSGITPGQ

SEQ ID NO: 314 - DNA, 25 bp, Artificial Sequence
  Primer Sequence
ggcgcagcagccagcgtcagcacca

SEQ ID NO: 315 - DNA, 25 bp, Artificial Sequence
  Primer Sequence
tggtgctgacgctggctgctgcgcc

SEQ ID NO: 316 - DNA, 906 bp, Homo sapiens
  misc_feature: Human Orai1 variant (N223S) coding sequence
atgcatccggagcccgccccgcccccgagccgcagcagtcccgagcttcccccaagcggcggcagcaccaccagcggcagccgccggagccgccgccgcagcggggacggggagcccccgggggccccgccaccgccgccgtccgccgtcacctacccggactggatcggccagagttactccgaggtgatgagcctcaacgagcactccatgcaggcgctgtcctggcgcaagctctacttgagccgcgccaagcttaaagcctccagccggacctcggctctgctctccggcttcgccatggtggcaatggtggaggtgcagctggacgctgaccacgactacccaccggggctgctcatcgccttcagtgcctgcaccacagtgctggtggctgtgcacctgtttgcgctcatgatcagcacctgcatcctgcccaacatcgaggcggtgagcaacgtgcacaatctcaactcggtcaaggagtccccccatgagcgcatgcaccgccacatcgagctggcctgggccttctccaccgtcatcggcacgctgctcttcctagctgaggtggtgctgctctgctgggtcaagttcttgcccctcaagaagcagccaggccagccaaggcccaccagcaagccccccgccagtggcgcagcagccagcgtcagcaccagcggcatcaccccgggccaggcagctgccatcgcctcgaccaccatcatggtgcccttcggcctgatctttatcgtcttcgccgtccacttctaccgctcactggttagccataagaccgaccgacagttccaggagctcaacgagctggcggagtttgcccgcttacaggaccagctggaccacagaggggaccaccccctgacgcccggcagccactatgcctag

SEQ ID NO: 317 - protein, 301 aa, Homo sapiens [sequence omitted]

SEQ ID NO: 318 - DNA, 972 bp, Artificial Sequence
  6x-His-human Orai1 coding sequence
atgaaacatcatcaccatcaccatcacatgcatccggagcccgccccgcccccgagccgcagcagtcccgagcttcccccaagcggcggcagcaccaccagcggcagccgccggagccgccgccgcagcggggacggggagcccccgggggccccgccaccgccgccgtccgccgtcacctacccggactggatcggccagagttactccgaggtgatgagcctcaacgagcactccatgcaggcgctgtcctggcgcaagctctacttgagccgcgccaagcttaaagcctccagccggacctcggctctgctctccggcttcgccatggtggcaatggtggaggtgcagctggacgctgaccacgactacccaccggggctgctcatcgccttcagtgcctgcaccacagtgctggtggctgtgcacctgtttgcgctcatgatcagcacctgcatcctgcccaacatcgaggcggtgagcaacgtgcacaatctcaactcggtcaaggagtccccccatgagcgcatgcaccgccacatcgagctggcctgggccttctccaccgtcatcggcacgctgctcttcctagctgaggtggtgctgctctgctgggtcaagttcttgcccctcaagaagcagccaggccagccaaggcccaccagcaagccccccgccagtggcgcagcagccaacgtcagcaccagcggcatcaccccgggccaggcagctgccatcgcctcgaccaccatcatggtgcccttcggcctgatctttatcgtcttcgccgtccacttctaccgctcactggttagccataagaccgaccgacagttccaggagctcaacgagctggcggagtttgcccgcttacaggaccagctggaccacagaggggaccaccccctgacgcccggcagccactatgcctagtaactcgaggatccgcggaaagaagaagaagaagaagaa

SEQ ID NO: 319 - protein, 310 aa, Artificial Sequence
  Synthetic Construct
MKHHHHHHHMHPEPAPPPSRSSPELPPSGGSTTSGSRRSRRRSGDGEPPGAPPPPPSAVTYPDWIGQSYSEVMSLNEHSMQALSWRKLYLSRAKLKASSRTSALLSGFAMVAMVEVQLDADHDYPPGLLIAFSACTTVLVAVHLFALMISTCILPNIEAVSNVHNLNSVKESPHERMHRHIELAWAFSTVIGTLLFLAEVVLLCWVKFLPLKKQPGQPRPTSKPPASGAAANVSTSGITPGQAAAIASTTIMVPFGLIFIVFAVHFYRSLVSHKTDRQFQELNELAEFARLQDQLDHRGDHPLTPGSHYA

SEQ ID NO: 320 - DNA, 35 bp, Artificial Sequence
  Primer Sequence
ctgacgcccggcagccactatgcctaggcggccgc

SEQ ID NO: 321 - protein, 6 aa, Artificial Sequence
  6x-His
HHHHHH

SEQ ID NO: 322 - protein, 217 aa, Artificial Sequence
  L4 amino acid seqence (minus signal sequence)
QSVLTQPPSVSGAPGQRVTISCTGSSSNIGAGYNVHWYQQFPRTDPKLLIYVYNIRPSGVPDRFSGSRSGTSASLAITGLQTEDEADYYCQSYDSSLSGVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 323 - protein, 217 aa, Artificial Sequence
  L5 amino acid sequence (minus signal sequence)
QSVLTQPPSVSGAPGQRVTISCTGSSSNIGTGYNVHWYQQFPRTDPKLLIYVYNIRPSGVPDRFSGSRSGTSASLAITGLQTEDEADYYCQSYDSSLSGVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 324 - protein, 217 aa, Artificial Sequence
  L6 amino acid sequence (minus signal sequence)
QSVLTQPPSVSGAPGQRVTISCTGSNSNIGTGYDVHWYQKLPGTAPRLLIYSHFNRPSGVPDRFSGSTSGTSASLAITGLQAEDEADYYCQSYDSSLSGSVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 325 - protein, 446 aa, Artificial Sequence
  H5 amino acid sequence (minus signal sequence)
QVQLQQWGAGLLKPSETLSLTCAVYGGSFSGYYWSWIRQPPGKGLEWIGEIDHSGRINYNPALKSRLTISVDTSKNQFSLKLSSVTAADTAVYYCARAGSGGYEDWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 326 - protein, 446 aa, Artificial Sequence
  H6 amino acid sequence (minus signal sequence)
QVQLQQWGAGLLKPSETLSLTCAVYGGSFSGYYWSWIRQPPGKGLEWIGEIDHSGSTNYNPALKSRLTISVDTSKNQFSLKLSSVTAADTAVYYCARAGSGGYEDWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 327 - protein, 446 aa, Artificial Sequence
  H7 amino acid sequence (minus signal sequence)
EVQLVESGGGLVQPGGSLRLSCAASGFTFSSYWMSWVRQAPGKGLEWVANIKHDGSEKYYVDSVKGRFTISRDNAKNSLHLQMISLRAEDTAVYYCAKRYSGGWTFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 328 - protein, 444 aa, Artificial Sequence
  H8 amino acid sequence (minus signal sequence)
QVQLVQSGAEVKKPGASVKVSCKASGYTFTDYYMHWVRQAPGQGLEWMGWINPNSGGTNYVQKFQDRVTMTRDTSITTAYMELTRLRSDDTAVYYCAREEGDYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 329 - protein, 20 aa, Artificial Sequence
  Signal peptide sequence
MEAPAQLLFLLLLWLPDTTG

SEQ ID NO: 330 - protein, 19 aa, Artificial Sequence
  Signal peptide sequence
MEWTWRVLFLVAAATGAHS

SEQ ID NO: 331 - protein, 20 aa, Artificial Sequence
  Signal peptide sequence
METPAQLLFLLLLWLPDTTG

SEQ ID NO: 332 - protein, 19 aa, Artificial Sequence
  Signal peptide sequence
MKHLWFFLLLVAAPRWVLS

SEQ ID NO: 333 - protein, 215 aa, Artificial Sequence
  L7 amino acid sequence
QSVLTQPPSVSGAPGQRVTISCTGSSSNIGAGFDVHWYQQLPVTAPKLLIYGNRNRPSGVPARFSGSKSGTSASLAITGLQAEDEAVYYCQSYDSSLTVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 334 - protein, 215 aa, Artificial Sequence
  L8 amino acid sequence
SCELTQSPSVSVSPGQTARITCSGDALPKKYACCYQQKSGQAPVLVVYDDHKRPSGIPERFSGSSSGTLATLIISGAQVEDEADSYCYSTDSSGNHSWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 335 - protein, 218 aa, Artificial Sequence
  L9 amino acid sequence
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDNRLSDSVVIGGGTKLAVQGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 336 - protein, 217 aa, Artificial Sequence
  L10 amino acid sequence
QSALTQPPSASGSPGQSVTSSCTGTSSDVGGYNYVSWYQQQPGKAPKLMIYEVSKRPSGVPDRFSGSKSGNTASLTVSGLQAEDEADYYFSSYAGSNNFDVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 337 - protein, 218 aa, Artificial Sequence
  L11 amino acid sequence
QSVLTQPPSVSGAPGQRVTISCTGSSSNIGAGYDVHWYQQSPGTAPKLLIYGNSNRPSGVPDRFSGSKSGTSASLAITGLQAEDEADYYCQSYDNRLSDSVVIGGGTKLTVQGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 338 - protein, 218 aa, Artificial Sequence
  L12 amino acid sequence
QSVLTQPPSVSGAPGQRVTISCTGSRSNIGAGYDVHWYQQLPRTAPKLLIYDNSNRPSGVPDRFSGSKSGSSASLAITGLQAEDEADYYCQSYDNSLSDSVLIGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 339 - protein, 217 aa, Artificial Sequence
  L13 amino acid sequence
QSALTQPPSASGSPGQSVTSSCTGTSSDVGGYNYVSWYQQHPGKAPKLMIYEVSKRPSGVPDWFSGSKSGNTASLTVSGLQAEDEADYYYNSYSGSNNFDVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 340 - protein, 218 aa, Artificial Sequence
  L14 amino acid sequence
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDNRLSDSVVIGGGTKLTVQGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 341 - protein, 217 aa, Artificial Sequence
  L15 amino acid sequence
QSVLTQPPSVSGAPGQRVTISCTGSSSNIGAGYDVHWYQQLPGTAPKLLIYGNSNRPSGVPDRFSGSKSGTSASLAITKFQAEDEAVYYCQSYGSGLSGVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 342 - protein, 215 aa, Artificial Sequence
  L16 amino acid sequence
QSVLTQPPSVSGAPGQRVTISCTGSNSNIGAGFDVHWYQQLPGTVPKLLIYGNNNRPSGVPDRFSGSKSGTSASLAITGLQAEDEADYYCQSYDSRLTVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 343 - protein, 446 aa, Artificial Sequence
  H9 amino acid sequence
QVQLVQSGAEVKKPGASVKVPCKASGYTFTDYYINWVRQAPGQGLEWMGWINPNNGGTNYAQKFQGRVTMTRDTSISTAYMELRRLRSDDTAVYYCARERGGYEDWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 344 - protein, 446 aa, Artificial Sequence
  H10 amino acid sequence
QVQLVQSGAEVKKPGASVKVSCKASGYTFTDYYMNWVRQAPGQGLEWMGWINPNSGGTKYAQKFQGRVTMTRDTSIRTAYMELSRLRSDDTAVYYCAREYGGNSDWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 345 - protein, 446 aa, Artificial Sequence
  H11 amino acid sequence
QVQLVQSGAEVKKPGASVKVSCKASGYTFTDYYMNWVRQAPGQGLEWMGWINPNSGGTHYAQKFQGRVTMTRDTSISTAYMELSRLRSDDTAVYYCAREYGGYSDWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 346 - protein, 446 aa, Artificial Sequence
  H12 amino acid sequence
QVQLVQSGAEVKKPGASVKVSCKASGYTFTDYYMNWVRQAPGQGLEWMGWINPNSGGTKYAQKFQGRVTMTRDTSIRTAYMELSRLRSDDTAVYYCSREYGGNSDWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 347 - protein, 446 aa, Artificial Sequence
  H13 amino acid sequence
QVQLVQSGAEVKKPGASVKVSCKASGYTFTDYYINWVRQAPGQGLEWMGWINPNSGGSSYAQKFQGRVTMTRDTSISTAHMELIRLRSDDTAVYYCARERGGIEDWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 348 - protein, 446 aa, Artificial Sequence
  H14 amino acid sequence
QVQLVQSGAEVKKPGASVKVSCKASGYTFTDYYINWVRQAPGQGLEWMGWINPNSGGTDYAQKFQGRVTMTRDTSIRTAYMELNRLTSDDTAVYYCAREYGGYEDWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK